SEQUENCE LISTING (27 sequences)

SEQ ID NO: 1 - RNA, 4443 nt, Artificial Sequence
  Chemically Synthesized Oligonucleotide
augcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaa

SEQ ID NO: 2 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 3 - RNA, 140 nt, Artificial Sequence
  Chemically synthesized oligonucleotides
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 4 - RNA, 105 nt, Artificial Sequence
  Chemically synthesized oligonucleotide
cggguggcaucccugugaccccuccccagugccucuccuggcccuggaaguugccacuccagugcccaccagccuuguccuaauaaaauuaaguugcaucaagcu

SEQ ID NO: 5 - RNA, 105 nt, Artificial Sequence
  Chemically synthesized oligonucleotide
ggguggcaucccugugaccccuccccagugccucuccuggcccuggaaguugccacuccagugcccaccagccuuguccuaauaaaauuaaguugcaucaaagcu

SEQ ID NO: 6 - RNA, 4688 nt, Artificial Sequence
  Chemically synthesized oligonucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacgaugcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaacggguggcaucccugugaccccuccccagugccucuccuggcccuggaaguugccacuccagugcccaccagccuuguccuaauaaaauuaaguugcaucaagcu

SEQ ID NO: 7 - RNA, 4688 nt, Artificial Sequence
  Chemically synthesized oligonucleotide
ggacagaucgccuggagacgccauccacgcuguuuugaccuccauagaagacaccgggaccgauccagccuccgcggccgggaacggugcauuggaacgcggauuccccgugccaagagugacucaccguccuugacacgaugcaacgcucuccucuugaaaaggccucggugguguccaagcucuucuucucguggacuagacccauccugagaaagggguacagacagcgcuuggagcuguccgauaucuaucaaaucccuuccguggacuccgcggacaaccuguccgagaagcucgagagagaaugggacagagaacucgccucaaagaagaacccgaagcugauuaaugcgcuuaggcggugcuuuuucuggcgguucauguucuacggcaucuuccucuaccugggagaggucaccaaggccgugcagccccuguugcugggacggauuauugccuccuacgaccccgacaacaaggaagaaagaagcaucgcuaucuacuugggcaucggucugugccugcuuuucaucguccggacccucuuguugcauccugcuauuuucggccugcaucacauuggcaugcagaugagaauugccauguuuucccugaucuacaagaaaacucugaagcucucgagccgcgugcuugacaagauuuccaucggccagcucgugucccugcucuccaacaaucugaacaaguucgacgagggccucgcccuggcccacuucguguggaucgccccucugcaaguggcgcuucugaugggccugaucugggagcugcugcaagccucggcauucugugggcuuggauuccugaucgugcuggcacuguuccaggccggacuggggcggaugaugaugaaguacagggaccagagagccggaaagauuuccgaacggcuggugaucacuucggaaaugaucgaaaacauccagucagugaaggccuacugcugggaagaggccauggaaaagaugauugaaaaccuccggcaaaccgagcugaagcugacccgcaaggccgcuuacgugcgcuauuucaacucguccgcuuucuucuucuccggguucuucgugguguuucucuccgugcuccccuacgcccugauuaagggaaucauccucaggaagaucuucaccaccauuuccuucuguaucgugcuccgcauggccgugacccggcaguucccaugggccgugcagacuugguacgacucccugggagccauuaacaagauccaggacuuccuucaaaagcaggaguacaagacccucgaguacaaccugacuacuaccgaggucgugauggaaaacgucaccgccuuuugggaggagggauuuggcgaacuguucgagaaggccaagcagaacaacaacaaccgcaagaccucgaacggugacgacucccucuucuuuucaaacuucagccugcucgggacgcccgugcugaaggacauuaacuucaagaucgaaagaggacagcuccuggcgguggccggaucgaccggagccggaaagacuucccugcugauggugaucaugggagagcuugaaccuagcgagggaaagaucaagcacuccggccgcaucagcuucuguagccaguuuuccuggaucaugcccggaaccauuaaggaaaacaucaucuucggcguguccuacgaugaauaccgcuaccgguccgugaucaaagccugccagcuggaagaggauauuucaaaguucgcggagaaagauaacaucgugcugggcgaaggggguauuaccuugucggggggccagcgggcuagaaucucgcuggccagagccguguauaaggacgccgaccuguaucuccuggacucccccuucggauaccuggacguccugaccgaaaaggagaucuucgaaucgugcgugugcaagcugauggcuaacaagacucgcauccucgugaccuccaaaauggagcaccugaagaaggcagacaagauucugauucugcaugagggguccuccuacuuuuacggcaccuucucggaguugcagaacuugcagcccgacuucucaucgaagcugauggguugcgacagcuucgaccaguucuccgccgaaagaaggaacucgauccugacggaaaccuugcaccgcuucucuuuggaaggcgacgccccugugucauggaccgagacuaagaagcagagcuucaagcagaccggggaauucggcgaaaagaggaagaacagcaucuugaaccccauuaacuccauccgcaaguucucaaucgugcaaaagacgccacugcagaugaacggcauugaggaggacuccgacgaaccccuugagaggcgccugucccuggugccggacagcgagcagggagaagccauccugccucggauuuccgugaucuccacugguccgacgcuccaagcccggcggcggcaguccgugcugaaccugaugacccacagcgugaaccagggccaaaacauucaccgcaagacuaccgcauccacccggaaagugucccuggcaccucaagcgaaucuuaccgagcucgacaucuacucccggagacugucgcaggaaaccgggcucgaaauuuccgaagaaaucaacgaggaggaucugaaagagugcuucuucgacgauauggagucgauacccgccgugacgacuuggaacacuuaucugcgguacaucacugugcacaagucauugaucuucgugcugauuuggugccuggugauuuuccuggccgaggucgcggccucacugguggugcucuggcuguugggaaacacgccucugcaagacaagggaaacuccacgcacucgagaaacaacagcuaugccgugauuaucacuuccaccuccucuuauuacguguucuacaucuacgucggaguggcggauacccugcucgcgauggguuucuucagaggacugccgcugguccacaccuugaucaccgucagcaagauucuucaccacaagauguugcauagcgugcugcaggcccccauguccacccucaacacucugaaggccggaggcauucugaacagauucuccaaggacaucgcuauccuggacgaucuccugccgcuuaccaucuuugacuucauccagcugcugcugaucgugauuggagcaaucgcagugguggcggugcugcagccuuacauuuucguggccacugugccggucauuguggcguucaucaugcugcgggccuacuuccuccaaaccagccagcagcugaagcaacuggaauccgagggacgaucccccaucuucacucaccuugugacgucguugaagggacuguggacccuccgggcuuucggacggcagcccuacuucgaaacccucuuccacaaggcccugaaccuccacaccgccaauugguuccuguaccuguccacccugcggugguuccagaugcgcaucgagaugauuuucgucaucuucuucaucgcggucacauucaucagcauccugacuaccggagagggagagggacgggucggaauaauccugacccucgccaugaacauuaugagcacccugcagugggcagugaacagcucgaucgacguggacagccugaugcgaagcgucagccgcguguucaaguucaucgacaugccuacugagggaaaacccacuaaguccacuaagcccuacaaaaauggccagcugagcaaggucaugaucaucgaaaacucccacgugaagaaggacgauauuuggcccuccggaggucaaaugaccgugaaggaccugaccgcaaaguacaccgagggaggaaacgccauucucgaaaacaucagcuucuccauuucgccgggacagcgggucggccuucucgggcggaccgguuccgggaagucaacucugcugucggcuuuccuccggcugcugaauaccgagggggaaauccaaauugacggcgugucuugggauuccauuacucugcagcaguggcggaaggccuucggcgugaucccccagaagguguucaucuucucggguaccuuccggaagaaccuggauccuuacgagcaguggagcgaccaagaaaucuggaaggucgccgacgaggucggccugcgcuccgugauugaacaauuuccuggaaagcuggacuucgugcucgucgacgggggauguguccugucgcacggacauaagcagcucaugugccucgcacgguccgugcucuccaaggccaagauucugcugcuggacgaaccuucggcccaccuggauccggucaccuaccagaucaucaggaggacccugaagcaggccuuugccgauugcaccgugauucucugcgagcaccgcaucgaggccaugcuggagugccagcaguuccuggucaucgaggagaacaagguccgccaauacgacuccauucaaaagcuccucaacgagcggucgcuguucagacaagcuauuucaccguccgauagagugaagcucuucccgcaucggaacagcucaaagugcaaaucgaagccgcagaucgcagccuugaaggaagagacugaggaagaggugcaggacacccggcuuuaaggguggcaucccugugaccccuccccagugccucuccuggcccuggaaguugccacuccagugcccaccagccuuguccuaauaaaauuaaguugcaucaaagcu

SEQ ID NO: 8 - DNA, 4443 bp, Artificial Sequence
  Artificially synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 9 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 10 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcaccttgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 11 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 12 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 13 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgccaactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 14 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaagccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 15 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggcttgaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 16 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtcagatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatatcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtccgtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatccaattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtactcacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagagtctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 17 - DNA, 4443 bp, Artificial Sequence
  Chemically synthesized oligonucleotide
atgcagaggagcccactggagaaagcctccgtggtgagtaaactcttttttagttggaccagacccatcctgcgaaaaggatacaggcagcgcctcgagttgtctgatatctaccagattccttctgtggactcagctgacaatttgagtgagaagctggagcgggagtgggatagagagctggcgagcaaaaaaaaccccaagcttatcaatgctctgcgccgctgctttttctggaggttcatgttttatgggatcttcctgtacctgggggaggtcaccaaagctgttcagccgctccttcttggccgcatcatcgccagctatgaccctgataataaagaagaaaggtctattgctatttatctgggaattggcctctgcttgctcttcatcgtccgcacccttctgctgcaccctgccatttttggccttcaccacatcggcatgcaaatgagaattgccatgttctccctcatttacaaaaagaccctgaaactttcctcaagagtgttagataaaatatccattggtcagctggtcagcctgctgtccaacaatcttaacaaatttgatgaaggcttggcgctggcccacttcgtgtggattgcacctctgcaggtggccctgttgatgggacttatatgggagctgcttcaagcctctgctttctgtgggctgggctttttgattgtactggcactttttcaggctgggctcggaagaatgatgatgaaatacagagatcagcgggccgggaagatttcagagcgacttgtgatcaccagtgaaatgattgaaaatattcagagcgtgaaagcctactgctgggaagaagccatggagaagatgattgagaacctgaggcagacagagctcaagctcactcggaaggctgcttatgttcgctatttcaacagcagcgccttcttcttcagtggcttctttgttgtcttcctgtctgttctgccatatgcactgataaaaggcattattttacgaaagatcttcaccaccatcagtttttgcatcgttctcaggatggccgtcacaagacagttcccctgggctgtgcagacctggtacgattccttgggggccatcaacaagattcaagatttcttgcaaaaacaagaatataaaactttagaatacaacctcaccaccactgaagtggtcatggaaaatgtgacagccttttgggaggagggttttggagaattgttcgagaaggcaaagcagaataacaacaacaggaagacgagcaatggggacgactctctcttcttcagcaacttttcactgctcgggacccctgtgttgaaagatataaacttcaagatcgagaggggccagctcttggctgtggcaggctccactggagctggtaaaacatctcttctcatggtgatcatgggggaactggagccttccgaaggaaaaatcaagcacagtgggagaatctcattctgcagccagttttcctggatcatgcccggcaccattaaggaaaacatcatatttggagtgtcctatgatgagtaccgctaccggtcagtcatcaaagcctgtcagttggaggaggacatctccaagtttgcagagaaagacaacattgtgcttggagaggggggtatcactctttctggaggacaaagagccaggatctctttggcccgggcagtctacaaggatgcagacctctacttgttggacagtcccttcggctacctcgacgtgctgactgaaaaagaaatttttgaaagctgtgtgtgcaaactgatggcaaacaagaccaggattcttgtcaccagcaagatggaacatctgaagaaagcggacaaaattctgattctgcatgaagggagctcctacttctatggaacatttagcgagcttcagaacctacagccagacttctcctccaaattaatgggctgtgactccttcgaccagttctctgcagaaagaagaaactctatactcacagagaccctccaccgcttctcccttgagggagatgccccagtttcttggacagaaaccaagaagcagtcctttaagcagactggcgagtttggtgaaaagaggaaaaattcaattctcaatcctattaacagtattcgcaagttcagcattgtccagaagacacccctccagatgaatggcatcgaagaagatagtgacgagccgctggagagacggctgagtctggtgccagattcagaacagggggaggccatcctgccccggatcagcgtcatttccacaggccccacattacaagcacggcgccggcagagtgttttaaatctcatgacccattcagtgaaccagggccaaaatatccacaggaagactacagcttctacccggaaagtgtctctggcccctcaggccaatctgaccgagctggacatctacagcaggaggctctcccaggaaacagggctggaaatatctgaagagattaatgaagaggatcttaaagagtgcttctttgatgacatggagagcatccccgcggtgaccacatggaacacctaccttagatatattactgtccacaagagcctcatatttgtcctcatctggtgcctggttattttcctcgctgaggtggcggccagtcttgttgtgctctggctgctgggcaacactcctctccaggacaagggcaatagtacacacagcagaaataattcttatgccgtcatcattacaagcacctccagctactacgtgttctacatctatgtgggcgtggctgacaccctcctggccatgggtttcttccggggcctgcctttggtgcacaccctcatcacagtgtcaaaaattctgcaccataaaatgcttcattctgtcctgcaggcacccatgagcactttgaacacattgaaggctggcggcatcctcaacagattttctaaagatattgctatcctggatgatctcctccccctgacaatctttgactttatccagcttctgctgatcgtgattggagccatagcagtggttgctgtcctgcagccctacatttttgtggccaccgtgcccgtgattgttgcctttattatgctcagagcttacttcctgcaaacttctcaacagctcaaacagctagaatctgagggccggagccccatttttacccacctggtgacttccctgaagggactgtggactctgagagcattcgggcgacagccttactttgagacactgttccacaaggccctgaacttgcacactgccaactggtttctttacctgagcacactccgctggttccagatgcggatagagatgatcttcgtcatcttttttatagctgtaaccttcatttctatccttacaacaggagaaggagagggcagggtgggaatcatcctcacgctggctatgaacataatgtccaccttgcagtgggccgtgaattccagtatagatgtggattctctaatgaggagtgtctcccgggtgtttaaattcattgatatgcctactgaggggaaacccaccaagtcaacaaaaccttataagaatggacagctgagcaaggtgatgataattgagaacagccacgtgaagaaggatgacatttggcccagcgggggccagatgactgtgaaggacctgacggccaagtacaccgaaggtggaaatgccattttggaaaacatcagcttctcaatctctcctgggcagagagttggattgctgggtcgcacgggcagcggcaaatcaaccctgctcagtgccttccttcggctcctgaatacagaaggcgaaatccaaattgacggggtgagctgggacagcatcaccctgcagcagtggagaaaagcatttggggtcattccacagaaagttttcatcttctctggcactttcagaaagaacctggacccctatgagcagtggagcgaccaggagatctggaaggttgcagatgaagttggcctgcggagtgtgatagaacaatttcctggcaagctggattttgtgctggtagatggaggctgcgtgctgtcccacggccacaaacagctgatgtgcctcgcccgctccgttctttcaaaggccaaaatcttgcttttggatgagcccagtgctcacctcgacccagtgacctatcagataatccgcaggaccttaaagcaagcttttgccgactgcaccgtcatactgtgtgagcaccggattgaagcaatgctggaatgccagcagtttctggtgatcgaggagaataaggtccggcagtacgacagcatccagaagttgttgaatgagcgcagccttttccgccaggccatctccccatctgacagagtcaagctgtttccacataggaactcctctaagtgcaagtccaagccccagatcgctgccctcaaggaggaaactgaggaagaggtgcaggatacccgcctgtga

SEQ ID NO: 18 - DNA, 4443 bp, Artificial Sequence
  Artificially synthesized oligonucleotide
atgcagagaagccccctggagaaggcctctgtggtgagcaagctgttcttcagctggaccagacccatcctgagaaagggctacagacagagactggagctgtctgacatctaccagatcccctctgtggactctgccgacaacctgtctgagaagctggagagagagtgggacagagagctggccagcaagaagaaccccaagctgatcaatgccctgagaagatgcttcttctggagattcatgttctatggcatcttcctgtacctgggagaggtgaccaaggccgtgcagcccctgctgctgggcaggatcattgccagctatgaccctgacaacaaggaggagagaagcattgccatctacctgggcattggcctgtgcctgctgttcattgtgagaaccctgctgctgcaccctgccatctttggcctgcaccacattggcatgcagatgagaattgccatgttcagcctgatctacaagaagaccctgaagctgagcagcagagtgctggacaagatcagcattggccagctggtgagcctgctgagcaacaacctgaacaagtttgatgagggcctggccctggcccactttgtgtggattgcccccctgcaggtggccctgctgatgggcctgatctgggagctgctgcaggcctctgccttctgtggcctgggcttcctgattgtgctggccctgttccaggccggcctgggcagaatgatgatgaagtacagagaccagagagccggcaagatctctgagagactggtgatcacctctgagatgattgagaacatccagtctgtgaaggcctactgctgggaggaggccatggagaagatgattgagaacctgagacagacagagctgaagctgaccaggaaggccgcctatgtgagatacttcaacagctctgccttcttcttctctggcttctttgtggtgttcctgtctgtgctgccctatgccctgatcaagggcatcatcctgaggaagatcttcaccaccatcagcttctgcattgtgctgaggatggccgtgaccaggcagttcccctgggccgtgcagacctggtatgacagcctgggggccatcaacaagatccaggacttcctgcagaagcaggagtacaagaccctggagtacaacctgaccaccacagaggtggtgatggagaatgtgacagccttctgggaggagggctttggagagctgtttgagaaggccaagcagaacaacaacaacagaaagaccagcaatggagatgacagcctgttcttcagcaacttcagcctgctgggcacccctgtgctgaaggacatcaacttcaagattgagaggggccagctgctggccgtggccggcagcacaggagccggcaagaccagcctgctgatggtgatcatgggagagctggagccctctgagggcaagatcaagcactctggcagaatcagcttctgcagccagttcagctggatcatgcctggcaccatcaaggagaacatcatctttggggtgagctatgatgagtacaggtacagatctgtgatcaaggcctgccagctggaggaggacatctccaagtttgccgagaaggacaacattgtgctgggggagggaggcatcaccctgtctgggggccagagagccagaatcagcctggccagagccgtgtacaaggatgccgacctgtacctgctggacagcccctttggctacctggatgtgctgacagagaaggagatctttgagagctgtgtgtgcaagctgatggccaacaagaccaggatcctggtgaccagcaagatggagcacctgaagaaggccgacaagatcctgatcctgcatgagggcagcagctacttctatggcaccttctctgagctgcagaacctgcagcctgacttcagcagcaagctgatgggctgtgacagctttgaccagttctctgctgagagaagaaacagcatcctgacagagaccctgcacaggttcagcctggagggggatgcccctgtgagctggacagagaccaagaagcagagcttcaagcagacaggagagtttggggagaagaggaagaacagcatcctgaaccccatcaacagcatcaggaagttcagcattgtgcagaagacccccctgcagatgaatggcattgaggaggactctgatgagcccctggagagaagactgagcctggtgccagactctgagcagggagaggccatcctgcccaggatctctgtgatcagcacaggccccaccctgcaggccagaagaagacagtctgtgctgaacctgatgacccactctgtgaaccagggccagaatatccacagaaagaccacagccagcaccagaaaggtgagcctggccccccaggccaacctgacagagctggacatctacagcagaaggctgagccaggagacaggcctggagatctctgaggagatcaatgaggaggacctgaaggagtgcttctttgatgacatggagagcatccctgccgtgaccacctggaacacctacctgagatacatcacagtgcacaagagcctgatctttgtgctgatctggtgcctggtgatcttcctggccgaggtggccgccagcctggtggtgctgtggctgctgggcaacacccccctgcaggacaagggcaacagcacccacagcagaaacaacagctatgctgtgatcatcaccagcaccagcagctactatgtgttctacatctatgtgggagtggctgacaccctgctggccatgggcttcttcagaggcctgcccctggtgcacaccctgatcacagtgagcaagatcctgcaccacaagatgctgcactctgtgctgcaggcccccatgagcaccctgaacaccctgaaggctggaggcatcctgaacagattcagcaaggacattgccatcctggatgacctgctgcccctgaccatctttgacttcatccagctgctgctgattgtgattggagccattgccgtggtggccgtgctgcagccctacatctttgtggccacagtgcctgtgattgtggccttcatcatgctgagggcctacttcctgcagaccagccagcagctgaagcagctggagtctgagggcagaagccccatcttcacccacctggtgaccagcctgaagggcctgtggaccctgagggcctttggcagacagccctactttgagaccctgttccacaaggccctgaacctgcacacagccaactggttcctgtacctgagcaccctgagatggttccagatgaggattgagatgatctttgtgatcttcttcattgccgtgaccttcatcagcatcctgaccacaggggagggcgagggcagagtgggcatcatcctgaccctggccatgaacatcatgagcaccctgcagtgggccgtgaacagcagcattgatgtggacagcctgatgagatctgtgagcagagtgttcaagttcattgacatgcccacagagggcaagcccaccaagagcaccaagccctacaagaatggccagctgagcaaggtgatgatcattgagaacagccatgtgaagaaggatgacatctggccctctggaggccagatgacagtgaaggacctgacagccaagtacacagaggggggcaatgccatcctggagaacatcagcttcagcatcagccctggccagagggtgggcctgctgggcagaacaggctctggcaagagcaccctgctgtctgccttcctgaggctgctgaacacagagggagagatccagattgatggggtgagctgggacagcatcaccctgcagcagtggaggaaggcctttggggtgatcccccagaaggtgttcatcttctctggcaccttcaggaagaacctggacccctatgagcagtggtctgaccaggagatctggaaggtggccgatgaggtgggcctgagatctgtgattgagcagttccctggcaagctggactttgtgctggtggatggaggctgtgtgctgagccatggccacaagcagctgatgtgcctggccagatctgtgctgagcaaggccaagatcctgctgctggatgagccctctgcccacctggaccctgtgacctaccagatcatcagaagaaccctgaagcaggcctttgccgactgcacagtgatcctgtgtgagcacagaattgaggccatgctggagtgccagcagttcctggtgattgaggagaacaaggtgaggcagtatgacagcatccagaagctgctgaatgagagaagcctgttcagacaggccatcagcccctctgacagagtgaagctgttcccccacaggaacagcagcaagtgcaagagcaagccccagattgccgccctgaaggaggagacagaggaggaggtgcaggacaccagactgtga

SEQ ID NO: 19 - DNA, 4443 bp, Artificial Sequence
  Chemically synthesized oligonucleotide
atgcagaggagccccctggagaaggccagcgtggtgagcaagctgttcttcagctggaccaggcccatcctgaggaagggctacaggcagaggctggagctgagcgacatctaccagatccccagcgtggacagcgccgacaacctgagcgagaagctggagagggagtgggacagggagctggccagcaagaagaaccccaagctgatcaacgccctgaggaggtgcttcttctggaggttcatgttctacggcatcttcctgtacctgggcgaggtgaccaaggccgtgcagcccctgctgctgggcaggatcatcgccagctacgaccccgacaacaaggaggagaggagcatcgccatctacctgggcatcggcctgtgcctgctgttcatcgtgaggaccctgctgctgcaccccgccatcttcggcctgcaccacatcggcatgcagatgaggatcgccatgttcagcctgatctacaagaagaccctgaagctgagcagcagggtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcaggatgatgatgaagtacagggaccagagggccggcaagatcagcgagaggctggtgatcaccagcgagatgatcgagaacatccagagcgtgaaggcctactgctgggaggaggccatggagaagatgatcgagaacctgaggcagaccgagctgaagctgaccaggaaggccgcctacgtgaggtacttcaacagcagcgccttcttcttcagcggcttcttcgtggtgttcctgagcgtgctgccctacgccctgatcaagggcatcatcctgaggaagatcttcaccaccatcagcttctgcatcgtgctgaggatggccgtgaccaggcagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacaggaagaccagcaacggcgacgacagcctgttcttcagcaacttcagcctgctgggcacccccgtgctgaaggacatcaacttcaagatcgagaggggccagctgctggccgtggccggcagcaccggcgccggcaagaccagcctgctgatggtgatcatgggcgagctggagcccagcgagggcaagatcaagcacagcggcaggatcagcttctgcagccagttcagctggatcatgcccggcaccatcaaggagaacatcatcttcggcgtgagctacgacgagtacaggtacaggagcgtgatcaaggcctgccagctggaggaggacatcagcaagttcgccgagaaggacaacatcgtgctgggcgagggcggcatcaccctgagcggcggccagagggccaggatcagcctggccagggccgtgtacaaggacgccgacctgtacctgctggacagccccttcggctacctggacgtgctgaccgagaaggagatcttcgagagctgcgtgtgcaagctgatggccaacaagaccaggatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagaggaggaacagcatcctgaccgagaccctgcacaggttcagcctggagggcgacgcccccgtgagctggaccgagaccaagaagcagagcttcaagcagaccggcgagttcggcgagaagaggaagaacagcatcctgaaccccatcaacagcatcaggaagttcagcatcgtgcagaagacccccctgcagatgaacggcatcgaggaggacagcgacgagcccctggagaggaggctgagcctggtgcccgacagcgagcagggcgaggccatcctgcccaggatcagcgtgatcagcaccggccccaccctgcaggccaggaggaggcagagcgtgctgaacctgatgacccacagcgtgaaccagggccagaacatccacaggaagaccaccgccagcaccaggaaggtgagcctggccccccaggccaacctgaccgagctggacatctacagcaggaggctgagccaggagaccggcctggagatcagcgaggagatcaacgaggaggacctgaaggagtgcttcttcgacgacatggagagcatccccgccgtgaccacctggaacacctacctgaggtacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcaggaacaacagctacgccgtgatcatcaccagcaccagcagctactacgtgttctacatctacgtgggcgtggccgacaccctgctggccatgggcttcttcaggggcctgcccctggtgcacaccctgatcaccgtgagcaagatcctgcaccacaagatgctgcacagcgtgctgcaggcccccatgagcaccctgaacaccctgaaggccggcggcatcctgaacaggttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagggcctacttcctgcagaccagccagcagctgaagcagctggagagcgagggcaggagccccatcttcacccacctggtgaccagcctgaagggcctgtggaccctgagggccttcggcaggcagccctacttcgagaccctgttccacaaggccctgaacctgcacaccgccaactggttcctgtacctgagcaccctgaggtggttccagatgaggatcgagatgatcttcgtgatcttcttcatcgccgtgaccttcatcagcatcctgaccaccggcgagggcgagggcagggtgggcatcatcctgaccctggccatgaacatcatgagcaccctgcagtgggccgtgaacagcagcatcgacgtggacagcctgatgaggagcgtgagcagggtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagggtgggcctgctgggcaggaccggcagcggcaagagcaccctgctgagcgccttcctgaggctgctgaacaccgagggcgagatccagatcgacggcgtgagctgggacagcatcaccctgcagcagtggaggaaggccttcggcgtgatcccccagaaggtgttcatcttcagcggcaccttcaggaagaacctggacccctacgagcagtggagcgaccaggagatctggaaggtggccgacgaggtgggcctgaggagcgtgatcgagcagttccccggcaagctggacttcgtgctggtggacggcggctgcgtgctgagccacggccacaagcagctgatgtgcctggccaggagcgtgctgagcaaggccaagatcctgctgctggacgagcccagcgcccacctggaccccgtgacctaccagatcatcaggaggaccctgaagcaggccttcgccgactgcaccgtgatcctgtgcgagcacaggatcgaggccatgctggagtgccagcagttcctggtgatcgaggagaacaaggtgaggcagtacgacagcatccagaagctgctgaacgagaggagcctgttcaggcaggccatcagccccagcgacagggtgaagctgttcccccacaggaacagcagcaagtgcaagagcaagccccagatcgccgccctgaaggaggagaccgaggaggaggtgcaggacaccaggctgtga

SEQ ID NO: 20 - DNA, 4443 bp, Artificial Sequence
  Chemically synthesized oligonucleotide
atgcagagatcccctctggagaaggcctcagtggtgtccaagcttttcttctcctggaccaggcccattttaagaaagggctacaggcagagacttgagctgtctgacatctatcagatcccttctgtggattctgctgacaatcttagtgaaaaattggaaagggagtgggacagagagctggcaagtaaaaagaaccccaagctgattaatgccctgaggcgctgctttttttggagattcatgttctatggcatattcctctaccttggagaagtaaccaaagctgtacagcctctcctccttggcagaatcattgcctcctatgatcctgataacaaggaggagagaagcatagccatctacctgggcattgggctgtgcctcttgtttattgtgaggacccttctcttgcaccctgccatctttggccttcatcacattggcatgcaaatgagaatagcaatgtttagtcttatttacaaaaaaacattaaaactctcttccagggtgttggacaagatcagtattggacaactggtcagcctgctgagcaacaacctgaacaagtttgatgaaggactggccctggcccactttgtctggattgccccccttcaggtggctcttttgatgggcctgatctgggaactcctgcaggcctctgccttctgtgggttaggcttcctgatagtgctagctctctttcaggcagggttgggtagaatgatgatgaagtacagagaccagagggctgggaagatatctgagaggctggtcattacttctgaaatgatagaaaacatccagtctgttaaagcttactgctgggaggaggctatggaaaagatgattgagaacttgaggcaaacagagctcaagctgactaggaaggcagcctatgtcaggtatttcaacagcagtgctttcttcttctcaggctttttcgtggtcttcttgagtgttctgccctatgccctcatcaaggggataattttgagaaagattttcaccactatttccttttgcattgtcctgaggatggctgtcaccaggcaattcccctgggctgtgcagacatggtatgactctctgggggccatcaacaaaatccaagatttcctgcagaagcaggagtacaagaccctggaatacaacctcaccaccacagaagttgtgatggagaatgtgactgcattctgggaggaaggatttggggagctgtttgagaaagcaaaacaaaacaataataacaggaaaaccagcaatggagatgactccctgttcttttccaacttctctttgttgggcacccctgtcctgaaagatataaactttaaaattgaaagagggcagctgttggcagttgctggctccacaggagctggaaaaacttcactactgatggtgatcatgggggagttagaaccctctgaagggaaaataaaacattctgggaggattagtttctgcagccagttcagctggatcatgcctgggaccattaaagaaaatattatatttggagtgagctatgatgaatatagatataggagtgtcatcaaagcctgtcagttggaggaagacatcagcaaatttgcagagaaagacaacattgttctgggtgaaggtggcatcaccctgtcaggagggcaaagggccaggatcagcttggccagagcagtctataaagatgctgatctgtacctcctggatagcccttttggctatctggatgttttgacagagaaggaaatttttgagtcctgtgtctgcaagttaatggcaaataaaacaaggatacttgtgacctcaaaaatggaacacctgaagaaggctgacaaaattctgatcctgcatgagggcagcagctacttttatggaacattttctgaactgcagaatttgcaaccagacttttcatcaaagctcatgggatgtgacagttttgatcagttttctgcagaaaggagaaactccattttgactgagaccctgcacaggttcagtctggagggggatgccccagtgagttggactgagacaaagaaacagagcttcaagcagactggagagtttggagaaaagaggaaaaactcaattctcaatcccatcaatagcatcaggaagttcagcatagttcagaagactcctttgcagatgaatgggattgaagaggactcagatgagcccctggaaaggagactctccttggtgccagattcagagcagggggaagccatactgccaaggatctctgtgatttctacagggcccaccctccaagcaagaaggagacagtcagttttaaacctgatgacccactctgtcaaccagggacagaacattcatagaaagacaacagcatctacaagaaaagtttcactggcccctcaagccaatttaactgaactagatatctacagcaggaggctcagccaagaaacaggcctggagatctcagaagaaataaatgaggaggatttgaaggaatgcttctttgatgatatggagagcatcccagctgtcacaacctggaacacctacctgagatacatcacagtgcacaaatccctcatctttgtacttatatggtgccttgtcatcttcttagctgaggtggctgcttccctggtggtgctgtggctgctgggaaacacacccctccaggataaagggaactctactcacagcaggaacaacagttatgctgtgatcatcaccagtacctcctcctactatgtgttctacatttatgttggagttgcagacacattgcttgccatgggtttttttagaggactccccctggtgcatactctcatcactgtttccaaaatccttcaccacaagatgctgcacagtgtactacaggctcccatgagcaccctcaacactcttaaagcaggaggaatcttgaacagatttagcaaggacattgcaattcttgatgacctgcttccactgaccatctttgacttcatccagcttctgctcattgtaattggtgccattgctgtggtagcagtgctccagccatatatttttgtggccactgtgcctgttattgtggccttcattatgttgagagcctacttcctgcagacctctcagcagctcaagcaacttgaaagtgagggcaggagccccatatttacacacttggtcacttccctcaaaggcctctggacactcagagcttttggaagacaaccttattttgaaactctcttccacaaggctctgaatctccacacagccaactggtttctgtatctttcaacactgcgctggttccagatgaggattgagatgatctttgttatcttcttcatagctgttaccttcatctctattctgacaactggtgagggggaagggagagtaggcatcatcctcacactagccatgaacataatgtctaccttacaatgggccgtgaacagctccatagatgtggacagcctcatgagaagtgtgtcaagagttttcaaattcattgacatgcccacagaaggcaaaccaaccaagagcacaaaaccctacaagaatggccagctgagtaaggtcatgatcattgaaaattctcatgtgaagaaggatgatatttggcccagtgggggccagatgacagtcaaggacctcactgccaaatacacagagggtggaaatgctatcctagagaacatctccttctccatctccccaggccaaagagttggcttgctgggcaggactggcagtggcaagtccaccttgctctcagcatttctcaggcttttaaatacagagggagagattcaaattgatggggtgtcttgggatagtataacacttcaacagtggaggaaagcctttggtgtgattcctcagaaagtgtttatcttctctggcactttcagaaaaaatctggacccctatgaacagtggagtgaccaggaaatctggaaggtggcagatgaagtgggcctaagatcagtcatagagcagtttcctggaaagttggattttgtgcttgtagatggaggctgtgtgctgtcccatggccataaacagctaatgtgcctggctaggtcagtgctgagcaaggccaagatcctgctgttagatgagccttcagcccatctggaccctgtgacataccagattatcagaagaactctgaagcaggcctttgctgactgcactgtcatcctgtgtgagcacagaattgaggccatgctggagtgccagcagttccttgttatagaagagaataaggttaggcagtatgacagcattcagaaactgctaaatgaaagatctctcttcaggcaagctatttcaccatctgatagagtgaaactttttccccacagaaattcctctaaatgtaaatctaagccccagatagctgccttgaaagaggagactgaagaagaagtccaggacaccagactgtga

SEQ ID NO: 21 - DNA, 4443 bp, Artificial Sequence
  Chemically synthesized oligonucleotide
atgcagagatccccgctggagaaggcatctgtggtgtcaaaactgttctttagctggacaaggcccatccttaggaaagggtacagacagaggttggagctgtcagacatatatcagatcccttcagtggactctgcagacaacctctctgaaaagctggagagggaatgggacagggaactggccagcaaaaaaaaccctaaactgattaatgccctgaggaggtgcttcttttggagattcatgttctatgggatcttcctttacctgggggaggtgactaaagctgttcagcctcttcttctggggaggattattgcctcctatgacccagacaacaaagaagaaagaagcatagccatttacttaggcataggcctctgcttgctcttcatagttagaaccctcctactccacccagccatctttggtctccaccacataggtatgcagatgagaatagcaatgttctccttgatctacaagaagaccctcaagctgtccagcagggtgctggacaagatctccataggccagttagtcagtctactgtccaataacttaaataagtttgatgagggactggcactggcacattttgtgtggattgcccccctccaagtggcccttcttatgggccttatctgggagctgttgcaggcctctgctttctgtggcctgggtttcctcatagtcctagccttattccaggctggactgggcagaatgatgatgaagtatagggaccaaagagcagggaagatttctgaaaggctggttataacttctgagatgattgagaacattcagtcagtgaaagcttactgctgggaagaagctatggaaaaaatgattgaaaatctcagacagactgaattaaagttgaccaggaaagctgcttatgtcagatacttcaactcctcagccttctttttttctggcttctttgttgtattcctttcagtcctcccctatgccctgattaagggcattatcttgaggaaaattttcacaaccatctccttttgtattgtcctcaggatggctgttacaaggcaatttccttgggctgtgcaaacttggtatgatagccttggagcaatcaacaagatccaggatttcctgcaaaagcaggagtacaagacattggaatacaaccttaccaccactgaggtggtgatggaaaatgtgactgccttctgggaggaggggtttggagagctgtttgagaaagccaaacagaacaacaacaatagaaagacctctaatggtgatgattccctgttcttttctaactttagtcttctggggaccccagttctgaaagatattaactttaaaattgaaaggggacagttgctggctgtggctgggtccactggggctgggaagacaagcctgctcatggtgatcatgggagagctggaacccagtgaaggaaagatcaaacactcaggcaggatctccttctgcagccagttctcatggattatgccaggcactattaaagaaaatatcatctttggtgtaagctatgatgagtacaggtatagatctgtaattaaagcctgccagctggaggaagacatctctaagtttgctgagaaggataacattgtgttgggggaagggggcatcaccctttctggtgggcagagggctaggatctcccttgctagggcagtatacaaggatgctgacttgtacctcttggatagtccttttggctacctagatgtgctgacagagaaagaaatatttgaaagctgtgtgtgtaagctcatggctaacaagaccaggatcctggtcaccagtaaaatggaacacctcaaaaaagcagacaagatccttattctccatgagggctcctcctacttctatgggaccttcagtgagctgcagaatctgcagccagacttctcctcaaaacttatgggctgtgactcctttgaccaattctctgcagaaagaaggaatagcatactgacagaaacactgcatagattctccctggaaggagatgccccagtgagttggacagaaaccaaaaagcagagcttcaagcagactggtgagtttggtgaaaagaggaagaattctatcctgaaccccatcaatagcatcaggaaatttagcatagtccaaaagacccccctccagatgaatggaatagaggaggatagtgatgagcctcttgagagaaggctgtccctggttccagacagtgaacagggtgaagccattcttccgaggatcagtgtcatctccactgggcccacattgcaggccagaagaagacagtctgttctgaatttgatgacacattctgtgaatcaaggccagaatatccatagaaaaaccactgccagcaccagaaaagtttctctagccccccaggctaacctgactgagttagacatctacagcagaaggctgagccaagagactggcttggaaatatctgaggagatcaatgaggaggacctcaaggagtgcttctttgatgacatggagtcaatccctgcagtcactacatggaacacttacctaaggtacatcacagttcataagagcctcatctttgtcctcatatggtgtctggtcatctttttagcagaagtggctgccagcctagttgtgctgtggttactgggcaatacacctcttcaggacaaaggcaatagcacacacagcagaaacaactcctatgcagtgatcatcacctctacaagctcttactatgtattctatatatatgtgggagtggcagatactctcctggccatgggattcttcaggggattacctctagttcacacattgatcacagtgtcaaaaattctccaccacaagatgttacacagtgtcctgcaagccccaatgtctactctgaacacacttaaggcaggtggaattttgaataggtttagcaaggacatagctatcctggatgatctcctccctctgaccatctttgacttcatccagttactgctcattgtaattggagccattgcagtggtagcagtcctacagccttacatttttgtggctactgttcctgttattgtggccttcattatgctaagagcttacttcctgcaaacaagccaacagttgaaacagctagaaagtgagggaaggtcccccatcttcacccacctggtgacatcactcaaggggctatggactcttagggcttttgggagacagccgtactttgagaccttattccataaggcccttaacctccatacagcaaactggttcttatacctgagtactctgaggtggtttcaaatgaggattgaaatgatttttgtgatcttcttcattgctgtgaccttcatctcaatcttgaccacaggagagggggagggcagggtgggcatcatactgaccttggccatgaacattatgtcaaccctgcagtgggctgtcaatagctccattgatgtggacagtctgatgaggagtgtctccagggtcttcaagtttattgacatgccaactgagggcaaacccaccaaaagcactaagccatataaaaatggccaactgtccaaagtgatgatcattgaaaattcacatgtaaagaaggatgatatctggccctctggaggacagatgacagtgaaagacctgactgccaagtacacagagggtggtaatgccattcttgagaacattagtttcagtatttccccggggcaaagggtgggcctccttggcagaacaggctctggcaagagtaccctgctgtcagcctttttaagactgttgaacactgagggagaaattcagattgatggtgtctcctgggatagcatcaccctccagcagtggagaaaagcttttggagtgatcccgcaaaaggttttcatcttttcaggcaccttccggaagaacctggacccctatgagcagtggtctgaccaggaaatatggaaggtagctgatgaagttgggcttaggtcagtcatagagcagttcccaggcaaactggactttgtcctggtggatggtggatgtgtactgagtcatgggcacaaacagctgatgtgcctagccaggtctgtgctcagcaaggcaaagatattgctgcttgatgaacccagtgcccatctggacccagtcacatatcagatcatcagaagaacattgaagcaggcctttgctgattgcacagttatcctctgtgagcacaggattgaggccatgctggagtgccagcagtttctggtgattgaggagaataaagtaaggcagtatgactccatccagaagctgctcaatgaaagaagcctctttagacaagctatctccccctcagacagggtcaaattgttccctcacagaaacagcagcaagtgcaagagcaagccccaaattgcagccttgaaagaggagacagaggaagaggtgcaggacaccagactctga

SEQ ID NO: 22 - DNA, 4443 bp, Artificial Sequence
  Chemically synthesized oligonucleotide
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagagaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcagaagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 23 - DNA, 4443 bp, Artificial Sequence
  Chemically synthesized oligonucleotide
atgcagcgcagccccctggagaaggccagcgtggtgagcaagctgttcttcagctggacccgccccatcctgcgcaagggctaccgccagcgcctggagctgagcgacatctaccagatccccagcgtggacagcgccgacaacctgagcgagaagctggagcgcgagtgggaccgcgagctggccagcaagaagaaccccaagctgatcaacgccctgcgccgctgcttcttctggcgcttcatgttctacggcatcttcctgtacctgggcgaggtgaccaaggccgtgcagcccctgctgctgggccgcatcatcgccagctacgaccccgacaacaaggaggagcgcagcatcgccatctacctgggcatcggcctgtgcctgctgttcatcgtgcgcaccctgctgctgcaccccgccatcttcggcctgcaccacatcggcatgcagatgcgcatcgccatgttcagcctgatctacaagaagaccctgaagctgagcagccgcgtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggccgcatgatgatgaagtaccgcgaccagcgcgccggcaagatcagcgagcgcctggtgatcaccagcgagatgatcgagaacatccagagcgtgaaggcctactgctgggaggaggccatggagaagatgatcgagaacctgcgccagaccgagctgaagctgacccgcaaggccgcctacgtgcgctacttcaacagcagcgccttcttcttcagcggcttcttcgtggtgttcctgagcgtgctgccctacgccctgatcaagggcatcatcctgcgcaagatcttcaccaccatcagcttctgcatcgtgctgcgcatggccgtgacccgccagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaaccgcaagaccagcaacggcgacgacagcctgttcttcagcaacttcagcctgctgggcacccccgtgctgaaggacatcaacttcaagatcgagcgcggccagctgctggccgtggccggcagcaccggcgccggcaagaccagcctgctgatggtgatcatgggcgagctggagcccagcgagggcaagatcaagcacagcggccgcatcagcttctgcagccagttcagctggatcatgcccggcaccatcaaggagaacatcatcttcggcgtgagctacgacgagtaccgctaccgcagcgtgatcaaggcctgccagctggaggaggacatcagcaagttcgccgagaaggacaacatcgtgctgggcgagggcggcatcaccctgagcggcggccagcgcgcccgcatcagcctggcccgcgccgtgtacaaggacgccgacctgtacctgctggacagccccttcggctacctggacgtgctgaccgagaaggagatcttcgagagctgcgtgtgcaagctgatggccaacaagacccgcatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagcgccgcaacagcatcctgaccgagaccctgcaccgcttcagcctggagggcgacgcccccgtgagctggaccgagaccaagaagcagagcttcaagcagaccggcgagttcggcgagaagcgcaagaacagcatcctgaaccccatcaacagcatccgcaagttcagcatcgtgcagaagacccccctgcagatgaacggcatcgaggaggacagcgacgagcccctggagcgccgcctgagcctggtgcccgacagcgagcagggcgaggccatcctgccccgcatcagcgtgatcagcaccggccccaccctgcaggcccgccgccgccagagcgtgctgaacctgatgacccacagcgtgaaccagggccagaacatccaccgcaagaccaccgccagcacccgcaaggtgagcctggccccccaggccaacctgaccgagctggacatctacagccgccgcctgagccaggagaccggcctggagatcagcgaggagatcaacgaggaggacctgaaggagtgcttcttcgacgacatggagagcatccccgccgtgaccacctggaacacctacctgcgctacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagccgcaacaacagctacgccgtgatcatcaccagcaccagcagctactacgtgttctacatctacgtgggcgtggccgacaccctgctggccatgggcttcttccgcggcctgcccctggtgcacaccctgatcaccgtgagcaagatcctgcaccacaagatgctgcacagcgtgctgcaggcccccatgagcaccctgaacaccctgaaggccggcggcatcctgaaccgcttcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgcgcgcctacttcctgcagaccagccagcagctgaagcagctggagagcgagggccgcagccccatcttcacccacctggtgaccagcctgaagggcctgtggaccctgcgcgccttcggccgccagccctacttcgagaccctgttccacaaggccctgaacctgcacaccgccaactggttcctgtacctgagcaccctgcgctggttccagatgcgcatcgagatgatcttcgtgatcttcttcatcgccgtgaccttcatcagcatcctgaccaccggcgagggcgagggccgcgtgggcatcatcctgaccctggccatgaacatcatgagcaccctgcagtgggccgtgaacagcagcatcgacgtggacagcctgatgcgcagcgtgagccgcgtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagcgcgtgggcctgctgggccgcaccggcagcggcaagagcaccctgctgagcgccttcctgcgcctgctgaacaccgagggcgagatccagatcgacggcgtgagctgggacagcatcaccctgcagcagtggcgcaaggccttcggcgtgatcccccagaaggtgttcatcttcagcggcaccttccgcaagaacctggacccctacgagcagtggagcgaccaggagatctggaaggtggccgacgaggtgggcctgcgcagcgtgatcgagcagttccccggcaagctggacttcgtgctggtggacggcggctgcgtgctgagccacggccacaagcagctgatgtgcctggcccgcagcgtgctgagcaaggccaagatcctgctgctggacgagcccagcgcccacctggaccccgtgacctaccagatcatccgccgcaccctgaagcaggccttcgccgactgcaccgtgatcctgtgcgagcaccgcatcgaggccatgctggagtgccagcagttcctggtgatcgaggagaacaaggtgcgccagtacgacagcatccagaagctgctgaacgagcgcagcctgttccgccaggccatcagccccagcgaccgcgtgaagctgttcccccaccgcaacagcagcaagtgcaagagcaagccccagatcgccgccctgaaggaggagaccgaggaggaggtgcaggacacccgcctgtaa

SEQ ID NO: 24 - DNA, 4443 bp, Artificial Sequence
  Chemically synthesized oligonucleotide
atgcagagaagccccctggagaaggccagcgtggtgagcaagctgttcttcagctggaccagacccatcctgagaaagggctacagacagagactggagctgagcgacatctaccagatccccagcgtggacagcgccgacaacctgagcgagaagctggagagagagtgggacagagagctggccagcaagaagaaccccaagctgatcaacgccctgagaagatgcttcttctggagattcatgttctacggcatcttcctgtacctgggcgaggtgaccaaggccgtgcagcccctgctgctgggcagaatcatcgccagctacgaccccgacaacaaggaggagagaagcatcgccatctacctgggcatcggcctgtgcctgctgttcatcgtgagaaccctgctgctgcaccccgccatcttcggcctgcaccacatcggcatgcagatgagaatcgccatgttcagcctgatctacaagaagaccctgaagctgagcagcagagtgctggacaagatcagcatcggccagctggtgagcctgctgagcaacaacctgaacaagttcgacgagggcctggccctggcccacttcgtgtggatcgcccccctgcaggtggccctgctgatgggcctgatctgggagctgctgcaggccagcgccttctgcggcctgggcttcctgatcgtgctggccctgttccaggccggcctgggcagaatgatgatgaagtacagggaccagagagccggcaagatcagcgagagactggtgatcaccagcgagatgatcgagaacatccagagcgtgaaggcctactgctgggaggaggccatggagaagatgatcgagaacctgagacagaccgagctgaagctgaccagaaaggccgcctacgtgagatacttcaacagcagcgccttcttcttcagcggcttcttcgtggtgttcctgagcgtgctgccctacgccctgatcaagggcatcatcctgagaaagatcttcaccaccatcagcttctgcatcgtgctgagaatggccgtgaccagacagttcccctgggccgtgcagacctggtacgacagcctgggcgccatcaacaagatccaggacttcctgcagaagcaggagtacaagaccctggagtacaacctgaccaccaccgaggtggtgatggagaacgtgaccgccttctgggaggagggcttcggcgagctgttcgagaaggccaagcagaacaacaacaacagaaagaccagcaacggcgacgacagcctgttcttcagcaacttcagcctgctgggcacccccgtgctgaaggacatcaacttcaagatcgagagaggccagctgctggccgtggccggcagcaccggcgccggcaagaccagcctgctgatggtgatcatgggcgagctggagcccagcgagggcaagatcaagcacagcggcagaatcagcttctgcagccagttcagctggatcatgcccggcaccatcaaggagaacatcatcttcggcgtgagctacgacgagtacagatacagaagcgtgatcaaggcctgccagctggaggaggacatcagcaagttcgccgagaaggacaacatcgtgctgggcgagggcggcatcaccctgagcggcggccagagagccagaatcagcctggccagagccgtgtacaaggacgccgacctgtacctgctggacagccccttcggctacctggacgtgctgaccgagaaggagatcttcgagagctgcgtgtgcaagctgatggccaacaagaccagaatcctggtgaccagcaagatggagcacctgaagaaggccgacaagatcctgatcctgcacgagggcagcagctacttctacggcaccttcagcgagctgcagaacctgcagcccgacttcagcagcaagctgatgggctgcgacagcttcgaccagttcagcgccgagagaagaaacagcatcctgaccgagaccctgcacagattcagcctggagggcgacgcccccgtgagctggaccgagaccaagaagcagagcttcaagcagaccggcgagttcggcgagaagagaaagaacagcatcctgaaccccatcaacagcatcagaaagttcagcatcgtgcagaagacccccctgcagatgaacggcatcgaggaggacagcgacgagcccctggagagaagactgagcctggtgcccgacagcgagcagggcgaggccatcctgcccagaatcagcgtgatcagcaccggccccaccctgcaggccagaagaagacagagcgtgctgaacctgatgacccacagcgtgaaccagggccagaacatccacagaaagaccaccgccagcaccagaaaggtgagcctggccccccaggccaacctgaccgagctggacatctacagcagaagactgagccaggagaccggcctggagatcagcgaggagatcaacgaggaggacctgaaggagtgcttcttcgacgacatggagagcatccccgccgtgaccacctggaacacctacctgagatacatcaccgtgcacaagagcctgatcttcgtgctgatctggtgcctggtgatcttcctggccgaggtggccgccagcctggtggtgctgtggctgctgggcaacacccccctgcaggacaagggcaacagcacccacagcagaaacaacagctacgccgtgatcatcaccagcaccagcagctactacgtgttctacatctacgtgggcgtggccgacaccctgctggccatgggcttcttcagaggcctgcccctggtgcacaccctgatcaccgtgagcaagatcctgcaccacaagatgctgcacagcgtgctgcaggcccccatgagcaccctgaacaccctgaaggccggcggcatcctgaacagattcagcaaggacatcgccatcctggacgacctgctgcccctgaccatcttcgacttcatccagctgctgctgatcgtgatcggcgccatcgccgtggtggccgtgctgcagccctacatcttcgtggccaccgtgcccgtgatcgtggccttcatcatgctgagagcctacttcctgcagaccagccagcagctgaagcagctggagagcgagggcaggagccccatcttcacccacctggtgaccagcctgaagggcctgtggaccctgagagccttcggcagacagccctacttcgagaccctgttccacaaggccctgaacctgcacaccgccaactggttcctgtacctgagcaccctgagatggttccagatgagaatcgagatgatcttcgtgatcttcttcatcgccgtgaccttcatcagcatcctgaccaccggcgagggcgagggcagagtgggcatcatcctgaccctggccatgaacatcatgagcaccctgcagtgggccgtgaacagcagcatcgacgtggacagcctgatgagaagcgtgagcagagtgttcaagttcatcgacatgcccaccgagggcaagcccaccaagagcaccaagccctacaagaacggccagctgagcaaggtgatgatcatcgagaacagccacgtgaagaaggacgacatctggcccagcggcggccagatgaccgtgaaggacctgaccgccaagtacaccgagggcggcaacgccatcctggagaacatcagcttcagcatcagccccggccagagagtgggcctgctgggcagaaccggcagcggcaagagcaccctgctgagcgccttcctgagactgctgaacaccgagggcgagatccagatcgacggcgtgagctgggacagcatcaccctgcagcagtggagaaaggccttcggcgtgatcccccagaaggtgttcatcttcagcggcaccttcagaaagaacctggacccctacgagcagtggagcgaccaggagatctggaaggtggccgacgaggtgggcctgagaagcgtgatcgagcagttccccggcaagctggacttcgtgctggtggacggcggctgcgtgctgagccacggccacaagcagctgatgtgcctggccagaagcgtgctgagcaaggccaagatcctgctgctggacgagcccagcgcccacctggaccccgtgacctaccagatcatcagaagaaccctgaagcaggccttcgccgactgcaccgtgatcctgtgcgagcacagaatcgaggccatgctggagtgccagcagttcctggtgatcgaggagaacaaggtgagacagtacgacagcatccagaagctgctgaacgagagaagcctgttcagacaggccatcagccccagcgacagagtgaagctgttcccccacagaaacagcagcaagtgcaagagcaagccccagatcgccgccctgaaggaggagaccgaggaggaggtgcaggacaccagactgtga

SEQ ID NO: 25 - DNA, 4443 bp, Artificial Sequence
  Chemically synthesized oligonucleotide
atgcagaggtcacctctggaaaaggctagcgtggtcagcaagctatttttttcctggacccgcccgatactcaggaagggctaccgacagcggctggagctgagtgacatttatcagattccctccgtcgattccgctgacaacctgtctgagaaactggagcgggaatgggatagggaactggcgtccaaaaaaaaccccaaactcatcaatgcactccgcagatgcttcttctggcggtttatgttttatggcatattcctgtatctgggggaggtgacgaaagccgtgcagccgctgctgcttggtcgcattatcgcgtcatacgatccagataacaaggaggaaagaagtatcgctatctatctcgggatagggctgtgcctgctcttcattgtgcggactcttctcttgcaccccgccattttcggtctgcatcatataggtatgcagatgagaattgcgatgttctcattgatttacaaaaaaacgcttaagctaagttcaagggtgctagataagatatcgatcggccagctggtgtctctgcttagcaacaacctcaataaattcgacgaaggccttgcactggcccacttcgtgtggatcgcccctctgcaggtggctctgctgatggggttaatatgggagctgttgcaggcctccgctttttgtggcctggggtttctcatcgtgttggccttgtttcaggcagggctgggacgtatgatgatgaaatatagggatcagagggctggcaaaatctctgagcgcctggttattacgagtgaaatgattgagaacatccagtcagtgaaggcctattgctgggaggaggccatggaaaaaatgattgagaacctacgccagactgagctgaagttaaccagaaaagccgcctatgtgcgctactttaacagtagcgcatttttcttctccggttttttcgtggtgtttcttagtgtgttgccgtatgccttaatcaagggaataatactccggaagattttcactaccatcagcttctgtatcgtgttgcggatggccgtcacccggcagtttccctgggcagtacagacttggtacgattctctcggagcaattaacaaaatccaagactttctacaaaagcaggagtacaagaccctggagtacaatctgaccaccacagaagtcgtaatggagaatgtaactgccttctgggaagagggctttggcgaactctttgaaaaggccaagcagaacaataacaaccggaagacctccaacggggacgacagcttatttttcagcaatttttctttgctcgggacccctgtactgaaagatattaactttaagatcgagcgcggacaactcctggctgtcgccggcagcactggagctggaaaaacatcactgcttatggtgataatgggagaactcgaaccaagcgagggaaaaataaagcactctggacggattagtttttgctcccagttctcgtggataatgcctggcaccattaaggagaatatcatctttggagtgagttacgacgaataccggtaccggtccgttatcaaggcttgtcaactcgaggaggacatttctaaattcgccgaaaaagataatatagtgctgggcgaaggaggcattacactgagcgggggtcagagagctcgaattagcctcgcccgagcagtctataaagacgccgatctttacctgctggattccccttttgggtatttggatgttctgacagagaaggaaatctttgaatcatgtgtctgtaaactgatggccaataagactaggattctagtgacttcgaaaatggagcacctgaaaaaagcggacaaaattctgatactccatgaagggtcttcctacttctacggcaccttctcagagttgcagaacttacaacctgatttttcatctaagcttatggggtgcgactcgtttgaccagttctccgctgaaagacgaaacagcatcttaacggaaactcttcacaggttctcattagagggagatgcgccggtgtcctggacagagacaaaaaaacagtctttcaaacagacaggagagtttggcgagaagagaaaaaactcaatcctcaatcccatcaattctattagaaagtttagcatcgtccaaaaaacaccattgcagatgaatgggattgaggaggacagtgatgagcctttggaacgaagactgtccctggtacccgatagcgaacagggtgaggccatccttcctaggatctcggtcataagtacagggcccacactgcaggccaggcgacgtcaaagtgtcctcaatcttatgacgcacagtgtgaatcaggggcagaacatccatcgtaagacgacagcttcaactcgaaaggtcagtctagctccacaagccaatcttacagagctggacatttattcccgccgcctcagtcaggagaccggattggaaatatcagaggaaattaatgaagaggatctgaaggaatgcttctttgatgacatggaatcgatccccgctgttactacctggaacacatatctgagatatattaccgtccataagagcttaatctttgtactgatatggtgcttggtgattttcctggcagaggttgcggcgagtttggtcgtgctatggctccttggaaacactcccctgcaggataaggggaactccactcatagcaggaataacagctatgccgtgatcatcacctctacctcctcttattacgtgttttacatatacgtcggtgttgcggataccctgttggcaatggggttctttagaggactacccctagttcacaccctgatcaccgtttcgaagatcttgcaccacaagatgcttcatagcgttctccaagctcctatgagcacccttaatacactgaaagcaggaggtatccttaaccgcttttccaaagacatcgctatactcgacgatttgctcccattgaccatcttcgacttcattcagctgctcctcattgtgatcggcgccattgccgtggtcgcagtgttacagccatatattttcgtagccaccgtgcccgtcatcgtggcatttatcatgctgcgcgcatatttcttacagacatctcagcaactgaagcagctggaatctgagggcagatctcctatttttacacacctggttaccagcctgaagggcctgtggaccctgcgtgctttcggtcgccaaccctactttgagactctcttccataaggctctgaatttacatactgccaattggttcctataccttagtacccttcggtggttccagatgcggatagaaatgatcttcgtgattttcttcatcgcagtcactttcatctctattttgacgaccggtgagggcgagggcagggtgggcatcattctgactttggccatgaacattatgtcaacactccagtgggccgttaattcaagcattgatgtggattccttgatgcgttccgtcagcagggtatttaaattcatagacatgcccaccgagggcaagccaacaaaatctaccaagccatacaaaaatggccaactaagcaaggtcatgattatcgagaattctcatgtgaaaaaggacgacatttggccttccgggggtcaaatgactgtaaaggacctgacggctaaatacactgagggcggtaatgctatcttggagaacatctctttcagcatctcccctggccagagagtgggactgctcgggcggacaggctccggaaagtctacgctcctttcagcattccttagacttctgaacaccgaaggtgagattcagattgacggggtctcttgggactccatcacacttcagcaatggaggaaggcattcggtgtaatcccccaaaaggtttttatcttctccggaacatttcgtaagaatctggacccgtacgagcagtggtcagatcaggagatctggaaagtagcagacgaggtcgggctacggagcgttattgaacagtttcctggcaaactggacttcgttttggtggacggaggctgtgtgctgagtcacggccataaacaactgatgtgcttagctaggtctgttctcagcaaggcaaagattttactgctggatgaaccaagcgcccaccttgatccagtgacatatcaaatcatcagaagaactcttaaacaggcgttcgccgactgcacagtgatcctgtgtgagcacagaatagaagccatgctggaatgtcaacagtttctcgtgattgaggagaacaaggtgcgccagtacgatagcatccagaagttactcaatgaaaggtcactcttcaggcaggccatctcacccagcgaccgcgttaagctgtttccacaccgaaacagttccaagtgcaaaagtaagccacagattgctgcactgaaggaagagacagaagaagaagttcaggacactcggctctga

SEQ ID NO: 26 - DNA, 4443 bp, Artificial Sequence; identical to SEQ ID NO: 8
  Chemically synthesized oligonucleotide

SEQ ID NO: 27 - DNA, 4443 bp, Artificial Sequence
  Chemically synthesized oligonucelotide
atgcaacggagtcctctggaaaaagcctctgtcgtatctaagcttttcttcagttggacacgcccgattttgagaaagggttatcggcaacgcttggaacttagtgacatctaccaaattccaagtgtagactcagccgataacttgagcgaaaagctcgaacgagagtgggatcgagaactggctagcaaaaaaaatcccaaactcataaatgccctgcgacgctgtttcttttggcgatttatgttttacggtattttcctttatttgggtgaggtcacgaaggctgtacagccactgctgctgggtcgcatcattgcctcttacgaccctgacaacaaagaggagcggtcaatagctatctaccttggtataggactttgcttgctcttcatagtccgcacgttgcttctccaccctgctatatttggtctccatcacattgggatgcaaatgcggatcgcgatgttcagtcttatatataaaaagactcttaaactttccagccgggttctggataagatctctattggtcaactggtatctcttttgtctaacaacctgaataagttcgacgagggccttgcattggcccattttgtatggattgcccctttgcaagtcgccctcctgatgggattgatctgggaactcctgcaagctagtgctttttgcggattgggattcctcatagtccttgcgctctttcaggcgggacttggacgcatgatgatgaagtatcgcgaccaacgagctggcaagatcagtgaacggcttgtaataaccagtgaaatgatagagaacatccagagcgtaaaagcttactgttgggaagaagcgatggaaaagatgattgagaaccttcgccagacagaacttaaacttacacgaaaggccgcttatgtccggtacttcaactcttcagcatttttttttagtggcttctttgtagtgttcctgtccgtccttccgtatgcacttatcaagggtataatacttaggaaaatcttcacaacaatcagtttttgcatagtccttcgcatggcagtaactcgccaatttccctgggcagttcagacgtggtacgactcacttggcgcaattaacaaaattcaagatttcctccaaaagcaagagtataaaaccttggaatacaaccttaccaccacagaagttgtaatggaaaatgtcacagccttctgggaggaaggtttcggcgaactttttgagaaggcgaagcaaaataacaataatcggaaaacatcaaacggtgacgattcactgttcttttctaactttagccttcttgggacgcccgtcctgaaggacataaactttaagattgaacggggtcaacttctcgcggtcgcagggagtactggagcggggaaaacgagcctgctgatggtgataatgggggagttggagccctcagaaggcaagatcaagcatagtggtagaattagcttctgcagtcaatttagttggattatgccgggcacgatcaaagaaaatataatctttggggtatcctacgatgaatacaggtaccgatcagtgataaaagcgtgccagcttgaagaagacatttcaaagtttgctgagaaggataatatcgtacttggagaaggaggtatcaccctgtctgggggtcaacgagcgaggatctccctggcacgcgccgtctacaaggacgcggacctctatctgttggattcaccgttcggatatttggacgtgcttacggagaaagaaatatttgagagctgtgtttgcaagctcatggcaaataaaaccagaatattggttacaagcaagatggagcatcttaagaaagcagataaaatcctgatattgcacgagggctcttcatacttctacgggacgttttctgagttgcagaacctccagccggatttcagctctaagctgatgggctgtgattcctttgatcagtttagtgcggaaagacgaaacagtatactcaccgaaacactgcacaggttctctctggagggcgacgccccggtttcctggacagagacgaagaagcagtccttcaaacagacaggcgagtttggggagaaaaggaaaaatagcatactcaacccgattaacagcattcgcaagttcagtatagtacaaaagaccccgttgcagatgaacggtatagaggaagattctgatgagccactggaaagacggctttctctcgttccggacagtgaacagggagaggcaatactgcctcggatcagcgttatctctacaggacctactttgcaagctcggcgccgacagtcagtcttgaatcttatgactcatagtgttaatcaaggccagaatatccatcgcaagaccaccgcaagtacaaggaaagtgagcttggcacctcaagcaaaccttactgaacttgatatctactcacggcgactttcacaggagaccggacttgaaattagtgaagaaattaacgaggaggacctcaaggagtgcttcttcgatgacatggaatcaatccccgcagtcacaacctggaacacttatctgaggtatataacagttcacaagagcctcatttttgtacttatttggtgtttggtaattttcctggcggaggttgctgcttctttggtcgtcctttggctcctcgggaatacaccgctccaagacaaaggcaactctacccatagtaggaacaattcatatgcagtgattataaccagtacatcatcttattacgttttctatatttatgtcggggtagctgacacgctgttggcgatgggcttctttaggggcctccccttggtacacacccttatcacggtgagtaaaatcctgcatcacaaaatgcttcattctgtactccaagcgccgatgagtacgcttaatacgctgaaagcaggagggatactgaatcggttcagcaaggacatcgccattctggatgacctgcttccattgacaatatttgatttcattcagctccttctcatagttattggagccatagcggtggtggctgtgcttcagccttatatattcgttgccacagttcccgttatagtggcatttataatgctcagggcctactttctccagacttcccagcagttgaagcaactcgaatcagaaggaaggtcacctattttcacacatcttgtgacttccttgaagggcttgtggacgctgcgggccttcggaagacaaccatattttgaaactctcttccacaaagctttgaatcttcatactgcgaactggttcctgtatttgagtactttgcgctggttccagatgaggatagaaatgatattcgttatcttctttatcgcggttacgttcataagtatcctcactacgggggagggtgagggtagagtgggcataatactgaccctcgccatgaacattatgtccaccctgcagtgggcggtaaacagcagcatagatgtggattctttgatgcgcagtgtgagcagggtttttaagtttatcgatatgccgacggaaggaaagcccactaaaagcacgaaaccctataaaaatggacagcttagcaaagtaatgataatcgagaatagccatgtgaaaaaggatgacatatggccttccggaggccaaatgactgttaaagatctgaccgctaaatataccgagggcggcaacgcaatactcgaaaacataagcttttccataagccccggccaacgcgtgggtcttctggggaggactggctccggaaaatcaacgttgcttagcgcgtttttgcggctccttaacactgaaggtgagatccaaatagatggcgttagttgggactctataacactgcaacaatggcggaaagctttcggcgtcatacctcagaaggtgttcatctttagcggaacgttcaggaagaacttggatccctacgaacaatggagtgatcaagaaatatggaaagtggcagatgaggtaggcttgcgcagtgtcattgaacaattcccagggaaactcgactttgtactggtggacggcggttgcgtcttgtcacacgggcacaaacagttgatgtgtttggcccgcagtgttttgtctaaggcgaagattctgttgctcgacgaaccgagtgctcatcttgatcccgtcacctaccaaatcatcagaaggacgttgaagcaagctttcgccgactgcactgtaatcctttgtgagcataggatcgaagcaatgctcgagtgccaacagttcttggttatagaggagaataaggttcggcaatacgactcaatacagaaactgcttaatgagcggtcactctttcgacaagctatctctcctagtgacagggtaaagctttttcctcatcggaattccagcaagtgtaagagtaaaccacagatcgccgcccttaaagaggagaccgaagaagaggtgcaggatacgagactttag

(19) United States
(12) Patent Application Publication
(10) Pub. No.: US 2024/0299489 A9
(43) Pub. Date: Sep. 12, 2024

(54) TREATMENT OF CYSTIC FIBROSIS BY DELIVERY OF CODON-OPTIMIZED mRNA ENCODING CFTR

(71) Applicant: Translate Bio, Inc., Lexington, MA (US)

(72) Inventors: Michael Heartlein, Lexington, MA (US); Frank DeRosa, Lexington, MA (US); Alan Kimura, Lexington, MA (US); Jonathan Abysalh, Lexington, MA (US); Anusha Dias, Lexington, MA (US); Shrirang Karve, Lexington, MA (US); Zarna Patel, Lexington, MA (US)

(21) Appl. No.: 17/449,679

(22) Filed: Oct. 1, 2021

Related U.S. Application Data
(62) Division of application No. 15/981,757, filed on May 16, 2018, now Pat. No. 11,173,190.
(60) Provisional application No. 62/507,061, filed on May 16, 2017.
(60) Provisional application No. 62/659,053, filed on Apr. 17, 2018.
(60) Provisional application No. 62/592,238, filed on Nov. 29, 2017.
(60) Provisional application No. 62/532,301, filed on Jul. 13, 2017.
(60) Provisional application No. 62/580,782, filed on Nov. 2, 2017.

(51) Int. Cl. A61K 38/17; A61K 9/00; A61K 9/127; A61K 48/00; A61P 11/12

(52) U.S. Cl. CPC A61K 38/1709; A61K 9/0078; A61K 9/127; A61K 48/0075; A61P 11/12; C12N 15/88

(57) ABSTRACT

The present invention provides, among other things, methods of treating cystic fibrosis, comprising a step of administering to a subject in need of treatment a composition comprising an mRNA encoding a Cystic Fibrosis Transmembrane Conductance Regulator (CFTR) protein, wherein the mRNA encoding the CFTR protein comprises a polynucleotide sequence at least 80% identical to SEQ ID NO: 1, wherein the mRNA is at a concentration of at least 0.4 mg/mL, and wherein the step of administering comprises inhalation.

### RELATED APPLICATIONS

This application claims priority to U.S. Provisional Applications Ser. No. 62/507,061, filed May 16, 2017; Ser. No. 62/532,301, filed on Jul. 13, 2017; Ser. No. 62/580,782, filed Nov. 2, 2017; Ser. No. 62/592,238, filed Nov. 29, 2017; and Ser. No. 62/659,053, filed Apr. 17, 2018, the disclosures in their entirety of all of which are hereby incorporated by reference.

### SEQUENCE LISTING

The present specification makes reference to a Sequence Listing (submitted electronically as a .txt file named “SL MRT-2005PCT-US” on May 16, 2018. The .txt file was generated May 15, 2018 and is 152,577 bytes in size. The entire contents of the Sequence Listing are herein incorporated by reference.

### BACKGROUND

Cystic fibrosis is an autosomal inherited disorder resulting from mutation of the CFTR gene, which encodes a chloride ion channel believed to be involved in regulation of multiple other ion channels and transport systems in epithelial cells. Loss of function of CFTR results in chronic lung disease, aberrant mucus production, and dramatically reduced life expectancy. See generally Rowe et al., New Engl. J. Med. 352, 1992-2001 (2005).

Currently there is no cure for cystic fibrosis (CF). The literature has documented numerous difficulties encountered in attempting to induce expression of CFTR in the lung. For example, viral vectors comprising CFTR DNA triggered immune responses and CF symptoms persisted after administration. Conese et al., J. Cyst. Fibros. 10 Suppl 2, S114-28 (2011); Rosenecker et al., Curr. Opin. Mol. Ther. 8, 439-45 (2006). Non-viral delivery of DNA, including CFTR DNA, has also been reported to trigger immune responses. Alton et al., Lancet 353, 947-54 (1999); Rosenecker et al., J Gene Med. 5, 49-60 (2003). Furthermore, non-viral DNA vectors encounter the additional problem that the machinery of the nuclear pore complex does not ordinarily import DNA into the nucleus, where transcription would occur. Pearson, Nature 460, 164-69 (2009).

### SUMMARY OF THE INVENTION

The present invention provides, among other things, methods of treating cystic fibrosis with an mRNA encoding a Cystic Fibrosis Transmembrane Conductance Regulator (CFTR) protein. In one aspect, the present invention provides methods of treating cystic fibrosis, comprising a step of administering to a subject in need of treatment a composition comprising an mRNA encoding a Cystic Fibrosis Transmembrane Conductance Regulator (CFTR) protein, wherein the mRNA encoding the CFTR protein comprises a polynucleotide sequence at least 80% identical to SEQ ID NO: 1. In some embodiments, the mRNA encoding CFTR is at a concentration of at least 0.4 mg/mL and the step of administering comprises inhalation. In some embodiments, the mRNA encoding CFTR is at a concentration of at least 0.5 mg/mL. In some embodiments, the mRNA encoding CFTR is at a concentration of at least 0.6 mg/mL. In some embodiments, the mRNA encoding CFTR is at a concentration ranging from 0.4 mg/mL to 0.8 mg/mL. In some embodiments, the dose is 24 mg or less per week of mRNA encoding CFTR. In some embodiments, the dose is 16 mg or less per week of mRNA encoding CFTR. In some embodiments, the dose is 8 mg or less per week of mRNA encoding CFTR.

In some embodiments, the mRNA encoding the CFTR protein comprises a polynucleotide sequence at least 85% identical to SEQ ID NO: 1. In some embodiments, the mRNA encoding the CFTR protein comprises a polynucleotide sequence at least 90% identical to SEQ ID NO: 1. In some embodiments, the mRNA encoding the CFTR protein comprises a polynucleotide sequence at least 95% identical to SEQ ID NO: 1. In some embodiments, the mRNA encoding the CFTR protein comprises a polynucleotide sequence at least 99% identical to SEQ ID NO: 1. In some embodiments, the mRNA encoding the CFTR protein comprises a polynucleotide sequence identical to SEQ ID NO: 1.

In some embodiments, the composition is nebulized prior to inhalation. In some embodiments, the composition is stored as a frozen, sterile suspension prior to administering. In some embodiments, the composition is stored in a single-use vial prior to administering. In some embodiments, the single-use vial comprises less than 5.0 mL of the composition. In some embodiments, the mRNA encoding the CFTR protein is at a dosage ranging from 8 mg to 24 mg.

In some embodiments, the mRNA encoding the CFTR protein further comprises a 5′ untranslated region (UTR) sequence of SEQ ID NO: 4. In some embodiments, the mRNA encoding the CFTR protein further comprises a 3′ untranslated region (UTR) sequence of SEQ ID NO: 5 or SEQ ID NO: 6.

In some embodiments, the mRNA encoding the CFTR protein is encapsulated within a nanoparticle. In some embodiments, the nanoparticle is a liposome. In some embodiments, the liposome comprises one or more cationic lipids, one or more non-cationic lipids, and one or more PEG-modified lipids. In some embodiments, the liposome comprises no more than three distinct lipid components. In some embodiments, one distinct lipid component is a sterol-based cationic lipid. In some embodiments, the liposome has a size less than about 100 nm. In some embodiments, the liposome has a size ranging from 40 nm to 60 nm. In some embodiments, the no more than three distinct lipid components are a cationic lipid, a non-cationic lipid and a PEG-modified lipid. In some embodiments, the liposome comprises imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K). In some embodiments, ICE and DOPE are present at a molar ratio of >1:1. In some embodiments, ICE and DMG-PEG-2K are present at a molar ratio of >10:1. In some embodiments, DOPE and DMG-PEG-2K are present at a molar ratio of >5:1.

In some embodiments, the single-use vial comprises between 3.0 and 4.0 mL of the composition. In some embodiments, the single-use vial comprises between 3.2 mL of the composition.

It is to be understood that all embodiments as described above are applicable to all aspects of the present invention. Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWING

The drawings are for illustration purposes only not for limitation.

FIG. 1 depicts the general structure of the composition comprising an mRNA encoding a CFTR protein and a simplified formulation process.

FIG. 2 depicts an exemplary graph of the number of copies of mRNA/gm in rat lung tissue after the rats were treated with hCFTR mRNA-loaded liposomes.

FIG. 3 depicts an exemplary graph of the number of copies of mRNA in primate (NHP) lung tissue after the NHPs were treated with hCFTR mRNA-loaded liposomes.

FIG. 4 depicts an exemplary graph showing increased chloride channel activity demonstrated after transfection with hCFTR mRNA-loaded liposomes.

FIG. 5 depicts exemplary IHC of hCFTR protein in lung tissue from a primate after a single dose of a composition comprising an mRNA encoding a CFTR protein.

FIG. 6 depicts an exemplary graph of fold-increase of copies of CO-hCFTR mRNA over endogenous levels of CFTR mRNA in primate lung tissue after a single dose of a composition comprising an mRNA encoding a CFTR protein.

FIG. 7 depicts an exemplary graph of percent of CO-hCFTR mRNA delivered to primate lung tissue as compared to airway tissue after a single dose of a composition comprising an mRNA encoding a CFTR protein.

FIG. 8A-C depicts expression of CFTR mRNA-derived protein in primate upper bronchial epithelial cells colocalized with endogenous membrane tight junction protein, ZO1 (which is present in the cell membrane), following treatment with nebulized CFTR mRNA encapsulated in a lipid nanoparticle, delivered by inhalation. FIG. 8A depicts microscopic immunostaining images of upper bronchial epithelial cells from a primate treated with buffer (10% trehalose) as a control; showing mRNA-derived CFTR protein staining (left), endogenous ZO1 protein staining (middle) and optical merge of mRNA-derived CFTR protein staining and endogenous ZO1 protein staining (right), with no visible mRNA-derived CFTR protein present in the cell membranes of upper bronchial epithelial cells. FIG. 8B depicts microscopic immunostaining images of upper bronchial epithelial cells from a primate treated with nebulized CFTR mRNA (500 μg/kg); showing mRNA-derived CFTR protein staining (left), endogenous ZO1 proten staining (middle) and optical merge of mRNA-derived CFTR protein staining and endogenous ZO1 protein staining (right), with mRNA-derived CFTR protein present in the cell membranes of upper bronchial epithelial cells. FIG. 8C depicts representative microscopic immunostaining images of upper bronchial epithelial cells from a primate treated with nebulized CFTR mRNA (1000 μg/kg); showing mRNA-derived CFTR protein staining (left), endogenous ZO1 protein staining (middle) and optical merge of mRNA-derived CFTR protein staining and endogenous ZO1 protein staining (right) with mRNA-derived CFTR protein present in the cell membranes of upper bronchial epithelial cells.

FIG. 9A-C depicts expression of CFTR mRNA-derived protein in primate lower airway epithelial cells colocalized with endogenous membrane tight junction protein, ZO1 (which is present in the cell membrane), following treatment with nebulized CFTR mRNA encapsulated in a lipid nanoparticle, delivered by inhalation. FIG. 9A depicts microscopic immunostaining images of lower airway epithelial cells from a primate treated with buffer (10% trehalose) as a control; showing mRNA-derived CFTR protein staining (left), endogenous ZO1 protein staining (middle) and optical merge of mRNA-derived CFTR protein and endogenous ZO1 protein staining (right), with no visible mRNA-derived CFTR protein present in the cell membranes of lower bronchial epithelial cells. FIG. 9B depicts microscopic immunostaining images of lower airway epithelial cells from a primate treated with nebulized CFTR mRNA (500 μg/kg); showing mRNA-derived CFTR protein staining (left), endogenous ZO1 protein staining (middle) and optical merge of mRNA-derived CFTR protein staining and endogenous ZO1 protein staining (right), with mRNA-derived CFTR protein present in the cell membranes of the lower bronchial epithelial cells. FIG. 9C depicts representative microscopic immunostaining images of lower airway epithelial cells from a primate treated with nebulized CFTR mRNA (1000 μg/kg); showing mRNA-derived CFTR protein staining (left), endogenous ZO1 protein staining (middle) and optical merge of mRNA-derived CFTR protein staining and endogenous ZO1 protein staining (right), with mRNA-derived CFTR protein present in the cell membranes of lower bronchial epithelial cells.

FIG. 10A-C depicts expression of CFTR mRNA-derived protein in cells of the alveolar region of a primate lung colocalized with endogenous membrane tight junction protein, ZO1 (which is present in the cell membrane), following treatment with nebulized CFTR mRNA encapsulated in a lipid nanoparticle, delivered by inhalation. FIG. 10A depicts microscopic immunostaining images from a primate treated with buffer (10% trehalose) as a control; showing mRNA-derived CFTR protein staining (left), endogenous ZO1 protein staining (middle) and optical merge of mRNA-derived CFTR protein and endogenous ZO1 protein staining (right), with no mRNA-derived CFTR protein present in the cell membranes of alveolar cells. FIG. 10B depicts microscopic immunostaining images from a primate treated with nebulized CFTR mRNA (500 μg/kg); showing mRNA-derived CFTR protein staining (left), endogenous ZO1 protein staining (middle) and optical merge of mRNA-derived CFTR protein and endogenous ZO1 protein staining (right), with mRNA-derived CFTR protein present in the cell membranes of alveolar cells. FIG. 10C shows representative microscopic immunostaining images from a primate treated with nebulized CFTR mRNA (1000 μg/kg); showing mRNA-derived CFTR protein staining (left), endogenous ZO1 protein staining (middle) and optical merge of mRNA-derived CFTR protein staining and endogenous ZO1 protein staining (right), with mRNA-derived CFTR protein present in the cell membranes of alveolar cells.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery. For example, delivery of mRNA encompasses situations in which an mRNA is delivered to a target tissue and the encoded protein is expressed and retained within the target tissue (also referred to as “local distribution” or “local delivery”), and situations in which an mRNA is delivered to a target tissue and the encoded protein is expressed and secreted into patient's circulation system (e.g., serum) and systematically distributed and taken up by other tissues (also referred to as “systemic distribution” or “systemic delivery). In some embodiments, delivery is pulmonary delivery, e.g., comprising nebulization.

Encapsulation: As used herein, the term “encapsulation,” or grammatical equivalent, refers to the process of confining an mRNA molecule within a nanoparticle.

Expression: As used herein, “expression” of a nucleic acid sequence refers to translation of an mRNA into a polypeptide, assemble multiple polypeptides (e.g., heavy chain or light chain of antibody) into an intact protein (e.g., antibody) and/or post-translational modification of a polypeptide or fully assembled protein (e.g., antibody). In this application, the terms “expression” and “production,” and grammatical equivalents, are used interchangeably.

Functional: As used herein, a “functional” biological molecule is a biological molecule in a form in which it exhibits a property and/or activity by which it is characterized.

Half-life: As used herein, the term “half-life” is the time required for a quantity such as nucleic acid or protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Isolated: As used herein, the term “isolated” refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from about 10%, about 20%, about 30%, about 40%, about 50%, about 60%, about 70%, about 80%, about 90%, about 91%, about 92%, about 93%, about 94%, about 95%, about 96%, about 97%, about 98%, about 99%, or more than about 99% of the other components with which they were initially associated. In some embodiments, isolated agents are about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. As used herein, calculation of percent purity of isolated substances and/or entities should not include excipients (e.g., buffer, solvent, water, etc.).

messenger RNA (mRNA): As used herein, the term “messenger RNA (mRNA)” refers to a polynucleotide that encodes at least one polypeptide. mRNA as used herein encompasses both modified and unmodified RNA. mRNA may contain one or more coding and non-coding regions. mRNA can be purified from natural sources, produced using recombinant expression systems and optionally purified, chemically synthesized, etc. Where appropriate, e.g., in the case of chemically synthesized molecules, mRNA can comprise nucleoside analogs such as analogs having chemically modified bases or sugars, backbone modifications, etc. An mRNA sequence is presented in the 5′ to 3′ direction unless otherwise indicated.

Nucleic acid: As used herein, the term “nucleic acid,” in its broadest sense, refers to any compound and/or substance that is or can be incorporated into a polynucleotide chain. In some embodiments, a nucleic acid is a compound and/or substance that is or can be incorporated into a polynucleotide chain via a phosphodiester linkage. In some embodiments, “nucleic acid” refers to individual nucleic acid residues (e.g., nucleotides and/or nucleosides). In some embodiments, “nucleic acid” refers to a polynucleotide chain comprising individual nucleic acid residues. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e., analogs having other than a phosphodiester backbone. For example, the so-called “peptide nucleic acids,” which are known in the art and have peptide bonds instead of phosphodiester bonds in the backbone, are considered within the scope of the present invention. The term “nucleotide sequence encoding an amino acid sequence” includes all nucleotide sequences that are degenerate versions of each other and/or encode the same amino acid sequence. Nucleotide sequences that encode proteins and/or RNA may include introns. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages). In some embodiments, the present invention is specifically directed to “unmodified nucleic acids,” meaning nucleic acids (e.g., polynucleotides and residues, including nucleotides and/or nucleosides) that have not been chemically modified in order to facilitate or achieve delivery. In some embodiments, the nucleotides T and U are used interchangeably in sequence descriptions.

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, methods of treating cystic fibrosis comprising a step of administering to a subject in need of treatment a composition comprising an mRNA encoding a Cystic Fibrosis Transmembrane Conductance Regulator (CFTR) protein, wherein the mRNA encoding the CFTR protein comprises a polynucleotide sequence at least 80% identical to SEQ ID NO: 1, wherein the mRNA is at a concentration of at least 0.4 mg/mL, and wherein the step of administering comprises inhalation.

Various aspects of the invention are described in detail in the following sections. The use of sections is not meant to limit the invention. Each section can apply to any aspect of the invention. In this application, the use of “or” means “and/or” unless stated otherwise.

Cystic Fibrosis

Cystic fibrosis, also known as mucoviscidosis, is an autosomal recessive genetic disorder that affects most critically the lungs, and also the pancreas, liver, and intestine (Gibson et al., Am J Respir Crit Care Med. (2003) 168(8):918-951; Ratjen et al., Lancet Lond Engl. (2003) 361(9358):681-689; O'Sullivan et al., Lancet Lond Engl. (2009) 373(9678):1891-1904). Cystic fibrosis is caused by mutations in the gene encoding for the cystic fibrosis transmembrane conductance regulator (CFTR) protein. This protein functions as a channel that transports chloride ions across the membrane of cells and is required to regulate the components of mucus, sweat, saliva, tears, and digestive enzymes. Disease-causing mutations in the CFTR protein cause dysfunction of its channel activity resulting in abnormal transport of chloride and sodium ions across the epithelium, leading to the thick, viscous secretions in the lung, pancreas and other organs characteristic of CF disease (O'Sulliven et al., Lancet Lond Engl. (2009) 373(9678):1891-1904; Rowe et al., N Engl J Med. (2005) 352(19):1992-2001). Most CF patients develop severe, chronic lung disease related to airway obstruction partly due to increased levels of sulfated mucins, inflammation, and recurrent infections that are eventually lethal; the median predicted survival age in the US is 40.7 years. Cystic fibrosis is the most frequent lethal genetic disease in the white population.

Symptoms often appear in infancy and childhood, with respiratory symptoms the most frequent followed by failure to thrive, steatorrhea, and meconium ileus (Gibson et al., Am J Respir Crit Care Med. (2003) 168(8):918-951). The most common complications of CF are pulmonary related and include blockages of the narrow passages of affected organs with thickened secretions. These blockages lead to remodeling and infection in the lung, cause damage in the pancreas due to accumulated digestive enzymes, and blockages of the intestines. Diabetes is the most common non-pulmonary complication and is a distinct entity known as CF-related diabetes.

The lungs of individuals with CF are colonized and infected by bacteria from an early age. This leads to chronic airway infection and inflammation, progressing to bronchiectasis, gas trapping, hypoxemia, and hypercarbia. Pulmonary insufficiency is responsible for 68.1% of CF-related deaths in the US. In the initial stage, common bacteria such as Staphylococcus aureus and Hemophilus influenzae colonize and infect the lungs. Eventually, Pseudomonas aeruginosa (and sometimes Burkholderia cepacia) dominates. By 18 years of age, 80% of patients with classic CF harbor P. aeruginosa, and 3.5% harbor B. cepacia. Once within the lungs, these bacteria adapt to the environment and develop resistance to commonly used antibiotics.

The underlying defect causing CF is abnormal epithelial anion transport due to the lack of expression or dysfunction of the CFTR protein. The CFTR protein primarily functions as a chloride channel in epithelial cell membranes; however, it also involved in a number of other cellular membrane functions such as inhibition of sodium transport through the epithelial sodium channel, regulation of the outwardly rectifying chloride channel, and regulation of adenosine triphosphate (ATP) channels (O'Sullivan et al., Lancet Lond Engl. (2009) 373(9678):1891-1904). CF is caused by mutations in the gene encoding for the CFTR protein, of which more than 1,500 disease-causing mutations have been identified (O′Sullivan et al., Lancet Lond Engl. (2009) 373(9678):1891-1904). The more common gene mutations result in the lack of synthesis of the CFTR protein (class I), defective processing and maturation of the CFTR protein (class II), or the expression of a CFTR protein defective in regulation, e.g., diminished ATP binding and hydrolysis (class III) (Rowe et al., N Engl J Med. (2005) 352(19):1992-2001). A deletion of phenylalanine at position 508 (F508del) is the most common CFTR mutation worldwide and is a class II defect in which the misfolded protein is rapidly degraded by the cell soon after synthesis (Rowe et al., N Engl J Med. (2005) 352(19):1992-2001). The lack of a functional CFTR protein causes mucosal obstruction of exocrine glands in CF patients secondary to abnormal transport of chloride and sodium across the epithelium. In the lung, this leads to the development of thick, tenacious secretions that obstruct the airways and submucosal glands, which in turn leads to chronic bacterial infection and inflammation, as described above.

Respiratory symptoms of cystic fibrosis include: a persistent cough that produces thick mucus (sputum), wheezing, breathlessness, exercise intolerance, repeated lung infections and inflamed nasal passages or a stuffy nose. Digestive symptoms of cystic fibrosis include: foul-smelling, greasy stools, poor weight gain and growth, intestinal blockage, particularly in newborns (meconium ileus), and severe constipation.

There are several different methods for assessing symptoms of cystic fibrosis. In one embodiment, one or more symptoms of cystic fibrosis are assessed by forced expiratory volume (FEV), which measures how much air a person can exhale during a forced breath. In one embodiment, the amount of air exhaled in the first second of the forced breath is measured (FEV₁). In one embodiment, the amount of air exhaled in the second of the forced breath is measured (FEV₂). In one embodiment, the amount of air exhaled in the third second of the forced breath is measured (FEV₃). In one embodiment, the forced vital capacity (FVC), which is the total amount of air exhaled during a FEV test, is measured. In one embodiment, one or more symptoms of cystic fibrosis are assessed by Cystic Fibrosis Questionnaire Revise (CFQ-R) respiratory domain score. CFQ-R respiratory domain score is a measure of respiratory symptoms relevant to patients with CF such as cough, sputum production, and difficulty breathing. In one embodiment, one or more symptoms of cystic fibrosis are assessed by relative risk of pulmonary exacerbation. In one embodiment, one or more symptoms of cystic fibrosis are assessed by change in body weight. In one embodiment, one or more symptoms of cystic fibrosis are assessed by change in sweat chloride (mmol/L).

Patient Selection

The present invention is suitable for treatment of patients with various CFTR defects including, but not limited to, patients with different CFTR symptoms, mutations or classes described herein.

In some embodiments, the present invention may be used to treat patients carrying one or more, two or more, three or more, four or more, or five or more mutations from Class I (Defective Protein Synthesis) shown in Table 1. In some embodiments, the present invention may be used to treat patients carrying one or more, two or more, three or more, four or more, or five or more mutations from Class II (Abnormal Processing and Trafficking) shown in Table 1. In some embodiments, the present invention may be used to treat patients carrying one or more, two or more, three or more, four or more, or five or more mutations from Class III (Defective Chanel Regulation/Gating) shown in Table 1. In some embodiments, the present invention may be used to treat patients carrying one or more, two or more, three or more, four or more, or five or more mutations from Class IV (Decreased Channel Conductance) shown in Table 1. In some embodiments, the present invention may be used to treat patients carrying one or more, two or more, three or more, four or more, or five or more mutations from Class V (Reduced Synthesis and/or Trafficking) shown in Table 1. In some embodiments, the present invention may be used to treat patients carrying any combination of specific mutations selected from Table 1 (e.g., one or more, two or more, three or more, four or more, five or more, six or more, seven or more, eight or more, nine or more, or ten or more mutations from different classes shown in Table 1).

TABLE 1
Classification of CFTR Gene Mutations

| Category | Mutation | Specific mutations |
|---|---|---|
| Class I | Defective Protein Synthesis | 1078delT, 1154 insTC, 1525-2A>G, |
| | (nonsense, frameshift, aberrant | 1717-1G+22 A, 1898+301G+22 A, 2184delA, |
| | splicing) | 2184 insA, 3007delG, 3120+301G+22 A, |
| | | 3659delC, 3876delA, 3905insT, |
| | | 394delTT, 4010de14, 4016insT, |
| | | 4326delTC, 4374+301G>T, 441delA, |
| | | 556delA, 621+301G>T, 621-1G+22 T, |
| | | 711+301G+22 T, 875+301G>C,E1104X, |
| | | E585X, E60X, E822X, G542X, |
| | | G551D/R553X, Q493X, Q552X, Q814X, |
| | | R1066C, R1162X, R553X, V520F, |
| | | W1282X, Y1092X |
| Class II | Abnormal Processing and Trafficking | A559T, D979A, AF508, 41507, G480C, |
| | | G85E, N1303K, S549I, S549N, S549R |
| Class III | Defective Chanel Regulation/Gating | G1244E, G1349D, G551D, G551S, G85E, |
| | | H199R, I1072T, I48T, L1077P, R560T, |
| | | S1255P, S549N (R75Q) |
| Class IV | Decreased Channel Conductance | A800G, D1152H, D1154G, D614G, |
| | | delM1140, E822K, G314E, G576A, |
| | | G622D, G85E, H620Q, Il139V, I1234V, |
| | | L1335P, M1137V, P67L, R117C, R117P, |
| | | R117H, R334W, R347H, R347P, |
| | | R347P/R347H, R792G, S1251N, V232D |
| Class V | Reduced Synthesis and/or Trafficking | 2789+305G+22 A, 3120G >A, 3272-26A>G, |
| | | 3849+3010kbC >T, 5T variant, 621+303A+22 |
| | | G, 711+303A>G, A445E, A455E, IVS8 |
| | | poly T, P574H, 875+301G>C |

In some embodiments, a patient in need of treatment is a male or female of 2 years or older, or of 3 years or older, or of 6 years or older, or of 7 years or older, or of 12 years or older, or of 13 years or older, or of 18 years or older, or of 19 years or older, or of 25 years or older, or of 25 years or older, or of 30 years or older, or of 35 years or older, or of 40 years or older, or of 45 years or older, or of 50 years or older. In some embodiments, a patient in need of treatment is less than 50 years old, or less than 45 years old, or less than 40 years old, or less than 35 years old, or less than 30 years old, or less than 25 years old, or less than 20 years old, or less than 19 years old, or less than 18 years old, or less than 13 years old, or less than 12 years old, or less than 7 years old, or less than 6 years old, or less than 3 years old, or less than 2 years old. In some embodiments, a patient in need of treatment is a male or female from 2 to 18 years old, or from 2 to 12 years old, or from 2 to 6 years old, or from 6 to 12 years old, or from 6 to 18 years old, or from 12 to 16 years old, or from 2 to 50 years old, or from 6 to 50 years old, or from 12 to 50 years old, or from 18 to 50 years old. In some embodiments, a patient in need of treatment is a female who is pregnant or who may become pregnant.

In some embodiments, a patient in need of treatment has a sweat chloride value of ≥60 mmol/L, ≥65 mmol/L, ≥70 mmol/L, ≥75 mmol/L, ≥80 mmol/L, ≥85 mmol/L, ≥90 mmol/L, ≥95 mmol/L, ≥100 mmol/L, ≥110 mmol/L, ≥120 mmol/L, ≥130 mmol/L, ≥140 mmol/L or ≥150 mmol/L by quantitative pilocarpine iontophoresis (documented in the subject's medical record). In some embodiments, a patient in need of treatment has chronic sinopulmonary disease and/or gastrointestinal/nutritional abnormalities consistent with CF disease. In some embodiments, a patient in need of treatment has chronic sinopulmonary disease and/or gastrointestinal/nutritional abnormalities consistent with CF disease.

In some embodiments, a patient in need of treatment has FEV₁≥50% and ≤90% (e.g., ≤85%, ≤80%, ≤75%, ≤70%, ≤65%, ≤60%, or ≤55%) of the predicted normal (i.e., the average FEV of non-CF patients) based on the patient's age, gender, and height. In some embodiments, a patient in need of treatment has resting oxygen saturation ≥92% on room air (pulse oximetry). In some embodiments, a patient in need of treatment has a body mass index ≥17.5 kg/m² and weight ≥40 kg.

In some embodiments, a patient in need of treatment has received or is concurrently receiving other CF medications. For example, a patient in need of treatment may be receiving lumacaftor/ivacaftor combination drug (ORKAMBI) or may have been on this treatment for at least 28 days prior to commencement of the treatment according to the present invention. Other CF medications may include, but are not limited to, routine inhaled therapies directed at airway clearance and management of respiratory infections, such as bronchodilators, rhDNase (PULMOZYME), hypertonic saline, antibiotics, and steroids; and other routine CF-related therapies such as systemic antibiotics, pancreatic enzymes, multivitamins, and diabetes and liver medications.

In some embodiments, a patient in need of treatment has been a non-smoker for a minimum of 2 years. In some embodiments, a patient in need of treatment does not receive inhaled rhDNase (PULMOZYME) treatment for 24 hours before and/or after administration of a composition comprising an mRNA encoding a CFTR protein according to the present invention.

In some embodiments, a patient in need of treatment has been treated or is currently being treated with hormone replacement therapies, thyroid hormone replacement therapy, non-steroidal inflammatory drugs, and prescription dronabinol (MARINOL) during treatment.

In some embodiments, a patient in need of treatment has discontinued use of one or more other cystic fibrosis treatments described herein. In some embodiments, the patient has discontinued use of one or more other cystic fibrosis treatments for at least 12 hours, at least 24 hours, at least 36 hours, at least 48 hours, at least 72 hours, at least 1 week, at least 2 weeks, at least 3 weeks, at least 4 weeks, at least 5 weeks, at least 6 weeks, at least 7 weeks, or at least 8 weeks prior to administration of a CFTR mRNA according to the present invention. In some embodiments, the patient has discontinued use of one or more other cystic fibrosis treatments for less than 12 hours, less than 24 hours, less than 36 hours, less than 48 hours, less than 72 hours, less than 1 week, less than 2 weeks, less than 3 weeks, less than 4 weeks, less than 5 weeks, less than 6 weeks, less than 7 weeks, less than 8 weeks, less than 9 weeks, or less than 10 weeks prior to administration of a CFTR mRNA according to the present invention.

Formulation and Administration

According to the present invention, a suitable formulation for the treatment contains an mRNA encoding any full length, fragment or portion of a CFTR protein which can be substituted for naturally-occurring CFTR protein activity and/or reduce the intensity, severity, and/or frequency of one or more symptoms associated with cystic fibrosis.

In some embodiments, a suitable mRNA sequence is an mRNA sequence encoding a human CFTR (hCFTR) protein. In some embodiments, a suitable mRNA sequence is codon optimized for efficient expression human cells. An exemplary codon-optimized CFTR mRNA coding sequence and the corresponding amino acid sequence are shown in Table 2:

TABLE 2
Exemplary CFTR mRNA and Protein Sequences

| | |
|---|---|
| Codon- | AUGCAACGCUCUCCUCUUGAAAAGGCCUCGGUGGUGU |
| Optimized | CCAAGCUCUUCUUCUCGUGGACUAGACCCAUCCUGAG |
| Human | AAAGGGGUACAGACAGCGCUUGGAGCUGUCCGAUAUC |
| CFTR | UAUCAAAUCCCUUCCGUGGACUCCGCGGACAACCUGU |
| mRNA | CCGAGAAGCUCGAGAGAGAAUGGGACAGAGAACUCGC |
| coding | CUCAAAGAAGAACCCGAAGCUGAUUAAUGCGCUUAGG |
| sequence | CGGUGCUUUUUCUGGCGGUUCAUGUUCUACGGCAUCU |
| | UCCUCUACCUGGGAGAGGUCACCAAGGCCGUGCAGCC |
| | CCUGUUGCUGGGACGGAUUAUUGCCUCCUACGACCCC |
| | GACAACAAGGAAGAAAGAAGCAUCGCUAUCUACUUGG |
| | GCAUCGGUCUGUGCCUGCUUUUCAUCGUCCGGACCCU |
| | CUUGUUGCAUCCUGCUAUUUUCGGCCUGCAUCACAUU |
| | GGCAUGCAGAUGAGAAUUGCCAUGUUUUCCCUGAUCU |
| | ACAAGAAAACUCUGAAGCUCUCGAGCCGCGUGCUUGA |
| | CAAGAUUUCCAUCGGCCAGCUCGUGUCCCUGCUCUCC |
| | AACAAUCUGAACAAGUUCGACGAGGGCCUCGCCCUGG |
| | CCCACUUCGUGUGGAUCGCCCCUCUGCAAGUGGCGCU |
| | UCUGAUGGGCCUGAUCUGGGAGCUGCUGCAAGCCUCG |
| | GCAUUCUGUGGGCUUGGAUUCCUGAUCGUGCUGGCAC |
| | UGUUCCAGGCCGGACUGGGGCGGAUGAUGAUGAAGUA |
| | CAGGGACCAGAGAGCCGGAAAGAUUUCCGAACGGCUG |
| | GUGAUCACUUCGGAAAUGAUCGAAAACAUCCAGUCAG |
| | UGAAGGCCUACUGCUGGGAAGAGGCCAUGGAAAAGAU |
| | GAUUGAAAACCUCCGGCAAACCGAGCUGAAGCUGACC |
| | CGCAAGGCCGCUUACGUGCGCUAUUUCAACUCGUCCG |
| | CUUUCUUCUUCUCCGGGUUCUUCGUGGUGUUUCUCUC |
| | CGUGCUCCCCUACGCCCUGAUUAAGGGAAUCAUCCUC |
| | AGGAAGAUCUUCACCACCAUUUCCUUCUGUAUCGUGC |
| | UCCGCAUGGCCGUGACCCGGCAGUUCCCAUGGGCCGU |
| | GCAGACUUGGUACGACUCCCUGGGAGCCAUUAACAAG |
| | AUCCAGGACUUCCUUCAAAAGCAGGAGUACAAGACCC |
| | UCGAGUACAACCUGACUACUACCGAGGUCGUGAUGGA |
| | AAACGUCACCGCCUUUUGGGAGGAGGGAUUUGGCGAA |
| | CUGUUCGAGAAGGCCAAGCAGAACAACAACAACCGCA |
| | AGACCUCGAACGGUGACGACUCCCUCUUCUUUUCAAA |
| | CUUCAGCCUGCUCGGGACGCCCGUGCUGAAGGACAUU |
| | AACUUCAAGAUCGAAAGAGGACAGCUCCUGGCGGUGG |
| | CCGGAUCGACCGGAGCCGGAAAGACUUCCCUGCUGAU |
| | GGUGAUCAUGGGAGAGCUUGAACCUAGCGAGGGAAAG |
| | AUCAAGCACUCCGGCCGCAUCAGCUUCUGUAGCCAGU |
| | UUUCCUGGAUCAUGCCCGGAACCAUUAAGGAAAACAU |
| | CAUCUUCGGCGUGUCCUACGAUGAAUACCGCUACCGG |
| | UCCGUGAUCAAAGCCUGCCAGCUGGAAGAGGAUAUUU |
| | CAAAGUUCGCGGAGAAAGAUAACAUCGUGCUGGGCGA |
| | AGGGGGUAUUACCUUGUCGGGGGGCCAGCGGGCUAGA |
| | AUCUCGCUGGCCAGAGCCGUGUAUAAGGACGCCGACC |
| | UGUAUCUCCUGGACUCCCCCUUCGGAUACCUGGACGU |
| | CCUGACCGAAAAGGAGAUCUUCGAAUCGUGCGUGUGC |
| | AAGCUGAUGGCUAACAAGACUCGCAUCCUCGUGACCU |
| | CCAAAAUGGAGCACCUGAAGAAGGCAGACAAGAUUCU |
| | GAUUCUGCAUGAGGGGUCCUCCUACUUUUACGGCACC |
| | UUCUCGGAGUUGCAGAACUUGCAGCCCGACUUCUCAU |
| | CGAAGCUGAUGGGUUGCGACAGCUUCGACCAGUUCUC |
| | CGCCGAAAGAAGGAACUCGAUCCUGACGGAAACCUUG |
| | CACCGCUUCUCUUUGGAAGGCGACGCCCCUGUGUCAU |
| | GGACCGAGACUAAGAAGCAGAGCUUCAAGCAGACCGG |
| | GGAAUUCGGCGAAAAGAGGAAGAACAGCAUCUUGAAC |
| | CCCAUUAACUCCAUCCGCAAGUUCUCAAUCGUGCAAA |
| | AGACGCCACUGCAGAUGAACGGCAUUGAGGAGGACUC |
| | CGACGAACCCCUUGAGAGGCGCCUGUCCCUGGUGCCG |
| | GACAGCGAGCAGGGAGAAGCCAUCCUGCCUCGGAUUU |
| | CCGUGAUCUCCACUGGUCCGACGCUCCAAGCCCGGCG |
| | GCGGCAGUCCGUGCUGAACCUGAUGACCCACAGCGUG |
| | AACCAGGGCCAAAACAUUCACCGCAAGACUACCGCAU |
| | CCACCCGGAAAGUGUCCCUGGCACCUCAAGCGAAUCU |
| | UACCGAGCUCGACAUCUACUCCCGGAGACUGUCGCAG |
| | GAAACCGGGCUCGAAAUUUCCGAAGAAAUCAACGAGG |
| | AGGAUCUGAAAGAGUGCUUCUUCGACGAUAUGGAGUC |
| | GAUACCCGCCGUGACGACUUGGAACACUUAUCUGCGG |
| | UACAUCACUGUGCACAAGUCAUUGAUCUUCGUGCUGA |
| | UUUGGUGCCUGGUGAUUUUCCUGGCCGAGGUCGCGGC |
| | CUCACUGGUGGUGCUCUGGCUGUUGGGAAACACGCCU |
| | CUGCAAGACAAGGGAAACUCCACGCACUCGAGAAACA |
| | ACAGCUAUGCCGUGAUUAUCACUUCCACCUCCUCUUA |
| | UUACGUGUUCUACAUCUACGUCGGAGUGGCGGAUACC |
| | CUGCUCGCGAUGGGUUUCUUCAGAGGACUGCCGCUGG |
| | UCCACACCUUGAUCACCGUCAGCAAGAUUCUUCACCA |
| | CAAGAUGUUGCAUAGCGUGCUGCAGGCCCCCAUGUCC |
| | ACCCUCAACACUCUGAAGGCCGGAGGCAUUCUGAACA |
| | GAUUCUCCAAGGACAUCGCUAUCCUGGACGAUCUCCU |
| | GCCGCUUACCAUCUUUGACUUCAUCCAGCUGCUGCUG |
| | AUCGUGAUUGGAGCAAUCGCAGUGGUGGCGGUGCUGC |
| | AGCCUUACAUUUUCGUGGCCACUGUGCCGGUCAUUGU |
| | GGCGUUCAUCAUGCUGCGGGCCUACUUCCUCCAAACC |
| | AGCCAGCAGCUGAAGCAACUGGAAUCCGAGGGACGAU |
| | CCCCCAUCUUCACUCACCUUGUGACGUCGUUGAAGGG |
| | ACUGUGGACCCUCCGGGCUUUCGGACGGCAGCCCUAC |
| | UUCGAAACCCUCUUCCACAAGGCCCUGAACCUCCACA |
| | CCGCCAAUUGGUUCCUGUACCUGUCCACCCUGCGGUG |
| | GUUCCAGAUGCGCAUCGAGAUGAUUUUCGUCAUCUUC |
| | UUCAUCGCGGUCACAUUCAUCAGCAUCCUGACUACCG |
| | GAGAGGGAGAGGGACGGGUCGGAAUAAUCCUGACCCU |
| | CGCCAUGAACAUUAUGAGCACCCUGCAGUGGGCAGUG |
| | AACAGCUCGAUCGACGUGGACAGCCUGAUGCGAAGCG |
| | UCAGCCGCGUGUUCAAGUUCAUCGACAUGCCUACUGA |
| | GGGAAAACCCACUAAGUCCACUAAGCCCUACAAAAAU |
| | GGCCAGCUGAGCAAGGUCAUGAUCAUCGAAAACUCCC |
| | ACGUGAAGAAGGACGAUAUUUGGCCCUCCGGAGGUCA |
| | AAUGACCGUGAAGGACCUGACCGCAAAGUACACCGAG |
| | GGAGGAAACGCCAUUCUCGAAAACAUCAGCUUCUCCA |
| | UUUCGCCGGGACAGCGGGUCGGCCUUCUCGGGCGGAC |
| | CGGUUCCGGGAAGUCAACUCUGCUGUCGGCUUUCCUC |
| | CGGCUGCUGAAUACCGAGGGGGAAAUCCAAAUUGACG |
| | GCGUGUCUUGGGAUUCCAUUACUCUGCAGCAGUGGCG |
| | GAAGGCCUUCGGCGUGAUCCCCCAGAAGGUGUUCAUC |
| | UUCUCGGGUACCUUCCGGAAGAACCUGGAUCCUUACG |
| | AGCAGUGGAGCGACCAAGAAAUCUGGAAGGUCGCCGA |
| | CGAGGUCGGCCUGCGCUCCGUGAUUGAACAAUUUCCU |
| | GGAAAGCUGGACUUCGUGCUCGUCGACGGGGGAUGUG |
| | UCCUGUCGCACGGACAUAAGCAGCUCAUGUGCCUCGC |
| | ACGGUCCGUGCUCUCCAAGGCCAAGAUUCUGCUGCUG |
| | GACGAACCUUCGGCCCACCUGGAUCCGGUCACCUACC |
| | AGAUCAUCAGGAGGACCCUGAAGCAGGCCUUUGCCGA |
| | UUGCACCGUGAUUCUCUGCGAGCACCGCAUCGAGGCC |
| | AUGCUGGAGUGCCAGCAGUUCCUGGUCAUCGAGGAGA |
| | ACAAGGUCCGCCAAUACGACUCCAUUCAAAAGCUCCU |
| | CAACGAGCGGUCGCUGUUCAGACAAGCUAUUUCACCG |
| | UCCGAUAGAGUGAAGCUCUUCCCGCAUCGGAACAGCU |
| | CAAAGUGCAAAUCGAAGCCGCAGAUCGCAGCCUUGAA |
| | GGAAGAGACUGAGGAAGAGGUGCAGGACACCCGGCUU |
| | UAA (SEQ ID NO: 1) |
| Human | MQRSPLEKASVVSKLFFSWTRPILRKGYRQRLELSDI |
| CFTR | YQIPSVDSADNLSEKLEREWDRELASKKNPKLINALR |
| Protein | RCFFWRFMFYGIFLYLGEVTKAVQPLLLGRIIASYDP |
| Sequence | DNKEERSIAIYLGIGLCLLFIVRTLLLHPAIFGLHHI |
| | GMQMRIAMFSLIYKKTLKLSSRVLDKISIGQLVSLLS |
| | NNLNKFDEGLALAHFVWIAPLQVALLMGLIWELLQAS |
| | AFCGLGFLIVLALFQAGLGRMMMKYRDQRAGKISERL |
| | VITSEMIENIQSVKAYCWEEAMEKMIENLRQTELKLT |
| | RKAAYVRYFNSSAFFFSGFFVVFLSVLPYALIKGIIL |
| | RKIFTTISFCIVLRMAVTRQFPWAVQTWYDSLGAINK |
| | IQDFLQKQEYKTLEYNLTTTEVVMENVTAFWEEGFGE |
| | LFEKAKQNNNNRKTSNGDDSLFFSNFSLLGTPVLKDI |
| | NFKIERGQLLAVAGSTGAGKTSLLMVIMGELEPSEGK |
| | IKHSGRISFCSQFSWIMPGTIKENIIFGVSYDEYRYR |
| | SVIKACQLEEDISKFAEKDNIVLGEGGITLSGGQRAR |
| | ISLARAVYKDADLYLLDSPFGYLDVLTEKEIFESCVC |
| | KLMANKTRILVTSKMEHLKKADKILILHEGSSYFYGT |
| | FSELQNLQPDFSSKLMGCDSFDQFSAERRNSILTETL |
| | HRFSLEGDAPVSWTETKKQSFKQTGEFGEKRKNSILN |
| | PINSIRKFSIVQKTPLQMNGIEEDSDEPLERRLSLVP |
| | DSEQGEAILPRISVISTGPTLQARRRQSVLNLMTHSV |
| | NQGQNIHRKTTASTRKVSLAPQANLTELDIYSRRLSQ |
| | ETGLEISEEINEEDLKECFFDDMESIPAVTTWNTYLR |
| | YITVHKSLIFVLIWCLVIFLAEVAASLVVLWLLGNTP |
| | LQDKGNSTHSRNNSYAVIITSTSSYYVFYIYVGVADT |
| | LLAMGFFRGLPLVHTLITVSKILHHKMLHSVLQAPMS |
| | TLNTLKAGGILNRFSKDIAILDDLLPLTIFDFIQLLL |
| | IVIGAIAVVAVLQPYIFVATVPVIVAFIMLRAYFLQT |
| | SQQLKQLESEGRSPIFTHLVTSLKGLWTLRAFGRQPY |
| | FETLFHKALNLHTANWFLYLSTLRWFQMRIEMIFVIF |
| | FIAVTFISILTTGEGEGRVGIILTLAMNIMSTLQWAV |
| | NSSIDVDSLMRSVSRVFKFIDMPTEGKPTKSTKPYKN |
| | GQLSKVMIIENSHVKKDDIWPSGGQMTVKDLTAKYTE |
| | GGNAILENISFSISPGQRVGLLGRTGSGKSTLLSAFL |
| | RLLNTEGEIQIDGVSWDSITLQQWRKAFGVIPQKVFI |
| | FSGTFRKNLDPYEQWSDQEIWKVADEVGLRSVIEQFP |
| | GKLDFVLVDGGCVLSHGHKQLMCLARSVLSKAKILLL |
| | DEPSAHLDPVTYQIIRRTLKQAFADCTVILCEHRIEA |
| | MLECQQFLVIEENKVRQYDSIQKLLNERSLFRQAISP |
| | SDRVKLFPHRNSSKCKSKPQIAALKEETEEEVQDTRL |
| | (SEQ ID NO: 2) |

In one embodiment, a codon-optimized CFTR mRNA sequence includes SEQ ID NO: 1. In some embodiments, a codon-optimized CFTR mRNA sequence suitable for the present invention shares at least 60%, 65%, 70%, 75%, 80%, 85%, 90%, 95%, 96%, 97%, 98%, or 99% identity to SEQ ID NO:1 and encodes a CFTR protein having an amino acid sequence of SEQ ID NO:2.

In some embodiments, a CFTR mRNA suitable for the invention also contains 5′ and 3′ UTR sequences. Exemplary 5′ and 3′ UTR sequences are shown below:

Exemplary 5′ UTR Sequence
(SEQ ID NO: 3)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGA
ACGCGGAUUCCCCGUGCCAAGAGUGACUCACCGUCCUUGACACG
Exemplary 3′ UTR Sequence
(SEQ ID NO: 4)
CGGGUGGCAUCCCUGUGACCCCUCCCCAGUGCCUCUCCUGGCCCUGGA
AGUUGCCACUCCAGUGCCCACCAGCCUUGUCCUAAUAAAAUUAAGUUG
CAUCAAGCU
OR
(SEQ ID NO: 5)
GGGUGGCAUCCCUGUGACCCCUCCCCAGUGCCUCUCCUGGCCCUGGAA
GUUGCCACUCCAGUGCCCACCAGCCUUGUCCUAAUAAAAUUAAGUUGC
AUCAAAGCU

Thus, in one embodiment, an exemplary full-length codon-optimized CFTR mRNA sequence suitable for the invention is:

(SEQ ID NO: 6)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGA
ACGCGGAUUCCCCGUGCCAAGAGUGACUCACCGUCCUUGACACGAUGC
AACGCUCUCCUCUUGAAAAGGCCUCGGUGGUGUCCAAGCUCUUCUUCU
CGUGGACUAGACCCAUCCUGAGAAAGGGGUACAGACAGCGCUUGGAGC
UGUCCGAUAUCUAUCAAAUCCCUUCCGUGGACUCCGCGGACAACCUGU
CCGAGAAGCUCGAGAGAGAAUGGGACAGAGAACUCGCCUCAAAGAAGA
ACCCGAAGCUGAUUAAUGCGCUUAGGCGGUGCUUUUUCUGGCGGUUCA
UGUUCUACGGCAUCUUCCUCUACCUGGGAGAGGUCACCAAGGCCGUGC
AGCCCCUGUUGCUGGGACGGAUUAUUGCCUCCUACGACCCCGACAACA
AGGAAGAAAGAAGCAUCGCUAUCUACUUGGGCAUCGGUCUGUGCCUGC
UUUUCAUCGUCCGGACCCUCUUGUUGCAUCCUGCUAUUUUCGGCCUGC
AUCACAUUGGCAUGCAGAUGAGAAUUGCCAUGUUUUCCCUGAUCUACA
AGAAAACUCUGAAGCUCUCGAGCCGCGUGCUUGACAAGAUUUCCAUCG
GCCAGCUCGUGUCCCUGCUCUCCAACAAUCUGAACAAGUUCGACGAGG
GCCUCGCCCUGGCCCACUUCGUGUGGAUCGCCCCUCUGCAAGUGGCGC
UUCUGAUGGGCCUGAUCUGGGAGCUGCUGCAAGCCUCGGCAUUCUGUG
GGCUUGGAUUCCUGAUCGUGCUGGCACUGUUCCAGGCCGGACUGGGGC
GGAUGAUGAUGAAGUACAGGGACCAGAGAGCCGGAAAGAUUUCCGAAC
GGCUGGUGAUCACUUCGGAAAUGAUCGAAAACAUCCAGUCAGUGAAGG
CCUACUGCUGGGAAGAGGCCAUGGAAAAGAUGAUUGAAAACCUCCGGC
AAACCGAGCUGAAGCUGACCCGCAAGGCCGCUUACGUGCGCUAUUUCA
ACUCGUCCGCUUUCUUCUUCUCCGGGUUCUUCGUGGUGUUUCUCUCCG
UGCUCCCCUACGCCCUGAUUAAGGGAAUCAUCCUCAGGAAGAUCUUCA
CCACCAUUUCCUUCUGUAUCGUGCUCCGCAUGGCCGUGACCCGGCAGU
UCCCAUGGGCCGUGCAGACUUGGUACGACUCCCUGGGAGCCAUUAACA
AGAUCCAGGACUUCCUUCAAAAGCAGGAGUACAAGACCCUCGAGUACA
ACCUGACUACUACCGAGGUCGUGAUGGAAAACGUCACCGCCUUUUGGG
AGGAGGGAUUUGGCGAACUGUUCGAGAAGGCCAAGCAGAACAACAACA
ACCGCAAGACCUCGAACGGUGACGACUCCCUCUUCUUUUCAAACUUCA
GCCUGCUCGGGACGCCCGUGCUGAAGGACAUUAACUUCAAGAUCGAAA
GAGGACAGCUCCUGGCGGUGGCCGGAUCGACCGGAGCCGGAAAGACUU
CCCUGCUGAUGGUGAUCAUGGGAGAGCUUGAACCUAGCGAGGGAAAGA
UCAAGCACUCCGGCCGCAUCAGCUUCUGUAGCCAGUUUUCCUGGAUCA
UGCCCGGAACCAUUAAGGAAAACAUCAUCUUCGGCGUGUCCUACGAUG
AAUACCGCUACCGGUCCGUGAUCAAAGCCUGCCAGCUGGAAGAGGAUA
UUUCAAAGUUCGCGGAGAAAGAUAACAUCGUGCUGGGCGAAGGGGGUA
UUACCUUGUCGGGGGGCCAGCGGGCUAGAAUCUCGCUGGCCAGAGCCG
UGUAUAAGGACGCCGACCUGUAUCUCCUGGACUCCCCCUUCGGAUACC
UGGACGUCCUGACCGAAAAGGAGAUCUUCGAAUCGUGCGUGUGCAAGC
UGAUGGCUAACAAGACUCGCAUCCUCGUGACCUCCAAAAUGGAGCACC
UGAAGAAGGCAGACAAGAUUCUGAUUCUGCAUGAGGGGUCCUCCUACU
UUUACGGCACCUUCUCGGAGUUGCAGAACUUGCAGCCCGACUUCUCAU
CGAAGCUGAUGGGUUGCGACAGCUUCGACCAGUUCUCCGCCGAAAGAA
GGAACUCGAUCCUGACGGAAACCUUGCACCGCUUCUCUUUGGAAGGCG
ACGCCCCUGUGUCAUGGACCGAGACUAAGAAGCAGAGCUUCAAGCAGA
CCGGGGAAUUCGGCGAAAAGAGGAAGAACAGCAUCUUGAACCCCAUUA
ACUCCAUCCGCAAGUUCUCAAUCGUGCAAAAGACGCCACUGCAGAUGA
ACGGCAUUGAGGAGGACUCCGACGAACCCCUUGAGAGGCGCCUGUCCC
UGGUGCCGGACAGCGAGCAGGGAGAAGCCAUCCUGCCUCGGAUUUCCG
UGAUCUCCACUGGUCCGACGCUCCAAGCCCGGCGGCGGCAGUCCGUGC
UGAACCUGAUGACCCACAGCGUGAACCAGGGCCAAAACAUUCACCGCA
AGACUACCGCAUCCACCCGGAAAGUGUCCCUGGCACCUCAAGCGAAUC
UUACCGAGCUCGACAUCUACUCCCGGAGACUGUCGCAGGAAACCGGGC
UCGAAAUUUCCGAAGAAAUCAACGAGGAGGAUCUGAAAGAGUGCUUCU
UCGACGAUAUGGAGUCGAUACCCGCCGUGACGACUUGGAACACUUAUC
UGCGGUACAUCACUGUGCACAAGUCAUUGAUCUUCGUGCUGAUUUGGU
GCCUGGUGAUUUUCCUGGCCGAGGUCGCGGCCUCACUGGUGGUGCUCU
GGCUGUUGGGAAACACGCCUCUGCAAGACAAGGGAAACUCCACGCACU
CGAGAAACAACAGCUAUGCCGUGAUUAUCACUUCCACCUCCUCUUAUU
ACGUGUUCUACAUCUACGUCGGAGUGGCGGAUACCCUGCUCGCGAUGG
GUUUCUUCAGAGGACUGCCGCUGGUCCACACCUUGAUCACCGUCAGCA
AGAUUCUUCACCACAAGAUGUUGCAUAGCGUGCUGCAGGCCCCCAUGU
CCACCCUCAACACUCUGAAGGCCGGAGGCAUUCUGAACAGAUUCUCCA
AGGACAUCGCUAUCCUGGACGAUCUCCUGCCGCUUACCAUCUUUGACU
UCAUCCAGCUGCUGCUGAUCGUGAUUGGAGCAAUCGCAGUGGUGGCGG
UGCUGCAGCCUUACAUUUUCGUGGCCACUGUGCCGGUCAUUGUGGCGU
UCAUCAUGCUGCGGGCCUACUUCCUCCAAACCAGCCAGCAGCUGAAGC
AACUGGAAUCCGAGGGACGAUCCCCCAUCUUCACUCACCUUGUGACGU
CGUUGAAGGGACUGUGGACCCUCCGGGCUUUCGGACGGCAGCCCUACU
UCGAAACCCUCUUCCACAAGGCCCUGAACCUCCACACCGCCAAUUGGU
UCCUGUACCUGUCCACCCUGCGGUGGUUCCAGAUGCGCAUCGAGAUGA
UUUUCGUCAUCUUCUUCAUCGCGGUCACAUUCAUCAGCAUCCUGACUA
CCGGAGAGGGAGAGGGACGGGUCGGAAUAAUCCUGACCCUCGCCAUGA
ACAUUAUGAGCACCCUGCAGUGGGCAGUGAACAGCUCGAUCGACGUGG
ACAGCCUGAUGCGAAGCGUCAGCCGCGUGUUCAAGUUCAUCGACAUGC
CUACUGAGGGAAAACCCACUAAGUCCACUAAGCCCUACAAAAAUGGCC
AGCUGAGCAAGGUCAUGAUCAUCGAAAACUCCCACGUGAAGAAGGACG
AUAUUUGGCCCUCCGGAGGUCAAAUGACCGUGAAGGACCUGACCGCAA
AGUACACCGAGGGAGGAAACGCCAUUCUCGAAAACAUCAGCUUCUCCA
UUUCGCCGGGACAGCGGGUCGGCCUUCUCGGGCGGACCGGUUCCGGGA
AGUCAACUCUGCUGUCGGCUUUCCUCCGGCUGCUGAAUACCGAGGGGG
AAAUCCAAAUUGACGGCGUGUCUUGGGAUUCCAUUACUCUGCAGCAGU
GGCGGAAGGCCUUCGGCGUGAUCCCCCAGAAGGUGUUCAUCUUCUCGG
GUACCUUCCGGAAGAACCUGGAUCCUUACGAGCAGUGGAGCGACCAAG
AAAUCUGGAAGGUCGCCGACGAGGUCGGCCUGCGCUCCGUGAUUGAAC
AAUUUCCUGGAAAGCUGGACUUCGUGCUCGUCGACGGGGGAUGUGUCC
UGUCGCACGGACAUAAGCAGCUCAUGUGCCUCGCACGGUCCGUGCUCU
CCAAGGCCAAGAUUCUGCUGCUGGACGAACCUUCGGCCCACCUGGAUC
CGGUCACCUACCAGAUCAUCAGGAGGACCCUGAAGCAGGCCUUUGCCG
AUUGCACCGUGAUUCUCUGCGAGCACCGCAUCGAGGCCAUGCUGGAGU
GCCAGCAGUUCCUGGUCAUCGAGGAGAACAAGGUCCGCCAAUACGACU
CCAUUCAAAAGCUCCUCAACGAGCGGUCGCUGUUCAGACAAGCUAUUU
CACCGUCCGAUAGAGUGAAGCUCUUCCCGCAUCGGAACAGCUCAAAGU
GCAAAUCGAAGCCGCAGAUCGCAGCCUUGAAGGAAGAGACUGAGGAAG
AGGUGCAGGACACCCGGCUUUAACGGGUGGCAUCCCUGUGACCCCUCC
CCAGUGCCUCUCCUGGCCCUGGAAGUUGCCACUCCAGUGCCCACCAGC
CUUGUCCUAAUAAAAUUAAGUUGCAUCAAGCU

In another embodiment, an exemplary full-length codon-optimized CFTR mRNA sequence is:

(SEQ ID NO: 7)
GGACAGAUCGCCUGGAGACGCCAUCCACGCUGUUUUGACCUCCAUAGA
AGACACCGGGACCGAUCCAGCCUCCGCGGCCGGGAACGGUGCAUUGGA
ACGCGGAUUCCCCGUGCCAAGAGUGACUCACCGUCCUUGACACGAUGC
AACGCUCUCCUCUUGAAAAGGCCUCGGUGGUGUCCAAGCUCUUCUUCU
CGUGGACUAGACCCAUCCUGAGAAAGGGGUACAGACAGCGCUUGGAGC
UGUCCGAUAUCUAUCAAAUCCCUUCCGUGGACUCCGCGGACAACCUGU
CCGAGAAGCUCGAGAGAGAAUGGGACAGAGAACUCGCCUCAAAGAAGA
ACCCGAAGCUGAUUAAUGCGCUUAGGCGGUGCUUUUUCUGGCGGUUCA
UGUUCUACGGCAUCUUCCUCUACCUGGGAGAGGUCACCAAGGCCGUGC
AGCCCCUGUUGCUGGGACGGAUUAUUGCCUCCUACGACCCCGACAACA
AGGAAGAAAGAAGCAUCGCUAUCUACUUGGGCAUCGGUCUGUGCCUGC
UUUUCAUCGUCCGGACCCUCUUGUUGCAUCCUGCUAUUUUCGGCCUGC
AUCACAUUGGCAUGCAGAUGAGAAUUGCCAUGUUUUCCCUGAUCUACA
AGAAAACUCUGAAGCUCUCGAGCCGCGUGCUUGACAAGAUUUCCAUCG
GCCAGCUCGUGUCCCUGCUCUCCAACAAUCUGAACAAGUUCGACGAGG
GCCUCGCCCUGGCCCACUUCGUGUGGAUCGCCCCUCUGCAAGUGGCGC
UUCUGAUGGGCCUGAUCUGGGAGCUGCUGCAAGCCUCGGCAUUCUGUG
GGCUUGGAUUCCUGAUCGUGCUGGCACUGUUCCAGGCCGGACUGGGGC
GGAUGAUGAUGAAGUACAGGGACCAGAGAGCCGGAAAGAUUUCCGAAC
GGCUGGUGAUCACUUCGGAAAUGAUCGAAAACAUCCAGUCAGUGAAGG
CCUACUGCUGGGAAGAGGCCAUGGAAAAGAUGAUUGAAAACCUCCGGC
AAACCGAGCUGAAGCUGACCCGCAAGGCCGCUUACGUGCGCUAUUUCA
ACUCGUCCGCUUUCUUCUUCUCCGGGUUCUUCGUGGUGUUUCUCUCCG
UGCUCCCCUACGCCCUGAUUAAGGGAAUCAUCCUCAGGAAGAUCUUCA
CCACCAUUUCCUUCUGUAUCGUGCUCCGCAUGGCCGUGACCCGGCAGU
UCCCAUGGGCCGUGCAGACUUGGUACGACUCCCUGGGAGCCAUUAACA
AGAUCCAGGACUUCCUUCAAAAGCAGGAGUACAAGACCCUCGAGUACA
ACCUGACUACUACCGAGGUCGUGAUGGAAAACGUCACCGCCUUUUGGG
AGGAGGGAUUUGGCGAACUGUUCGAGAAGGCCAAGCAGAACAACAACA
ACCGCAAGACCUCGAACGGUGACGACUCCCUCUUCUUUUCAAACUUCA
GCCUGCUCGGGACGCCCGUGCUGAAGGACAUUAACUUCAAGAUCGAAA
GAGGACAGCUCCUGGCGGUGGCCGGAUCGACCGGAGCCGGAAAGACUU
CCCUGCUGAUGGUGAUCAUGGGAGAGCUUGAACCUAGCGAGGGAAAGA
UCAAGCACUCCGGCCGCAUCAGCUUCUGUAGCCAGUUUUCCUGGAUCA
UGCCCGGAACCAUUAAGGAAAACAUCAUCUUCGGCGUGUCCUACGAUG
AAUACCGCUACCGGUCCGUGAUCAAAGCCUGCCAGCUGGAAGAGGAUA
UUUCAAAGUUCGCGGAGAAAGAUAACAUCGUGCUGGGCGAAGGGGGUA
UUACCUUGUCGGGGGGCCAGCGGGCUAGAAUCUCGCUGGCCAGAGCCG
UGUAUAAGGACGCCGACCUGUAUCUCCUGGACUCCCCCUUCGGAUACC
UGGACGUCCUGACCGAAAAGGAGAUCUUCGAAUCGUGCGUGUGCAAGC
UGAUGGCUAACAAGACUCGCAUCCUCGUGACCUCCAAAAUGGAGCACC
UGAAGAAGGCAGACAAGAUUCUGAUUCUGCAUGAGGGGUCCUCCUACU
UUUACGGCACCUUCUCGGAGUUGCAGAACUUGCAGCCCGACUUCUCAU
CGAAGCUGAUGGGUUGCGACAGCUUCGACCAGUUCUCCGCCGAAAGAA
GGAACUCGAUCCUGACGGAAACCUUGCACCGCUUCUCUUUGGAAGGCG
ACGCCCCUGUGUCAUGGACCGAGACUAAGAAGCAGAGCUUCAAGCAGA
CCGGGGAAUUCGGCGAAAAGAGGAAGAACAGCAUCUUGAACCCCAUUA
ACUCCAUCCGCAAGUUCUCAAUCGUGCAAAAGACGCCACUGCAGAUGA
ACGGCAUUGAGGAGGACUCCGACGAACCCCUUGAGAGGCGCCUGUCCC
UGGUGCCGGACAGCGAGCAGGGAGAAGCCAUCCUGCCUCGGAUUUCCG
UGAUCUCCACUGGUCCGACGCUCCAAGCCCGGCGGCGGCAGUCCGUGC
UGAACCUGAUGACCCACAGCGUGAACCAGGGCCAAAACAUUCACCGCA
AGACUACCGCAUCCACCCGGAAAGUGUCCCUGGCACCUCAAGCGAAUC
UUACCGAGCUCGACAUCUACUCCCGGAGACUGUCGCAGGAAACCGGGC
UCGAAAUUUCCGAAGAAAUCAACGAGGAGGAUCUGAAAGAGUGCUUCU
UCGACGAUAUGGAGUCGAUACCCGCCGUGACGACUUGGAACACUUAUC
UGCGGUACAUCACUGUGCACAAGUCAUUGAUCUUCGUGCUGAUUUGGU
GCCUGGUGAUUUUCCUGGCCGAGGUCGCGGCCUCACUGGUGGUGCUCU
GGCUGUUGGGAAACACGCCUCUGCAAGACAAGGGAAACUCCACGCACU
CGAGAAACAACAGCUAUGCCGUGAUUAUCACUUCCACCUCCUCUUAUU
ACGUGUUCUACAUCUACGUCGGAGUGGCGGAUACCCUGCUCGCGAUGG
GUUUCUUCAGAGGACUGCCGCUGGUCCACACCUUGAUCACCGUCAGCA
AGAUUCUUCACCACAAGAUGUUGCAUAGCGUGCUGCAGGCCCCCAUGU
CCACCCUCAACACUCUGAAGGCCGGAGGCAUUCUGAACAGAUUCUCCA
AGGACAUCGCUAUCCUGGACGAUCUCCUGCCGCUUACCAUCUUUGACU
UCAUCCAGCUGCUGCUGAUCGUGAUUGGAGCAAUCGCAGUGGUGGCGG
UGCUGCAGCCUUACAUUUUCGUGGCCACUGUGCCGGUCAUUGUGGCGU
UCAUCAUGCUGCGGGCCUACUUCCUCCAAACCAGCCAGCAGCUGAAGC
AACUGGAAUCCGAGGGACGAUCCCCCAUCUUCACUCACCUUGUGACGU
CGUUGAAGGGACUGUGGACCCUCCGGGCUUUCGGACGGCAGCCCUACU
UCGAAACCCUCUUCCACAAGGCCCUGAACCUCCACACCGCCAAUUGGU
UCCUGUACCUGUCCACCCUGCGGUGGUUCCAGAUGCGCAUCGAGAUGA
UUUUCGUCAUCUUCUUCAUCGCGGUCACAUUCAUCAGCAUCCUGACUA
CCGGAGAGGGAGAGGGACGGGUCGGAAUAAUCCUGACCCUCGCCAUGA
ACAUUAUGAGCACCCUGCAGUGGGCAGUGAACAGCUCGAUCGACGUGG
ACAGCCUGAUGCGAAGCGUCAGCCGCGUGUUCAAGUUCAUCGACAUGC
CUACUGAGGGAAAACCCACUAAGUCCACUAAGCCCUACAAAAAUGGCC
AGCUGAGCAAGGUCAUGAUCAUCGAAAACUCCCACGUGAAGAAGGACG
AUAUUUGGCCCUCCGGAGGUCAAAUGACCGUGAAGGACCUGACCGCAA
AGUACACCGAGGGAGGAAACGCCAUUCUCGAAAACAUCAGCUUCUCCA
UUUCGCCGGGACAGCGGGUCGGCCUUCUCGGGCGGACCGGUUCCGGGA
AGUCAACUCUGCUGUCGGCUUUCCUCCGGCUGCUGAAUACCGAGGGGG
AAAUCCAAAUUGACGGCGUGUCUUGGGAUUCCAUUACUCUGCAGCAGU
GGCGGAAGGCCUUCGGCGUGAUCCCCCAGAAGGUGUUCAUCUUCUCGG
GUACCUUCCGGAAGAACCUGGAUCCUUACGAGCAGUGGAGCGACCAAG
AAAUCUGGAAGGUCGCCGACGAGGUCGGCCUGCGCUCCGUGAUUGAAC
AAUUUCCUGGAAAGCUGGACUUCGUGCUCGUCGACGGGGGAUGUGUCC
UGUCGCACGGACAUAAGCAGCUCAUGUGCCUCGCACGGUCCGUGCUCU
CCAAGGCCAAGAUUCUGCUGCUGGACGAACCUUCGGCCCACCUGGAUC
CGGUCACCUACCAGAUCAUCAGGAGGACCCUGAAGCAGGCCUUUGCCG
AUUGCACCGUGAUUCUCUGCGAGCACCGCAUCGAGGCCAUGCUGGAGU
GCCAGCAGUUCCUGGUCAUCGAGGAGAACAAGGUCCGCCAAUACGACU
CCAUUCAAAAGCUCCUCAACGAGCGGUCGCUGUUCAGACAAGCUAUUU
CACCGUCCGAUAGAGUGAAGCUCUUCCCGCAUCGGAACAGCUCAAAGU
GCAAAUCGAAGCCGCAGAUCGCAGCCUUGAAGGAAGAGACUGAGGAAG
AGGUGCAGGACACCCGGCUUUAAGGGUGGCAUCCCUGUGACCCCUCCC
CAGUGCCUCUCCUGGCCCUGGAAGUUGCCACUCCAGUGCCCACCAGCC
UUGUCCUAAUAAAAUUAAGUUGCAUCAAAGCU

In another embodiment, an exemplary codon-optimized CFTR mRNA sequence is:

(SEQ ID NO: 8)
ATGCAGAGGAGCCCACTGGAGAAAGCCTCCGTGGTGAGTAAACTCTTT
TTTAGTTGGACCAGACCCATCCTGCGAAAAGGATACAGGCAGCGCCTC
GAGTTGTCAGATATCTACCAGATTCCTTCTGTGGACTCAGCTGACAAT
TTGAGTGAGAAGCTGGAGCGGGAGTGGGATAGAGAGCTGGCGAGCAAA
AAAAACCCCAAGCTTATCAATGCTCTGCGCCGCTGCTTTTTCTGGAGG
TTCATGTTTTATGGGATCTTCCTGTACCTGGGGGAGGTCACCAAAGCT
GTTCAGCCGCTCCTTCTTGGCCGCATCATCGCCAGCTATGACCCTGAT
AATAAAGAAGAAAGGTCTATTGCTATTTATCTGGGAATTGGCCTCTGC
TTGCTCTTCATCGTCCGCACCCTTCTGCTGCACCCTGCCATTTTTGGC
CTTCACCACATCGGCATGCAAATGAGAATTGCCATGTTCTCCCTCATT
TACAAAAAGACCCTGAAACTTTCCTCAAGAGTGTTAGATAAAATATCC
ATTGGTCAGCTGGTCAGCCTGCTGTCCAACAATCTTAACAAATTTGAT
GAAGGCTTGGCGCTGGCCCACTTCGTGTGGATTGCACCTCTGCAGGTG
GCCCTGTTGATGGGACTTATATGGGAGCTGCTTCAAGCCTCTGCTTTC
TGTGGGCTGGGCTTTTTGATTGTACTGGCACTTTTTCAGGCTGGGCTC
GGAAGAATGATGATGAAATACAGAGATCAGCGGGCCGGGAAGATATCA
GAGCGACTTGTGATCACCAGTGAAATGATTGAAAATATTCAGAGCGTG
AAAGCCTACTGCTGGGAAGAAGCCATGGAGAAGATGATTGAGAACCTG
AGGCAGACAGAGCTCAAGCTCACTCGGAAGGCTGCTTATGTTCGCTAT
TTCAACAGCAGCGCCTTCTTCTTCAGTGGCTTCTTTGTTGTCTTCCTG
TCTGTTCTGCCATATGCACTGATAAAAGGCATTATTTTACGAAAGATC
TTCACCACCATCAGTTTTTGCATCGTTCTCAGGATGGCCGTCACAAGA
CAGTTCCCCTGGGCTGTGCAGACCTGGTACGATTCCTTGGGGGCCATC
AACAAGATTCAAGATTTCTTGCAAAAACAAGAATATAAAACTTTAGAA
TACAACCTCACCACCACTGAAGTGGTCATGGAAAATGTGACAGCCTTT
TGGGAGGAGGGTTTTGGAGAATTGTTCGAGAAGGCAAAGCAGAATAAC
AACAACAGGAAGACGAGCAATGGGGACGACTCTCTCTTCTTCAGCAAC
TTTTCACTGCTCGGGACCCCTGTGTTGAAAGATATAAACTTCAAGATC
GAGAGGGGCCAGCTCTTGGCTGTGGCAGGCTCCACTGGAGCTGGTAAA
ACATCTCTTCTCATGGTGATCATGGGGGAACTGGAGCCTTCCGAAGGA
AAAATCAAGCACAGTGGGAGAATCTCATTCTGCAGCCAGTTTTCCTGG
ATCATGCCCGGCACCATTAAGGAAAACATCATATTTGGAGTGTCCTAT
GATGAGTACCGCTACCGGTCAGTCATCAAAGCCTGTCAGTTGGAGGAG
GACATCTCCAAGTTTGCAGAGAAAGACAACATTGTGCTTGGAGAGGGG
GGTATCACTCTTTCTGGAGGACAAAGAGCCAGGATCTCTTTGGCCCGG
GCAGTCTACAAGGATGCAGACCTCTACTTGTTGGACAGTCCCTTCGGC
TACCTCGACGTGCTGACTGAAAAAGAAATTTTTGAAAGCTGTGTGTGC
AAACTGATGGCAAACAAGACCAGGATTCTTGTCACCAGCAAGATGGAA
CATCTGAAGAAAGCGGACAAAATTCTGATTCTGCATGAAGGGAGCTCC
TACTTCTATGGAACATTTAGCGAGCTTCAGAACCTACAGCCAGACTTC
TCCTCCAAATTAATGGGCTGTGACTCCTTCGACCAGTTCTCTGCAGAA
AGAAGAAACTCTATACTCACAGAGACCCTCCACCGCTTCTCCCTTGAG
GGAGATGCCCCAGTTTCTTGGACAGAAACCAAGAAGCAGTCCTTTAAG
CAGACTGGCGAGTTTGGTGAAAAGAGGAAAAATTCAATTCTCAATCCA
ATTAACAGTATTCGCAAGTTCAGCATTGTCCAGAAGACACCCCTCCAG
ATGAATGGCATCGAAGAAGATAGTGACGAGCCGCTGGAGAGACGGCTG
AGTCTGGTGCCAGATTCAGAACAGGGGGAGGCCATCCTGCCCCGGATC
AGCGTCATTTCCACAGGCCCCACATTACAAGCACGGCGCCGGCAGAGT
GTTTTAAATCTCATGACCCATTCAGTGAACCAGGGCCAAAATATCCAC
AGGAAGACTACAGCTTCTACCCGGAAAGTGTCTCTGGCCCCTCAGGCC
AATCTGACCGAGCTGGACATCTACAGCAGGAGGCTCTCCCAGGAAACA
GGGCTGGAAATATCTGAAGAGATTAATGAAGAGGATCTTAAAGAGTGC
TTCTTTGATGACATGGAGAGCATCCCCGCGGTGACCACATGGAACACC
TACCTTAGATATATTACTGTCCACAAGAGCCTCATATTTGTCCTCATC
TGGTGCCTGGTTATTTTCCTCGCTGAGGTGGCGGCCAGTCTTGTTGTG
CTCTGGCTGCTGGGCAACACTCCTCTCCAGGACAAGGGCAATAGTACT
CACAGCAGAAATAATTCTTATGCCGTCATCATTACAAGCACCTCCAGC
TACTACGTGTTCTACATCTATGTGGGCGTGGCTGACACCCTCCTGGCC
ATGGGTTTCTTCCGGGGCCTGCCTTTGGTGCACACCCTCATCACAGTG
TCAAAAATTCTGCACCATAAAATGCTTCATTCTGTCCTGCAGGCACCC
ATGAGCACTTTGAACACATTGAAGGCTGGCGGCATCCTCAACAGATTT
TCTAAAGATATTGCTATCCTGGATGATCTCCTCCCCCTGACAATCTTT
GACTTTATCCAGCTTCTGCTGATCGTGATTGGAGCCATAGCAGTGGTT
GCTGTCCTGCAGCCCTACATTTTTGTGGCCACCGTGCCCGTGATTGTT
GCCTTTATTATGCTCAGAGCTTACTTCCTGCAAACTTCTCAACAGCTC
AAACAGCTAGAATCTGAGGGCCGGAGCCCCATTTTTACCCACCTGGTG
ACTTCCCTGAAGGGACTGTGGACTCTGAGAGCATTCGGGCGACAGCCT
TACTTTGAGACACTGTTCCACAAGGCCCTGAACTTGCACACTGCCAAC
TGGTTTCTTTACCTGAGCACACTCCGCTGGTTCCAGATGCGGATAGAG
ATGATCTTCGTCATCTTTTTTATAGCTGTAACCTTCATTTCTATCCTT
ACAACAGGAGAAGGAGAGGGCAGGGTGGGAATCATCCTCACGCTGGCT
ATGAACATAATGTCCACCTTGCAGTGGGCCGTGAATTCCAGTATAGAT
GTGGATTCTCTAATGAGGAGTGTCTCCCGGGTGTTTAAATTCATTGAT
ATGCCTACTGAGGGGAAACCCACCAAGTCAACAAAACCTTATAAGAAT
GGACAGCTGAGCAAGGTGATGATAATTGAGAACAGCCACGTGAAGAAG
GATGACATTTGGCCCAGCGGGGGCCAGATGACTGTGAAGGACCTGACG
GCCAAGTACACCGAAGGTGGAAATGCCATTTTGGAAAACATCAGCTTC
TCAATCTCTCCTGGGCAGAGAGTTGGATTGCTGGGTCGCACGGGCAGC
GGCAAATCAACCCTGCTCAGTGCCTTCCTTCGGCTCCTGAATACAGAA
GGCGAAATCCAAATTGACGGGGTGAGCTGGGACAGCATCACCCTGCAG
CAGTGGAGAAAAGCATTTGGGGTCATTCCACAGAAAGTTTTCATCTTC
TCTGGCACTTTCAGAAAGAACCTGGACCCCTATGAGCAGTGGAGCGAC
CAGGAGATCTGGAAGGTTGCAGATGAAGTTGGCCTGCGGAGTGTGATA
GAACAATTTCCTGGCAAGCTGGATTTTGTGCTGGTAGATGGAGGCTGC
GTGCTGTCCCACGGCCACAAACAGCTGATGTGCCTCGCCCGCTCCGTT
CTTTCAAAGGCCAAAATCTTGCTTTTGGATGAGCCCAGTGCTCACCTC
GACCCAGTGACCTATCAGATAATCCGCAGGACCTTAAAGCAAGCTTTT
GCCGACTGCACCGTCATACTGTGTGAGCACCGGATTGAAGCAATGCTG
GAATGCCAGCAGTTTCTGGTGATCGAGGAGAATAAGGTCCGGCAGTAC
GACAGCATCCAGAAGTTGTTGAATGAGCGCAGCCTTTTCCGCCAGGCC
ATCTCCCCATCTGACAGAGTCAAGCTGTTTCCACATAGGAACTCCTCT
AAGTGCAAGTCCAAGCCCCAGATCGCTGCCCTCAAGGAGGAAACTGAG
GAAGAGGTGCAGGATACCCGCCTGTGA

In another embodiment, an exemplary codon-optimized CFTR mRNA sequence is:

(SEQ ID NO: 9)
ATGCAGAGGAGCCCACTGGAGAAAGCCTCCGTGGTGAGTAAACTCTTT
TTTAGTTGGACCAGACCCATCCTGCGAAAAGGATACAGGCAGCGCCTC
GAGTTGTCTGATATCTACCAGATTCCTTCTGTGGACTCAGCTGACAAT
TTGAGTGAGAAGCTGGAGCGGGAGTGGGATAGAGAGCTGGCGAGCAAA
AAAAACCCCAAGCTTATCAATGCTCTGCGCCGCTGCTTTTTCTGGAGG
TTCATGTTTTATGGGATCTTCCTGTACCTGGGGGAGGTCACCAAAGCT
GTTCAGCCGCTCCTTCTTGGCCGCATCATCGCCAGCTATGACCCTGAT
AATAAAGAAGAAAGGTCTATTGCTATTTATCTGGGAATTGGCCTCTGC
TTGCTCTTCATCGTCCGCACCCTTCTGCTGCACCCTGCCATTTTTGGC
CTTCACCACATCGGCATGCAAATGAGAATTGCCATGTTCTCCCTCATT
TACAAAAAGACCCTGAAACTTTCCTCAAGAGTGTTAGATAAAATATCC
ATTGGTCAGCTGGTCAGCCTGCTGTCCAACAATCTTAACAAATTTGAT
GAAGGCTTGGCGCTGGCCCACTTCGTGTGGATTGCACCTCTGCAGGTG
GCCCTGTTGATGGGACTTATATGGGAGCTGCTTCAAGCCTCTGCTTTC
TGTGGGCTGGGCTTTTTGATTGTACTGGCACTTTTTCAGGCTGGGCTC
GGAAGAATGATGATGAAATACAGAGATCAGCGGGCCGGGAAGATTTCA
GAGCGACTTGTGATCACCAGTGAAATGATTGAAAATATTCAGAGCGTG
AAAGCCTACTGCTGGGAAGAAGCCATGGAGAAGATGATTGAGAACCTG
AGGCAGACAGAGCTCAAGCTCACTCGGAAGGCTGCTTATGTTCGCTAT
TTCAACAGCAGCGCCTTCTTCTTCAGTGGCTTCTTTGTTGTCTTCCTG
TCTGTTCTGCCATATGCACTGATAAAAGGCATTATTTTACGAAAGATC
TTCACCACCATCAGTTTTTGCATCGTTCTCAGGATGGCCGTCACAAGA
CAGTTCCCCTGGGCTGTGCAGACCTGGTACGATTCCTTGGGGGCCATC
AACAAGATTCAAGATTTCTTGCAAAAACAAGAATATAAAACTTTAGAA
TACAACCTCACCACCACTGAAGTGGTCATGGAAAATGTGACAGCCTTT
TGGGAGGAGGGTTTTGGAGAATTGTTCGAGAAGGCAAAGCAGAATAAC
AACAACAGGAAGACGAGCAATGGGGACGACTCTCTCTTCTTCAGCAAC
TTTTCACTGCTCGGGACCCCTGTGTTGAAAGATATAAACTTCAAGATC
GAGAGGGGCCAGCTCTTGGCTGTGGCAGGCTCCACTGGAGCTGGTAAA
ACATCTCTTCTCATGGTGATCATGGGGGAACTGGAGCCTTCCGAAGGA
AAAATCAAGCACAGTGGGAGAATCTCATTCTGCAGCCAGTTTTCCTGG
ATCATGCCCGGCACCATTAAGGAAAACATCATATTTGGAGTGTCCTAT
GATGAGTACCGCTACCGGTCAGTCATCAAAGCCTGTCAGTTGGAGGAG
GACATCTCCAAGTTTGCAGAGAAAGACAACATTGTGCTTGGAGAGGGG
GGTATCACTCTTTCTGGAGGACAAAGAGCCAGGATCTCTTTGGCCCGG
GCAGTCTACAAGGATGCAGACCTCTACTTGTTGGACAGTCCCTTCGGC
TACCTCGACGTGCTGACTGAAAAAGAAATTTTTGAAAGCTGTGTGTGC
AAACTGATGGCAAACAAGACCAGGATTCTTGTCACCAGCAAGATGGAA
CATCTGAAGAAAGCGGACAAAATTCTGATTCTGCATGAAGGGAGCTCC
TACTTCTATGGAACATTTAGCGAGCTTCAGAACCTACAGCCAGACTTC
TCCTCCAAATTAATGGGCTGTGACTCCTTCGACCAGTTCTCTGCAGAA
AGAAGAAACTCTATACTCACAGAGACCCTCCACCGCTTCTCCCTTGAG
GGAGATGCCCCAGTTTCTTGGACAGAAACCAAGAAGCAGTCCTTTAAG
CAGACTGGCGAGTTTGGTGAAAAGAGGAAAAATTCAATTCTCAATCCA
ATTAACAGTATTCGCAAGTTCAGCATTGTCCAGAAGACACCCCTCCAG
ATGAATGGCATCGAAGAAGATAGTGACGAGCCGCTGGAGAGACGGCTG
AGTCTGGTGCCAGATTCAGAACAGGGGGAGGCCATCCTGCCCCGGATC
AGCGTCATTTCCACAGGCCCCACATTACAAGCACGGCGCCGGCAGAGT
GTTTTAAATCTCATGACCCATTCAGTGAACCAGGGCCAAAATATCCAC
AGGAAGACTACAGCTTCTACCCGGAAAGTGTCTCTGGCCCCTCAGGCC
AATCTGACCGAGCTGGACATCTACAGCAGGAGGCTCTCCCAGGAAACA
GGGCTGGAAATATCTGAAGAGATTAATGAAGAGGATCTTAAAGAGTGC
TTCTTTGATGACATGGAGAGCATCCCCGCGGTGACCACATGGAACACC
TACCTTAGATATATTACTGTCCACAAGAGCCTCATATTTGTCCTCATC
TGGTGCCTGGTTATTTTCCTCGCTGAGGTGGCGGCCAGTCTTGTTGTG
CTCTGGCTGCTGGGCAACACTCCTCTCCAGGACAAGGGCAATAGTACT
CACAGCAGAAATAATTCTTATGCCGTCATCATTACAAGCACCTCCAGC
TACTACGTGTTCTACATCTATGTGGGCGTGGCTGACACCCTCCTGGCC
ATGGGTTTCTTCCGGGGCCTGCCTTTGGTGCACACCCTCATCACAGTG
TCAAAAATTCTGCACCATAAAATGCTTCATTCTGTCCTGCAGGCACCC
ATGAGCACTTTGAACACATTGAAGGCTGGCGGCATCCTCAACAGATTT
TCTAAAGATATTGCTATCCTGGATGATCTCCTCCCCCTGACAATCTTT
GACTTTATCCAGCTTCTGCTGATCGTGATTGGAGCCATAGCAGTGGTT
GCTGTCCTGCAGCCCTACATTTTTGTGGCCACCGTGCCCGTGATTGTT
GCCTTTATTATGCTCAGAGCTTACTTCCTGCAAACTTCTCAACAGCTC
AAACAGCTAGAATCTGAGGGCCGGAGCCCCATTTTTACCCACCTGGTG
ACTTCCCTGAAGGGACTGTGGACTCTGAGAGCATTCGGGCGACAGCCT
TACTTTGAGACACTGTTCCACAAGGCCCTGAACTTGCACACTGCCAAC
TGGTTTCTTTACCTGAGCACACTCCGCTGGTTCCAGATGCGGATAGAG
ATGATCTTCGTCATCTTTTTTATAGCTGTAACCTTCATTTCTATCCTT
ACAACAGGAGAAGGAGAGGGCAGGGTGGGAATCATCCTCACGCTGGCT
ATGAACATAATGTCCACCTTGCAGTGGGCCGTGAATTCCAGTATAGAT
GTGGATTCTCTAATGAGGAGTGTCTCCCGGGTGTTTAAATTCATTGAT
ATGCCTACTGAGGGGAAACCCACCAAGTCAACAAAACCTTATAAGAAT
GGACAGCTGAGCAAGGTGATGATAATTGAGAACAGCCACGTGAAGAAG
GATGACATTTGGCCCAGCGGGGGCCAGATGACTGTGAAGGACCTGACG
GCCAAGTACACCGAAGGTGGAAATGCCATTTTGGAAAACATCAGCTTC
TCAATCTCTCCTGGGCAGAGAGTTGGATTGCTGGGTCGCACGGGCAGC
GGCAAATCAACCCTGCTCAGTGCCTTCCTTCGGCTCCTGAATACAGAA
GGCGAAATCCAAATTGACGGGGTGAGCTGGGACAGCATCACCCTGCAG
CAGTGGAGAAAAGCATTTGGGGTCATTCCACAGAAAGTTTTCATCTTC
TCTGGCACTTTCAGAAAGAACCTGGACCCCTATGAGCAGTGGAGCGAC
CAGGAGATCTGGAAGGTTGCAGATGAAGTTGGCCTGCGGAGTGTGATA
GAACAATTTCCTGGCAAGCTGGATTTTGTGCTGGTAGATGGAGGCTGC
GTGCTGTCCCACGGCCACAAACAGCTGATGTGCCTCGCCCGCTCCGTT
CTTTCAAAGGCCAAAATCTTGCTTTTGGATGAGCCCAGTGCTCACCTT
GACCCAGTGACCTATCAGATAATCCGCAGGACCTTAAAGCAAGCTTTT
GCCGACTGCACCGTCATACTGTGTGAGCACCGGATTGAAGCAATGCTG
GAATGCCAGCAGTTTCTGGTGATCGAGGAGAATAAGGTCCGGCAGTAC
GACAGCATCCAGAAGTTGTTGAATGAGCGCAGCCTTTTCCGCCAGGCC
ATCTCCCCATCTGACAGAGTCAAGCTGTTTCCACATAGGAACTCCTCT
AAGTGCAAGTCCAAGCCCCAGATCGCTGCCCTCAAGGAGGAAACTGAG
GAAGAGGTGCAGGATACCCGCCTGTGA

In yet another embodiment, an exemplary codon-optimized CFTR mRNA sequence is:

(SEQ ID NO: 10)
ATGCAGAGGAGCCCACTGGAGAAAGCCTCCGTGGTGAGTAAACTCTTT
TTTAGTTGGACCAGACCCATCCTGCGAAAAGGATACAGGCAGCGCCTC
GAGTTGTCAGATATCTACCAGATTCCTTCTGTGGACTCAGCTGACAAT
TTGAGTGAGAAGCTGGAGCGGGAGTGGGATAGAGAGCTGGCGAGCAAA
AAAAACCCCAAGCTTATCAATGCTCTGCGCCGCTGCTTTTTCTGGAGG
TTCATGTTTTATGGGATCTTCCTGTACCTGGGGGAGGTCACCAAAGCT
GTTCAGCCGCTCCTTCTTGGCCGCATCATCGCCAGCTATGACCCTGAT
AATAAAGAAGAAAGGTCTATTGCTATTTATCTGGGAATTGGCCTCTGC
TTGCTCTTCATCGTCCGCACCCTTCTGCTGCACCCTGCCATTTTTGGC
CTTCACCACATCGGCATGCAAATGAGAATTGCCATGTTCTCCCTCATT
TACAAAAAGACCCTGAAACTTTCCTCAAGAGTGTTAGATAAAATATCC
ATTGGTCAGCTGGTCAGCCTGCTGTCCAACAATCTTAACAAATTTGAT
GAAGGCTTGGCGCTGGCCCACTTCGTGTGGATTGCACCTCTGCAGGTG
GCCCTGTTGATGGGACTTATATGGGAGCTGCTTCAAGCCTCTGCTTTC
TGTGGGCTGGGCTTTTTGATTGTACTGGCACTTTTTCAGGCTGGGCTC
GGAAGAATGATGATGAAATACAGAGATCAGCGGGCCGGGAAGATATCA
GAGCGACTTGTGATCACCAGTGAAATGATTGAAAATATTCAGAGCGTG
AAAGCCTACTGCTGGGAAGAAGCCATGGAGAAGATGATTGAGAACCTG
AGGCAGACAGAGCTCAAGCTCACTCGGAAGGCTGCTTATGTTCGCTAT
TTCAACAGCAGCGCCTTCTTCTTCAGTGGCTTCTTTGTTGTCTTCCTG
TCTGTTCTGCCATATGCACTGATAAAAGGCATTATTTTACGAAAGATC
TTCACCACCATCAGTTTTTGCATCGTTCTCAGGATGGCCGTCACAAGA
CAGTTCCCCTGGGCTGTGCAGACCTGGTACGATTCCTTGGGGGCCATC
AACAAGATTCAAGATTTCTTGCAAAAACAAGAATATAAAACTTTAGAA
TACAACCTCACCACCACTGAAGTGGTCATGGAAAATGTGACAGCCTTT
TGGGAGGAGGGTTTTGGAGAATTGTTCGAGAAGGCAAAGCAGAATAAC
AACAACAGGAAGACGAGCAATGGGGACGACTCTCTCTTCTTCAGCAAC
TTTTCACTGCTCGGGACCCCTGTGTTGAAAGATATAAACTTCAAGATC
GAGAGGGGCCAGCTCTTGGCTGTGGCAGGCTCCACTGGAGCTGGTAAA
ACATCTCTTCTCATGGTGATCATGGGGGAACTGGAGCCTTCCGAAGGA
AAAATCAAGCACAGTGGGAGAATCTCATTCTGCAGCCAGTTTTCCTGG
ATCATGCCCGGCACCATTAAGGAAAACATCATATTTGGAGTGTCCTAT
GATGAGTACCGCTACCGGTCAGTCATCAAAGCCTGTCAGTTGGAGGAG
GACATCTCCAAGTTTGCAGAGAAAGACAACATTGTGCTTGGAGAGGGG
GGTATCACTCTTTCTGGAGGACAAAGAGCCAGGATCTCTTTGGCCCGG
GCAGTCTACAAGGATGCAGACCTCTACTTGTTGGACAGTCCCTTCGGC
TACCTCGACGTGCTGACTGAAAAAGAAATTTTTGAAAGCTGTGTGTGC
AAACTGATGGCAAACAAGACCAGGATTCTTGTCACCAGCAAGATGGAA
CATCTGAAGAAAGCGGACAAAATTCTGATTCTGCATGAAGGGAGCTCC
TACTTCTATGGAACATTTAGCGAGCTTCAGAACCTACAGCCAGACTTC
TCCTCCAAATTAATGGGCTGTGACTCCTTCGACCAGTTCTCTGCAGAA
AGAAGAAACTCTATACTCACAGAGACCCTCCACCGCTTCTCCCTTGAG
GGAGATGCCCCAGTTTCTTGGACAGAAACCAAGAAGCAGTCCTTTAAG
CAGACTGGCGAGTTTGGTGAAAAGAGGAAAAATTCAATTCTCAATCCA
ATTAACAGTATTCGCAAGTTCAGCATTGTCCAGAAGACACCCCTCCAG
ATGAATGGCATCGAAGAAGATAGTGACGAGCCGCTGGAGAGACGGCTG
AGTCTGGTGCCAGATTCAGAACAGGGGGAGGCCATCCTGCCCCGGATC
AGCGTCATTTCCACAGGCCCCACATTACAAGCACGGCGCCGGCAGAGT
GTTTTAAATCTCATGACCCATTCAGTGAACCAGGGCCAAAATATCCAC
AGGAAGACTACAGCTTCTACCCGGAAAGTGTCTCTGGCCCCTCAGGCC
AATCTGACCGAGCTGGACATCTACAGCAGGAGGCTCTCCCAGGAAACA
GGGCTTGAAATATCTGAAGAGATTAATGAAGAGGATCTTAAAGAGTGC
TTCTTTGATGACATGGAGAGCATCCCCGCGGTGACCACATGGAACACC
TACCTTAGATATATTACTGTCCACAAGAGCCTCATATTTGTCCTCATC
TGGTGCCTGGTTATTTTCCTCGCTGAGGTGGCGGCCAGTCTTGTTGTG
CTCTGGCTGCTGGGCAACACTCCTCTCCAGGACAAGGGCAATAGTACA
CACAGCAGAAATAATTCTTATGCCGTCATCATTACAAGCACCTCCAGC
TACTACGTGTTCTACATCTATGTGGGCGTGGCTGACACCCTCCTGGCC
ATGGGTTTCTTCCGGGGCCTGCCTTTGGTGCACACCCTCATCACAGTG
TCAAAAATTCTGCACCATAAAATGCTTCATTCTGTCCTGCAGGCACCC
ATGAGCACTTTGAACACATTGAAGGCTGGCGGCATCCTCAACAGATTT
TCTAAAGATATTGCTATCCTGGATGATCTCCTCCCCCTGACAATCTTT
GACTTTATCCAGCTTCTGCTGATCGTGATTGGAGCCATAGCAGTGGTT
GCTGTCCTGCAGCCCTACATTTTTGTGGCCACCGTGCCCGTGATTGTT
GCCTTTATTATGCTCAGAGCTTACTTCCTGCAAACTTCTCAACAGCTC
AAACAGCTAGAATCTGAGGGCCGGAGCCCCATTTTTACCCACCTGGTG
ACTTCCCTGAAGGGACTGTGGACTCTGAGAGCATTCGGGCGACAGCCT
TACTTTGAGACACTGTTCCACAAGGCCCTGAACTTGCACACTGCCAAC
TGGTTTCTTTACCTGAGCACACTCCGCTGGTTCCAGATGCGGATAGAG
ATGATCTTCGTCATCTTTTTTATAGCTGTAACCTTCATTTCTATCCTT
ACAACAGGAGAAGGAGAGGGCAGGGTGGGAATCATCCTCACGCTGGCT
ATGAACATAATGTCCACCTTGCAGTGGGCCGTGAATTCCAGTATAGAT
GTGGATTCTCTAATGAGGAGTGTCTCCCGGGTGTTTAAATTCATTGAT
ATGCCTACTGAGGGGAAACCCACCAAGTCAACAAAACCTTATAAGAAT
GGACAGCTGAGCAAGGTGATGATAATTGAGAACAGCCACGTGAAGAAG
GATGACATTTGGCCCAGCGGGGGCCAGATGACTGTGAAGGACCTGACG
GCCAAGTACACCGAAGGTGGAAATGCCATTTTGGAAAACATCAGCTTC
TCAATCTCTCCTGGGCAGAGAGTTGGATTGCTGGGTCGCACGGGCAGC
GGCAAATCAACCCTGCTCAGTGCCTTCCTTCGGCTCCTGAATACAGAA
GGCGAAATCCAAATTGACGGGGTGAGCTGGGACAGCATCACCCTGCAG
CAGTGGAGAAAAGCATTTGGGGTCATTCCACAGAAAGTTTTCATCTTC
TCTGGCACTTTCAGAAAGAACCTGGACCCCTATGAGCAGTGGAGCGAC
CAGGAGATCTGGAAGGTTGCAGATGAAGTTGGCCTGCGGAGTGTGATA
GAACAATTTCCTGGCAAGCTGGATTTTGTGCTGGTAGATGGAGGCTGC
GTGCTGTCCCACGGCCACAAACAGCTGATGTGCCTCGCCCGCTCCGTT
CTTTCAAAGGCCAAAATCTTGCTTTTGGATGAGCCCAGTGCTCACCTT
GACCCAGTGACCTATCAGATAATCCGCAGGACCTTAAAGCAAGCTTTT
GCCGACTGCACCGTCATACTGTGTGAGCACCGGATTGAAGCAATGCTG
GAATGCCAGCAGTTTCTGGTGATCGAGGAGAATAAGGTCCGGCAGTAC
GACAGCATCCAGAAGTTGTTGAATGAGCGCAGCCTTTTCCGCCAGGCC
ATCTCCCCATCTGACAGAGTCAAGCTGTTTCCACATAGGAACTCCTCT
AAGTGCAAGTCCAAGCCCCAGATCGCTGCCCTCAAGGAGGAAACTGAG
GAAGAGGTGCAGGATACCCGCCTGTGA.

In yet another embodiment, an exemplary codon-optimized CFTR mRNA sequence is:

(SEQ ID NO: 11)
ATGCAGAGGAGCCCACTGGAGAAAGCCTCCGTGGTGAGTAAACTCTTT
TTTAGTTGGACCAGACCCATCCTGCGAAAAGGATACAGGCAGCGCCTC
GAGTTGTCAGATATCTACCAGATTCCTTCTGTGGACTCAGCTGACAAT
TTGAGTGAGAAGCTGGAGCGGGAGTGGGATAGAGAGCTGGCGAGCAAA
AAAAACCCCAAGCTTATCAATGCTCTGCGCCGCTGCTTTTTCTGGAGG
TTCATGTTTTATGGGATCTTCCTGTACCTGGGGGAGGTCACCAAAGCT
GTTCAGCCGCTCCTTCTTGGCCGCATCATCGCCAGCTATGACCCTGAT
AATAAAGAAGAAAGGTCTATTGCTATTTATCTGGGAATTGGCCTCTGC
TTGCTCTTCATCGTCCGCACCCTTCTGCTGCACCCTGCCATTTTTGGC
CTTCACCACATCGGCATGCAAATGAGAATTGCCATGTTCTCCCTCATT
TACAAAAAGACCCTGAAACTTTCCTCAAGAGTGTTAGATAAAATATCC
ATTGGTCAGCTGGTCAGCCTGCTGTCCAACAATCTTAACAAATTTGAT
GAAGGCTTGGCGCTGGCCCACTTCGTGTGGATTGCACCTCTGCAGGTG
GCCCTGTTGATGGGACTTATATGGGAGCTGCTTCAAGCCTCTGCTTTC
TGTGGGCTGGGCTTTTTGATTGTACTGGCACTTTTTCAGGCTGGGCTC
GGAAGAATGATGATGAAATACAGAGATCAGCGGGCCGGGAAGATATCA
GAGCGACTTGTGATCACCAGTGAAATGATTGAAAATATTCAGAGCGTG
AAAGCCTACTGCTGGGAAGAAGCCATGGAGAAGATGATTGAGAACCTG
AGGCAGACAGAGCTCAAGCTCACTCGGAAGGCTGCTTATGTTCGCTAT
TTCAACAGCAGCGCCTTCTTCTTCAGTGGCTTCTTTGTTGTCTTCCTG
TCTGTTCTGCCATATGCACTGATAAAAGGCATTATTTTACGAAAGATC
TTCACCACCATCAGTTTTTGCATCGTTCTCAGGATGGCCGTCACAAGA
CAGTTCCCCTGGGCTGTGCAGACCTGGTACGATTCCTTGGGGGCCATC
AACAAGATTCAAGATTTCTTGCAAAAACAAGAATATAAAACTTTAGAA
TACAACCTCACCACCACTGAAGTGGTCATGGAAAATGTGACAGCCTTT
TGGGAGGAGGGTTTTGGAGAATTGTTCGAGAAGGCAAAGCAGAATAAC
AACAACAGGAAGACGAGCAATGGGGACGACTCTCTCTTCTTCAGCAAC
TTTTCACTGCTCGGGACCCCTGTGTTGAAAGATATAAACTTCAAGATC
GAGAGGGGCCAGCTCTTGGCTGTGGCAGGCTCCACTGGAGCTGGTAAA
ACATCTCTTCTCATGGTGATCATGGGGGAACTGGAGCCTTCCGAAGGA
AAAATCAAGCACAGTGGGAGAATCTCATTCTGCAGCCAGTTTTCCTGG
ATCATGCCCGGCACCATTAAGGAAAACATCATATTTGGAGTGTCCTAT
GATGAGTACCGCTACCGGTCAGTCATCAAAGCCTGTCAGTTGGAGGAG
GACATCTCCAAGTTTGCAGAGAAAGACAACATTGTGCTTGGAGAGGGG
GGTATCACTCTTTCTGGAGGACAAAGAGCCAGGATCTCTTTGGCCCGG
GCAGTCTACAAGGATGCAGACCTCTACTTGTTGGACAGTCCCTTCGGC
TACCTCGACGTGCTGACTGAAAAAGAAATTTTTGAAAGCTGTGTGTGC
AAACTGATGGCAAACAAGACCAGGATTCTTGTCACCAGCAAGATGGAA
CATCTGAAGAAAGCGGACAAAATTCTGATTCTGCATGAAGGGAGCTCC
TACTTCTATGGAACATTTAGCGAGCTTCAGAACCTACAGCCAGACTTC
TCCTCCAAATTAATGGGCTGTGACTCCTTCGACCAGTTCTCTGCAGAA
AGAAGAAACTCTATACTCACAGAGACCCTCCACCGCTTCTCCCTTGAG
GGAGATGCCCCAGTTTCTTGGACAGAAACCAAGAAGCAGTCCTTTAAG
CAGACTGGCGAGTTTGGTGAAAAGAGGAAAAATTCAATTCTCAATCCA
ATTAACAGTATTCGCAAGTTCAGCATTGTCCAGAAGACACCCCTCCAG
ATGAATGGCATCGAAGAAGATAGTGACGAGCCGCTGGAGAGACGGCTG
AGTCTGGTGCCAGATTCAGAACAGGGGGAGGCCATCCTGCCCCGGATC
AGCGTCATTTCCACAGGCCCCACATTACAAGCACGGCGCCGGCAGAGT
GTTTTAAATCTCATGACCCATTCAGTGAACCAGGGCCAAAATATCCAC
AGGAAGACTACAGCTTCTACCCGGAAAGTGTCTCTGGCCCCTCAGGCC
AATCTGACCGAGCTGGACATCTACAGCAGGAGGCTCTCCCAGGAAACA
GGGCTGGAAATATCTGAAGAGATTAATGAAGAGGATCTTAAAGAGTGC
TTCTTTGATGACATGGAGAGCATCCCCGCGGTGACCACATGGAACACC
TACCTTAGATATATTACTGTCCACAAGAGCCTCATATTTGTCCTCATC
TGGTGCCTGGTTATTTTCCTCGCTGAGGTGGCGGCCAGTCTTGTTGTG
CTCTGGCTGCTGGGCAACACTCCTCTCCAGGACAAGGGCAATAGTACT
CACAGCAGAAATAATTCTTATGCCGTCATCATTACAAGCACCTCCAGC
TACTACGTGTTCTACATCTATGTGGGCGTGGCTGACACCCTCCTGGCC
ATGGGTTTCTTCCGGGGCCTGCCTTTGGTGCACACCCTCATCACAGTG
TCAAAAATTCTGCACCATAAAATGCTTCATTCTGTCCTGCAGGCACCC
ATGAGCACTTTGAACACATTGAAGGCTGGCGGCATCCTCAACAGATTT
TCTAAAGATATTGCTATCCTGGATGATCTCCTCCCCCTGACAATCTTT
GACTTTATCCAGCTTCTGCTGATCGTGATTGGAGCCATAGCAGTGGTT
GCTGTCCTGCAGCCCTACATTTTTGTGGCCACCGTGCCCGTGATTGTT
GCCTTTATTATGCTCAGAGCTTACTTCCTGCAAACTTCTCAACAGCTC
AAACAGCTAGAATCTGAGGGCCGGAGCCCCATTTTTACCCACCTGGTG
ACTTCCCTGAAGGGACTGTGGACTCTGAGAGCATTCGGGCGACAGCCT
TACTTTGAGACACTGTTCCACAAGGCCCTGAACTTGCACACTGCCAAC
TGGTTTCTTTACCTGAGCACACTCCGCTGGTTCCAGATGCGGATAGAG
ATGATCTTCGTCATCTTTTTTATAGCTGTAACCTTCATTTCTATCCTT
ACAACAGGAGAAGGAGAGGGCAGGGTGGGAATCATCCTCACGCTGGCT
ATGAACATAATGTCCACCTTGCAGTGGGCCGTGAATTCCAGTATAGAT
GTGGATTCTCTAATGAGGAGTGTCTCCCGGGTGTTTAAATTCATTGAT
ATGCCAACTGAGGGGAAACCCACCAAGTCAACAAAACCTTATAAGAAT
GGACAGCTGAGCAAGGTGATGATAATTGAGAACAGCCACGTGAAGAAG
GATGACATTTGGCCCAGCGGGGGCCAGATGACTGTGAAGGACCTGACG
GCCAAGTACACCGAAGGTGGAAATGCCATTTTGGAAAACATCAGCTTC
TCAATCTCTCCTGGGCAGAGAGTTGGATTGCTGGGTCGCACGGGCAGC
GGCAAATCAACCCTGCTCAGTGCCTTCCTTCGGCTCCTGAATACAGAA
GGCGAAATCCAAATTGACGGGGTGAGCTGGGACAGCATCACCCTGCAG
CAGTGGAGAAAAGCATTTGGGGTCATTCCACAGAAAGTTTTCATCTTC
TCTGGCACTTTCAGAAAGAACCTGGACCCCTATGAGCAGTGGAGCGAC
CAGGAGATCTGGAAGGTTGCAGATGAAGTTGGCCTGCGGAGTGTGATA
GAACAATTTCCTGGCAAGCTGGATTTTGTGCTGGTAGATGGAGGCTGC
GTGCTGTCCCACGGCCACAAACAGCTGATGTGCCTCGCCCGCTCCGTT
CTTTCAAAGGCCAAAATCTTGCTTTTGGATGAGCCCAGTGCTCACCTC
GACCCAGTGACCTATCAGATAATCCGCAGGACCTTAAAGCAAGCTTTT
GCCGACTGCACCGTCATACTGTGTGAGCACCGGATTGAAGCAATGCTG
GAATGCCAGCAGTTTCTGGTGATCGAGGAGAATAAGGTCCGGCAGTAC
GACAGCATCCAGAAGTTGTTGAATGAGCGCAGCCTTTTCCGCCAGGCC
ATCTCCCCATCTGACAGAGTCAAGCTGTTTCCACATAGGAACTCCTCT
AAGTGCAAGTCCAAGCCCCAGATCGCTGCCCTCAAGGAGGAAACTGAG
GAAGAGGTGCAGGATACCCGCCTGTGA.

In yet another embodiment, an exemplary codon-optimized CFTR mRNA sequence is:

(SEQ ID NO: 12)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 13)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATTTCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCAACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 14)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCTGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCCGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAGCCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 15)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTTGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACACACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In yet another embodiment, an exemplary codon-optimized CFTR mRNA sequence is:

(SEQ ID NO: 16)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCCGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAGTCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 17)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCTGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATTTCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCTATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACACACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 18)
ATGCAGAGAAGCCCCCTGGAGAAGGCCTCTGTGGTGAGCAAGCTGTTCTTCAGCTG
GACCAGACCCATCCTGAGAAAGGGCTACAGACAGAGACTGGAGCTGTCTGACATCT
ACCAGATCCCCTCTGTGGACTCTGCCGACAACCTGTCTGAGAAGCTGGAGAGAGAG
TGGGACAGAGAGCTGGCCAGCAAGAAGAACCCCAAGCTGATCAATGCCCTGAGAA
GATGCTTCTTCTGGAGATTCATGTTCTATGGCATCTTCCTGTACCTGGGAGAGGTGAC
CAAGGCCGTGCAGCCCCTGCTGCTGGGCAGGATCATTGCCAGCTATGACCCTGACA
ACAAGGAGGAGAGAAGCATTGCCATCTACCTGGGCATTGGCCTGTGCCTGCTGTTCA
TTGTGAGAACCCTGCTGCTGCACCCTGCCATCTTTGGCCTGCACCACATTGGCATGC
AGATGAGAATTGCCATGTTCAGCCTGATCTACAAGAAGACCCTGAAGCTGAGCAGC
AGAGTGCTGGACAAGATCAGCATTGGCCAGCTGGTGAGCCTGCTGAGCAACAACCT
GAACAAGTTTGATGAGGGCCTGGCCCTGGCCCACTTTGTGTGGATTGCCCCCCTGCA
GGTGGCCCTGCTGATGGGCCTGATCTGGGAGCTGCTGCAGGCCTCTGCCTTCTGTGG
CCTGGGCTTCCTGATTGTGCTGGCCCTGTTCCAGGCCGGCCTGGGCAGAATGATGAT
GAAGTACAGAGACCAGAGAGCCGGCAAGATCTCTGAGAGACTGGTGATCACCTCTG
AGATGATTGAGAACATCCAGTCTGTGAAGGCCTACTGCTGGGAGGAGGCCATGGAG
AAGATGATTGAGAACCTGAGACAGACAGAGCTGAAGCTGACCAGGAAGGCCGCCTA
TGTGAGATACTTCAACAGCTCTGCCTTCTTCTTCTCTGGCTTCTTTGTGGTGTTCCTGT
CTGTGCTGCCCTATGCCCTGATCAAGGGCATCATCCTGAGGAAGATCTTCACCACCA
TCAGCTTCTGCATTGTGCTGAGGATGGCCGTGACCAGGCAGTTCCCCTGGGCCGTGC
AGACCTGGTATGACAGCCTGGGGGCCATCAACAAGATCCAGGACTTCCTGCAGAAG
CAGGAGTACAAGACCCTGGAGTACAACCTGACCACCACAGAGGTGGTGATGGAGAA
TGTGACAGCCTTCTGGGAGGAGGGCTTTGGAGAGCTGTTTGAGAAGGCCAAGCAGA
ACAACAACAACAGAAAGACCAGCAATGGAGATGACAGCCTGTTCTTCAGCAACTTC
AGCCTGCTGGGCACCCCTGTGCTGAAGGACATCAACTTCAAGATTGAGAGGGGCCA
GCTGCTGGCCGTGGCCGGCAGCACAGGAGCCGGCAAGACCAGCCTGCTGATGGTGA
TCATGGGAGAGCTGGAGCCCTCTGAGGGCAAGATCAAGCACTCTGGCAGAATCAGC
TTCTGCAGCCAGTTCAGCTGGATCATGCCTGGCACCATCAAGGAGAACATCATCTTT
GGGGTGAGCTATGATGAGTACAGGTACAGATCTGTGATCAAGGCCTGCCAGCTGGA
GGAGGACATCTCCAAGTTTGCCGAGAAGGACAACATTGTGCTGGGGGAGGGAGGCA
TCACCCTGTCTGGGGGCCAGAGAGCCAGAATCAGCCTGGCCAGAGCCGTGTACAAG
GATGCCGACCTGTACCTGCTGGACAGCCCCTTTGGCTACCTGGATGTGCTGACAGAG
AAGGAGATCTTTGAGAGCTGTGTGTGCAAGCTGATGGCCAACAAGACCAGGATCCT
GGTGACCAGCAAGATGGAGCACCTGAAGAAGGCCGACAAGATCCTGATCCTGCATG
AGGGCAGCAGCTACTTCTATGGCACCTTCTCTGAGCTGCAGAACCTGCAGCCTGACT
TCAGCAGCAAGCTGATGGGCTGTGACAGCTTTGACCAGTTCTCTGCTGAGAGAAGA
AACAGCATCCTGACAGAGACCCTGCACAGGTTCAGCCTGGAGGGGGATGCCCCTGT
GAGCTGGACAGAGACCAAGAAGCAGAGCTTCAAGCAGACAGGAGAGTTTGGGGAG
AAGAGGAAGAACAGCATCCTGAACCCCATCAACAGCATCAGGAAGTTCAGCATTGT
GCAGAAGACCCCCCTGCAGATGAATGGCATTGAGGAGGACTCTGATGAGCCCCTGG
AGAGAAGACTGAGCCTGGTGCCAGACTCTGAGCAGGGAGAGGCCATCCTGCCCAGG
ATCTCTGTGATCAGCACAGGCCCCACCCTGCAGGCCAGAAGAAGACAGTCTGTGCT
GAACCTGATGACCCACTCTGTGAACCAGGGCCAGAATATCCACAGAAAGACCACAG
CCAGCACCAGAAAGGTGAGCCTGGCCCCCCAGGCCAACCTGACAGAGCTGGACATC
TACAGCAGAAGGCTGAGCCAGGAGACAGGCCTGGAGATCTCTGAGGAGATCAATGA
GGAGGACCTGAAGGAGTGCTTCTTTGATGACATGGAGAGCATCCCTGCCGTGACCA
CCTGGAACACCTACCTGAGATACATCACAGTGCACAAGAGCCTGATCTTTGTGCTGA
TCTGGTGCCTGGTGATCTTCCTGGCCGAGGTGGCCGCCAGCCTGGTGGTGCTGTGGC
TGCTGGGCAACACCCCCCTGCAGGACAAGGGCAACAGCACCCACAGCAGAAACAAC
AGCTATGCTGTGATCATCACCAGCACCAGCAGCTACTATGTGTTCTACATCTATGTG
GGAGTGGCTGACACCCTGCTGGCCATGGGCTTCTTCAGAGGCCTGCCCCTGGTGCAC
ACCCTGATCACAGTGAGCAAGATCCTGCACCACAAGATGCTGCACTCTGTGCTGCAG
GCCCCCATGAGCACCCTGAACACCCTGAAGGCTGGAGGCATCCTGAACAGATTCAG
CAAGGACATTGCCATCCTGGATGACCTGCTGCCCCTGACCATCTTTGACTTCATCCA
GCTGCTGCTGATTGTGATTGGAGCCATTGCCGTGGTGGCCGTGCTGCAGCCCTACAT
CTTTGTGGCCACAGTGCCTGTGATTGTGGCCTTCATCATGCTGAGGGCCTACTTCCTG
CAGACCAGCCAGCAGCTGAAGCAGCTGGAGTCTGAGGGCAGAAGCCCCATCTTCAC
CCACCTGGTGACCAGCCTGAAGGGCCTGTGGACCCTGAGGGCCTTTGGCAGACAGC
CCTACTTTGAGACCCTGTTCCACAAGGCCCTGAACCTGCACACAGCCAACTGGTTCC
TGTACCTGAGCACCCTGAGATGGTTCCAGATGAGGATTGAGATGATCTTTGTGATCT
TCTTCATTGCCGTGACCTTCATCAGCATCCTGACCACAGGGGAGGGCGAGGGCAGA
GTGGGCATCATCCTGACCCTGGCCATGAACATCATGAGCACCCTGCAGTGGGCCGTG
AACAGCAGCATTGATGTGGACAGCCTGATGAGATCTGTGAGCAGAGTGTTCAAGTT
CATTGACATGCCCACAGAGGGCAAGCCCACCAAGAGCACCAAGCCCTACAAGAATG
GCCAGCTGAGCAAGGTGATGATCATTGAGAACAGCCATGTGAAGAAGGATGACATC
TGGCCCTCTGGAGGCCAGATGACAGTGAAGGACCTGACAGCCAAGTACACAGAGGG
GGGCAATGCCATCCTGGAGAACATCAGCTTCAGCATCAGCCCTGGCCAGAGGGTGG
GCCTGCTGGGCAGAACAGGCTCTGGCAAGAGCACCCTGCTGTCTGCCTTCCTGAGGC
TGCTGAACACAGAGGGAGAGATCCAGATTGATGGGGTGAGCTGGGACAGCATCACC
CTGCAGCAGTGGAGGAAGGCCTTTGGGGTGATCCCCCAGAAGGTGTTCATCTTCTCT
GGCACCTTCAGGAAGAACCTGGACCCCTATGAGCAGTGGTCTGACCAGGAGATCTG
GAAGGTGGCCGATGAGGTGGGCCTGAGATCTGTGATTGAGCAGTTCCCTGGCAAGC
TGGACTTTGTGCTGGTGGATGGAGGCTGTGTGCTGAGCCATGGCCACAAGCAGCTGA
TGTGCCTGGCCAGATCTGTGCTGAGCAAGGCCAAGATCCTGCTGCTGGATGAGCCCT
CTGCCCACCTGGACCCTGTGACCTACCAGATCATCAGAAGAACCCTGAAGCAGGCC
TTTGCCGACTGCACAGTGATCCTGTGTGAGCACAGAATTGAGGCCATGCTGGAGTGC
CAGCAGTTCCTGGTGATTGAGGAGAACAAGGTGAGGCAGTATGACAGCATCCAGAA
GCTGCTGAATGAGAGAAGCCTGTTCAGACAGGCCATCAGCCCCTCTGACAGAGTGA
AGCTGTTCCCCCACAGGAACAGCAGCAAGTGCAAGAGCAAGCCCCAGATTGCCGCC
CTGAAGGAGGAGACAGAGGAGGAGGTGCAGGACACCAGACTGTGA.

In yet another embodiment, an exemplary codon-optimized CFTR mRNA sequence is:

(SEQ ID NO: 19)
ATGCAGAGGAGCCCCCTGGAGAAGGCCAGCGTGGTGAGCAAGCTGTTCTTCAGCTG
GACCAGGCCCATCCTGAGGAAGGGCTACAGGCAGAGGCTGGAGCTGAGCGACATCT
ACCAGATCCCCAGCGTGGACAGCGCCGACAACCTGAGCGAGAAGCTGGAGAGGGA
GTGGGACAGGGAGCTGGCCAGCAAGAAGAACCCCAAGCTGATCAACGCCCTGAGG
AGGTGCTTCTTCTGGAGGTTCATGTTCTACGGCATCTTCCTGTACCTGGGCGAGGTG
ACCAAGGCCGTGCAGCCCCTGCTGCTGGGCAGGATCATCGCCAGCTACGACCCCGA
CAACAAGGAGGAGAGGAGCATCGCCATCTACCTGGGCATCGGCCTGTGCCTGCTGT
TCATCGTGAGGACCCTGCTGCTGCACCCCGCCATCTTCGGCCTGCACCACATCGGCA
TGCAGATGAGGATCGCCATGTTCAGCCTGATCTACAAGAAGACCCTGAAGCTGAGC
AGCAGGGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGGATGAT
GATGAAGTACAGGGACCAGAGGGCCGGCAAGATCAGCGAGAGGCTGGTGATCACC
AGCGAGATGATCGAGAACATCCAGAGCGTGAAGGCCTACTGCTGGGAGGAGGCCAT
GGAGAAGATGATCGAGAACCTGAGGCAGACCGAGCTGAAGCTGACCAGGAAGGCC
GCCTACGTGAGGTACTTCAACAGCAGCGCCTTCTTCTTCAGCGGCTTCTTCGTGGTGT
TCCTGAGCGTGCTGCCCTACGCCCTGATCAAGGGCATCATCCTGAGGAAGATCTTCA
CCACCATCAGCTTCTGCATCGTGCTGAGGATGGCCGTGACCAGGCAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGGAAGACCAGCAACGGCGACGACAGCCTGTTCTTCAGC
AACTTCAGCCTGCTGGGCACCCCCGTGCTGAAGGACATCAACTTCAAGATCGAGAG
GGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
GATCAGCTTCTGCAGCCAGTTCAGCTGGATCATGCCCGGCACCATCAAGGAGAACA
TCATCTTCGGCGTGAGCTACGACGAGTACAGGTACAGGAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGGGCCAGGATCAGCCTGGCCAGGGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGGATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGGAGGAACAGCATCCTGACCGAGACCCTGCACAGGTTCAGCCTGGAGGGCGA
CGCCCCCGTGAGCTGGACCGAGACCAAGAAGCAGAGCTTCAAGCAGACCGGCGAGT
TCGGCGAGAAGAGGAAGAACAGCATCCTGAACCCCATCAACAGCATCAGGAAGTTC
AGCATCGTGCAGAAGACCCCCCTGCAGATGAACGGCATCGAGGAGGACAGCGACG
AGCCCCTGGAGAGGAGGCTGAGCCTGGTGCCCGACAGCGAGCAGGGCGAGGCCATC
CTGCCCAGGATCAGCGTGATCAGCACCGGCCCCACCCTGCAGGCCAGGAGGAGGCA
GAGCGTGCTGAACCTGATGACCCACAGCGTGAACCAGGGCCAGAACATCCACAGGA
AGACCACCGCCAGCACCAGGAAGGTGAGCCTGGCCCCCCAGGCCAACCTGACCGAG
CTGGACATCTACAGCAGGAGGCTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGGTACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGGAACAACAGCTACGCCGTGATCATCACCAGCACCAGCAGCTACTACGTGTTC
TACATCTACGTGGGCGTGGCCGACACCCTGCTGGCCATGGGCTTCTTCAGGGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGGTTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
GGCCTACTTCCTGCAGACCAGCCAGCAGCTGAAGCAGCTGGAGAGCGAGGGCAGGA
GCCCCATCTTCACCCACCTGGTGACCAGCCTGAAGGGCCTGTGGACCCTGAGGGCCT
TCGGCAGGCAGCCCTACTTCGAGACCCTGTTCCACAAGGCCCTGAACCTGCACACCG
CCAACTGGTTCCTGTACCTGAGCACCCTGAGGTGGTTCCAGATGAGGATCGAGATGA
TCTTCGTGATCTTCTTCATCGCCGTGACCTTCATCAGCATCCTGACCACCGGCGAGG
GCGAGGGCAGGGTGGGCATCATCCTGACCCTGGCCATGAACATCATGAGCACCCTG
CAGTGGGCCGTGAACAGCAGCATCGACGTGGACAGCCTGATGAGGAGCGTGAGCAG
GGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGGGTGGGCCTGCTGGGCAGGACCGGCAGCGGCAAGAGCACCCTGCTGAGC
GCCTTCCTGAGGCTGCTGAACACCGAGGGCGAGATCCAGATCGACGGCGTGAGCTG
GGACAGCATCACCCTGCAGCAGTGGAGGAAGGCCTTCGGCGTGATCCCCCAGAAGG
TGTTCATCTTCAGCGGCACCTTCAGGAAGAACCTGGACCCCTACGAGCAGTGGAGC
GACCAGGAGATCTGGAAGGTGGCCGACGAGGTGGGCCTGAGGAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGGAGCGTGCTGAGCAAGGCCAAGATCCT
GCTGCTGGACGAGCCCAGCGCCCACCTGGACCCCGTGACCTACCAGATCATCAGGA
GGACCCTGAAGCAGGCCTTCGCCGACTGCACCGTGATCCTGTGCGAGCACAGGATC
GAGGCCATGCTGGAGTGCCAGCAGTTCCTGGTGATCGAGGAGAACAAGGTGAGGCA
GTACGACAGCATCCAGAAGCTGCTGAACGAGAGGAGCCTGTTCAGGCAGGCCATCA
GCCCCAGCGACAGGGTGAAGCTGTTCCCCCACAGGAACAGCAGCAAGTGCAAGAGC
AAGCCCCAGATCGCCGCCCTGAAGGAGGAGACCGAGGAGGAGGTGCAGGACACCA
GGCTGTGA.

In another embodiment, an exemplary codon-optimized CFTR mRNA sequence is:

(SEQ ID NO: 20)
ATGCAGAGATCCCCTCTGGAGAAGGCCTCAGTGGTGTCCAAGCTTTTCTTCTCCTGG
ACCAGGCCCATTTTAAGAAAGGGCTACAGGCAGAGACTTGAGCTGTCTGACATCTAT
CAGATCCCTTCTGTGGATTCTGCTGACAATCTTAGTGAAAAATTGGAAAGGGAGTGG
GACAGAGAGCTGGCAAGTAAAAAGAACCCCAAGCTGATTAATGCCCTGAGGCGCTG
CTTTTTTTGGAGATTCATGTTCTATGGCATATTCCTCTACCTTGGAGAAGTAACCAAA
GCTGTACAGCCTCTCCTCCTTGGCAGAATCATTGCCTCCTATGATCCTGATAACAAG
GAGGAGAGAAGCATAGCCATCTACCTGGGCATTGGGCTGTGCCTCTTGTTTATTGTG
AGGACCCTTCTCTTGCACCCTGCCATCTTTGGCCTTCATCACATTGGCATGCAAATGA
GAATAGCAATGTTTAGTCTTATTTACAAAAAAACATTAAAACTCTCTTCCAGGGTGT
TGGACAAGATCAGTATTGGACAACTGGTCAGCCTGCTGAGCAACAACCTGAACAAG
TTTGATGAAGGACTGGCCCTGGCCCACTTTGTCTGGATTGCCCCCCTTCAGGTGGCTC
TTTTGATGGGCCTGATCTGGGAACTCCTGCAGGCCTCTGCCTTCTGTGGGTTAGGCTT
CCTGATAGTGCTAGCTCTCTTTCAGGCAGGGTTGGGTAGAATGATGATGAAGTACAG
AGACCAGAGGGCTGGGAAGATATCTGAGAGGCTGGTCATTACTTCTGAAATGATAG
AAAACATCCAGTCTGTTAAAGCTTACTGCTGGGAGGAGGCTATGGAAAAGATGATT
GAGAACTTGAGGCAAACAGAGCTCAAGCTGACTAGGAAGGCAGCCTATGTCAGGTA
TTTCAACAGCAGTGCTTTCTTCTTCTCAGGCTTTTTCGTGGTCTTCTTGAGTGTTCTGC
CCTATGCCCTCATCAAGGGGATAATTTTGAGAAAGATTTTCACCACTATTTCCTTTTG
CATTGTCCTGAGGATGGCTGTCACCAGGCAATTCCCCTGGGCTGTGCAGACATGGTA
TGACTCTCTGGGGGCCATCAACAAAATCCAAGATTTCCTGCAGAAGCAGGAGTACA
AGACCCTGGAATACAACCTCACCACCACAGAAGTTGTGATGGAGAATGTGACTGCA
TTCTGGGAGGAAGGATTTGGGGAGCTGTTTGAGAAAGCAAAACAAAACAATAATAA
CAGGAAAACCAGCAATGGAGATGACTCCCTGTTCTTTTCCAACTTCTCTTTGTTGGG
CACCCCTGTCCTGAAAGATATAAACTTTAAAATTGAAAGAGGGCAGCTGTTGGCAGT
TGCTGGCTCCACAGGAGCTGGAAAAACTTCACTACTGATGGTGATCATGGGGGAGTT
AGAACCCTCTGAAGGGAAAATAAAACATTCTGGGAGGATTAGTTTCTGCAGCCAGT
TCAGCTGGATCATGCCTGGGACCATTAAAGAAAATATTATATTTGGAGTGAGCTATG
ATGAATATAGATATAGGAGTGTCATCAAAGCCTGTCAGTTGGAGGAAGACATCAGC
AAATTTGCAGAGAAAGACAACATTGTTCTGGGTGAAGGTGGCATCACCCTGTCAGG
AGGGCAAAGGGCCAGGATCAGCTTGGCCAGAGCAGTCTATAAAGATGCTGATCTGT
ACCTCCTGGATAGCCCTTTTGGCTATCTGGATGTTTTGACAGAGAAGGAAATTTTTG
AGTCCTGTGTCTGCAAGTTAATGGCAAATAAAACAAGGATACTTGTGACCTCAAAA
ATGGAACACCTGAAGAAGGCTGACAAAATTCTGATCCTGCATGAGGGCAGCAGCTA
CTTTTATGGAACATTTTCTGAACTGCAGAATTTGCAACCAGACTTTTCATCAAAGCTC
ATGGGATGTGACAGTTTTGATCAGTTTTCTGCAGAAAGGAGAAACTCCATTTTGACT
GAGACCCTGCACAGGTTCAGTCTGGAGGGGGATGCCCCAGTGAGTTGGACTGAGAC
AAAGAAACAGAGCTTCAAGCAGACTGGAGAGTTTGGAGAAAAGAGGAAAAACTCA
ATTCTCAATCCCATCAATAGCATCAGGAAGTTCAGCATAGTTCAGAAGACTCCTTTG
CAGATGAATGGGATTGAAGAGGACTCAGATGAGCCCCTGGAAAGGAGACTCTCCTT
GGTGCCAGATTCAGAGCAGGGGGAAGCCATACTGCCAAGGATCTCTGTGATTTCTAC
AGGGCCCACCCTCCAAGCAAGAAGGAGACAGTCAGTTTTAAACCTGATGACCCACT
CTGTCAACCAGGGACAGAACATTCATAGAAAGACAACAGCATCTACAAGAAAAGTT
TCACTGGCCCCTCAAGCCAATTTAACTGAACTAGATATCTACAGCAGGAGGCTCAGC
CAAGAAACAGGCCTGGAGATCTCAGAAGAAATAAATGAGGAGGATTTGAAGGAAT
GCTTCTTTGATGATATGGAGAGCATCCCAGCTGTCACAACCTGGAACACCTACCTGA
GATACATCACAGTGCACAAATCCCTCATCTTTGTACTTATATGGTGCCTTGTCATCTT
CTTAGCTGAGGTGGCTGCTTCCCTGGTGGTGCTGTGGCTGCTGGGAAACACACCCCT
CCAGGATAAAGGGAACTCTACTCACAGCAGGAACAACAGTTATGCTGTGATCATCA
CCAGTACCTCCTCCTACTATGTGTTCTACATTTATGTTGGAGTTGCAGACACATTGCT
TGCCATGGGTTTTTTTAGAGGACTCCCCCTGGTGCATACTCTCATCACTGTTTCCAAA
ATCCTTCACCACAAGATGCTGCACAGTGTACTACAGGCTCCCATGAGCACCCTCAAC
ACTCTTAAAGCAGGAGGAATCTTGAACAGATTTAGCAAGGACATTGCAATTCTTGAT
GACCTGCTTCCACTGACCATCTTTGACTTCATCCAGCTTCTGCTCATTGTAATTGGTG
CCATTGCTGTGGTAGCAGTGCTCCAGCCATATATTTTTGTGGCCACTGTGCCTGTTAT
TGTGGCCTTCATTATGTTGAGAGCCTACTTCCTGCAGACCTCTCAGCAGCTCAAGCA
ACTTGAAAGTGAGGGCAGGAGCCCCATATTTACACACTTGGTCACTTCCCTCAAAGG
CCTCTGGACACTCAGAGCTTTTGGAAGACAACCTTATTTTGAAACTCTCTTCCACAA
GGCTCTGAATCTCCACACAGCCAACTGGTTTCTGTATCTTTCAACACTGCGCTGGTTC
CAGATGAGGATTGAGATGATCTTTGTTATCTTCTTCATAGCTGTTACCTTCATCTCTA
TTCTGACAACTGGTGAGGGGGAAGGGAGAGTAGGCATCATCCTCACACTAGCCATG
AACATAATGTCTACCTTACAATGGGCCGTGAACAGCTCCATAGATGTGGACAGCCTC
ATGAGAAGTGTGTCAAGAGTTTTCAAATTCATTGACATGCCCACAGAAGGCAAACC
AACCAAGAGCACAAAACCCTACAAGAATGGCCAGCTGAGTAAGGTCATGATCATTG
AAAATTCTCATGTGAAGAAGGATGATATTTGGCCCAGTGGGGGCCAGATGACAGTC
AAGGACCTCACTGCCAAATACACAGAGGGTGGAAATGCTATCCTAGAGAACATCTC
CTTCTCCATCTCCCCAGGCCAAAGAGTTGGCTTGCTGGGCAGGACTGGCAGTGGCAA
GTCCACCTTGCTCTCAGCATTTCTCAGGCTTTTAAATACAGAGGGAGAGATTCAAAT
TGATGGGGTGTCTTGGGATAGTATAACACTTCAACAGTGGAGGAAAGCCTTTGGTGT
GATTCCTCAGAAAGTGTTTATCTTCTCTGGCACTTTCAGAAAAAATCTGGACCCCTAT
GAACAGTGGAGTGACCAGGAAATCTGGAAGGTGGCAGATGAAGTGGGCCTAAGATC
AGTCATAGAGCAGTTTCCTGGAAAGTTGGATTTTGTGCTTGTAGATGGAGGCTGTGT
GCTGTCCCATGGCCATAAACAGCTAATGTGCCTGGCTAGGTCAGTGCTGAGCAAGGC
CAAGATCCTGCTGTTAGATGAGCCTTCAGCCCATCTGGACCCTGTGACATACCAGAT
TATCAGAAGAACTCTGAAGCAGGCCTTTGCTGACTGCACTGTCATCCTGTGTGAGCA
CAGAATTGAGGCCATGCTGGAGTGCCAGCAGTTCCTTGTTATAGAAGAGAATAAGG
TTAGGCAGTATGACAGCATTCAGAAACTGCTAAATGAAAGATCTCTCTTCAGGCAAG
CTATTTCACCATCTGATAGAGTGAAACTTTTTCCCCACAGAAATTCCTCTAAATGTAA
ATCTAAGCCCCAGATAGCTGCCTTGAAAGAGGAGACTGAAGAAGAAGTCCAGGACA
CCAGACTGTGA.

In another embodiment, an exemplary codon-optimized CFTR mRNA sequence is:

(SEQ ID NO: 21)
ATGCAGAGATCCCCGCTGGAGAAGGCATCTGTGGTGTCAAAACTGTTCTTTAGCTGG
ACAAGGCCCATCCTTAGGAAAGGGTACAGACAGAGGTTGGAGCTGTCAGACATATA
TCAGATCCCTTCAGTGGACTCTGCAGACAACCTCTCTGAAAAGCTGGAGAGGGAAT
GGGACAGGGAACTGGCCAGCAAAAAAAACCCTAAACTGATTAATGCCCTGAGGAGG
TGCTTCTTTTGGAGATTCATGTTCTATGGGATCTTCCTTTACCTGGGGGAGGTGACTA
AAGCTGTTCAGCCTCTTCTTCTGGGGAGGATTATTGCCTCCTATGACCCAGACAACA
AAGAAGAAAGAAGCATAGCCATTTACTTAGGCATAGGCCTCTGCTTGCTCTTCATAG
TTAGAACCCTCCTACTCCACCCAGCCATCTTTGGTCTCCACCACATAGGTATGCAGA
TGAGAATAGCAATGTTCTCCTTGATCTACAAGAAGACCCTCAAGCTGTCCAGCAGGG
TGCTGGACAAGATCTCCATAGGCCAGTTAGTCAGTCTACTGTCCAATAACTTAAATA
AGTTTGATGAGGGACTGGCACTGGCACATTTTGTGTGGATTGCCCCCCTCCAAGTGG
CCCTTCTTATGGGCCTTATCTGGGAGCTGTTGCAGGCCTCTGCTTTCTGTGGCCTGGG
TTTCCTCATAGTCCTAGCCTTATTCCAGGCTGGACTGGGCAGAATGATGATGAAGTA
TAGGGACCAAAGAGCAGGGAAGATTTCTGAAAGGCTGGTTATAACTTCTGAGATGA
TTGAGAACATTCAGTCAGTGAAAGCTTACTGCTGGGAAGAAGCTATGGAAAAAATG
ATTGAAAATCTCAGACAGACTGAATTAAAGTTGACCAGGAAAGCTGCTTATGTCAG
ATACTTCAACTCCTCAGCCTTCTTTTTTTCTGGCTTCTTTGTTGTATTCCTTTCAGTCC
TCCCCTATGCCCTGATTAAGGGCATTATCTTGAGGAAAATTTTCACAACCATCTCCTT
TTGTATTGTCCTCAGGATGGCTGTTACAAGGCAATTTCCTTGGGCTGTGCAAACTTG
GTATGATAGCCTTGGAGCAATCAACAAGATCCAGGATTTCCTGCAAAAGCAGGAGT
ACAAGACATTGGAATACAACCTTACCACCACTGAGGTGGTGATGGAAAATGTGACT
GCCTTCTGGGAGGAGGGGTTTGGAGAGCTGTTTGAGAAAGCCAAACAGAACAACAA
CAATAGAAAGACCTCTAATGGTGATGATTCCCTGTTCTTTTCTAACTTTAGTCTTCTG
GGGACCCCAGTTCTGAAAGATATTAACTTTAAAATTGAAAGGGGACAGTTGCTGGCT
GTGGCTGGGTCCACTGGGGCTGGGAAGACAAGCCTGCTCATGGTGATCATGGGAGA
GCTGGAACCCAGTGAAGGAAAGATCAAACACTCAGGCAGGATCTCCTTCTGCAGCC
AGTTCTCATGGATTATGCCAGGCACTATTAAAGAAAATATCATCTTTGGTGTAAGCT
ATGATGAGTACAGGTATAGATCTGTAATTAAAGCCTGCCAGCTGGAGGAAGACATC
TCTAAGTTTGCTGAGAAGGATAACATTGTGTTGGGGGAAGGGGGCATCACCCTTTCT
GGTGGGCAGAGGGCTAGGATCTCCCTTGCTAGGGCAGTATACAAGGATGCTGACTT
GTACCTCTTGGATAGTCCTTTTGGCTACCTAGATGTGCTGACAGAGAAAGAAATATT
TGAAAGCTGTGTGTGTAAGCTCATGGCTAACAAGACCAGGATCCTGGTCACCAGTA
AAATGGAACACCTCAAAAAAGCAGACAAGATCCTTATTCTCCATGAGGGCTCCTCCT
ACTTCTATGGGACCTTCAGTGAGCTGCAGAATCTGCAGCCAGACTTCTCCTCAAAAC
TTATGGGCTGTGACTCCTTTGACCAATTCTCTGCAGAAAGAAGGAATAGCATACTGA
CAGAAACACTGCATAGATTCTCCCTGGAAGGAGATGCCCCAGTGAGTTGGACAGAA
ACCAAAAAGCAGAGCTTCAAGCAGACTGGTGAGTTTGGTGAAAAGAGGAAGAATTC
TATCCTGAACCCCATCAATAGCATCAGGAAATTTAGCATAGTCCAAAAGACCCCCCT
CCAGATGAATGGAATAGAGGAGGATAGTGATGAGCCTCTTGAGAGAAGGCTGTCCC
TGGTTCCAGACAGTGAACAGGGTGAAGCCATTCTTCCGAGGATCAGTGTCATCTCCA
CTGGGCCCACATTGCAGGCCAGAAGAAGACAGTCTGTTCTGAATTTGATGACACATT
CTGTGAATCAAGGCCAGAATATCCATAGAAAAACCACTGCCAGCACCAGAAAAGTT
TCTCTAGCCCCCCAGGCTAACCTGACTGAGTTAGACATCTACAGCAGAAGGCTGAGC
CAAGAGACTGGCTTGGAAATATCTGAGGAGATCAATGAGGAGGACCTCAAGGAGTG
CTTCTTTGATGACATGGAGTCAATCCCTGCAGTCACTACATGGAACACTTACCTAAG
GTACATCACAGTTCATAAGAGCCTCATCTTTGTCCTCATATGGTGTCTGGTCATCTTT
TTAGCAGAAGTGGCTGCCAGCCTAGTTGTGCTGTGGTTACTGGGCAATACACCTCTT
CAGGACAAAGGCAATAGCACACACAGCAGAAACAACTCCTATGCAGTGATCATCAC
CTCTACAAGCTCTTACTATGTATTCTATATATATGTGGGAGTGGCAGATACTCTCCTG
GCCATGGGATTCTTCAGGGGATTACCTCTAGTTCACACATTGATCACAGTGTCAAAA
ATTCTCCACCACAAGATGTTACACAGTGTCCTGCAAGCCCCAATGTCTACTCTGAAC
ACACTTAAGGCAGGTGGAATTTTGAATAGGTTTAGCAAGGACATAGCTATCCTGGAT
GATCTCCTCCCTCTGACCATCTTTGACTTCATCCAGTTACTGCTCATTGTAATTGGAG
CCATTGCAGTGGTAGCAGTCCTACAGCCTTACATTTTTGTGGCTACTGTTCCTGTTAT
TGTGGCCTTCATTATGCTAAGAGCTTACTTCCTGCAAACAAGCCAACAGTTGAAACA
GCTAGAAAGTGAGGGAAGGTCCCCCATCTTCACCCACCTGGTGACATCACTCAAGG
GGCTATGGACTCTTAGGGCTTTTGGGAGACAGCCGTACTTTGAGACCTTATTCCATA
AGGCCCTTAACCTCCATACAGCAAACTGGTTCTTATACCTGAGTACTCTGAGGTGGT
TTCAAATGAGGATTGAAATGATTTTTGTGATCTTCTTCATTGCTGTGACCTTCATCTC
AATCTTGACCACAGGAGAGGGGGAGGGCAGGGTGGGCATCATACTGACCTTGGCCA
TGAACATTATGTCAACCCTGCAGTGGGCTGTCAATAGCTCCATTGATGTGGACAGTC
TGATGAGGAGTGTCTCCAGGGTCTTCAAGTTTATTGACATGCCAACTGAGGGCAAAC
CCACCAAAAGCACTAAGCCATATAAAAATGGCCAACTGTCCAAAGTGATGATCATT
GAAAATTCACATGTAAAGAAGGATGATATCTGGCCCTCTGGAGGACAGATGACAGT
GAAAGACCTGACTGCCAAGTACACAGAGGGTGGTAATGCCATTCTTGAGAACATTA
GTTTCAGTATTTCCCCGGGGCAAAGGGTGGGCCTCCTTGGCAGAACAGGCTCTGGCA
AGAGTACCCTGCTGTCAGCCTTTTTAAGACTGTTGAACACTGAGGGAGAAATTCAGA
TTGATGGTGTCTCCTGGGATAGCATCACCCTCCAGCAGTGGAGAAAAGCTTTTGGAG
TGATCCCGCAAAAGGTTTTCATCTTTTCAGGCACCTTCCGGAAGAACCTGGACCCCT
ATGAGCAGTGGTCTGACCAGGAAATATGGAAGGTAGCTGATGAAGTTGGGCTTAGG
TCAGTCATAGAGCAGTTCCCAGGCAAACTGGACTTTGTCCTGGTGGATGGTGGATGT
GTACTGAGTCATGGGCACAAACAGCTGATGTGCCTAGCCAGGTCTGTGCTCAGCAA
GGCAAAGATATTGCTGCTTGATGAACCCAGTGCCCATCTGGACCCAGTCACATATCA
GATCATCAGAAGAACATTGAAGCAGGCCTTTGCTGATTGCACAGTTATCCTCTGTGA
GCACAGGATTGAGGCCATGCTGGAGTGCCAGCAGTTTCTGGTGATTGAGGAGAATA
AAGTAAGGCAGTATGACTCCATCCAGAAGCTGCTCAATGAAAGAAGCCTCTTTAGA
CAAGCTATCTCCCCCTCAGACAGGGTCAAATTGTTCCCTCACAGAAACAGCAGCAA
GTGCAAGAGCAAGCCCCAAATTGCAGCCTTGAAAGAGGAGACAGAGGAAGAGGTG
CAGGACACCAGACTCTGA.

In another embodiment, an exemplary codon-optimized CFTR mRNA sequence is:

(SEQ ID NO: 22)
ATGCAGAGAAGCCCCCTGGAGAAGGCCAGCGTGGTGAGCAAGCTGTTCTTCAGCTG
GACCAGACCCATCCTGAGAAAGGGCTACAGACAGAGACTGGAGCTGAGCGACATCT
ACCAGATCCCCAGCGTGGACAGCGCCGACAACCTGAGCGAGAAGCTGGAGAGAGA
GTGGGACAGAGAGCTGGCCAGCAAGAAGAACCCCAAGCTGATCAACGCCCTGAGA
AGATGCTTCTTCTGGAGATTCATGTTCTACGGCATCTTCCTGTACCTGGGCGAGGTG
ACCAAGGCCGTGCAGCCCCTGCTGCTGGGCAGAATCATCGCCAGCTACGACCCCGA
CAACAAGGAGGAGAGAAGCATCGCCATCTACCTGGGCATCGGCCTGTGCCTGCTGT
TCATCGTGAGAACCCTGCTGCTGCACCCCGCCATCTTCGGCCTGCACCACATCGGCA
TGCAGATGAGAATCGCCATGTTCAGCCTGATCTACAAGAAGACCCTGAAGCTGAGC
AGCAGAGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGAATGAT
GATGAAGTACAGAGACCAGAGAGCCGGCAAGATCAGCGAGAGACTGGTGATCACC
AGCGAGATGATCGAGAACATCCAGAGCGTGAAGGCCTACTGCTGGGAGGAGGCCAT
GGAGAAGATGATCGAGAACCTGAGACAGACCGAGCTGAAGCTGACCAGAAAGGCC
GCCTACGTGAGATACTTCAACAGCAGCGCCTTCTTCTTCAGCGGCTTCTTCGTGGTGT
TCCTGAGCGTGCTGCCCTACGCCCTGATCAAGGGCATCATCCTGAGAAAGATCTTCA
CCACCATCAGCTTCTGCATCGTGCTGAGAATGGCCGTGACCAGACAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGAAAGACCAGCAACGGCGACGACAGCCTGTTCTTCAGC
AACTTCAGCCTGCTGGGCACCCCCGTGCTGAAGGACATCAACTTCAAGATCGAGAG
AGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
AATCAGCTTCTGCAGCCAGTTCAGCTGGATCATGCCCGGCACCATCAAGGAGAACA
TCATCTTCGGCGTGAGCTACGACGAGTACAGATACAGAAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGAGCCAGAATCAGCCTGGCCAGAGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGAATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGAAGAAACAGCATCCTGACCGAGACCCTGCACAGATTCAGCCTGGAGGGCGA
CGCCCCCGTGAGCTGGACCGAGACCAAGAAGCAGAGCTTCAAGCAGACCGGCGAGT
TCGGCGAGAAGAGAAAGAACAGCATCCTGAACCCCATCAACAGCATCAGAAAGTTC
AGCATCGTGCAGAAGACCCCCCTGCAGATGAACGGCATCGAGGAGGACAGCGACG
AGCCCCTGGAGAGAAGACTGAGCCTGGTGCCCGACAGCGAGCAGGGCGAGGCCATC
CTGCCCAGAATCAGCGTGATCAGCACCGGCCCCACCCTGCAGGCCAGAAGAAGACA
GAGCGTGCTGAACCTGATGACCCACAGCGTGAACCAGGGCCAGAACATCCACAGAA
AGACCACCGCCAGCACCAGAAAGGTGAGCCTGGCCCCCCAGGCCAACCTGACCGAG
CTGGACATCTACAGCAGAAGACTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGATACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGAAACAACAGCTACGCCGTGATCATCACCAGCACCAGCAGCTACTACGTGTTC
TACATCTACGTGGGCGTGGCCGACACCCTGCTGGCCATGGGCTTCTTCAGAGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGATTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
AGCCTACTTCCTGCAGACCAGCCAGCAGCTGAAGCAGCTGGAGAGCGAGGGCAGAA
GCCCCATCTTCACCCACCTGGTGACCAGCCTGAAGGGCCTGTGGACCCTGAGAGCCT
TCGGCAGACAGCCCTACTTCGAGACCCTGTTCCACAAGGCCCTGAACCTGCACACCG
CCAACTGGTTCCTGTACCTGAGCACCCTGAGATGGTTCCAGATGAGAATCGAGATGA
TCTTCGTGATCTTCTTCATCGCCGTGACCTTCATCAGCATCCTGACCACCGGCGAGG
GCGAGGGCAGAGTGGGCATCATCCTGACCCTGGCCATGAACATCATGAGCACCCTG
CAGTGGGCCGTGAACAGCAGCATCGACGTGGACAGCCTGATGAGAAGCGTGAGCAG
AGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGAGTGGGCCTGCTGGGCAGAACCGGCAGCGGCAAGAGCACCCTGCTGAGC
GCCTTCCTGAGACTGCTGAACACCGAGGGCGAGATCCAGATCGACGGCGTGAGCTG
GGACAGCATCACCCTGCAGCAGTGGAGAAAGGCCTTCGGCGTGATCCCCCAGAAGG
TGTTCATCTTCAGCGGCACCTTCAGAAAGAACCTGGACCCCTACGAGCAGTGGAGC
GACCAGGAGATCTGGAAGGTGGCCGACGAGGTGGGCCTGAGAAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGAAGCGTGCTGAGCAAGGCCAAGATCCT
GCTGCTGGACGAGCCCAGCGCCCACCTGGACCCCGTGACCTACCAGATCATCAGAA
GAACCCTGAAGCAGGCCTTCGCCGACTGCACCGTGATCCTGTGCGAGCACAGAATC
GAGGCCATGCTGGAGTGCCAGCAGTTCCTGGTGATCGAGGAGAACAAGGTGAGACA
GTACGACAGCATCCAGAAGCTGCTGAACGAGAGAAGCCTGTTCAGACAGGCCATCA
GCCCCAGCGACAGAGTGAAGCTGTTCCCCCACAGAAACAGCAGCAAGTGCAAGAGC
AAGCCCCAGATCGCCGCCCTGAAGGAGGAGACCGAGGAGGAGGTGCAGGACACCA
GACTGTGA.

In another embodiment, an exemplary codon-optimized CFTR mRNA sequence is:

(SEQ ID NO: 23)
ATGCAGCGCAGCCCCCTGGAGAAGGCCAGCGTGGTGAGCAAGCTGTTCTTCAGCTG
GACCCGCCCCATCCTGCGCAAGGGCTACCGCCAGCGCCTGGAGCTGAGCGACATCT
ACCAGATCCCCAGCGTGGACAGCGCCGACAACCTGAGCGAGAAGCTGGAGCGCGA
GTGGGACCGCGAGCTGGCCAGCAAGAAGAACCCCAAGCTGATCAACGCCCTGCGCC
GCTGCTTCTTCTGGCGCTTCATGTTCTACGGCATCTTCCTGTACCTGGGCGAGGTGAC
CAAGGCCGTGCAGCCCCTGCTGCTGGGCCGCATCATCGCCAGCTACGACCCCGACA
ACAAGGAGGAGCGCAGCATCGCCATCTACCTGGGCATCGGCCTGTGCCTGCTGTTCA
TCGTGCGCACCCTGCTGCTGCACCCCGCCATCTTCGGCCTGCACCACATCGGCATGC
AGATGCGCATCGCCATGTTCAGCCTGATCTACAAGAAGACCCTGAAGCTGAGCAGC
CGCGTGCTGGACAAGATCAGCATCGGCCAGCTGGTGAGCCTGCTGAGCAACAACCT
GAACAAGTTCGACGAGGGCCTGGCCCTGGCCCACTTCGTGTGGATCGCCCCCCTGCA
GGTGGCCCTGCTGATGGGCCTGATCTGGGAGCTGCTGCAGGCCAGCGCCTTCTGCGG
CCTGGGCTTCCTGATCGTGCTGGCCCTGTTCCAGGCCGGCCTGGGCCGCATGATGAT
GAAGTACCGCGACCAGCGCGCCGGCAAGATCAGCGAGCGCCTGGTGATCACCAGCG
AGATGATCGAGAACATCCAGAGCGTGAAGGCCTACTGCTGGGAGGAGGCCATGGAG
AAGATGATCGAGAACCTGCGCCAGACCGAGCTGAAGCTGACCCGCAAGGCCGCCTA
CGTGCGCTACTTCAACAGCAGCGCCTTCTTCTTCAGCGGCTTCTTCGTGGTGTTCCTG
AGCGTGCTGCCCTACGCCCTGATCAAGGGCATCATCCTGCGCAAGATCTTCACCACC
ATCAGCTTCTGCATCGTGCTGCGCATGGCCGTGACCCGCCAGTTCCCCTGGGCCGTG
CAGACCTGGTACGACAGCCTGGGCGCCATCAACAAGATCCAGGACTTCCTGCAGAA
GCAGGAGTACAAGACCCTGGAGTACAACCTGACCACCACCGAGGTGGTGATGGAGA
ACGTGACCGCCTTCTGGGAGGAGGGCTTCGGCGAGCTGTTCGAGAAGGCCAAGCAG
AACAACAACAACCGCAAGACCAGCAACGGCGACGACAGCCTGTTCTTCAGCAACTT
CAGCCTGCTGGGCACCCCCGTGCTGAAGGACATCAACTTCAAGATCGAGCGCGGCC
AGCTGCTGGCCGTGGCCGGCAGCACCGGCGCCGGCAAGACCAGCCTGCTGATGGTG
ATCATGGGCGAGCTGGAGCCCAGCGAGGGCAAGATCAAGCACAGCGGCCGCATCA
GCTTCTGCAGCCAGTTCAGCTGGATCATGCCCGGCACCATCAAGGAGAACATCATCT
TCGGCGTGAGCTACGACGAGTACCGCTACCGCAGCGTGATCAAGGCCTGCCAGCTG
GAGGAGGACATCAGCAAGTTCGCCGAGAAGGACAACATCGTGCTGGGCGAGGGCG
GCATCACCCTGAGCGGCGGCCAGCGCGCCCGCATCAGCCTGGCCCGCGCCGTGTAC
AAGGACGCCGACCTGTACCTGCTGGACAGCCCCTTCGGCTACCTGGACGTGCTGACC
GAGAAGGAGATCTTCGAGAGCTGCGTGTGCAAGCTGATGGCCAACAAGACCCGCAT
CCTGGTGACCAGCAAGATGGAGCACCTGAAGAAGGCCGACAAGATCCTGATCCTGC
ACGAGGGCAGCAGCTACTTCTACGGCACCTTCAGCGAGCTGCAGAACCTGCAGCCC
GACTTCAGCAGCAAGCTGATGGGCTGCGACAGCTTCGACCAGTTCAGCGCCGAGCG
CCGCAACAGCATCCTGACCGAGACCCTGCACCGCTTCAGCCTGGAGGGCGACGCCC
CCGTGAGCTGGACCGAGACCAAGAAGCAGAGCTTCAAGCAGACCGGCGAGTTCGGC
GAGAAGCGCAAGAACAGCATCCTGAACCCCATCAACAGCATCCGCAAGTTCAGCAT
CGTGCAGAAGACCCCCCTGCAGATGAACGGCATCGAGGAGGACAGCGACGAGCCCC
TGGAGCGCCGCCTGAGCCTGGTGCCCGACAGCGAGCAGGGCGAGGCCATCCTGCCC
CGCATCAGCGTGATCAGCACCGGCCCCACCCTGCAGGCCCGCCGCCGCCAGAGCGT
GCTGAACCTGATGACCCACAGCGTGAACCAGGGCCAGAACATCCACCGCAAGACCA
CCGCCAGCACCCGCAAGGTGAGCCTGGCCCCCCAGGCCAACCTGACCGAGCTGGAC
ATCTACAGCCGCCGCCTGAGCCAGGAGACCGGCCTGGAGATCAGCGAGGAGATCAA
CGAGGAGGACCTGAAGGAGTGCTTCTTCGACGACATGGAGAGCATCCCCGCCGTGA
CCACCTGGAACACCTACCTGCGCTACATCACCGTGCACAAGAGCCTGATCTTCGTGC
TGATCTGGTGCCTGGTGATCTTCCTGGCCGAGGTGGCCGCCAGCCTGGTGGTGCTGT
GGCTGCTGGGCAACACCCCCCTGCAGGACAAGGGCAACAGCACCCACAGCCGCAAC
AACAGCTACGCCGTGATCATCACCAGCACCAGCAGCTACTACGTGTTCTACATCTAC
GTGGGCGTGGCCGACACCCTGCTGGCCATGGGCTTCTTCCGCGGCCTGCCCCTGGTG
CACACCCTGATCACCGTGAGCAAGATCCTGCACCACAAGATGCTGCACAGCGTGCT
GCAGGCCCCCATGAGCACCCTGAACACCCTGAAGGCCGGCGGCATCCTGAACCGCT
TCAGCAAGGACATCGCCATCCTGGACGACCTGCTGCCCCTGACCATCTTCGACTTCA
TCCAGCTGCTGCTGATCGTGATCGGCGCCATCGCCGTGGTGGCCGTGCTGCAGCCCT
ACATCTTCGTGGCCACCGTGCCCGTGATCGTGGCCTTCATCATGCTGCGCGCCTACTT
CCTGCAGACCAGCCAGCAGCTGAAGCAGCTGGAGAGCGAGGGCCGCAGCCCCATCT
TCACCCACCTGGTGACCAGCCTGAAGGGCCTGTGGACCCTGCGCGCCTTCGGCCGCC
AGCCCTACTTCGAGACCCTGTTCCACAAGGCCCTGAACCTGCACACCGCCAACTGGT
TCCTGTACCTGAGCACCCTGCGCTGGTTCCAGATGCGCATCGAGATGATCTTCGTGA
TCTTCTTCATCGCCGTGACCTTCATCAGCATCCTGACCACCGGCGAGGGCGAGGGCC
GCGTGGGCATCATCCTGACCCTGGCCATGAACATCATGAGCACCCTGCAGTGGGCC
GTGAACAGCAGCATCGACGTGGACAGCCTGATGCGCAGCGTGAGCCGCGTGTTCAA
GTTCATCGACATGCCCACCGAGGGCAAGCCCACCAAGAGCACCAAGCCCTACAAGA
ACGGCCAGCTGAGCAAGGTGATGATCATCGAGAACAGCCACGTGAAGAAGGACGA
CATCTGGCCCAGCGGCGGCCAGATGACCGTGAAGGACCTGACCGCCAAGTACACCG
AGGGCGGCAACGCCATCCTGGAGAACATCAGCTTCAGCATCAGCCCCGGCCAGCGC
GTGGGCCTGCTGGGCCGCACCGGCAGCGGCAAGAGCACCCTGCTGAGCGCCTTCCT
GCGCCTGCTGAACACCGAGGGCGAGATCCAGATCGACGGCGTGAGCTGGGACAGCA
TCACCCTGCAGCAGTGGCGCAAGGCCTTCGGCGTGATCCCCCAGAAGGTGTTCATCT
TCAGCGGCACCTTCCGCAAGAACCTGGACCCCTACGAGCAGTGGAGCGACCAGGAG
ATCTGGAAGGTGGCCGACGAGGTGGGCCTGCGCAGCGTGATCGAGCAGTTCCCCGG
CAAGCTGGACTTCGTGCTGGTGGACGGCGGCTGCGTGCTGAGCCACGGCCACAAGC
AGCTGATGTGCCTGGCCCGCAGCGTGCTGAGCAAGGCCAAGATCCTGCTGCTGGAC
GAGCCCAGCGCCCACCTGGACCCCGTGACCTACCAGATCATCCGCCGCACCCTGAA
GCAGGCCTTCGCCGACTGCACCGTGATCCTGTGCGAGCACCGCATCGAGGCCATGCT
GGAGTGCCAGCAGTTCCTGGTGATCGAGGAGAACAAGGTGCGCCAGTACGACAGCA
TCCAGAAGCTGCTGAACGAGCGCAGCCTGTTCCGCCAGGCCATCAGCCCCAGCGAC
CGCGTGAAGCTGTTCCCCCACCGCAACAGCAGCAAGTGCAAGAGCAAGCCCCAGAT
CGCCGCCCTGAAGGAGGAGACCGAGGAGGAGGTGCAGGACACCCGCCTGTAA.

In yet another embodiment, an exemplary codon-optimized CFTR mRNA sequence is:

(SEQ ID NO: 24)
ATGCAGAGAAGCCCCCTGGAGAAGGCCAGCGTGGTGAGCAAGCTGTTCTTCAGCTG
GACCAGACCCATCCTGAGAAAGGGCTACAGACAGAGACTGGAGCTGAGCGACATCT
ACCAGATCCCCAGCGTGGACAGCGCCGACAACCTGAGCGAGAAGCTGGAGAGAGA
GTGGGACAGAGAGCTGGCCAGCAAGAAGAACCCCAAGCTGATCAACGCCCTGAGA
AGATGCTTCTTCTGGAGATTCATGTTCTACGGCATCTTCCTGTACCTGGGCGAGGTG
ACCAAGGCCGTGCAGCCCCTGCTGCTGGGCAGAATCATCGCCAGCTACGACCCCGA
CAACAAGGAGGAGAGAAGCATCGCCATCTACCTGGGCATCGGCCTGTGCCTGCTGT
TCATCGTGAGAACCCTGCTGCTGCACCCCGCCATCTTCGGCCTGCACCACATCGGCA
TGCAGATGAGAATCGCCATGTTCAGCCTGATCTACAAGAAGACCCTGAAGCTGAGC
AGCAGAGTGCTGGACAAGATCAGCATCGGCCAGCTGGTGAGCCTGCTGAGCAACAA
CCTGAACAAGTTCGACGAGGGCCTGGCCCTGGCCCACTTCGTGTGGATCGCCCCCCT
GCAGGTGGCCCTGCTGATGGGCCTGATCTGGGAGCTGCTGCAGGCCAGCGCCTTCTG
CGGCCTGGGCTTCCTGATCGTGCTGGCCCTGTTCCAGGCCGGCCTGGGCAGAATGAT
GATGAAGTACAGGGACCAGAGAGCCGGCAAGATCAGCGAGAGACTGGTGATCACC
AGCGAGATGATCGAGAACATCCAGAGCGTGAAGGCCTACTGCTGGGAGGAGGCCAT
GGAGAAGATGATCGAGAACCTGAGACAGACCGAGCTGAAGCTGACCAGAAAGGCC
GCCTACGTGAGATACTTCAACAGCAGCGCCTTCTTCTTCAGCGGCTTCTTCGTGGTGT
TCCTGAGCGTGCTGCCCTACGCCCTGATCAAGGGCATCATCCTGAGAAAGATCTTCA
CCACCATCAGCTTCTGCATCGTGCTGAGAATGGCCGTGACCAGACAGTTCCCCTGGG
CCGTGCAGACCTGGTACGACAGCCTGGGCGCCATCAACAAGATCCAGGACTTCCTG
CAGAAGCAGGAGTACAAGACCCTGGAGTACAACCTGACCACCACCGAGGTGGTGAT
GGAGAACGTGACCGCCTTCTGGGAGGAGGGCTTCGGCGAGCTGTTCGAGAAGGCCA
AGCAGAACAACAACAACAGAAAGACCAGCAACGGCGACGACAGCCTGTTCTTCAGC
AACTTCAGCCTGCTGGGCACCCCCGTGCTGAAGGACATCAACTTCAAGATCGAGAG
AGGCCAGCTGCTGGCCGTGGCCGGCAGCACCGGCGCCGGCAAGACCAGCCTGCTGA
TGGTGATCATGGGCGAGCTGGAGCCCAGCGAGGGCAAGATCAAGCACAGCGGCAG
AATCAGCTTCTGCAGCCAGTTCAGCTGGATCATGCCCGGCACCATCAAGGAGAACA
TCATCTTCGGCGTGAGCTACGACGAGTACAGATACAGAAGCGTGATCAAGGCCTGC
CAGCTGGAGGAGGACATCAGCAAGTTCGCCGAGAAGGACAACATCGTGCTGGGCGA
GGGCGGCATCACCCTGAGCGGCGGCCAGAGAGCCAGAATCAGCCTGGCCAGAGCCG
TGTACAAGGACGCCGACCTGTACCTGCTGGACAGCCCCTTCGGCTACCTGGACGTGC
TGACCGAGAAGGAGATCTTCGAGAGCTGCGTGTGCAAGCTGATGGCCAACAAGACC
AGAATCCTGGTGACCAGCAAGATGGAGCACCTGAAGAAGGCCGACAAGATCCTGAT
CCTGCACGAGGGCAGCAGCTACTTCTACGGCACCTTCAGCGAGCTGCAGAACCTGC
AGCCCGACTTCAGCAGCAAGCTGATGGGCTGCGACAGCTTCGACCAGTTCAGCGCC
GAGAGAAGAAACAGCATCCTGACCGAGACCCTGCACAGATTCAGCCTGGAGGGCGA
CGCCCCCGTGAGCTGGACCGAGACCAAGAAGCAGAGCTTCAAGCAGACCGGCGAGT
TCGGCGAGAAGAGAAAGAACAGCATCCTGAACCCCATCAACAGCATCAGAAAGTTC
AGCATCGTGCAGAAGACCCCCCTGCAGATGAACGGCATCGAGGAGGACAGCGACG
AGCCCCTGGAGAGAAGACTGAGCCTGGTGCCCGACAGCGAGCAGGGCGAGGCCATC
CTGCCCAGAATCAGCGTGATCAGCACCGGCCCCACCCTGCAGGCCAGAAGAAGACA
GAGCGTGCTGAACCTGATGACCCACAGCGTGAACCAGGGCCAGAACATCCACAGAA
AGACCACCGCCAGCACCAGAAAGGTGAGCCTGGCCCCCCAGGCCAACCTGACCGAG
CTGGACATCTACAGCAGAAGACTGAGCCAGGAGACCGGCCTGGAGATCAGCGAGG
AGATCAACGAGGAGGACCTGAAGGAGTGCTTCTTCGACGACATGGAGAGCATCCCC
GCCGTGACCACCTGGAACACCTACCTGAGATACATCACCGTGCACAAGAGCCTGAT
CTTCGTGCTGATCTGGTGCCTGGTGATCTTCCTGGCCGAGGTGGCCGCCAGCCTGGT
GGTGCTGTGGCTGCTGGGCAACACCCCCCTGCAGGACAAGGGCAACAGCACCCACA
GCAGAAACAACAGCTACGCCGTGATCATCACCAGCACCAGCAGCTACTACGTGTTC
TACATCTACGTGGGCGTGGCCGACACCCTGCTGGCCATGGGCTTCTTCAGAGGCCTG
CCCCTGGTGCACACCCTGATCACCGTGAGCAAGATCCTGCACCACAAGATGCTGCAC
AGCGTGCTGCAGGCCCCCATGAGCACCCTGAACACCCTGAAGGCCGGCGGCATCCT
GAACAGATTCAGCAAGGACATCGCCATCCTGGACGACCTGCTGCCCCTGACCATCTT
CGACTTCATCCAGCTGCTGCTGATCGTGATCGGCGCCATCGCCGTGGTGGCCGTGCT
GCAGCCCTACATCTTCGTGGCCACCGTGCCCGTGATCGTGGCCTTCATCATGCTGAG
AGCCTACTTCCTGCAGACCAGCCAGCAGCTGAAGCAGCTGGAGAGCGAGGGCAGGA
GCCCCATCTTCACCCACCTGGTGACCAGCCTGAAGGGCCTGTGGACCCTGAGAGCCT
TCGGCAGACAGCCCTACTTCGAGACCCTGTTCCACAAGGCCCTGAACCTGCACACCG
CCAACTGGTTCCTGTACCTGAGCACCCTGAGATGGTTCCAGATGAGAATCGAGATGA
TCTTCGTGATCTTCTTCATCGCCGTGACCTTCATCAGCATCCTGACCACCGGCGAGG
GCGAGGGCAGAGTGGGCATCATCCTGACCCTGGCCATGAACATCATGAGCACCCTG
CAGTGGGCCGTGAACAGCAGCATCGACGTGGACAGCCTGATGAGAAGCGTGAGCAG
AGTGTTCAAGTTCATCGACATGCCCACCGAGGGCAAGCCCACCAAGAGCACCAAGC
CCTACAAGAACGGCCAGCTGAGCAAGGTGATGATCATCGAGAACAGCCACGTGAAG
AAGGACGACATCTGGCCCAGCGGCGGCCAGATGACCGTGAAGGACCTGACCGCCAA
GTACACCGAGGGCGGCAACGCCATCCTGGAGAACATCAGCTTCAGCATCAGCCCCG
GCCAGAGAGTGGGCCTGCTGGGCAGAACCGGCAGCGGCAAGAGCACCCTGCTGAGC
GCCTTCCTGAGACTGCTGAACACCGAGGGCGAGATCCAGATCGACGGCGTGAGCTG
GGACAGCATCACCCTGCAGCAGTGGAGAAAGGCCTTCGGCGTGATCCCCCAGAAGG
TGTTCATCTTCAGCGGCACCTTCAGAAAGAACCTGGACCCCTACGAGCAGTGGAGC
GACCAGGAGATCTGGAAGGTGGCCGACGAGGTGGGCCTGAGAAGCGTGATCGAGC
AGTTCCCCGGCAAGCTGGACTTCGTGCTGGTGGACGGCGGCTGCGTGCTGAGCCAC
GGCCACAAGCAGCTGATGTGCCTGGCCAGAAGCGTGCTGAGCAAGGCCAAGATCCT
GCTGCTGGACGAGCCCAGCGCCCACCTGGACCCCGTGACCTACCAGATCATCAGAA
GAACCCTGAAGCAGGCCTTCGCCGACTGCACCGTGATCCTGTGCGAGCACAGAATC
GAGGCCATGCTGGAGTGCCAGCAGTTCCTGGTGATCGAGGAGAACAAGGTGAGACA
GTACGACAGCATCCAGAAGCTGCTGAACGAGAGAAGCCTGTTCAGACAGGCCATCA
GCCCCAGCGACAGAGTGAAGCTGTTCCCCCACAGAAACAGCAGCAAGTGCAAGAGC
AAGCCCCAGATCGCCGCCCTGAAGGAGGAGACCGAGGAGGAGGTGCAGGACACCA
GACTGTGA.

In another embodiment, an exemplary codon-optimized CFTR mRNA sequence is:

(SEQ ID NO: 25)
ATGCAGAGGTCACCTCTGGAAAAGGCTAGCGTGGTCAGCAAGCTATTTTTTTCCTGG
ACCCGCCCGATACTCAGGAAGGGCTACCGACAGCGGCTGGAGCTGAGTGACATTTA
TCAGATTCCCTCCGTCGATTCCGCTGACAACCTGTCTGAGAAACTGGAGCGGGAATG
GGATAGGGAACTGGCGTCCAAAAAAAACCCCAAACTCATCAATGCACTCCGCAGAT
GCTTCTTCTGGCGGTTTATGTTTTATGGCATATTCCTGTATCTGGGGGAGGTGACGAA
AGCCGTGCAGCCGCTGCTGCTTGGTCGCATTATCGCGTCATACGATCCAGATAACAA
GGAGGAAAGAAGTATCGCTATCTATCTCGGGATAGGGCTGTGCCTGCTCTTCATTGT
GCGGACTCTTCTCTTGCACCCCGCCATTTTCGGTCTGCATCATATAGGTATGCAGATG
AGAATTGCGATGTTCTCATTGATTTACAAAAAAACGCTTAAGCTAAGTTCAAGGGTG
CTAGATAAGATATCGATCGGCCAGCTGGTGTCTCTGCTTAGCAACAACCTCAATAAA
TTCGACGAAGGCCTTGCACTGGCCCACTTCGTGTGGATCGCCCCTCTGCAGGTGGCT
CTGCTGATGGGGTTAATATGGGAGCTGTTGCAGGCCTCCGCTTTTTGTGGCCTGGGG
TTTCTCATCGTGTTGGCCTTGTTTCAGGCAGGGCTGGGACGTATGATGATGAAATAT
AGGGATCAGAGGGCTGGCAAAATCTCTGAGCGCCTGGTTATTACGAGTGAAATGAT
TGAGAACATCCAGTCAGTGAAGGCCTATTGCTGGGAGGAGGCCATGGAAAAAATGA
TTGAGAACCTACGCCAGACTGAGCTGAAGTTAACCAGAAAAGCCGCCTATGTGCGC
TACTTTAACAGTAGCGCATTTTTCTTCTCCGGTTTTTTCGTGGTGTTTCTTAGTGTGTT
GCCGTATGCCTTAATCAAGGGAATAATACTCCGGAAGATTTTCACTACCATCAGCTT
CTGTATCGTGTTGCGGATGGCCGTCACCCGGCAGTTTCCCTGGGCAGTACAGACTTG
GTACGATTCTCTCGGAGCAATTAACAAAATCCAAGACTTTCTACAAAAGCAGGAGT
ACAAGACCCTGGAGTACAATCTGACCACCACAGAAGTCGTAATGGAGAATGTAACT
GCCTTCTGGGAAGAGGGCTTTGGCGAACTCTTTGAAAAGGCCAAGCAGAACAATAA
CAACCGGAAGACCTCCAACGGGGACGACAGCTTATTTTTCAGCAATTTTTCTTTGCT
CGGGACCCCTGTACTGAAAGATATTAACTTTAAGATCGAGCGCGGACAACTCCTGGC
TGTCGCCGGCAGCACTGGAGCTGGAAAAACATCACTGCTTATGGTGATAATGGGAG
AACTCGAACCAAGCGAGGGAAAAATAAAGCACTCTGGACGGATTAGTTTTTGCTCC
CAGTTCTCGTGGATAATGCCTGGCACCATTAAGGAGAATATCATCTTTGGAGTGAGT
TACGACGAATACCGGTACCGGTCCGTTATCAAGGCTTGTCAACTCGAGGAGGACATT
TCTAAATTCGCCGAAAAAGATAATATAGTGCTGGGCGAAGGAGGCATTACACTGAG
CGGGGGTCAGAGAGCTCGAATTAGCCTCGCCCGAGCAGTCTATAAAGACGCCGATC
TTTACCTGCTGGATTCCCCTTTTGGGTATTTGGATGTTCTGACAGAGAAGGAAATCTT
TGAATCATGTGTCTGTAAACTGATGGCCAATAAGACTAGGATTCTAGTGACTTCGAA
AATGGAGCACCTGAAAAAAGCGGACAAAATTCTGATACTCCATGAAGGGTCTTCCT
ACTTCTACGGCACCTTCTCAGAGTTGCAGAACTTACAACCTGATTTTTCATCTAAGCT
TATGGGGTGCGACTCGTTTGACCAGTTCTCCGCTGAAAGACGAAACAGCATCTTAAC
GGAAACTCTTCACAGGTTCTCATTAGAGGGAGATGCGCCGGTGTCCTGGACAGAGA
CAAAAAAACAGTCTTTCAAACAGACAGGAGAGTTTGGCGAGAAGAGAAAAAACTC
AATCCTCAATCCCATCAATTCTATTAGAAAGTTTAGCATCGTCCAAAAAACACCATT
GCAGATGAATGGGATTGAGGAGGACAGTGATGAGCCTTTGGAACGAAGACTGTCCC
TGGTACCCGATAGCGAACAGGGTGAGGCCATCCTTCCTAGGATCTCGGTCATAAGTA
CAGGGCCCACACTGCAGGCCAGGCGACGTCAAAGTGTCCTCAATCTTATGACGCAC
AGTGTGAATCAGGGGCAGAACATCCATCGTAAGACGACAGCTTCAACTCGAAAGGT
CAGTCTAGCTCCACAAGCCAATCTTACAGAGCTGGACATTTATTCCCGCCGCCTCAG
TCAGGAGACCGGATTGGAAATATCAGAGGAAATTAATGAAGAGGATCTGAAGGAAT
GCTTCTTTGATGACATGGAATCGATCCCCGCTGTTACTACCTGGAACACATATCTGA
GATATATTACCGTCCATAAGAGCTTAATCTTTGTACTGATATGGTGCTTGGTGATTTT
CCTGGCAGAGGTTGCGGCGAGTTTGGTCGTGCTATGGCTCCTTGGAAACACTCCCCT
GCAGGATAAGGGGAACTCCACTCATAGCAGGAATAACAGCTATGCCGTGATCATCA
CCTCTACCTCCTCTTATTACGTGTTTTACATATACGTCGGTGTTGCGGATACCCTGTT
GGCAATGGGGTTCTTTAGAGGACTACCCCTAGTTCACACCCTGATCACCGTTTCGAA
GATCTTGCACCACAAGATGCTTCATAGCGTTCTCCAAGCTCCTATGAGCACCCTTAA
TACACTGAAAGCAGGAGGTATCCTTAACCGCTTTTCCAAAGACATCGCTATACTCGA
CGATTTGCTCCCATTGACCATCTTCGACTTCATTCAGCTGCTCCTCATTGTGATCGGC
GCCATTGCCGTGGTCGCAGTGTTACAGCCATATATTTTCGTAGCCACCGTGCCCGTC
ATCGTGGCATTTATCATGCTGCGCGCATATTTCTTACAGACATCTCAGCAACTGAAG
CAGCTGGAATCTGAGGGCAGATCTCCTATTTTTACACACCTGGTTACCAGCCTGAAG
GGCCTGTGGACCCTGCGTGCTTTCGGTCGCCAACCCTACTTTGAGACTCTCTTCCATA
AGGCTCTGAATTTACATACTGCCAATTGGTTCCTATACCTTAGTACCCTTCGGTGGTT
CCAGATGCGGATAGAAATGATCTTCGTGATTTTCTTCATCGCAGTCACTTTCATCTCT
ATTTTGACGACCGGTGAGGGCGAGGGCAGGGTGGGCATCATTCTGACTTTGGCCATG
AACATTATGTCAACACTCCAGTGGGCCGTTAATTCAAGCATTGATGTGGATTCCTTG
ATGCGTTCCGTCAGCAGGGTATTTAAATTCATAGACATGCCCACCGAGGGCAAGCCA
ACAAAATCTACCAAGCCATACAAAAATGGCCAACTAAGCAAGGTCATGATTATCGA
GAATTCTCATGTGAAAAAGGACGACATTTGGCCTTCCGGGGGTCAAATGACTGTAA
AGGACCTGACGGCTAAATACACTGAGGGCGGTAATGCTATCTTGGAGAACATCTCTT
TCAGCATCTCCCCTGGCCAGAGAGTGGGACTGCTCGGGCGGACAGGCTCCGGAAAG
TCTACGCTCCTTTCAGCATTCCTTAGACTTCTGAACACCGAAGGTGAGATTCAGATT
GACGGGGTCTCTTGGGACTCCATCACACTTCAGCAATGGAGGAAGGCATTCGGTGTA
ATCCCCCAAAAGGTTTTTATCTTCTCCGGAACATTTCGTAAGAATCTGGACCCGTAC
GAGCAGTGGTCAGATCAGGAGATCTGGAAAGTAGCAGACGAGGTCGGGCTACGGA
GCGTTATTGAACAGTTTCCTGGCAAACTGGACTTCGTTTTGGTGGACGGAGGCTGTG
TGCTGAGTCACGGCCATAAACAACTGATGTGCTTAGCTAGGTCTGTTCTCAGCAAGG
CAAAGATTTTACTGCTGGATGAACCAAGCGCCCACCTTGATCCAGTGACATATCAAA
TCATCAGAAGAACTCTTAAACAGGCGTTCGCCGACTGCACAGTGATCCTGTGTGAGC
ACAGAATAGAAGCCATGCTGGAATGTCAACAGTTTCTCGTGATTGAGGAGAACAAG
GTGCGCCAGTACGATAGCATCCAGAAGTTACTCAATGAAAGGTCACTCTTCAGGCA
GGCCATCTCACCCAGCGACCGCGTTAAGCTGTTTCCACACCGAAACAGTTCCAAGTG
CAAAAGTAAGCCACAGATTGCTGCACTGAAGGAAGAGACAGAAGAAGAAGTTCAG
GACACTCGGCTCTGA.

In another embodiment, an exemplary codon-optimized CFTR mRNA sequence is:

(SEQ ID NO: 26)
ATGCAGAGGAGCCCACTGGAGAAAGCCTCCGTGGTGAGTAAACTCTTTTTTAGTTGG
ACCAGACCCATCCTGCGAAAAGGATACAGGCAGCGCCTCGAGTTGTCAGATATCTA
CCAGATTCCTTCTGTGGACTCAGCTGACAATTTGAGTGAGAAGCTGGAGCGGGAGTG
GGATAGAGAGCTGGCGAGCAAAAAAAACCCCAAGCTTATCAATGCTCTGCGCCGCT
GCTTTTTCTGGAGGTTCATGTTTTATGGGATCTTCCTGTACCTGGGGGAGGTCACCAA
AGCTGTTCAGCCGCTCCTTCTTGGCCGCATCATCGCCAGCTATGACCCTGATAATAA
AGAAGAAAGGTCTATTGCTATTTATCTGGGAATTGGCCTCTGCTTGCTCTTCATCGTC
CGCACCCTTCTGCTGCACCCTGCCATTTTTGGCCTTCACCACATCGGCATGCAAATG
AGAATTGCCATGTTCTCCCTCATTTACAAAAAGACCCTGAAACTTTCCTCAAGAGTG
TTAGATAAAATATCCATTGGTCAGCTGGTCAGCCTGCTGTCCAACAATCTTAACAAA
TTTGATGAAGGCTTGGCGCTGGCCCACTTCGTGTGGATTGCACCTCTGCAGGTGGCC
CTGTTGATGGGACTTATATGGGAGCTGCTTCAAGCCTCTGCTTTCTGTGGGCTGGGCT
TTTTGATTGTACTGGCACTTTTTCAGGCTGGGCTCGGAAGAATGATGATGAAATACA
GAGATCAGCGGGCCGGGAAGATATCAGAGCGACTTGTGATCACCAGTGAAATGATT
GAAAATATTCAGAGCGTGAAAGCCTACTGCTGGGAAGAAGCCATGGAGAAGATGAT
TGAGAACCTGAGGCAGACAGAGCTCAAGCTCACTCGGAAGGCTGCTTATGTTCGCT
ATTTCAACAGCAGCGCCTTCTTCTTCAGTGGCTTCTTTGTTGTCTTCCTGTCTGTTCTG
CCATATGCACTGATAAAAGGCATTATTTTACGAAAGATCTTCACCACCATCAGTTTT
TGCATCGTTCTCAGGATGGCCGTCACAAGACAGTTCCCCTGGGCTGTGCAGACCTGG
TACGATTCCTTGGGGGCCATCAACAAGATTCAAGATTTCTTGCAAAAACAAGAATAT
AAAACTTTAGAATACAACCTCACCACCACTGAAGTGGTCATGGAAAATGTGACAGC
CTTTTGGGAGGAGGGTTTTGGAGAATTGTTCGAGAAGGCAAAGCAGAATAACAACA
ACAGGAAGACGAGCAATGGGGACGACTCTCTCTTCTTCAGCAACTTTTCACTGCTCG
GGACCCCTGTGTTGAAAGATATAAACTTCAAGATCGAGAGGGGCCAGCTCTTGGCT
GTGGCAGGCTCCACTGGAGCTGGTAAAACATCTCTTCTCATGGTGATCATGGGGGAA
CTGGAGCCTTCCGAAGGAAAAATCAAGCACAGTGGGAGAATCTCATTCTGCAGCCA
GTTTTCCTGGATCATGCCCGGCACCATTAAGGAAAACATCATATTTGGAGTGTCCTA
TGATGAGTACCGCTACCGGTCAGTCATCAAAGCCTGTCAGTTGGAGGAGGACATCTC
CAAGTTTGCAGAGAAAGACAACATTGTGCTTGGAGAGGGGGGTATCACTCTTTCTGG
AGGACAAAGAGCCAGGATCTCTTTGGCCCGGGCAGTCTACAAGGATGCAGACCTCT
ACTTGTTGGACAGTCCCTTCGGCTACCTCGACGTGCTGACTGAAAAAGAAATTTTTG
AAAGCTGTGTGTGCAAACTGATGGCAAACAAGACCAGGATTCTTGTCACCAGCAAG
ATGGAACATCTGAAGAAAGCGGACAAAATTCTGATTCTGCATGAAGGGAGCTCCTA
CTTCTATGGAACATTTAGCGAGCTTCAGAACCTACAGCCAGACTTCTCCTCCAAATT
AATGGGCTGTGACTCCTTCGACCAGTTCTCTGCAGAAAGAAGAAACTCTATACTCAC
AGAGACCCTCCACCGCTTCTCCCTTGAGGGAGATGCCCCAGTTTCTTGGACAGAAAC
CAAGAAGCAGTCCTTTAAGCAGACTGGCGAGTTTGGTGAAAAGAGGAAAAATTCAA
TTCTCAATCCAATTAACAGTATTCGCAAGTTCAGCATTGTCCAGAAGACACCCCTCC
AGATGAATGGCATCGAAGAAGATAGTGACGAGCCGCTGGAGAGACGGCTGAGTCTG
GTGCCAGATTCAGAACAGGGGGAGGCCATCCTGCCCCGGATCAGCGTCATTTCCAC
AGGCCCCACATTACAAGCACGGCGCCGGCAGAGTGTTTTAAATCTCATGACCCATTC
AGTGAACCAGGGCCAAAATATCCACAGGAAGACTACAGCTTCTACCCGGAAAGTGT
CTCTGGCCCCTCAGGCCAATCTGACCGAGCTGGACATCTACAGCAGGAGGCTCTCCC
AGGAAACAGGGCTGGAAATATCTGAAGAGATTAATGAAGAGGATCTTAAAGAGTGC
TTCTTTGATGACATGGAGAGCATCCCCGCGGTGACCACATGGAACACCTACCTTAGA
TATATTACTGTCCACAAGAGCCTCATATTTGTCCTCATCTGGTGCCTGGTTATTTTCC
TCGCTGAGGTGGCGGCCAGTCTTGTTGTGCTCTGGCTGCTGGGCAACACTCCTCTCC
AGGACAAGGGCAATAGTACTCACAGCAGAAATAATTCTTATGCCGTCATCATTACA
AGCACCTCCAGCTACTACGTGTTCTACATCTATGTGGGCGTGGCTGACACCCTCCTG
GCCATGGGTTTCTTCCGGGGCCTGCCTTTGGTGCACACCCTCATCACAGTGTCAAAA
ATTCTGCACCATAAAATGCTTCATTCTGTCCTGCAGGCACCCATGAGCACTTTGAAC
ACATTGAAGGCTGGCGGCATCCTCAACAGATTTTCTAAAGATATTGCTATCCTGGAT
GATCTCCTCCCCCTGACAATCTTTGACTTTATCCAGCTTCTGCTGATCGTGATTGGAG
CCATAGCAGTGGTTGCTGTCCTGCAGCCCTACATTTTTGTGGCCACCGTGCCCGTGAT
TGTTGCCTTTATTATGCTCAGAGCTTACTTCCTGCAAACTTCTCAACAGCTCAAACAG
CTAGAATCTGAGGGCCGGAGCCCCATTTTTACCCACCTGGTGACTTCCCTGAAGGGA
CTGTGGACTCTGAGAGCATTCGGGCGACAGCCTTACTTTGAGACACTGTTCCACAAG
GCCCTGAACTTGCACACTGCCAACTGGTTTCTTTACCTGAGCACACTCCGCTGGTTCC
AGATGCGGATAGAGATGATCTTCGTCATCTTTTTTATAGCTGTAACCTTCATTTCTAT
CCTTACAACAGGAGAAGGAGAGGGCAGGGTGGGAATCATCCTCACGCTGGCTATGA
ACATAATGTCCACCTTGCAGTGGGCCGTGAATTCCAGTATAGATGTGGATTCTCTAA
TGAGGAGTGTCTCCCGGGTGTTTAAATTCATTGATATGCCTACTGAGGGGAAACCCA
CCAAGTCAACAAAACCTTATAAGAATGGACAGCTGAGCAAGGTGATGATAATTGAG
AACAGCCACGTGAAGAAGGATGACATTTGGCCCAGCGGGGGCCAGATGACTGTGAA
GGACCTGACGGCCAAGTACACCGAAGGTGGAAATGCCATTTTGGAAAACATCAGCT
TCTCAATCTCTCCTGGGCAGAGAGTTGGATTGCTGGGTCGCACGGGCAGCGGCAAAT
CAACCCTGCTCAGTGCCTTCCTTCGGCTCCTGAATACAGAAGGCGAAATCCAAATTG
ACGGGGTGAGCTGGGACAGCATCACCCTGCAGCAGTGGAGAAAAGCATTTGGGGTC
ATTCCACAGAAAGTTTTCATCTTCTCTGGCACTTTCAGAAAGAACCTGGACCCCTAT
GAGCAGTGGAGCGACCAGGAGATCTGGAAGGTTGCAGATGAAGTTGGCCTGCGGAG
TGTGATAGAACAATTTCCTGGCAAGCTGGATTTTGTGCTGGTAGATGGAGGCTGCGT
GCTGTCCCACGGCCACAAACAGCTGATGTGCCTCGCCCGCTCCGTTCTTTCAAAGGC
CAAAATCTTGCTTTTGGATGAGCCCAGTGCTCACCTCGACCCAGTGACCTATCAGAT
AATCCGCAGGACCTTAAAGCAAGCTTTTGCCGACTGCACCGTCATACTGTGTGAGCA
CCGGATTGAAGCAATGCTGGAATGCCAGCAGTTTCTGGTGATCGAGGAGAATAAGG
TCCGGCAGTACGACAGCATCCAGAAGTTGTTGAATGAGCGCAGCCTTTTCCGCCAGG
CCATCTCCCCATCTGACAGAGTCAAGCTGTTTCCACATAGGAACTCCTCTAAGTGCA
AGTCCAAGCCCCAGATCGCTGCCCTCAAGGAGGAAACTGAGGAAGAGGTGCAGGAT
ACCCGCCTGTGA.

In another embodiment, an exemplary codon-optimized CFTR mRNA sequence is:

(SEQ ID NO: 27)
ATGCAACGGAGTCCTCTGGAAAAAGCCTCTGTCGTATCTAAGCTTTTCTTCAGTTGG
ACACGCCCGATTTTGAGAAAGGGTTATCGGCAACGCTTGGAACTTAGTGACATCTAC
CAAATTCCAAGTGTAGACTCAGCCGATAACTTGAGCGAAAAGCTCGAACGAGAGTG
GGATCGAGAACTGGCTAGCAAAAAAAATCCCAAACTCATAAATGCCCTGCGACGCT
GTTTCTTTTGGCGATTTATGTTTTACGGTATTTTCCTTTATTTGGGTGAGGTCACGAA
GGCTGTACAGCCACTGCTGCTGGGTCGCATCATTGCCTCTTACGACCCTGACAACAA
AGAGGAGCGGTCAATAGCTATCTACCTTGGTATAGGACTTTGCTTGCTCTTCATAGT
CCGCACGTTGCTTCTCCACCCTGCTATATTTGGTCTCCATCACATTGGGATGCAAATG
CGGATCGCGATGTTCAGTCTTATATATAAAAAGACTCTTAAACTTTCCAGCCGGGTT
CTGGATAAGATCTCTATTGGTCAACTGGTATCTCTTTTGTCTAACAACCTGAATAAGT
TCGACGAGGGCCTTGCATTGGCCCATTTTGTATGGATTGCCCCTTTGCAAGTCGCCCT
CCTGATGGGATTGATCTGGGAACTCCTGCAAGCTAGTGCTTTTTGCGGATTGGGATT
CCTCATAGTCCTTGCGCTCTTTCAGGCGGGACTTGGACGCATGATGATGAAGTATCG
CGACCAACGAGCTGGCAAGATCAGTGAACGGCTTGTAATAACCAGTGAAATGATAG
AGAACATCCAGAGCGTAAAAGCTTACTGTTGGGAAGAAGCGATGGAAAAGATGATT
GAGAACCTTCGCCAGACAGAACTTAAACTTACACGAAAGGCCGCTTATGTCCGGTA
CTTCAACTCTTCAGCATTTTTTTTTAGTGGCTTCTTTGTAGTGTTCCTGTCCGTCCTTC
CGTATGCACTTATCAAGGGTATAATACTTAGGAAAATCTTCACAACAATCAGTTTTT
GCATAGTCCTTCGCATGGCAGTAACTCGCCAATTTCCCTGGGCAGTTCAGACGTGGT
ACGACTCACTTGGCGCAATTAACAAAATTCAAGATTTCCTCCAAAAGCAAGAGTATA
AAACCTTGGAATACAACCTTACCACCACAGAAGTTGTAATGGAAAATGTCACAGCC
TTCTGGGAGGAAGGTTTCGGCGAACTTTTTGAGAAGGCGAAGCAAAATAACAATAA
TCGGAAAACATCAAACGGTGACGATTCACTGTTCTTTTCTAACTTTAGCCTTCTTGGG
ACGCCCGTCCTGAAGGACATAAACTTTAAGATTGAACGGGGTCAACTTCTCGCGGTC
GCAGGGAGTACTGGAGCGGGGAAAACGAGCCTGCTGATGGTGATAATGGGGGAGTT
GGAGCCCTCAGAAGGCAAGATCAAGCATAGTGGTAGAATTAGCTTCTGCAGTCAAT
TTAGTTGGATTATGCCGGGCACGATCAAAGAAAATATAATCTTTGGGGTATCCTACG
ATGAATACAGGTACCGATCAGTGATAAAAGCGTGCCAGCTTGAAGAAGACATTTCA
AAGTTTGCTGAGAAGGATAATATCGTACTTGGAGAAGGAGGTATCACCCTGTCTGG
GGGTCAACGAGCGAGGATCTCCCTGGCACGCGCCGTCTACAAGGACGCGGACCTCT
ATCTGTTGGATTCACCGTTCGGATATTTGGACGTGCTTACGGAGAAAGAAATATTTG
AGAGCTGTGTTTGCAAGCTCATGGCAAATAAAACCAGAATATTGGTTACAAGCAAG
ATGGAGCATCTTAAGAAAGCAGATAAAATCCTGATATTGCACGAGGGCTCTTCATAC
TTCTACGGGACGTTTTCTGAGTTGCAGAACCTCCAGCCGGATTTCAGCTCTAAGCTG
ATGGGCTGTGATTCCTTTGATCAGTTTAGTGCGGAAAGACGAAACAGTATACTCACC
GAAACACTGCACAGGTTCTCTCTGGAGGGCGACGCCCCGGTTTCCTGGACAGAGAC
GAAGAAGCAGTCCTTCAAACAGACAGGCGAGTTTGGGGAGAAAAGGAAAAATAGC
ATACTCAACCCGATTAACAGCATTCGCAAGTTCAGTATAGTACAAAAGACCCCGTTG
CAGATGAACGGTATAGAGGAAGATTCTGATGAGCCACTGGAAAGACGGCTTTCTCT
CGTTCCGGACAGTGAACAGGGAGAGGCAATACTGCCTCGGATCAGCGTTATCTCTAC
AGGACCTACTTTGCAAGCTCGGCGCCGACAGTCAGTCTTGAATCTTATGACTCATAG
TGTTAATCAAGGCCAGAATATCCATCGCAAGACCACCGCAAGTACAAGGAAAGTGA
GCTTGGCACCTCAAGCAAACCTTACTGAACTTGATATCTACTCACGGCGACTTTCAC
AGGAGACCGGACTTGAAATTAGTGAAGAAATTAACGAGGAGGACCTCAAGGAGTGC
TTCTTCGATGACATGGAATCAATCCCCGCAGTCACAACCTGGAACACTTATCTGAGG
TATATAACAGTTCACAAGAGCCTCATTTTTGTACTTATTTGGTGTTTGGTAATTTTCC
TGGCGGAGGTTGCTGCTTCTTTGGTCGTCCTTTGGCTCCTCGGGAATACACCGCTCCA
AGACAAAGGCAACTCTACCCATAGTAGGAACAATTCATATGCAGTGATTATAACCA
GTACATCATCTTATTACGTTTTCTATATTTATGTCGGGGTAGCTGACACGCTGTTGGC
GATGGGCTTCTTTAGGGGCCTCCCCTTGGTACACACCCTTATCACGGTGAGTAAAAT
CCTGCATCACAAAATGCTTCATTCTGTACTCCAAGCGCCGATGAGTACGCTTAATAC
GCTGAAAGCAGGAGGGATACTGAATCGGTTCAGCAAGGACATCGCCATTCTGGATG
ACCTGCTTCCATTGACAATATTTGATTTCATTCAGCTCCTTCTCATAGTTATTGGAGC
CATAGCGGTGGTGGCTGTGCTTCAGCCTTATATATTCGTTGCCACAGTTCCCGTTATA
GTGGCATTTATAATGCTCAGGGCCTACTTTCTCCAGACTTCCCAGCAGTTGAAGCAA
CTCGAATCAGAAGGAAGGTCACCTATTTTCACACATCTTGTGACTTCCTTGAAGGGC
TTGTGGACGCTGCGGGCCTTCGGAAGACAACCATATTTTGAAACTCTCTTCCACAAA
GCTTTGAATCTTCATACTGCGAACTGGTTCCTGTATTTGAGTACTTTGCGCTGGTTCC
AGATGAGGATAGAAATGATATTCGTTATCTTCTTTATCGCGGTTACGTTCATAAGTA
TCCTCACTACGGGGGAGGGTGAGGGTAGAGTGGGCATAATACTGACCCTCGCCATG
AACATTATGTCCACCCTGCAGTGGGCGGTAAACAGCAGCATAGATGTGGATTCTTTG
ATGCGCAGTGTGAGCAGGGTTTTTAAGTTTATCGATATGCCGACGGAAGGAAAGCC
CACTAAAAGCACGAAACCCTATAAAAATGGACAGCTTAGCAAAGTAATGATAATCG
AGAATAGCCATGTGAAAAAGGATGACATATGGCCTTCCGGAGGCCAAATGACTGTT
AAAGATCTGACCGCTAAATATACCGAGGGCGGCAACGCAATACTCGAAAACATAAG
CTTTTCCATAAGCCCCGGCCAACGCGTGGGTCTTCTGGGGAGGACTGGCTCCGGAAA
ATCAACGTTGCTTAGCGCGTTTTTGCGGCTCCTTAACACTGAAGGTGAGATCCAAAT
AGATGGCGTTAGTTGGGACTCTATAACACTGCAACAATGGCGGAAAGCTTTCGGCGT
CATACCTCAGAAGGTGTTCATCTTTAGCGGAACGTTCAGGAAGAACTTGGATCCCTA
CGAACAATGGAGTGATCAAGAAATATGGAAAGTGGCAGATGAGGTAGGCTTGCGCA
GTGTCATTGAACAATTCCCAGGGAAACTCGACTTTGTACTGGTGGACGGCGGTTGCG
TCTTGTCACACGGGCACAAACAGTTGATGTGTTTGGCCCGCAGTGTTTTGTCTAAGG
CGAAGATTCTGTTGCTCGACGAACCGAGTGCTCATCTTGATCCCGTCACCTACCAAA
TCATCAGAAGGACGTTGAAGCAAGCTTTCGCCGACTGCACTGTAATCCTTTGTGAGC
ATAGGATCGAAGCAATGCTCGAGTGCCAACAGTTCTTGGTTATAGAGGAGAATAAG
GTTCGGCAATACGACTCAATACAGAAACTGCTTAATGAGCGGTCACTCTTTCGACAA
GCTATCTCTCCTAGTGACAGGGTAAAGCTTTTTCCTCATCGGAATTCCAGCAAGTGT
AAGAGTAAACCACAGATCGCCGCCCTTAAAGAGGAGACCGAAGAAGAGGTGCAGG
ATACGAGACTTTAG.

In some embodiments, a codon-optimized CFTR mRNA sequence suitable for the present invention shares at least 60%, 65%, 70%, 75%, 80%, 85%, 90%, 95%, 96%, 97%, 98%, or 99% identity to SEQ ID NO:6 or SEQ ID NO:7 and encodes a CFTR protein having an amino acid sequence of SEQ ID NO:2.

In some embodiments, a suitable mRNA sequence may be an mRNA sequence encoding a homolog or an analog of human CFTR (hCFTR) protein. For example, a homolog or an analog of hCFTR protein may be a modified hCFTR protein containing one or more amino acid substitutions, deletions, and/or insertions as compared to a wild-type or naturally-occurring hCFTR protein while retaining substantial hCFTR protein activity. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 2. In some embodiments, an mRNA suitable for the present invention encodes a protein substantially identical to hCFTR protein. In some embodiments, an mRNA suitable for the present invention encodes an amino acid sequence at least 50%, 55%, 60%, 65%, 70%, 75%, 80%, 85%, 90%, 91%, 92%, 93%, 94%, 95%, 96%, 97%, 98%, 99% or more identical to SEQ ID NO: 2. In some embodiments, an mRNA suitable for the present invention encodes a fragment or a portion of hCFTR protein. In some embodiments, an mRNA suitable for the present invention encodes a fragment or a portion of hCFTR protein, wherein the fragment or portion of the protein still maintains CFTR activity similar to that of the wild-type protein. Thus, in some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical SEQ ID NO: 1, SEQ ID NO: 6 or SEQ ID NO: 7.

In some embodiments, an mRNA suitable for the present invention has a nucleotide sequence at least 50%, 55%, 60%, 65%, 70%, 75%, 80%, 85%, 90%, 91%, 92%, 93%, 94%, 95%, 96%, 97%, 98%, 99% or more identical to any one of SEQ ID NO: 8, SEQ ID NO: 29, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, or SEQ ID NO: 27.

In some embodiments, a suitable mRNA encodes a fusion protein comprising a full length, fragment or portion of an hCFTR protein fused to another protein (e.g., an N or C terminal fusion). In some embodiments, the protein fused to the mRNA encoding a full length, fragment or portion of an hCFTR protein encodes a signal or a cellular targeting sequence.

mRNAs according to the present invention may be synthesized according to any of a variety of known methods. For example, mRNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7, or SP6 RNA polymerase), DNAse I, pyrophosphatase, and/or RNAse inhibitor. The exact conditions will vary according to the specific application.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, mRNAs (e.g., mRNAs encoding CFTR) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Additional cap structures are described in published US Application No. US 2016/0032356 and U.S. Provisional Application 62/464,327, filed Feb. 27, 2017, which are incorporated herein by reference.

In some embodiments, mRNAs (e.g., mRNAs encoding CFTR) include a 3′ tail structure. Typically, a tail structure includes a poly(A) and/or poly(C) tail. A poly-A or poly-C tail on the 3′ terminus of mRNA typically includes at least 50 adenosine or cytosine nucleotides, at least 150 adenosine or cytosine nucleotides, at least 200 adenosine or cytosine nucleotides, at least 250 adenosine or cytosine nucleotides, at least 300 adenosine or cytosine nucleotides, at least 350 adenosine or cytosine nucleotides, at least 400 adenosine or cytosine nucleotides, at least 450 adenosine or cytosine nucleotides, at least 500 adenosine or cytosine nucleotides, at least 550 adenosine or cytosine nucleotides, at least 600 adenosine or cytosine nucleotides, at least 650 adenosine or cytosine nucleotides, at least 700 adenosine or cytosine nucleotides, at least 750 adenosine or cytosine nucleotides, at least 800 adenosine or cytosine nucleotides, at least 850 adenosine or cytosine nucleotides, at least 900 adenosine or cytosine nucleotides, at least 950 adenosine or cytosine nucleotides, or at least 1 kb adenosine or cytosine nucleotides, respectively. In some embodiments, a poly-A or poly-C tail may be about 10 to 800 adenosine or cytosine nucleotides (e.g., about 10 to 200 adenosine or cytosine nucleotides, about 10 to 300 adenosine or cytosine nucleotides, about 10 to 400 adenosine or cytosine nucleotides, about 10 to 500 adenosine or cytosine nucleotides, about 10 to 550 adenosine or cytosine nucleotides, about 10 to 600 adenosine or cytosine nucleotides, about 50 to 600 adenosine or cytosine nucleotides, about 100 to 600 adenosine or cytosine nucleotides, about 150 to 600 adenosine or cytosine nucleotides, about 200 to 600 adenosine or cytosine nucleotides, about 250 to 600 adenosine or cytosine nucleotides, about 300 to 600 adenosine or cytosine nucleotides, about 350 to 600 adenosine or cytosine nucleotides, about 400 to 600 adenosine or cytosine nucleotides, about 450 to 600 adenosine or cytosine nucleotides, about 500 to 600 adenosine or cytosine nucleotides, about 10 to 150 adenosine or cytosine nucleotides, about 10 to 100 adenosine or cytosine nucleotides, about 20 to 70 adenosine or cytosine nucleotides, or about 20 to 60 adenosine or cytosine nucleotides) respectively. In some embodiments, a tail structure includes is a combination of poly(A) and poly(C) tails with various lengths described herein. In some embodiments, a tail structure includes at least 50%, 55%, 65%, 70%, 75%, 80%, 85%, 90%, 92%, 94%, 95%, 96%, 97%, 98%, or 99% adenosine nucleotides. In some embodiments, a tail structure includes at least 50%, 55%, 65%, 70%, 75%, 80%, 85%, 90%, 92%, 94%, 95%, 96%, 97%, 98%, or 99% cytosine nucleotides.

Modified mRNA

A CFTR mRNA may contain only naturally-occurring nucleotides (or unmodified nucleotides). In some embodiments, however, a suitable CFTR mRNA may contain backbone modifications, sugar modifications and/or base modifications. For example, modified nucleotides may include, but not be limited to, modified purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g., from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, mRNAs (e.g., mRNAs encoding CFTR) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, mRNAs (e.g., mRNAs encoding CFTR) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, mRNAs encoding CFTR are unmodified.

Delivery Vehicles

According to the present invention, mRNA encoding a CFTR protein (e.g., a full length, fragment, or portion of a CFTR protein) as described herein may be delivered as naked mRNA (unpackaged) or via delivery vehicles. As used herein, the terms “delivery vehicle,” “transfer vehicle,” “nanoparticle” or grammatical equivalent, are used interchangeably.

Delivery vehicles can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition. A particular delivery vehicle is selected based upon its ability to facilitate the transfection of a nucleic acid to a target cell.

According to various embodiments, suitable delivery vehicles include, but are not limited to polymer based carriers, such as polyethyleneimine (PEI), lipid nanoparticles (LNPs) and liposomes, nanoliposomes, ceramide-containing nanoliposomes, proteoliposomes, both natural and synthetically-derived exosomes, natural, synthetic and semi-synthetic lamellar bodies, nanoparticulates, calcium phosphor-silicate nanoparticulates, calcium phosphate nanoparticulates, silicon dioxide nanoparticulates, nanocrystalline particulates, semiconductor nanoparticulates, poly(D-arginine), sol-gels, nanodendrimers, starch-based delivery systems, micelles, emulsions, niosomes, multi-domain-block polymers (vinyl polymers, polypropyl acrylic acid polymers, dynamic polyconjugates), dry powder formulations, plasmids, viruses, calcium phosphate nucleotides, aptamers, peptides and other vectorial tags.

Liposomal Delivery Vehicles

In some embodiments, a suitable delivery vehicle is a liposomal delivery vehicle, e.g., a lipid nanoparticle (LNP) or liposome. In some embodiments, liposomes may comprise one or more cationic lipids. In some embodiments, a liposome comprises one or more cationic lipids, one or more non-cationic lipids, one or more cholesterol-based lipids and one or more PEG-modified lipids. In some embodiments, a liposome comprises one or more cationic lipids, one or more non-cationic lipids, and one or more PEG-modified lipids. In some embodiments, a liposome comprises no more than four distinct lipid components. In some embodiments, a liposome comprises no more than three distinct lipid components. In some embodiments, one distinct lipid component is a sterol-based cationic lipid.

As used herein, the term “cationic lipids” refers to any of a number of lipid and lipidoid species that have a net positive charge at a selected pH, such as at physiological pH. Several cationic lipids have been described in the literature, many of which are commercially available.

Suitable cationic lipids for use in the compositions and methods of the invention include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

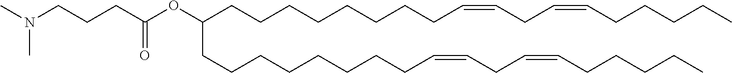 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present invention include ionizable cationic lipids as described in International Patent Publication WO 2013/149140, which is incorporated herein by reference. In some embodiments, the compositions and methods of the present invention include a cationic lipid of one of the following formulas:

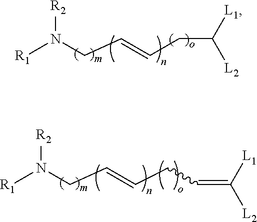

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present invention include the cationic lipid (15Z, 18Z)-N,N-dimethyl-6-(9Z,12Z)-octadeca-9,12-dien-1-yl) tetracosa-15,18-dien-1-amine (“HGT5000”), having a compound structure of:

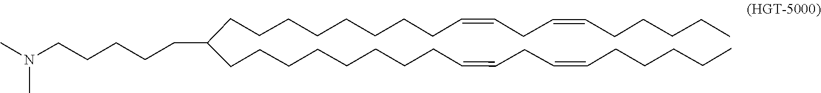 [SMILES: CCCCC/C=C\C/C=C\CCCCCCCCC(CCCCCCCC/C=C\C/C=C\CCCCC)CCCCCN(C)C]

and pharmaceutically acceptable salts thereof. In certain embodiments, the compositions and methods of the present invention include the cationic lipid (15Z, 18Z)-N,N-dimethyl-6-((9Z,12Z)-octadeca-9,12-dien-1-yl) tetracosa-4,15,18-trien-1-amine (“HGT5001”), having a compound structure of:

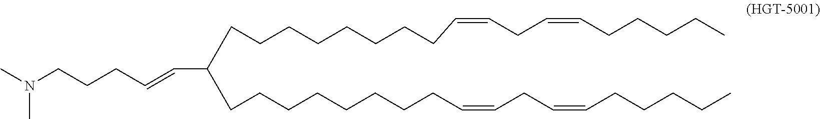 [SMILES: CCCCC/C=C\C/C=C\CCCCCCCCC(/C=C/CCCN(C)C)CCCCCCCC/C=C\C/C=C\CCCCC]

and pharmaceutically acceptable salts thereof. In certain embodiments, the compositions and methods of the present invention include the cationic lipid and (15Z,18Z)-N,N-dimethyl-6-((9Z,12Z)-octadeca-9,12-dien-1-yl) tetracosa-5,15,18-trien-1-amine (“HGT5002”), having a compound structure of:

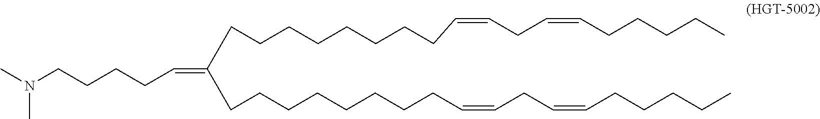 [SMILES: CCCCC/C=C\C/C=C\CCCCCCCCC(=CCCCCN(C)C)CCCCCCCC/C=C\C/C=C\CCCCC]

and pharmaceutically acceptable salts thereof.

Other suitable cationic lipids for use in the compositions and methods of the invention include cationic lipids described as aminoalcohol lipidoids in International Patent Publication WO 2010/053572, which is incorporated herein by reference. In certain embodiments, the compositions and methods of the present invention include a cationic lipid having a compound structure of:

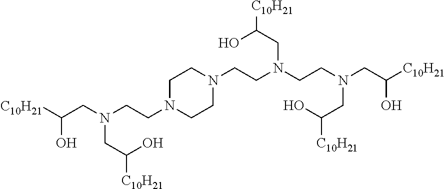

and pharmaceutically acceptable salts thereof.

Other suitable cationic lipids for use in the compositions and methods of the invention include the cationic lipids as described in International Patent Publication WO 2016/118725, which is incorporated herein by reference. In certain embodiments, the compositions and methods of the present invention include a cationic lipid having a compound structure of:

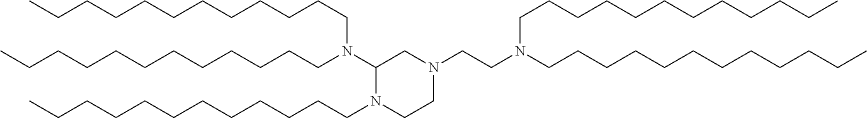 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the invention include the cationic lipids as described in International Patent Publication WO 2016/118724, which is incorporated herein by reference. In certain embodiments, the compositions and methods of the present invention include a cationic lipid having a compound structure of:

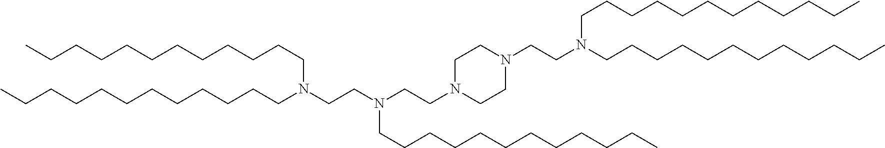 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the invention include a cationic lipid having the formula of 14,25-ditridecyl 15,18,21,24-tetraaza-octatriacontane, and pharmaceutically acceptable salts thereof.

Other suitable cationic lipids for use in the compositions and methods of the invention include the cationic lipids as described in International Patent Publications WO 2013/063468 and WO 2016/205691, each of which are incorporated herein by reference. In some embodiments, the compositions and methods of the present invention include a cationic lipid of the following formula:

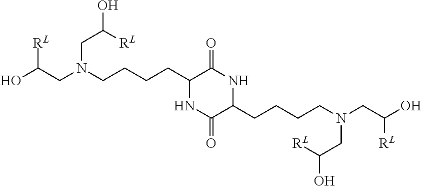

or pharmaceutically acceptable salts thereof, wherein each instance of R^{L} is independently optionally substituted C₆-C₄₀ alkenyl. In certain embodiments, the compositions and methods of the present invention include a cationic lipid having a compound structure of:

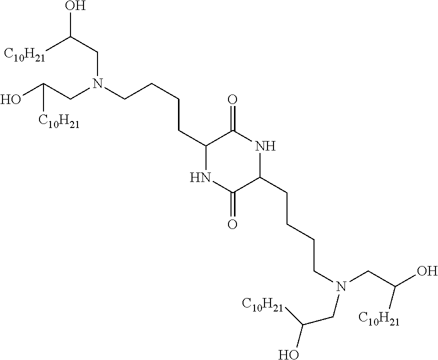

and pharmaceutically acceptable salts thereof. In certain embodiments, the compositions and methods of the present invention include a cationic lipid having a compound structure of:

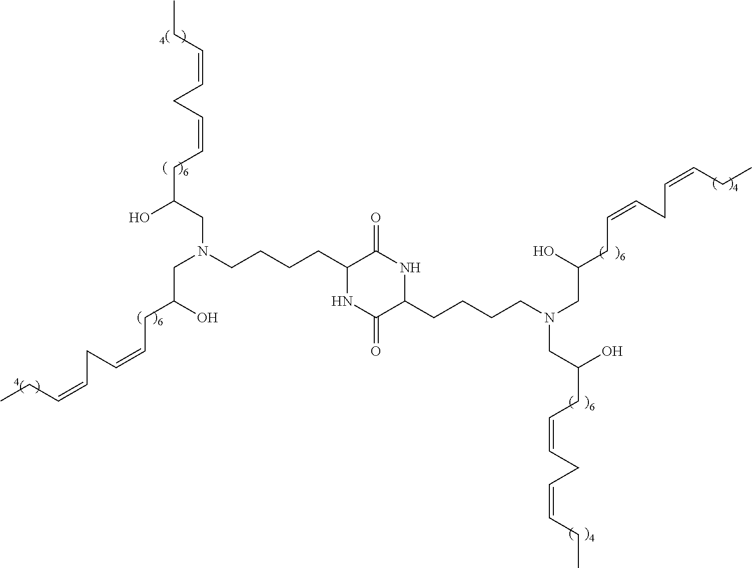

and pharmaceutically acceptable salts thereof. In certain embodiments, the compositions and methods of the present invention include a cationic lipid having a compound structure of:

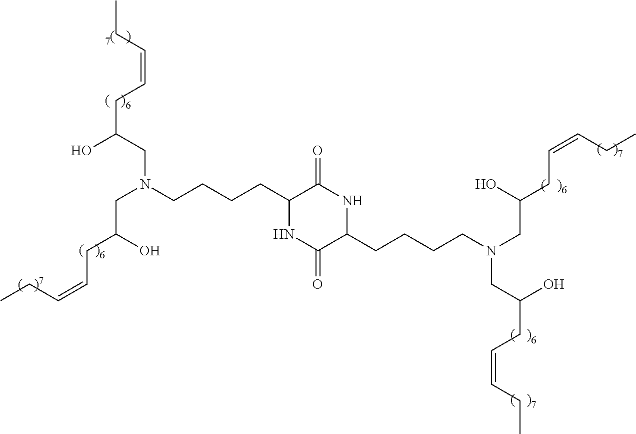

and pharmaceutically acceptable salts thereof. In certain embodiments, the compositions and methods of the present invention include a cationic lipid having a compound structure of:

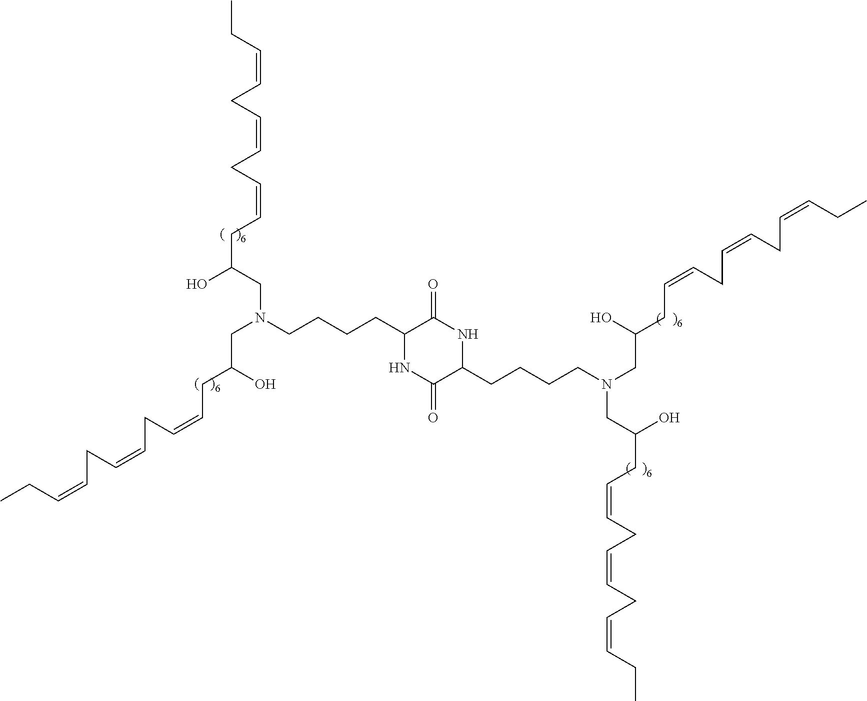

and pharmaceutically acceptable salts thereof.

Other suitable cationic lipids for use in the compositions and methods of the invention include the cationic lipids as described in International Patent Publication WO 2015/184256, which is incorporated herein by reference. In some embodiments, the compositions and methods of the present invention include a cationic lipid of the following formula:

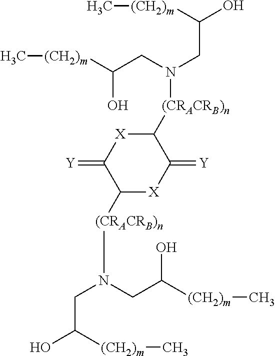

or a pharmaceutically acceptable salt thereof, wherein each X independently is O or S; each Y independently is O or S; each m independently is 0 to 20; each n independently is 1 to 6; each RA is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each R_{B} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present invention include a cationic lipid, “Target 23”, having a compound structure of:

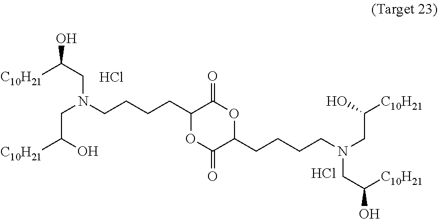

and pharmaceutically acceptable salts thereof.

Other suitable cationic lipids for use in the compositions and methods of the invention include the cationic lipids as described in International Patent Publication WO 2016/004202, which is incorporated herein by reference. In some embodiments, the compositions and methods of the present invention include a cationic lipid having the compound structure:

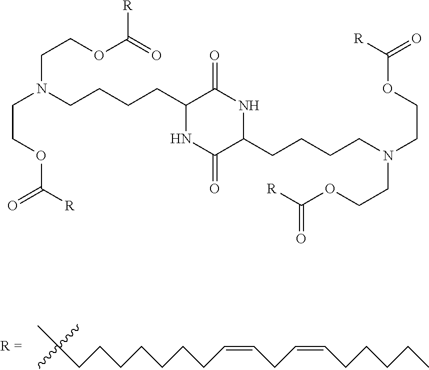

or a pharmaceutically acceptable salt thereof. In some embodiments, the compositions and methods of the present invention include a cationic lipid having the compound structure:

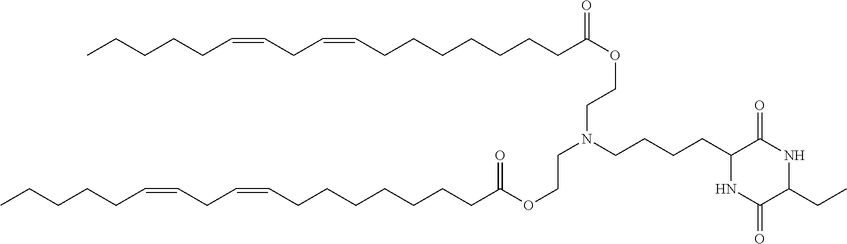 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present invention include a cationic lipid having the compound structure:

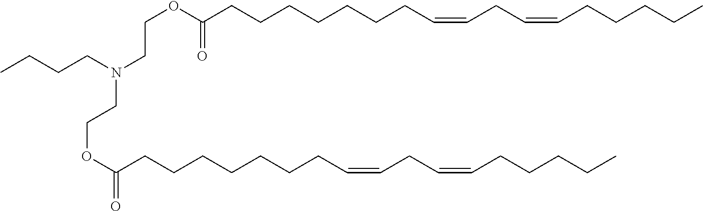 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present invention include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present invention include a cationic lipid having a compound structure of:

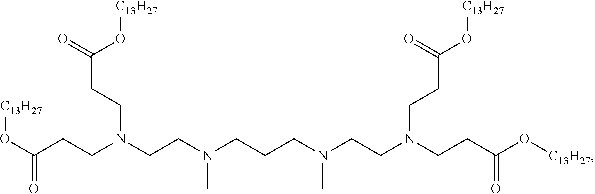

and pharmaceutically acceptable salts thereof.

Other suitable cationic lipids for use in the compositions and methods of the invention include the cationic lipids as described in International Patent Publication WO 2015/199952, which is incorporated herein by reference. In some embodiments, the compositions and methods of the present invention include a cationic lipid having the compound structure:

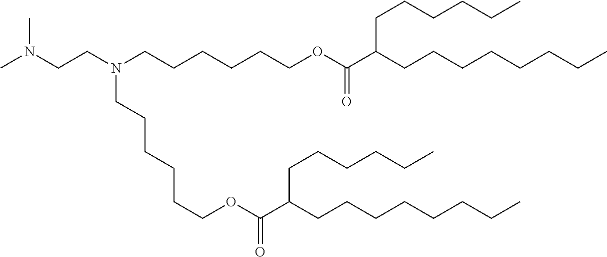 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

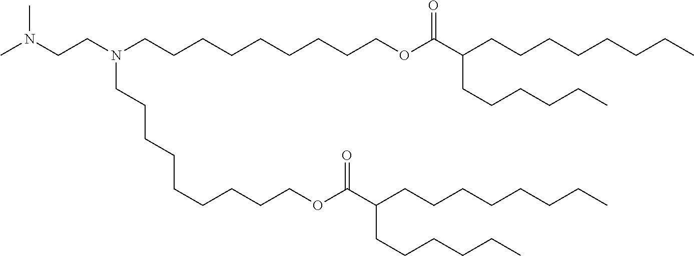 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present invention include a cationic lipid having the compound structure:

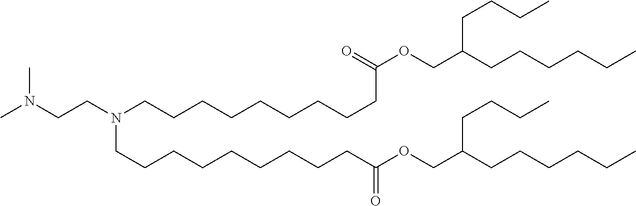 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present invention include a cationic lipid having the compound structure:

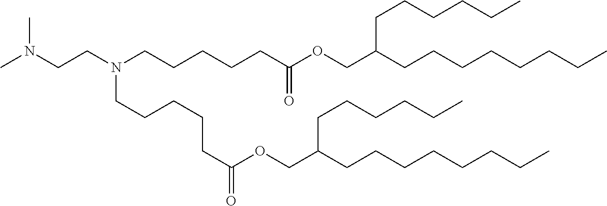 [SMILES: CCCCCCCCC(CCCCCC)COC(=O)CCCCCN(CCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

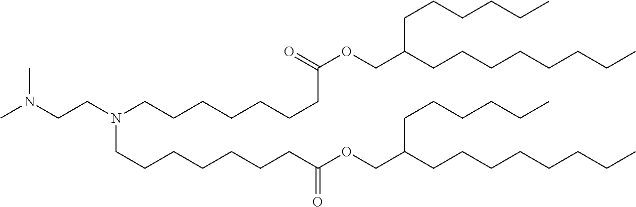 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

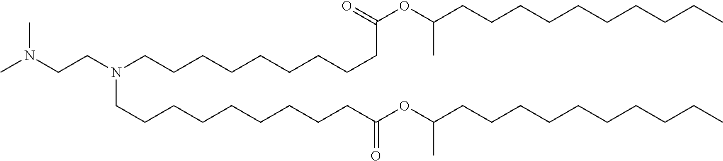 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present invention include a cationic lipid having the compound structure:

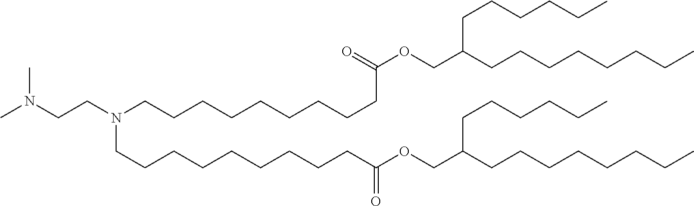 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present invention include a cationic lipid having the compound structure:

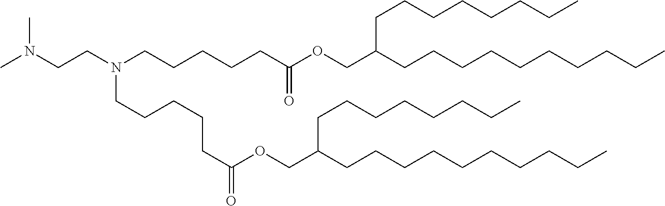 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

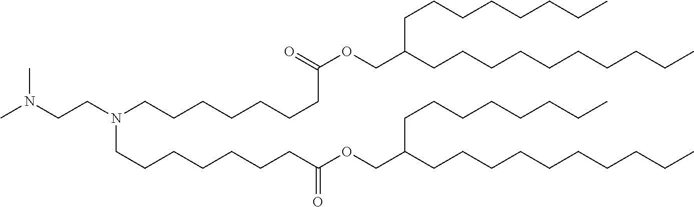 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present invention include a cationic lipid having the compound structure:

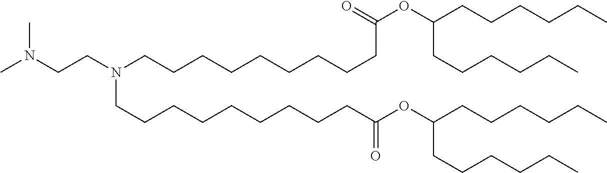 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present invention include a cationic lipid having the compound structure:

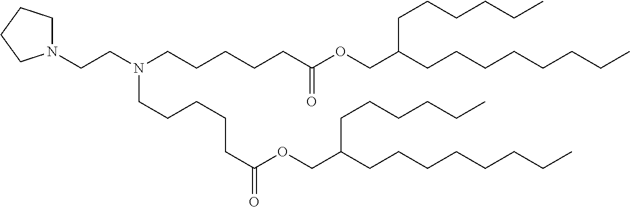 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present invention include a cationic lipid having the compound structure:

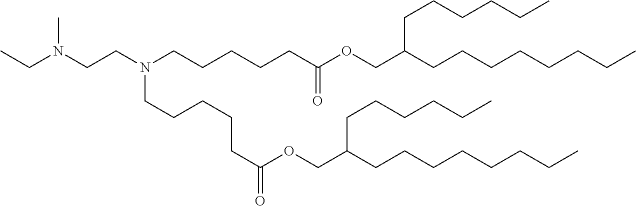 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present invention include a cationic lipid having the compound structure:

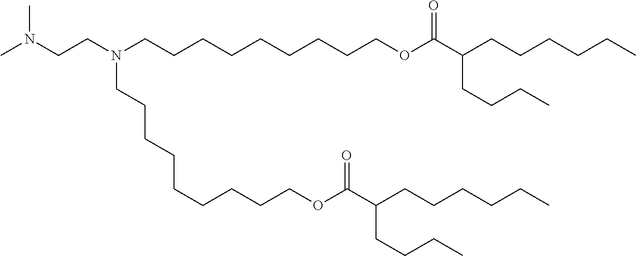 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the invention include the cationic lipids as described in International Patent Publication WO 2017/004143, which is incorporated herein by reference. In some embodiments, the compositions and methods of the present invention include a cationic lipid having the compound structure:

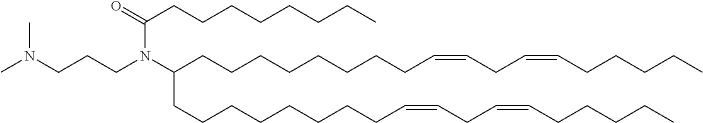 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present invention include a cationic lipid having the compound structure:

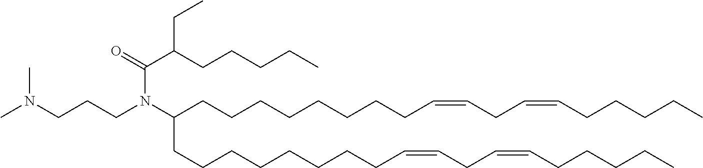 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present invention include a cationic lipid having the compound structure:

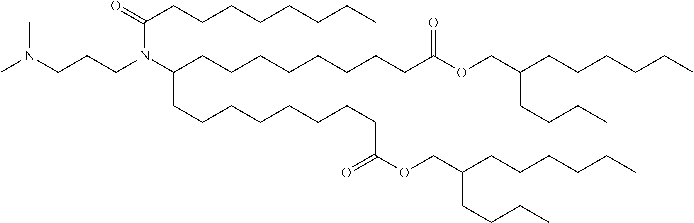 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

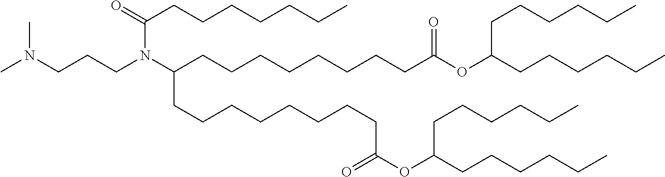 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

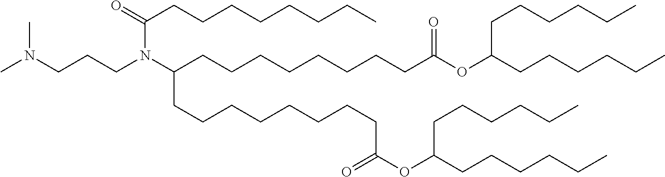 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present invention include a cationic lipid having the compound structure:

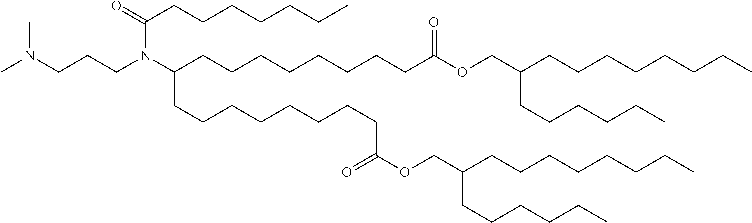 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

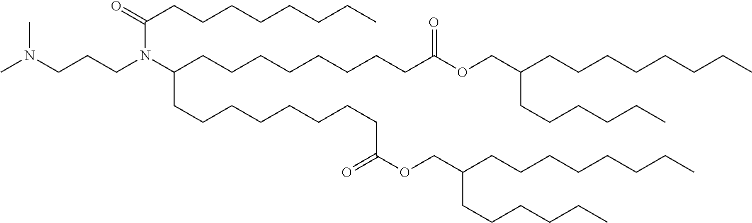 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present invention include a cationic lipid having the compound structure:

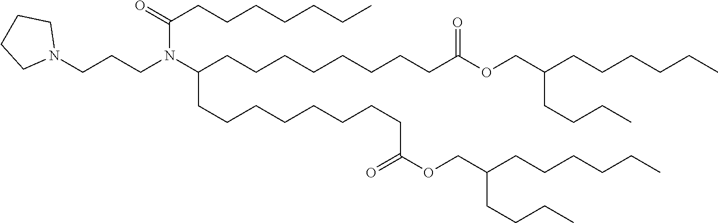 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

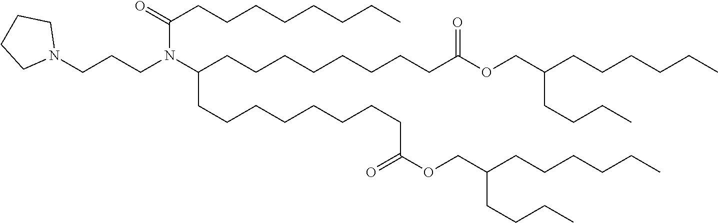 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present invention include a cationic lipid having the compound structure:

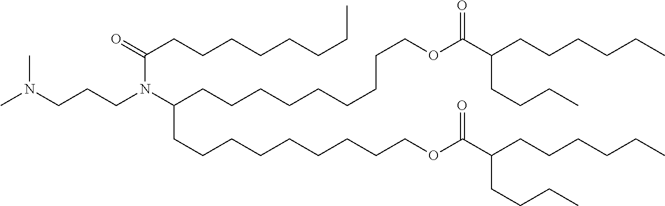 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present invention include a cationic lipid having the compound structure:

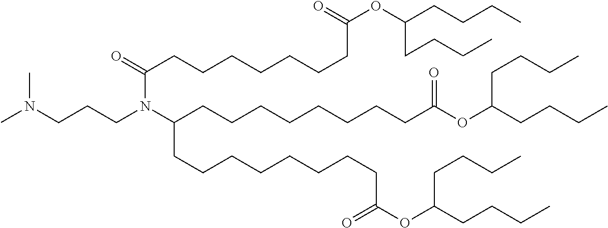 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present invention include a cationic lipid having the compound structure:

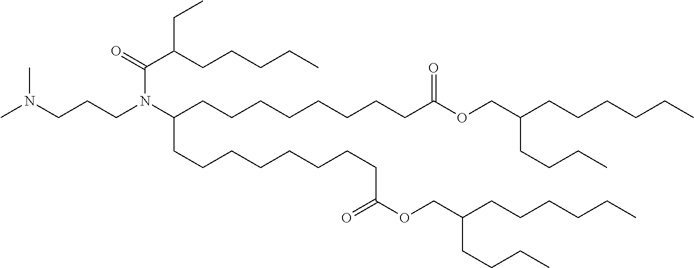 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present invention include a cationic lipid having the compound structure:

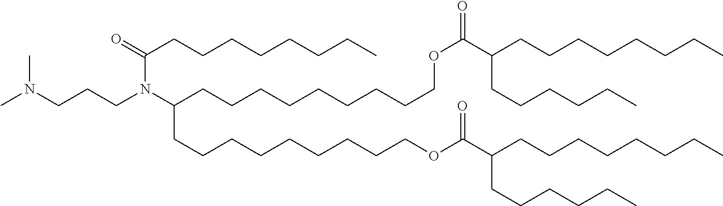 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present invention include a cationic lipid having the compound structure:

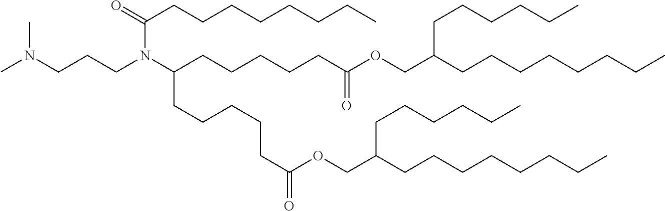 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present invention include a cationic lipid having the compound structure:

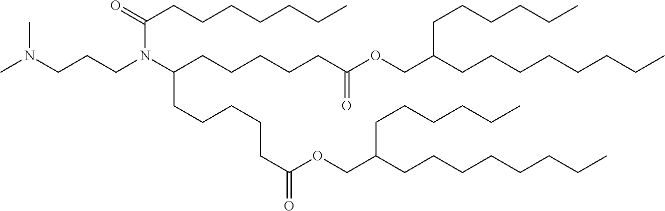 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present invention include a cationic lipid having the compound structure:

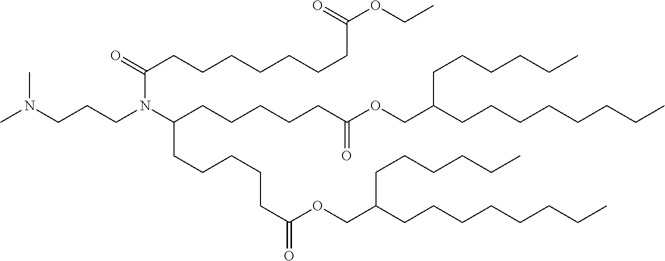 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present invention include a cationic lipid having the compound structure:

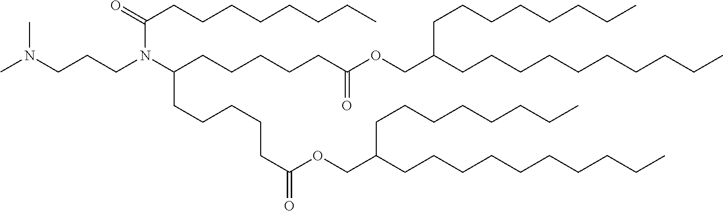 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

Other suitable cationic lipids for use in the compositions and methods of the invention include the cationic lipids as described in International Patent Publication WO 2017/075531, which is incorporated herein by reference. In some embodiments, the compositions and methods of the present invention include a cationic lipid of the following formula:

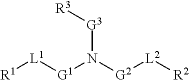

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; R^{a} is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵ C(═O)R⁴; R⁴ is C₁-C₁₂ alkyl; R⁵ is H or C₁-C₆ alkyl; and x is 0, 1 or 2.

Other suitable cationic lipids for use in the compositions and methods of the invention include the cationic lipids as described in International Patent Publication WO 2017/117528, which is incorporated herein by reference. In some embodiments, the compositions and methods of the present invention include a cationic lipid having the compound structure:

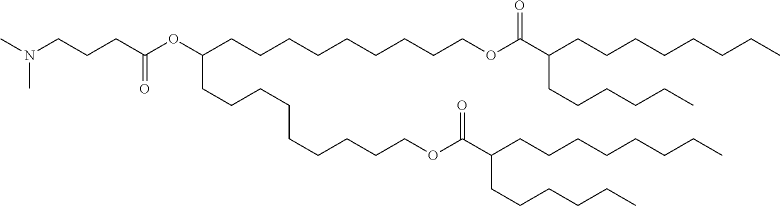 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present invention include a cationic lipid having the compound structure:

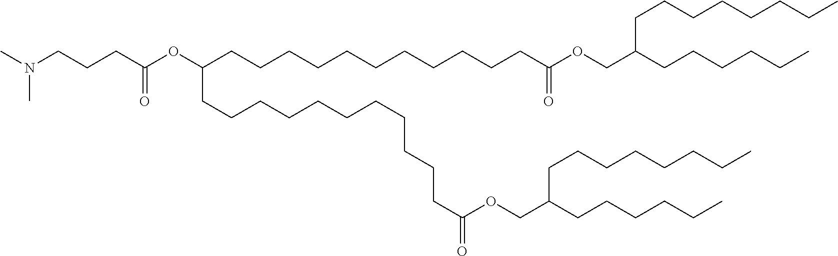 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present invention include a cationic lipid having the compound structure:

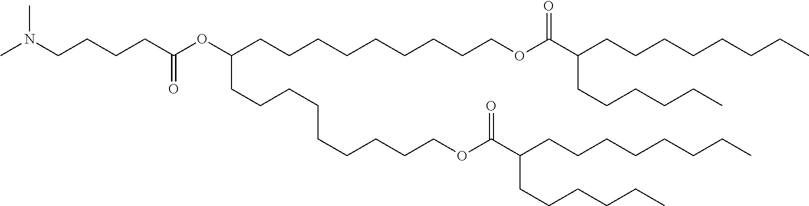 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the invention include the cationic lipids as described in International Patent Publication WO 2017/049245, which is incorporated herein by reference. In some embodiments, the cationic lipids of the compositions and methods of the present invention include a compound of one of the following formulas:

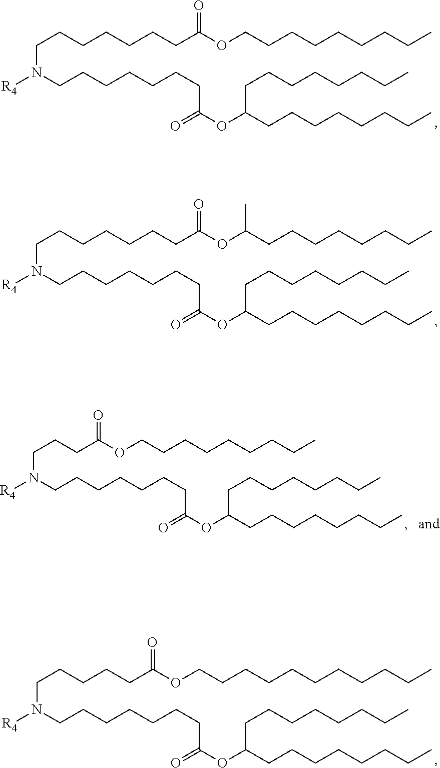

and pharmaceutically acceptable salts thereof. For any one of these four formulas, R4 is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; and n is 1, 2, or 3. In certain embodiments, the compositions and methods of the present invention include a cationic lipid having a compound structure of:

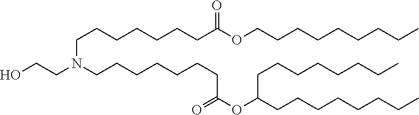 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

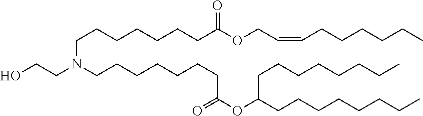 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

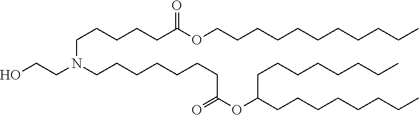 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present invention include a cationic lipid having a compound structure of:

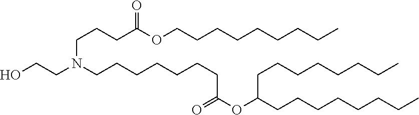 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the invention include the cationic lipids as described in International Patent Publication WO 2017/173054 and WO 2015/095340, each of which is incorporated herein by reference. In certain embodiments, the compositions and methods of the present invention include a cationic lipid having a compound structure of:

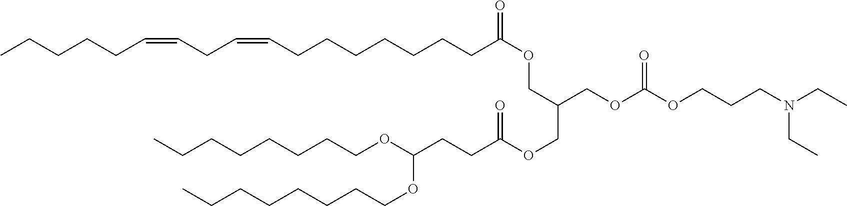 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof. In certain embodiments, the compositions and methods of the present invention include a cationic lipid having a compound structure of:

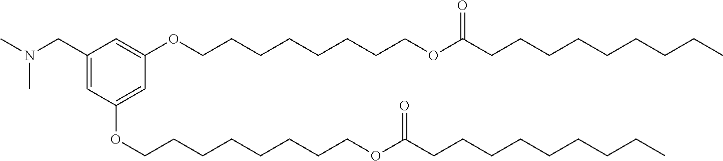 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present invention include a cationic lipid having a compound structure of:

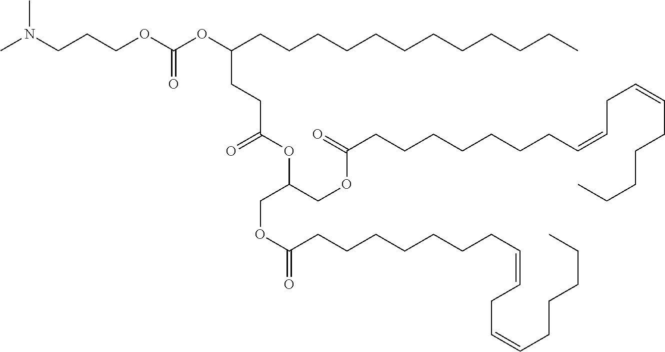 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present invention include a cationic lipid having a compound structure of:

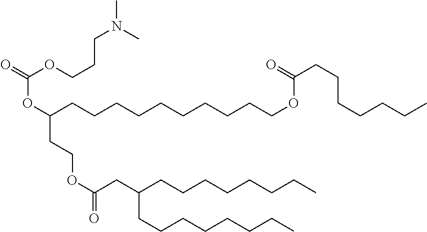 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the present invention include cleavable cationic lipids as described in International Patent Publication WO 2012/170889, which is incorporated herein by reference. In some embodiments, the compositions and methods of the present invention include a cationic lipid of the following formula:

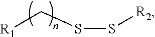

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

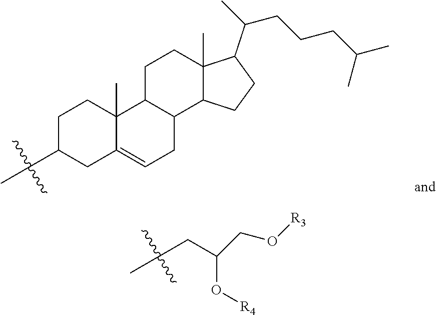

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present invention include a cationic lipid, “HGT4001”, having a compound structure of:

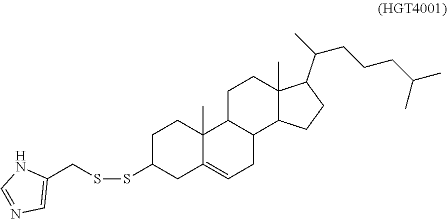 [SMILES: CC(C)CCCC(C)C1CCC2C3CC=C4CC(SSCc5cnc[nH]5)CCC4(C)C3CCC12C]

and pharmaceutically acceptable salts thereof. In certain embodiments, the compositions and methods of the present invention include a cationic lipid, “HGT4002”, having a compound structure of:

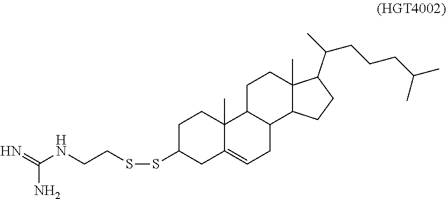 [SMILES: CC(C)CCCC(C)C1CCC2C3CC=C4CC(SSCCNC(=N)N)CCC4(C)C3CCC12C]

and pharmaceutically acceptable salts thereof. In certain embodiments, the compositions and methods of the present invention include a cationic lipid, “HGT4003”, having a compound structure of:

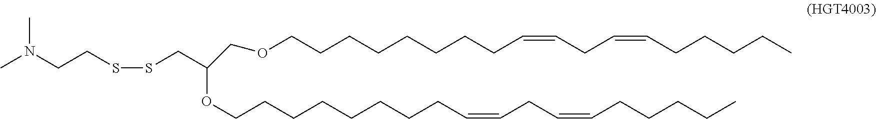 [SMILES: CCCCC/C=C\C/C=C\CCCCCCCCOCC(CSSCCN(C)C)OCCCCCCCC/C=C\C/C=C\CCCCC]

and pharmaceutically acceptable salts thereof. In certain embodiments, the compositions and methods of the present invention include a cationic lipid, “HGT4004”, having a compound structure of:

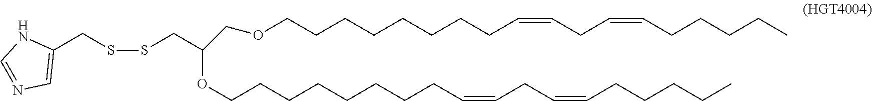 [SMILES: CCCCC/C=C\C/C=C\CCCCCCCCOCC(CSSCc1cnc[nH]1)OCCCCCCCC/C=C\C/C=C\CCCCC]

and pharmaceutically acceptable salts thereof. In certain embodiments, the compositions and methods of the present invention include a cationic lipid “HGT4005”, having a compound structure of:

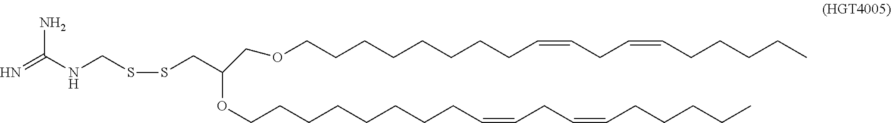 [SMILES: CCCCC/C=C\C/C=C\CCCCCCCCOCC(CSSCNC(=N)N)OCCCCCCCC/C=C\C/C=C\CCCCC]

and pharmaceutically acceptable salts thereof.

In some embodiments, the compositions and methods of the present invention include the cationic lipid, N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”).

Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylarnrnonium bromide (“DDAB”); N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethy 1-1-(cis,cis-9′, 1-2′-octadecadienoxy)propane (“CpLinDMA”); N,N-dimethyl-3,4-dioleyloxybenzylamine (“DMOBA”); 1 ,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl-3-dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl-3-[(9Z, 12Z)-octadeca-9, 12-dien-1 -yloxy]propane-1-amine (“Octyl-CLinDMA”); (2R)-2-((8-[(3beta)-cholest-5-en-3-yloxy]octyl)oxy)-N, N-dimethyl-3-[(9Z, 12Z)-octadeca-9, 12-dien-1-yloxy]propan-1 -amine (“Octyl-CLinDMA (2R)”); (2S)-2-((8-[(3P)-cholest-5-en-3-yloxy]octyl)oxy)-N, fsl-dimethyh3-[(9Z, 12Z)-octadeca-9, 12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,1 2-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more cationic lipids suitable for the compositions and methods of the present invention include 2,2-Dilinoley1-4-dimethylaminoethyl-[1,3]-dioxolane (“XTC”); (3aR,5s,6aS)-N,N-dimethyl-2,2-di((9Z,12Z)-octadeca-9,12-dienyl)tetrahydro-3aH-cyclopenta[d] [1,3]dioxol-5-amine (“ALNY-100”) and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present invention include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present invention include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present invention include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle

In some embodiments, sterol-based cationic lipids may be use instead or in addition to cationic lipids described herein. Suitable sterol-based cationic lipids are dialkylamino-, imidazole-, and guanidinium-containing sterol-based cationic lipids. For example, certain embodiments are directed to a composition comprising one or more sterol-based cationic lipids comprising an imidazole, for example, the imidazole cholesterol ester or “ICE” lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by structure (I) below. In certain embodiments, a lipid nanoparticle for delivery of RNA (e.g., mRNA) encoding a functional protein may comprise one or more imidazole-based cationic lipids, for example, the imidazole cholesterol ester or “ICE” lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate, as represented by the following structure:

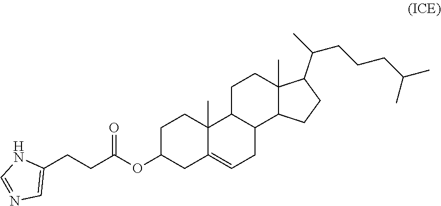 [SMILES: CC(C)CCCC(C)C1CCC2C3CC=C4CC(OC(=O)CCc5cnc[nH]5)CCC4(C)C3CCC12C]

In some embodiments, the percentage of cationic lipid in a liposome may be greater than 10%, greater than 20%, greater than 30%, greater than 40%, greater than 50%, greater than 60%, or greater than 70%. In some embodiments, cationic lipid(s) constitute(s) about 30-50% (e.g., about 30-45%, about 30-40%, about 35-50%, about 35-45%, or about 35-40%) of the liposome by weight. In some embodiments, the cationic lipid (e.g., ICE lipid) constitutes about 30%, about 35%, about 40%, about 45%, or about 50% of the liposome by molar ratio.

As used herein, the phrase “non-cationic lipid” refers to any neutral, zwitterionic or anionic lipid. As used herein, the phrase “anionic lipid” refers to any of a number of lipid species that carry a net negative charge at a selected H, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

In some embodiments, such non-cationic lipids may be used alone, but are preferably used in combination with other lipids, for example, cationic lipids. In some embodiments, the non-cationic lipid may comprise a molar ratio of about 5% to about 90%, or about 10% to about 70% of the total lipid present in a liposome. In some embodiments, a non-cationic lipid is a neutral lipid, i.e., a lipid that does not carry a net charge in the conditions under which the composition is formulated and/or administered. In some embodiments, the percentage of non-cationic lipid in a liposome may be greater than 5%, greater than 10%, greater than 20%, greater than 30%, or greater than 40%.

Suitable cholesterol-based cationic lipids include, for example, DC-Choi (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE. In some embodiments, the cholesterol-based lipid may comprise a molar ration of about 2% to about 30%, or about 5% to about 20% of the total lipid present in a liposome. In some embodiments, the percentage of cholesterol-based lipid in the lipid nanoparticle may be greater than 5%, greater than 10%, greater than 20%, greater than 30%, or greater than 40%.

The use of polyethylene glycol (PEG)-modified phospholipids and derivatized lipids such as derivatized cerarmides (PEG-CER), including N-Octanoyl-Sphingosine-1-[Succinyl(Methoxy Polyethylene Glycol)-2000] (C8 PEG-2000 ceramide) is also contemplated by the present invention, either alone or preferably in combination with other lipid formulations together which comprise the transfer vehicle (e.g., a lipid nanoparticle). Contemplated PEG-modified lipids include, but are not limited to, a polyethylene glycol chain of up to S kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target tissues, (Klibanov et al. (1990) FEBS Letters, 268 (1): 235-237), or they may be selected to rapidly exchange out of the formulation in vivo (see U.S. Pat. No. 5,885,613). Particularly useful exchangeable lipids are PEG-ceramides having shorter acyl chains (e.g., C14 or C18). The PEG-modified phospholipid and derivitized lipids of the present invention may comprise a molar ratio from about 0% to about 20%, about 0.5% to about 20%, about 1% to about 15%, about 4% to about 10%, or about 2% of the total lipid present in the liposomal transfer vehicle.

According to various embodiments, the selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the mRNA to be delivered. Additional considerations include, for example, the saturation of the alkyl chain, as well as the size, charge, pH, pKa, fusogenicity and toxicity of the selected lipid(s). Thus the molar ratios may be adjusted accordingly.

In some embodiments, a suitable delivery vehicle is formulated using a polymer as a carrier, alone or in combination with other carriers including various lipids described herein. Thus, in some embodiments, liposomal delivery vehicles, as used herein, also encompass nanoparticles comprising polymers. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, protamine, PEGylated protamine, PLL, PEGylated PLL and polyethylenimine (PEI). When PEI is present, it may be branched PEI of a molecular weight ranging from 10 to 40 kDa, e.g., 25 kDa branched PEI (Sigma #408727).

A suitable liposome for the present invention may include one or more of any of the cationic lipids, non-cationic lipids, cholesterol lipids, PEG-modified lipids and/or polymers described herein at various ratios. As non-limiting examples, a suitable liposome formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; ICE, DOPE, cholesterol and DMG-PEG2K; or ICE, DOPE, and DMG-PEG2K.

In various embodiments, cationic lipids (e.g., cKK-E12, C12-200, ICE, and/or HGT4003) constitute about 30-60% (e.g., about 30-55%, about 30-50%, about 30-45%, about 30-40%, about 35-50%, about 35-45%, or about 35-40%) of the liposome by molar ratio. In some embodiments, the percentage of cationic lipids (e.g., cKK-E12, C12-200, ICE, and/or HGT4003) is or greater than about 30%, about 35%, about 40%, about 45%, about 50%, about 55%, or about 60% of the liposome by molar ratio.

In some embodiments, the ratio of cationic lipid(s) to non-cationic lipid(s) to cholesterol-based lipid(s) to PEG-modified lipid(s) may be between about 30-60:25-35:20-30:1-15, respectively. In some embodiments, the ratio of cationic lipid(s) to non-cationic lipid(s) to cholesterol-based lipid(s) to PEG-modified lipid(s) is approximately 40:30:20:10, respectively. In some embodiments, the ratio of cationic lipid(s) to non-cationic lipid(s) to cholesterol-based lipid(s) to PEG-modified lipid(s) is approximately 40:30:25:5, respectively. In some embodiments, the ratio of cationic lipid(s) to non-cationic lipid(s) to cholesterol-based lipid(s) to PEG-modified lipid(s) is approximately 40:32:25:3, respectively. In some embodiments, the ratio of cationic lipid(s) to non-cationic lipid(s) to cholesterol-based lipid(s) to PEG-modified lipid(s) is approximately 50:25:20:5. In some embodiments, the ratio of sterol lipid(s) to non-cationic lipid(s) to PEG-modified lipid(s) is 50:45:5. In some embodiments, the ratio of sterol lipid(s) to non-cationic lipid(s) to PEG-modified lipid(s) is 50:40:10. In some embodiments, the ratio of sterol lipid(s) to non-cationic lipid(s) to PEG-modified lipid(s) is 55:40:5. In some embodiments, the ratio of sterol lipid(s) to non-cationic lipid(s) to PEG-modified lipid(s) is 55:35:10. In some embodiments, the ratio of sterol lipid(s) to non-cationic lipid(s) to PEG-modified lipid(s) is 60:35:5. In some embodiments, the ratio of sterol lipid(s) to non-cationic lipid(s) to PEG-modified lipid(s) is 60:30:10.

In some embodiments, a suitable liposome for the present invention comprises ICE and DOPE at an ICE:DOPE molar ratio of >1:1. In some embodiments, the ICE:DOPE molar ratio is <2.5:1. In some embodiments, the ICE:DOPE molar ratio is between 1:1 and 2.5:1. In some embodiments, the ICE:DOPE molar ratio is approximately 1.5:1. In some embodiments, the ICE:DOPE molar ratio is approximately 1.7:1. In some embodiments, the ICE:DOPE molar ratio is approximately 2:1. In some embodiments, a suitable liposome for the present invention comprises ICE and DMG-PEG-2K at an ICE:DMG-PEG-2K molar ratio of >10:1. In some embodiments, the ICE:DMG-PEG-2K molar ratio is <16:1. In some embodiments, the ICE:DMG-PEG-2K molar ratio is approximately 12:1. In some embodiments, the ICE:DMG-PEG-2K molar ratio is approximately 14:1. In some embodiments, a suitable liposome for the present invention comprises DOPE and DMG-PEG-2K at a DOPE: DMG-PEG-2K molar ratio of >5:1. In some embodiments, the DOPE: DMG-PEG-2K molar ratio is <11:1. In some embodiments, the DOPE: DMG-PEG-2K molar ratio is approximately 7:1. In some embodiments, the DOPE: DMG-PEG-2K molar ratio is approximately 10:1. In some embodiments, a suitable liposome for the present invention comprises ICE, DOPE and DMG-PEG-2K at an ICE:DOPE:DMG-PEG-2K molar ratio of 50:45:5. In some embodiments, a suitable liposome for the present invention comprises ICE, DOPE and DMG-PEG-2K at an ICE:DOPE:DMG-PEG-2K molar ratio of 50:40:10. In some embodiments, a suitable liposome for the present invention comprises ICE, DOPE and DMG-PEG-2K at an ICE:DOPE:DMG-PEG-2K molar ratio of 55:40:5. In some embodiments, a suitable liposome for the present invention comprises ICE, DOPE and DMG-PEG-2K at an ICE:DOPE:DMG-PEG-2K molar ratio of 55:35:10. In some embodiments, a suitable liposome for the present invention comprises ICE, DOPE and DMG-PEG-2K at an ICE:DOPE:DMG-PEG-2K molar ratio of 60:35:5. In some embodiments, a suitable liposome for the present invention comprises ICE, DOPE and DMG-PEG-2K at an ICE:DOPE:DMG-PEG-2K molar ratio of 60:30:10.

The liposomal transfer vehicles for use in the compositions of the invention can be prepared by various techniques which are presently known in the art. Various methods are described in published U.S. Application No. US 2011/0244026, published U.S. Application No. US 2016/0038432 and provisional U.S. Application No. 62/580,155, filed Nov. 1, 2017 and can be used to practice the present invention, all of which are incorporated herein by reference.

Briefly, the process of preparing CFTR-mRNA lipid nanoparticles includes a step of heating a first set of one or more solutions with a first set of one or more solutions (i.e., applying heat from a heat source to the solutions) to a temperature (or to maintain at a temperature) greater than ambient temperature. The first set of one more solutions can include a non-aqueous solution comprising the lipids used to form the lipid nanoparticle, and/or a solution comprising pre-formed lipid nanoparticles. The second set of one or more solutions can include an aqueous solution of the CFTR mRNA and/or a solution comprising the lipid nanoparticle encapsulated mRNA. In certain embodiments, the process includes a step of heating to a temperature (or to maintain at a temperature) greater than ambient temperature a first solution comprising pre-formed lipid nanoparticles with a second aqueous solution comprising CFTR mRNA In some embodiments, the process includes the step of heating one or both of the mRNA solution and the pre-formed lipid nanoparticle solution, prior to the mixing step. In some embodiments, the process includes heating one or more one or more of the solution comprising the pre-formed lipid nanoparticles, the solution comprising the mRNA and the solution comprising the lipid nanoparticle encapsulated mRNA, during the mixing step. In some embodiments, the process includes the step of heating the lipid nanoparticle encapsulated mRNA, after the mixing step. In some embodiments, the temperature to which one or more of the solutions is heated (or at which one or more of the solutions is maintained) is or is greater than about 30° C., 37° C., 40° C., 45° C., 50° C., 55° C., 60° C., 65° C., or 70° C. In some embodiments, the temperature to which one or more of the solutions is heated ranges from about 25-70° C., about 30-70° C., about 35-70° C., about 40-70° C., about 45-70° C., about 50-70° C., or about 60-70° C. In some embodiments, the temperature greater than ambient temperature to which one or more of the solutions is heated is about 65° C.

To facilitate expression of mRNA in vivo, delivery vehicles such as liposomes can be formulated in combination with one or more additional nucleic acids, carriers, targeting ligands or stabilizing reagents, or in pharmacological compositions where it is mixed with suitable excipients. Techniques for formulation and administration of drugs may be found in “Remington's Pharmaceutical Sciences,” Mack Publishing Co., Easton, Pa., latest edition.

As used herein, the term “therapeutically effective amount” is largely determined based on the total amount of the therapeutic agent contained in the pharmaceutical compositions of the present invention. Generally, a therapeutically effective amount is sufficient to achieve a meaningful benefit to the subject (e.g., treating, modulating, curing, preventing and/or ameliorating cystic fibrosis). For example, a therapeutically effective amount may be an amount sufficient to achieve a desired therapeutic and/or prophylactic effect.

In some embodiments, the composition comprising an mRNA encoding CFTR comprises mRNA at a concentration of at least 0.1 mg/mL. In some embodiments, the composition comprising an mRNA encoding CFTR comprises mRNA at a concentration of at least 0.2 mg/mL. In some embodiments, the composition comprising an mRNA encoding CFTR comprises mRNA at a concentration of at least 0.3 mg/mL. In some embodiments, the composition comprising an mRNA encoding CFTR comprises mRNA at a concentration of at least 0.4 mg/mL. In some embodiments, the mRNA encoding a CFTR protein is at a concentration of at least 0.5 mg/mL. In some embodiments, the mRNA encoding a CFTR protein is at a concentration of at least 0.6 mg/mL. In some embodiments, the mRNA encoding a CFTR protein is at a concentration of at least 0.7 mg/mL. In some embodiments, the mRNA encoding a CFTR protein is at a concentration of at least 0.8 mg/mL. In some embodiments, the mRNA encoding a CFTR protein is at a concentration of at least 0.9 mg/mL. In some embodiments, the mRNA encoding a CFTR protein is at a concentration of at least 1.0 mg/mL. In some embodiments, the mRNA encoding a CFTR protein is at a concentration of at least 2.0 mg/mL. In some embodiments, the mRNA encoding a CFTR protein is at a concentration of at least 3.0 mg/mL. In some embodiments, the mRNA encoding a CFTR protein is at a concentration of at least 4.0 mg/mL. In some embodiments, the mRNA encoding a CFTR protein is at a concentration of at least 5.0 mg/mL. In some embodiments, the mRNA encoding a CFTR protein is at a concentration of at least 6.0 mg/mL. In some embodiments, the mRNA encoding a CFTR protein is at a concentration of at least 7.0 mg/mL. In some embodiments, the mRNA encoding a CFTR protein is at a concentration of at least 8.0 mg/mL. In some embodiments, the mRNA encoding a CFTR protein is at a concentration of at least 9.0 mg/mL. In some embodiments, the mRNA encoding a CFTR protein is at a concentration of at least 10.0 mg/mL. In some embodiments, the mRNA encoding a CFTR protein is at a concentration ranging from 0.1 mg/mL to 10.0 mg/mL.

In some embodiments, the composition comprising an mRNA encoding CFTR is formulated with a diluent. In some embodiments, the diluent is selected from a group consisting of DMSO, ethylene glycol, glycerol, 2-Methyl-2,4-pentanediol (MPD), propylene glycol, sucrose, and trehalose. In some embodiments, the formulation comprises 1%, 2%, 3%, 4%, 5%, 6%, 7%, 8%, 9%, 10%, 11%, 12%, 13%, 14%, 15%, 16%, 17%, 18%, 19% or 20% diluent.

Pulmonary Delivery

A CFTR mRNA may be formulated for delivery via different administration routes including, but not limited to, oral, rectal, vaginal, transmucosal, or intestinal administration; parenteral delivery, including intradermal, transdermal (topical), intramuscular, subcutaneous, intramedullary injections, as well as intrathecal, direct intraventricular, intravenous, intraperitoneal, and/or intranasal administration. In some embodiments, a CFTR mRNA is formulated for pulmonary delivery. As used herein, pulmonary delivery refers to delivery to lung via, e.g., nasal cavity, trachea, bronchi, bronchioles, and/or other pulmonary system. In particular embodiments, a CFTR mRNA is formulated for nebulization. In these embodiments, the delivery vehicle may be in an aerosolized composition which can be inhaled.

In some embodiments, CFTR mRNA dry powder is formed by lyophilization of the mRNA-lipid complex. Applicant hereby fully incorporates by reference their earlier patent application Ser. No. 14/124615 filed on Jun. 8, 2012, which was granted a U.S. Pat. No. 9,717,690 on Aug. 1, 2017. The lyophilized dry powder is suitable for long term storage. It can be reconstituted with purified water for administration to a subject in need thereof In certain embodiments, upon reconstitution with an appropriate rehydration media (e.g., purified water, deionized water, 5% dextrose, 10% trehalose and/or normal saline, the reconstituted composition demonstrates pharmacological or biological activity comparable with that observed prior to lyophilization. For example, in certain embodiments, the pharmacological and biological activity of an encapsulated polynucleotide is equivalent to that observed prior to lyophilization of the composition; or alternatively demonstrates a negligible reduction in pharmacological and biological activity (e.g. less than about a 1%, 2%, 2.5%, 3%, 4%, 5%, 6%, 7%, 8% 9% or 10% reduction in the biological or pharmacological activity of an encapsulated polynucleotide).

In certain embodiments, the pharmaceutical compositions comprising lyophilized nanoparticles or lipid nanoparticle delivery vehicles are characterized as being stable (e.g., as stable as pharmaceutical compositions comprising an equivalent unlyophilized vehicles). Lyophilization of the lipid nanoparticles does not appreciably change or alter the particle size of the lipid nanoparticles following lyophilizaiton and/or reconstitution. For example, disclosed herein are pharmaceutical compositions comprising lyophilized lipid delivery vehicles, wherein upon reconstitution (e.g., with purified water) the lipid nanoparticles do not flocculate or aggregate, or alternatively demonstrated limited or negligible flocculation or aggregation (e.g., as determined by the particle size of the reconstituted lipid nanoparticles).

Accordingly, in certain embodiments, upon reconstitution of a lyophilized lipid nanoparticle the lipid nanoparticles have a Dv₅₀ of less than about 500 nm (e.g., less than about 300 nm, 200 nm, 150 nm, 125 nm, 120 nm, 100 nm, 75 nm, 50 nm, 25 nm, or smaller). Similarly, in certain embodiments, upon reconstitution of a lyophilized lipid nanoparticle the lipid nanoparticles have a Dv₉₀ of less than about 750 nm (e.g., less than about 700 nm, 500 nm, 300 nm, 200 nm, 150 nm, 125 nm, 100 nm, 75 nm, 50 nm, 25 nm, or smaller).

In other embodiments, the pharmaceutical compositions comprising lyophilized lipid delivery vehicles are characterized as having a polydispersion index of less than about 1 (e.g., less than 0.95, 0.9, 0.8, 0.75, 0.7, 0.6, 0.5, 0.4, 0.3, 0.25, 0.2, 0.1, 0.05, or less). In some embodiments, the pharmaceutical compositions comprising lyophilized lipid delivery vehicles demonstrate a reduced tendency to flocculate or otherwise aggregate (e.g., during lyophilization or upon reconstitution). For example, upon reconstitution the lipid delivery vehicles may have an average particle size (Zave) of less than 500 nm (e.g., less than about 400 nm, 300 nm, 200 nm, 175 nm, 150 nm, 125 nm, 100 nm, 75 nm, 50 nm, 25 nm, or smaller in a PBS solution).

In some embodiments, the lyophilized lipid delivery vehicles (e.g., lyophilized lipid nanoparticles) further comprise or are alternatively prepared using one or more lyoprotectants (e.g., sugars and/or carbohydrates). In certain embodiments, the inclusion of one or more lyoprotectants in the lipid nanoparticle may improve or otherwise enhance the stability of the lyophilized lipid delivery vehicles (e.g., under normal storage conditions) and/or facilitate reconstitution of the lyophilized lipid delivery vehicles using a rehydration media, thereby preparing an aqueous formulation. For example, in certain embodiments the lipid nanoparticles are prepared and prior to lyophilization the buffer present in the liposomal formulation may be replaced (e.g., via centrifugation) with a lyoprotectant such as a sucrose solution or suspension (e.g., an aqueous solution comprising between about 1-50% or 10-25% sucrose). In some embodiments, the lyoprotectant in trehalose. In some embodiments, the lyoprotectant comprises 10-50%, or 10-25% or 10-20% or 10-15% trehalose. Other lyoprotectants that may be used to prepare the lyophilized compositions described herein include, for example, dextran (e.g., 1.5 kDa, 5 kDa and/or 40 kDa) and inulin (e.g., 1.8 kDa and/or 4 kDa). The lyophilized lipid delivery vehicles have an encapsulation efficiency of greater than about 80%.

A pharmaceutical composition comprising a lyophilized lipid nanoparticle comprising CFTR-encoding mRNA is stable at 4° C. for at least 1 month, at least 2 months, at least 3 months, at least 4 months, at least 5 months, at least 6 months, or for at least 1 year. In some embodiments, the lyophilized lipid delivery vehicles may be stored under refrigeration and remain stable (e.g., as demonstrated by minimal or no losses in their intended pharmaceutical or biological activity) for extended periods of time (e.g., stable for at least about 1, 2, 3, 4, 5, 6, 9, 12, 18, 24, 36 months or longer upon storage at about 4° C.). In other embodiments, the lyophilized lipid delivery vehicles may be stored without refrigeration and remain stable for extended periods of time (e.g., stable for at least about 1, 2, 3, 4, 5, 6, 9, 12, 18, 24, 36 months or longer upon storage at about 25° C.).

The pharmaceutical composition in lyophilized form can be stored in frozen condition for 1, 2, 3, 4, 5 or 10 years without loss of pharmacological or biological activity.

Accordingly, also provided herein are methods for treating disease in a subject by administering an effective amount of pharmaceutical compositions comprising lyophilized CFTR mRNA-lipid delivery vehicles to a subject (e.g., upon reconstitution with a rehydrating media such as sterile water for injection).

In some embodiments, the formulation is administered by a metered-dose inhaler.

In some embodiments, the formulation is administered by a nebulizer.

Suitable CFTR mRNA formulation for nebulization may be stored as a frozen liquid, or sterile liquid, or lyophilized or dry powder and reconstituted prior to nebulization. In some embodiments, the composition is stored in a single-use vial prior to nebulization. In some embodiments, the single-use vial comprises 50 mL or less of the composition. In some embodiments, the single-use vial comprises 40 mL or less of the composition. In some embodiments, the single-use vial comprises 30 mL or less of the composition. In some embodiments, the single-use vial comprises 20 mL or less of the composition. In some embodiments, the single-use vial comprises 10 mL or less of the composition. In some embodiments, the single-use vial comprises 9.0 mL or less of the composition. In some embodiments, the single-use vial comprises 8.0 mL or less of the composition. In some embodiments, the single-use vial comprises 7.0 mL or less of the composition. In some embodiments, the single-use vial comprises 6.0 mL or less of the composition. In some embodiments, the single-use vial comprises 5.0 mL or less of the composition. In some embodiments, the single-use vial comprises between 4.0 mL and 5.0 mL of the composition. In some embodiments, the single-use vial comprises 3.2 mL of the composition.

In some embodiments, pulmonary delivery involves inhalation (e.g., for nasal, tracheal, or bronchial delivery). In some embodiments, the CFTR mRNA formulation is nebulized prior to inhalation. Nebulization can be achieved by any nebulizer known in the art. A nebulizer transforms a liquid to a mist so that it can be inhaled more easily into the lungs. Nebulizers are effective for infants, children and adults. Nebulizers are able to nebulize large doses of inhaled medications. One type of nebulizer is a jet nebulizer, which comprises tubing connected to a compressor, which causes compressed air or oxygen to flow at a high velocity through a liquid medicine to turn it into an aerosol, which is then inhaled by the patient. Another type of nebulizer is the ultrasonic wave nebulizer, which comprises an electronic oscillator that generates a high frequency ultrasonic wave, which causes the mechanical vibration of a piezoelectric element, which is in contact with a liquid reservoir. The high frequency vibration of the liquid is sufficient to produce a vapor mist. Exemplary ultrasonic wave nebulizers are the Omron NE-U17 and the Beurer Nebulizer IH30. A third type of nebulizer comprises vibrating mesh technology (VMT). VMT comprises mesh/membrane with 1000-7000 holes that vibrates at the top of a liquid reservoir and thereby pressures out a mist of very fine droplets through the holes in the mesh/membrane. Exemplary VMT nebulizers include Pari eFlow, Respironics i-Neb, Beurer Nebulizer IH50, Aerogen Aeroneb and Philips InnoSpire Go.

In some embodiments, the nebulization volume is at a volume ranging from 13.0 mL to 42.0 mL. In some embodiments, the nebulization volume is at a volume less than or equal to 13.9 mL. In some embodiments, the nebulization volume is at a volume less than or equal to 16.0 mL. In some embodiments, the nebulization volume is at a volume less than or equal to 18.0 mL. In some embodiments, the nebulization volume is at a volume less than or equal to 20.0 mL. In some embodiments, the nebulization volume is at a volume less than or equal to 22.0 mL. In some embodiments, the nebulization volume is at a volume less than or equal to 24.0 mL. In some embodiments, the nebulization volume is at a volume less than or equal to 26.0 mL. In some embodiments, the nebulization volume is at a volume less than or equal to 27.9 mL. In some embodiments, the nebulization volume is at a volume less than or equal to 30.0 mL. In some embodiments, the nebulization volume is at a volume less than or equal to 32.0 mL. In some embodiments, the nebulization volume is at a volume less than or equal to 34.0 mL. In some embodiments, the nebulization volume is at a volume less than or equal to 36.0 mL. In some embodiments, the nebulization volume is at a volume less than or equal to 38.0 mL. In some embodiments, the nebulization volume is at a volume less than or equal to 40.0 mL. In some embodiments, the nebulization volume is at a volume less than or equal to 41.8 mL.

In some embodiments, the duration of nebulization ranges from 1 minute to 150 minutes. In some embodiments, the duration of nebulization is less than or equal to 1 minute. In some embodiments, the duration of nebulization is less than or equal to 2 minutes. In some embodiments, the duration of nebulization is less than or equal to 3 minutes. In some embodiments, the duration of nebulization is less than or equal to 6 minutes. In some embodiments, the duration of nebulization is less than or equal to 9 minutes. In some embodiments, the duration of nebulization is less than or equal to 12 minutes. In some embodiments, the duration of nebulization is less than or equal to 15 minutes. In some embodiments, the duration of nebulization is less than or equal to 18 minutes. In some embodiments, the duration of nebulization is less than or equal to 21 minutes. In some embodiments, the duration of nebulization is less than or equal to 24 minutes. In some embodiments, the duration of nebulization is less than or equal to 27 minutes. In some embodiments, the duration of nebulization is less than or equal to 30 minutes. In some embodiments, the duration of nebulization is less than or equal to 33 minutes. In some embodiments, the duration of nebulization is less than or equal to 36 minutes. In some embodiments, the duration of nebulization is less than or equal to 40 minutes. In some embodiments, the duration of nebulization is less than or equal to 45 minutes. In some embodiments, the duration of nebulization is less than or equal to 50 minutes. In some embodiments, the duration of nebulization is less than or equal to 55 minutes. In some embodiments, the duration of nebulization is less than or equal to 60 minutes. In some embodiments, the duration of nebulization is less than or equal to 67 minutes. In some embodiments, the duration of nebulization is less than or equal to 70 minutes. In some embodiments, the duration of nebulization is less than or equal to 80 minutes. In some embodiments, the duration of nebulization is less than or equal to 90 minutes. In some embodiments, the duration of nebulization is less than or equal to 100 minutes. In some embodiments, the duration of nebulization is less than or equal to 110 minutes. In some embodiments, the duration of nebulization is less than or equal to 120 minutes. In some embodiments, the duration of nebulization is less than or equal to 130 minutes. In some embodiments, the duration of nebulization is less than or equal to 140 minutes. In some embodiments, the duration of nebulization is less than or equal to 150 minutes.

In some embodiments, the number of nebulizers used during a single nebulization session ranges from 2-8. In some embodiments, 1 nebulizer is used during a single nebulization session. In some embodiments, 2 nebulizers are used during a single nebulization session. In some embodiments, 3 nebulizers are used during a single nebulization session. In some embodiments, 4 nebulizers are used during a single nebulization session. In some embodiments, 5 nebulizers are used during a single nebulization session. In some embodiments, 6 nebulizers are used during a single nebulization session. In some embodiments, 7 nebulizers are used during a single nebulization session. In some embodiments, 8 nebulizers are used during a single nebulization session.

Pharmacokinetics and Tissue Distribution

According to the present invention, administration of a formulation comprising a CFTR mRNA results in delivery of the mRNA and encoded CFTR protein in various targets tissues described herein. In particular, administration of a formulation comprising a CFTR mRNA according to the present invention results in a therapeutically or clinically effective level or activity of CFTR in the target tissue. In various embodiments, a target tissue includes lung, pancreas, kidney, liver, spleen, testes/ovaries, salivary glands, sweat glands, heart and brain. In some embodiments, a target tissue is lung. In some embodiments, a target tissue is the upper (i.e., superior) lobe of the right or left lung. In some embodiments, a target tissue is the lower (i.e., inferior) lobe of the right or left lung. In some embodiments, a target tissue is the middle lobe of the right lung.

In some embodiments, a target tissue is the apical segment of the right lung or the apicoposterior segment of the left lung. In some embodiments, a target tissue is the posterior segment of the right lung. In some embodiments, a target tissue is the anterior segment of the right or left lung. In some embodiments, a target tissue is the superior segment of the right or left lung. In some embodiments, a target tissue is the lateral basal segment of the right or left lung. In some embodiments, a target tissue is the anterior basal segment of the right lung. In some embodiments, a target tissue is the anteromedial basal segment of the left lung. In some embodiments, a target tissue is the lateral segment of the right lung. In some embodiments, a target tissue is the medial segment of the right lung. In some embodiments, a target tissue is the superior lingular segment of the left lung. In some embodiments, a target tissue is the inferior lingular segment of the left lung. In some embodiments, a target tissue is the posterior basal segment of the right or left lung. In some embodiments, a target tissue is the medial basal segment of the right lung.

In particular embodiments, a target tissue is epithelial cells in the lung. In some embodiments, a target tissue is smooth muscle cells in the lung. In some embodiment, a target tissue is pancreatic duct epithelial cells. In some embodiment, a target tissue is bile-duct epithelial cells. In some embodiment, a target tissue is epithelial cells of the salivary glands. In some embodiment, a target tissue is renal epithelial cells. In some embodiment, a target tissue is beta-S cells in sweat gland secretory coils of sweat glands. In some embodiment, a target tissue is epithelial cells of the reproductive tract.

In some embodiments, a CFTR mRNA delivered according to the present invention achieves a level of CFTR protein expression or activity that is at least 5%, 10%, 20%, 30%, 40%, 50%, 60%, 70%, 80%, 90%, or 95% of the normal level of CFTR protein expression or activity in a target tissue described herein. In some embodiments, a CFTR mRNA delivered according to the present invention achieves a level of CFTR protein expression or activity that is increased by at least 1-fold, 2-fold, 3-fold, 4-fold, 5-fold, 6-fold, 7-fold, 8-fold, 9-fold or 10-fold as compared to a control (e.g., endogenous level of protein or activity without or before the treatment according to the invention, or a historical reference level) in a target tissue described herein.

In general, a CFTR mRNA delivered according to the present invention have sufficiently long half time in a target tissue described herein. In some embodiments, a CFTR mRNA delivered according to the present invention has a half-life of at least approximately 30 minutes, 45 minutes, 60 minutes, 90 minutes, 2 hours, 3 hours, 4 hours, 5 hours, 6 hours, 7 hours, 8 hours, 9 hours, 10 hours, 12 hours, 16 hours, 18 hours, 20 hours, 25 hours, 30 hours, 35 hours, 40 hours, 3 days, 7 days, 14 days, 21 days or a month. In some embodiments, a CFTR mRNA delivered according to the present invention results in detectable CFTR protein level or activity in a target tissue (e.g., the lung) or bloodstream after 12 hours, 24 hours, 30 hours, 36 hours, 42 hours, 48 hours, 54 hours, 60 hours, 66 hours, 72 hours, 78 hours, 84 hours, 90 hours, 96 hours, 102 hours, a week, two weeks, three weeks, or a month following administration. Detectable level or activity may be determined using various methods known in the art.

In some embodiments, a CFTR mRNA delivered according to the present invention results in increased CFTR protein level or activity in upper lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in lower lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in middle lobe lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in distal lung tissues by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 200-fold, 300-fold, 400-fold, or 500-fold as compared to a control (e.g., endogenous level of protein or activity without or before the treatment according to the invention, or a historical reference level).

In some embodiments, a CFTR mRNA delivered according to the present invention results in increased CFTR protein level or activity in distal peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 200-fold, or 300-fold as compared to a control (e.g., endogenous level of protein or activity without or before the treatment according to the invention, or a historical reference level).

In some embodiments, a CFTR mRNA delivered according to the present invention results in increased CFTR protein level or activity in lateral peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in increased CFTR protein level or activity in medial peripheral lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, or 1000-fold as compared to a control (e.g., endogenous level of protein or activity without or before the treatment according to the invention, or a historical reference level).

In some embodiments, a CFTR mRNA delivered according to the present invention results in increased CFTR protein level or activity in middle lung tissue by e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 150-fold, 200-fold, 250-fold, 300-fold, 350-fold, 400-fold, 450-fold, or 500-fold as compared to a control (e.g., endogenous level of protein or activity without or before the treatment according to the invention, or a historical reference level).

In some embodiments, a CFTR mRNA delivered according to the present invention results in increased CFTR protein level or activity in proximal lung tissue by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

In some embodiments, a CFTR mRNA delivered according to the present invention results in detectable CFTR protein or activity in the larynx, trachea, nasal turbinate, and/or bronchoalveolar lavage fluid (BALF). In some embodiments, a CFTR mRNA delivered according to the present invention results in detectable CFTR protein or activity in blood. In some embodiments, a CFTR mRNA delivered according to the present invention results in detectable CFTR protein or activity in lung, pancreas, kidney, liver, spleen, testes/ovaries, salivary glands, sweat glands, heart and brain.

In some embodiments, a CFTR mRNA delivered according to the present invention results in increased CFTR protein level or activity in larynx, trachea, tracheobronchial lymph node, and/or blood by, e.g., at least approximately 10%, 20%, 30%, 40%, 50%, 60%, 70%, 80%, 90%, 1-fold, 2-fold, 3-fold, 4-fold, 5-fold, 10-fold, 20-fold, 30-fold, 40-fold, 50-fold, 100-fold, 500-fold, 1000-fold, or 1500-fold as compared to a control (e.g., endogenous level of protein or activity without or before the treatment according to the invention, or a historical reference level).

The CFTR mRNA expression may be detected or quantified by qPCR on RNA purified from tissue samples. The CFTR protein expression may be determined by measuring immune responses to CFTR protein. In some embodiments, IgG antibody to CFTR protein is measured by an enzyme-linked immunosorbent assay in collected serum samples. In some embodiments, CFTR-specific T cell responses are assessed using collected peripheral blood mononuclear cells. In some embodiments, T cell responses to CFTR are measured by a human interferon-γ enzyme-linked immunospot assay as described by Calcedo et al. (Calcedo et al., Hum Gene Ther Clin Dev. (2013) 24:108-15). Qualitative assessment of CFTR protein may also be performed by Western blot analysis. The CFTR protein activity may be measured by CFTR chloride channel activity in appropriate tissue cells. A stable potential with the mean value of a 10 second scoring interval after perfusion of solution is recorded. CFTR activity is estimated by the change in potential difference following perfusion with chloride-free isoproterenol. Various other methods are known in the art and may be used to determine the CFTR mRNA and CFTR protein expression or activity.

Therapeutic Efficacy

According to the present invention, a CFTR mRNA is delivered to a CF patient in need of treatment at a therapeutically effective dose and an administration interval for a treatment period sufficient to improve, stabilize or reduce one or more symptoms of cystic fibrosis relative to a control. The terms “treat” or “treatment”, as used in the context of cystic fibrosis herein, refers to amelioration of one or more symptoms associated with cystic fibrosis, prevention or delay of the onset of one or more symptoms of cystic fibrosis, and/or lessening of the severity or frequency of one or more symptoms of cystic fibrosis.

In some embodiments, a therapeutically effective dose of a CFTR mRNA is or greater than about 2 mg, 4 mg, 6 mg, 8 mg, 10 mg, 12 mg, 14 mg, 16 mg, 18 mg, 20 mg, 22 mg, 24 mg, 26 mg, 28 mg, 30 mg, 32 mg, 34 mg, 36 mg, 38 mg, or 40 mg per dose or equivalent thereof. In some embodiments, a therapeutically effective dose of a CFTR mRNA is or less than about 50 mg, 48 mg, 46 mg, 44 mg, 42 mg, 40 mg, 38 mg, 36 mg, 34 mg, 32 mg, 30 mg, 28 mg, 26 mg, 24 mg, 22 mg, 20 mg, 18 mg, 16 mg, 14 mg, 12 mg, 10 mg, 8 mg, 6 mg or 4 mg per dose or equivalent thereof. In some embodiments, a therapeutically effective dose of a CFTR mRNA is about 2-50 mg, 4-45 mg, 4-40 mg, 6-40 mg, 6-38 mg, 6-36 mg, 6-34 mg, 6-32 mg, 6-30 mg, 6-28 mg, 6-26 mg, 6-24 mg, 6-22 mg, 6-20 mg, 6-18 mg, 6-16 mg, 8-50 mg, 8-45 mg, 8-40 mg, 8-38 mg, 8-36 mg, 8-34 mg, 8-32 mg, 8-30 mg, 8-28 mg, 8-26 mg, 8-24 mg, 8-22 mg, or 8-20 mg per dose or equivalent thereof.

In some embodiments, a therapeutically effective dose of a CFTR mRNA is administered daily, twice a week, weekly, once every two weeks, once every three weeks, once every four weeks, monthly, once every two months, once every three months.

In some embodiments, a therapeutically effective dose of a CFTR mRNA is administered for a period of at least two weeks, three weeks, four weeks, a month, two months, three months, four months, five months, six months, seven months, eight months, nine months, ten months, eleven months, twelve months, 1 year, 2 years, 3 years, 4 years, 5 years, or 10 years.

Typically, the therapeutic effect of administration of a CFTR mRNA on a cystic fibrosis patient is measured relative to a control. In some embodiments, a control is the severity of one or more symptoms in the same patient before the treatment. In some embodiments, a control is indicative of a historical reference level of one or more symptoms in CF patients. In some embodiments, a control is indicative of a normal level of ability, physical conditions or biomarker corresponding to the one or more symptoms being measured.

In some embodiments, the therapeutic effect of administration of a CFTR mRNA according to the present invention is measured by a score on a Cystic Fibrosis Questionnaire Revise (CFQ-R) respiratory domain. In some embodiments, the therapeutic effect of administration of a CFTR mRNA according to the present invention is measured by a sweat chloride value. In some embodiments, the therapeutic effect of administration of a CFTR mRNA according to the present invention is measured by a body mass index and/or body weight. In some embodiments, the therapeutic effect of administration of a CFTR mRNA according to the present invention is measured by onset or severity of pulmonary exacerbation.

In some embodiments, the therapeutic effect of administration of a CFTR mRNA according to the present invention is measured by minute volume, respiratory rate, and/or tidal volume. In some embodiments, the therapeutic effect of administration of a CFTR mRNA according to the present invention on the respiratory system is determined by performing spirometry and assessing the following parameters: forced expiratory volume in 1 second (FEV₁): absolute volume (L) and percent based on the patient's age, gender, and height, forced vital capacity (FVC): absolute volume (L) and percent based on the patient's age, gender, and height, FEV₁/FVC: ratio and percent based on the patient's age, gender, and height, and/or forced expiratory flow over the middle one-half of the FVC (FEF_{25-75%}): absolute volume (L) and percent based on the patient's age, gender, and height. In some embodiments, the parameters can be normalized using the ERS Global Lung Function Initiative (GLI) prediction equations. In some embodiments, the therapeutic effect of administration of a CFTR mRNA according to the present invention on the respiratory system is determined by chest x-ray.

In some embodiments, administration of a CFTR mRNA according to the present invention results in a change in the CFQ-R respiratory domain score by at least 1, at least 2, at least 3, at least 4, at least 5, at least 6, at least 7, at least 8, at least 9, at least 10, at least 11, at least 12, at least 13, at least 14, or at least 15 points relative to a control. In some embodiments, administration of a CFTR mRNA according to the present invention results in a change in the CFQ-R respiratory domain score by at least 1%, 2%, 3%, 4%, 5%, 6%, 7%, 8%, 9%, 10%, 11%, 12%, 13%, 14%, 15%, 16%, 17%, 18%, 19%, or 20% relative to a control.

In some embodiments, administration of a CFTR mRNA according to the present invention results in amelioration, prevention or delay in onset of pulmonary exacerbation. As used herein, pulmonary exacerbation refers to one or more of the following sino-pulmonary signs/symptoms: change in sputum, new or increased hemoptysis, increased cough, increased dyspnea, malaise/fatigue/lethargy, temperature >38° C. (˜100.4° F.), anorexia/weight loss, sinus pain/tenderness, change in sinus discharge, change in physical chest exam, decrease in pulmonary function and radiographic indication of pulmonary infection.

In some embodiments, administration of a CFTR mRNA according to the present invention results in prevention or reduced inflammation associated with pulmonary exacerbation. For example, administration of a CFTR mRNA according to the present invention results in reduced expression of markers of inflammation and/or lung damage, including but not limited to, C-reactive protein, white cell counts, interleukin-8, neutrophil elastase alpha 1-antiprotease complexes and matrix metalloproteins, in blood or serum as compared to a control indicative of the corresponding level of relevant markers in a CF patient without treatment. Additionally or alternatively, administration of a CFTR mRNA according to the present invention results in reduced sputum concentrations of bioactive lipid mediators, such as the cysteinyl leukotrienes and prostaglandin-E2, or sputum cell counts as compared to a control indicative of the corresponding level of relevant markers in a CF patient without treatment.

In some embodiments, administration of a CFTR mRNA according to the present invention results in a weight gain of at least 1 pound, at least 2 pounds, at least 3 pounds, at least 4 pounds, at least 5 pounds, at least 6 pounds, at least 7 pounds, at least 8 pounds, at least 9 pounds, at least 10 pounds, at least 11 pounds, at least 12 pounds, at least 13 pounds, at least 14 pounds or at least 15 pounds as compared to pre-treatment body weight.

In some embodiments, a CFTR mRNA is administered in combination with one or more CFTR potentiators and/or correctors. In some embodiments, a CFTR mRNA is administered in combination with one or more CFTR potentiators. In some embodiments, a CFTR mRNA is administered in combination with one or more CFTR correctors. Suitable CFTR potentiators and/or correctors include ivacaftor (trade name Kalydeco), lumacaftor (in a combination with ivacaftor sold under the trade name Orkambi) or the combination of ivacaftor and lumacaftor. Additional suitable correctors include tezacaftor, VX-659 and VX-445. In some embodiments, a CFTR mRNA is administered in combination with one or more other CF treatment such as hormone replacement therapies, thyroid hormone replacement therapy, non-steroidal inflammatory drugs, and prescription dronabinol (MARINOL) during treatment.

In some embodiments, CFTR potentiators and/or correctors and/or other cystic fibrosis treatments may be administered prior to, concurrently or subsequent to the administration of a CFTR mRNA according to the present invention. For example, CFTR potentiators and/or correctors and/or other cystic fibrosis treatments may be administered at 1 hour or longer, at 2 hours or longer, at 4 hours or longer, at 6 hours or longer, at 8 hours or longer, at 10 hours or longer, at 12 hours or longer, at 18 hours or longer, at 24 hours or longer, at 36 hours or longer, at 48 hours or longer, at 72 hours or longer, at 1 week or longer, at 2 weeks or longer, at 3 weeks or longer, or at 1 month or longer prior to or following administration of a CFTR mRNA according to the invention.

### EXAMPLES

While certain compounds, compositions and methods of the present invention have been described with specificity in accordance with certain embodiments, the following examples serve only to illustrate the compounds of the invention and are not intended to limit the same.

### Example 1

### Formulation of mRNA/Liposome Composition

Codon-optimized Human Cystic Fibrosis Transmembrane Conductance Regulator (CFTR) messenger RNA was synthesized by in vitro transcription from a plasmid DNA template encoding the gene, which was followed by the addition of a 5′ cap structure (Cap 1) (Fechter, P.; Brownlee, G. G. “Recognition of mRNA cap structures by viral and cellular proteins” J. Gen. Virology 2005, 86, 1239-1249) and a 3′ poly(A) tail of approximately 250 nucleotides in length as determined by gel electrophoresis. The mRNA encoding CFTR protein also comprised 5′ and 3′ untranslated regions (UTRs).

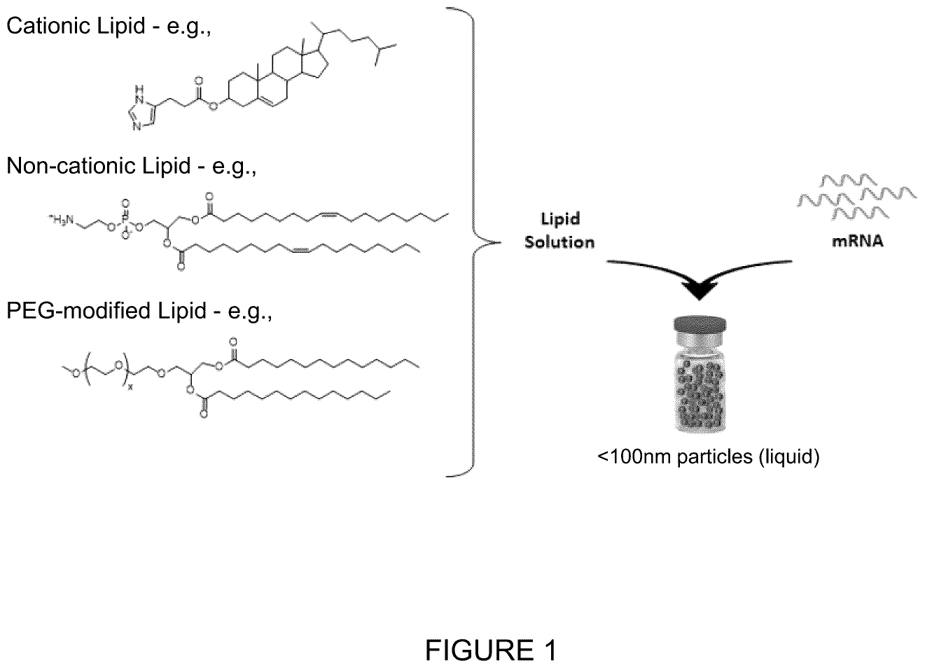

An aqueous-based solution comprising the exemplary mRNA encoding CFTR protein was combined with an ethanol-based lipid solution, isolated and dialyzed into the final formulation appropriate for storage at −80° C. FIG. 1 shows an exemplary generalized schematic of the formulation.

The lipid solution contained three lipid components to form lipid nanoparticles. The three biodegradable components all contributed to the final drug product characteristics. The first component was the ionizable lipid, imidazole cholesterol ester (ICE). This afforded a positively charged environment at low pH which facilitates efficient encapsulation of the negatively charged mRNA. It may also play a key role in cell surface interaction to allow for cellular uptake. The second component of the LNP was 1,2-dioleoyl-sn-glycero-3-phosphoethanolamine (DOPE). DOPE is a zwitterionic lipid that has been reported to have fusogenic properties to enhance uptake and release of the drug payload. The final component was a PEGylated (i.e., PEG-modified) lipid known as 1,2-dimyristoyl-sn-glycerol, methoxypolyethylene glycol (DMG-PEG-2K). The addition of this PEGylated lipid provided control over particle size and stability of the nanoparticle and may provide enhanced mucopentrating properties for lung uptake. The nominal nitrogen/phosphorus (N/P) charge ratio of the LNP was 4 and the average particle size range for the mRNA encapsulated in the LNP was 40-60 nm.

### Example 2

### Dosing of CFTR mRNA/Liposome Composition

These studies evaluated a CFTR mRNA/liposome composition in a Sprague-Dawley rat model. No adverse effects on the central nervous system (CNS), cardiovascular (CV) system or respiration were observed.

### CNS Evaluations

Neurobehavioral evaluations were performed on 6 males/group prior to dosing and on Day 1 (4 and 24 hours post-dosing). Temperature, humidity, noise level, and illumination of each room were measured and recorded to ensure that variations in environmental conditions were minimal during all evaluations. There were no effects on neurobehavior related to treatment with hCFTR mRNA-loaded ICE-based liposomes or ICE-based liposome vehicle control at inhaled doses up to 6.70 mg/kg hCFTR mRNA-loaded ICE-based liposomes.

### Cardiovascular Evaluations

Male Sprague-Dawley rats (n=5/group), which had previously been implanted with telemetry devices, were dosed via nose-only inhalation with hCFTR mRNA-loaded ICE-based liposomes for up to 6 hours at target doses of 0 (0.9% Saline control) or 0.7, 3.75 or 6.4 mg/kg of hCFTR mRNA-loaded ICE-based liposomes or ICE-based liposome vehicle control (non-mRNA containing LNP suspended in a solution containing 10% trehalose) (achieved doses of 0, 0.86, 3.52, 6.02 or 0 mg/kg, respectively). For target doses at 0.7 and 3.75 mg/kg, air was administered following the end of exposure to make the total restrained time equivalent among all doses. Each dose day was followed by a minimum 7-day washout period. All animals were returned to the colony upon completion of all evaluations.

Telemetry parameters included cardiovascular parameters (systolic, diastolic, mean blood pressure, pulse pressure, heart rate and electrocardiographic parameters [PR, QRS, QT, QTc]), activity, and body temperature. Other parameters evaluated during the study were viability, clinical observations and body weight. Mean aerosol concentrations and estimated total delivered doses of hCFTR mRNA-loaded ICE-based liposomes and ICE-based liposome vehicle (total lipid) are summarized in Table 3.

TABLE 3
Mean hCFTR mRNA-Loaded Liposome and Liposome
Aerosol Concentrations and Estimated Total Delivered Doses

| hCFTR mRNA-loaded Liposome | | hCFTR mRNA-loaded Liposome | |
|---|---|---|---|
| Aerosol Concentration (ug/L) | | Delivered Dose (mg/kg) | |
| | | Exposure Duration | Mean |
| Treatment | Achieved | (mins) | Achieveda |
| Control | 0 | 360 | 0 |
| Vehicle | 0 | 360 | 0 |
| Low | 30.5 | 40 | 0.86 |
| Mid | 23.7 | 210 | 3.52 |
| High | 23.7 | 360 | 6.02 |

a Overall mean of each individual animal for each treatment group. This dose reflects the total delivered dose of hCFTR mRNA.

In addition to this study in rats, similar cardiovascular evaluations were also performed as repeat-dose studies in rats and monkeys. In those repeat-dose studies, no test article-related effects were observed on any CV parameters evaluated up to the highest doses evaluated (6.7 mg/kg in rats and 0.691 mg/kg in monkeys).

Respiratory Evaluations

Respiratory effects of hCFTR mRNA-loaded ICE-based liposomes were evaluated as part of the single-dose and repeat-dose studies in rats and monkeys. An increase in minute volume was observed after inhalation administration of hCFTR mRNA-loaded ICE-based liposomes to Sprague Dawley rats, as well as respiratory rate and tidal volume, in all dose groups up to 6.4 mg/kg hCFTR mRNA-loaded ICE-based liposomes, as well as in 10% trehalose controls.

No effects were observed on respiratory parameters, including respiration rate, tidal volume and derived minute volume after inhalation administration of hCFTR mRNA-loaded ICE-based liposomes to Sprague-Dawley rats at repeat doses up to 6.7 mg/kg or cynomolgus monkeys at single or repeat doses up to 0.85 mg/kg or 0.691 mg/kg, respectively.

There is minimal toxicological concern regarding ICE, DOPE, and DMG-PEG 2000 as components of the composition developed for inhalation administration. In an in silico genotoxicity evaluation, ICE is predicted to be negative for bacterial mutagenicity. This is consistent with the negative mutagenicity/genotoxicity data that are available for imidazole and propionic acid, the 2 components of the imidazolepropionic acid moiety of ICE, and for cholesterol. DOPE is a variant of the glycerophospholipid, phosphatidylethanolamine, which is a component of lung surfactant. Degradation of DOPE would be expected to follow a similar path as for other glycerophospholipids, with the ultimate formation of ethanolamine and oleic acid, both of which are present in the circulation of infants and adults. DMG PEG 2000 is anticipated to have low toxicity based on information for the anticipated metabolic breakdown products PEG 2000 and myristic acid. There is minimal concern for local or systemic toxicity based on data from studies with PEGs of various sizes, while myristic acid is a fatty acid that is present in most animal and vegetable fats and is present in the circulation of infants and adults.

### Example 3

### Pharmacokinetics

In the study in this Example, Sprague-Dawley rats or monkeys were dosed for up to 6 hours (for rats) or for up to 2 hours (for monkeys) via inhalation with hCFTR mRNA-loaded ICE-based liposomes or with the ICE-based liposomes alone, and then sacrificed 24 hours later to measure the levels of hCFTR in various tissues. The target doses for are shown in Tables 4-6. The actual doses measured were 420, 630 and 850 μg/kg for hCFTR mRNA-loaded ICE-based liposomes administered to monkeys (corresponding to the target doses in the header of Table 4); 0.77, 4.05 and 6.70 mg/kg for hCFTR mRNA-loaded ICE-based liposomes administered to rats (corresponding to the target doses in the header of Table 5); and 0.77, 4.05 and 6.70 mg/kg for ICE-based liposomes administered to rats (corresponding to the target doses in the header of Table 6). Detailed tissue distribution results are presented below in Tables 4, 5, and 6.

TABLE 4
Mean Concentrations of hCFTR mRNA in Monkey Tissues and Blood 24 Hours
Post-Inhalation of hCFTR mRNA-Loaded Liposomes

| | Males | | | | Females | | | |
|---|---|---|---|---|---|---|---|---|
| | | Target Inhaled Dose | | | | Target Inhaled Dose | | |
| | | 500 | 750 | 1000 | | 500 | 750 | 1000 |
| Tissue ^{a} | Vehicle | μg/kg | μg/kg | μg/kg | Vehicle | μg/kg | μg/kg | μg/kg |
| Brain | BQL | 3.91 | 0.0968 | BQL | BQL | 2.05 | 0.110 | 1.18 |
| Heart | BQL | BQL | 0.165 | BQL | BQL | BQL | BQL | 0.537 |
| Kidney | BQL | BQL | BQL | 0.0523 | BQL | BQL | BQL | 0.231 |
| Larynx | 0.166 | BQL | 5.54 | 1.87 | BQL | 5.15 | 3.37 | 2.59 |
| Liver | BQL | BQL | 0.0670 | 1.27 | BQL | 0.917 | 0.147 | 8.48 |
| Lung (Average) | 0.110 | 208 | 67.2 | 82.1 | 0.426 | 819 | 1390 | 1880 |
| Spleen | BQL | 0.420 | BQL | BQL | BQL | BQL | 0.307 | 1.85 |
| Testis | BQL | BQL | BQL | 0.0912 | — | — | — | — |
| Ovary | — | — | — | — | BQL | 0.596 | BQL | 0.976 |
| Trachea | BQL | 0.176 | 0.993 | 1.65 | 0.0657 | 1.54 | 14.4 | 4.72 |
| Tracheobronchial | BQL | 30.9 | 0.284 | 70.3 | BQL | 0.147 | 468 | 5.28 |
| LN | | | | | | | | |
| Blood | 0.0501 | BQL | 0.0433 | 0.0120 | 0.0188 | 0.00351 | 0.385 | 0.0121 |

BQL: Below limit of quantification
^{a} Levels in tissue expressed as 10⁶ × copies/gm and levels in blood as 10⁶ × copies/mL, to express levels in comparable masses since 1 mL of blood ~1 gm.

TABLE 5
Mean hCFTR mRNA Concentrations in Rat Tissues and Blood 24 hours Post
hCFTR mRNA-Loaded Liposome Doses of 0.7, 3.75 or 6.4 mg/kg

| | Males | | | | Females | | | |
|---|---|---|---|---|---|---|---|---|
| | | 0.7 | 3.75 | 6.4 | | 0.7 | 3.75 | 6.4 |
| Tissue | Vehicle | mg/kg | mg/kg | mg/kg | Vehicle | mg/kg | mg/kg | mg/kg |
| Brain | BLQ | BLQ | BLQ | 55.4 | BLQ | BLQ | BLQ | 32.4 |
| Heart | BLQ | BLQ | BLQ | 17.0 | BLQ | BLQ | BLQ | 41.1 |
| Kidney | BLQ | BLQ | BLQ | 0.37 | BLQ | BLQ | BLQ | 0.95 |
| Larynx | 0.20 | BLQ | BLQ | 4178 | BLQ | BLQ | BLQ | 1410 |
| Liver | NC | BLQ | BLQ | 2.3 | BLQ | BLQ | BLQ | 9.75 |
| Lung | .061 | 2057 | 59,094 | 156130 | BLQ | 1361 | 33,649 | 180,000 |
| Nasal Turbinate | .12 | BLQ | BLQ | 792 | BLQ | BLQ | BLQ | 1450 |
| Spleen | BLQ | BLQ | BLQ | 3.8 | BLQ | BLQ | BLQ | 1.09 |
| Testis | 0.08 | BLQ | BLQ | 9.5 | BLQ | BLQ | BLQ | BLQ |
| Ovary | BLQ | BLQ | BLQ | BLQ | BLQ | BLQ | BLQ | 6.99 |
| Trachea | BLQ | BLQ | BLQ | 2980 | BLQ | BLQ | BLQ | 787 |
| Tracheobronchial | BLQ | BLQ | BLQ | 108 | BLQ | BLQ | BLQ | 1.48 |
| LN | | | | | | | | |
| Blood | 0.076 | 0.68 | 14.7 | 0.20 | .016 | 0.29 | 133 | 1.48 |

BLQ: below level of quantitation
Concentrations in tissues (copies × 106/g)/concentrations in blood (copies × 106/mL), with the assumption that 1 mL of blood ≈1 g.

TABLE 6
Mean ICE Concentrations in Rat Tissues (μg/g) and Blood (μg/mL) 24 hours Post-Inhalation
Dosing with hCFTR mRNA-Loaded Liposomes (Doses of 0.7, 3.75 or 6.4 mg/kg)

| | Males | | | | Females | | | |
|---|---|---|---|---|---|---|---|---|
| | | 0.7 | 3.75 | 6.4 | | 0.7 | 3.75 | 6.4 |
| Tissue | Vehicle | mg/kg | mg/kg | mg/kg | Vehicle | mg/kg | mg/kg | mg/kg |
| Brain | BLQ | BLQ | BLQ | BLQ | BLQ | BLQ | BLQ | BLQ |
| Heart | BLQ | BLQ | BLQ | BLQ | BLQ | BLQ | BLQ | BLQ |
| Kidney | BLQ | BLQ | BLQ | BLQ | BLQ | BLQ | BLQ | BLQ |
| Larynx | 1.57 | 2.61 | BLQ | 1.57 | BLQ | BLQ | BLQ | 1.41 |
| Liver | BLQ | BLQ | BLQ | BLQ | BLQ | BLQ | BLQ | BLQ |
| Lung | 317 | 20.3 | 139 | 245 | 293 | 22.5 | 147 | 317 |
| Nasal Turbinate | 0.290 | BLQ | BLQ | 0.796 | BLQ | BLQ | BLQ | 0.724 |
| Spleen | NA | BLQ | BLQ | BLQ | BLQ | BLQ | BLQ | BLQ |
| Testis | BLQ | BLQ | BLQ | BLQ | BLQ | BLQ | BLQ | BLQ |
| Ovary | BLQ | BLQ | BLQ | BLQ | BLQ | BLQ | BLQ | BLQ |
| Trachea | 6.27 | BLQ | BLQ | 3.65 | BLQ | BLQ | BLQ | 2.40 |
| Tracheobronchial | BLQ | BLQ | BLQ | BLQ | BLQ | BLQ | BLQ | BLQ |
| LN | | | | | | | | |
| Blood | BLQ | BLQ | BLQ | BLQ | BLQ | BLQ | BLQ | BLQ |

BLQ: below level of quantitation
Concentrations in tissues (copies × 106/g)/concentrations in blood (copies × 106/mL), with the assumption that 1 mL of blood ≈1 g.

These data show high levels of mRNA in lung tissue and associated respiratory tract issues such as larynx, trachea, tracheobronchial lymph nodes, and nasal turbinates with lower or background levels in heart, brain, liver, kidney, spleen, testis, and ovary, particularly at lower doses of administration, with the highest dose showing hCFTR mRNA distribution across various tissues. Lung levels were high and dose-responsive in both rats and NHP, with the highest levels seen at 6.4 mg/kg in rats.

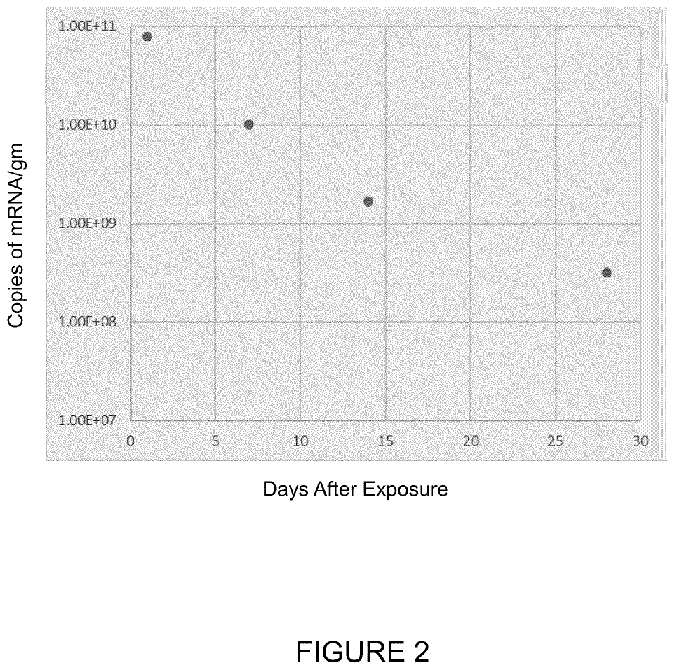

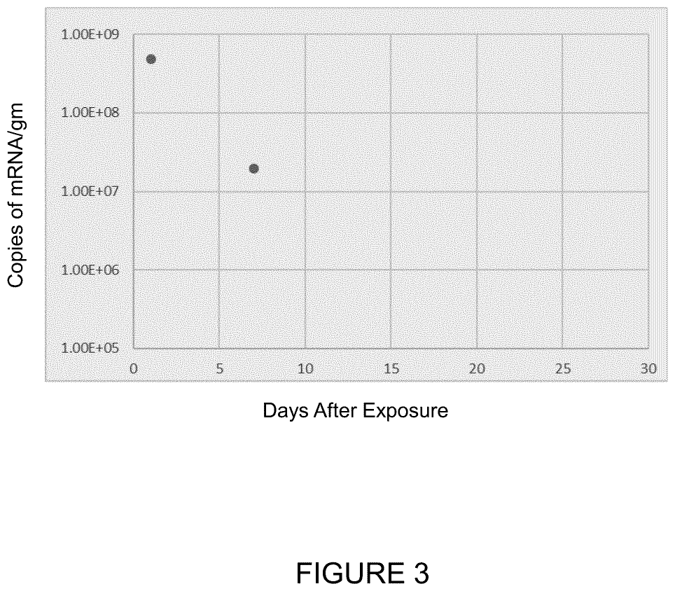

Kinetics of lung clearance of mRNA was measured reliably in rats since more sacrifice times could be used than for monkeys. FIG. 2 indicates a single component exponential decay with a half-life of approximately 2-3 days. Only two data points were available for NHP (FIG. 3) and these data appear consistent with the rat data in view of differences in dose.

As shown in Table 5, levels of mRNA in the lung were dose-dependent in a relatively linear manner. Lung tissue measurements made after a 28 day recovery period at the end of the 29-day study showed a decline in exposure of approximately 100-fold, similar to that seen 28 days after the single dose study.

The toxicokinetics of ICE liposomes were also examined. There were no measurable levels of ICE liposomes in whole blood. There were, however, measurable and dose-responsive levels of ICE liposomes in the lung tissue in rats (Table 6).

### Example 4

### In Vitro Activity of hCFTR in Human Bronchial Epithelial Cells

This Example illustrates a study where hCFTR mRNA was transfected into cultured human bronchial epithelial cells, whereupon the protein expressed from transfected hCFTR mRNA provided a significant increase in chloride transport across the bronchial epithelial cell membrane compared to buffer, thereby demonstrating the functional efficacy of the transfected mRNA. The changes in chloride transport across the bronchial epithelial cell membrane was measured by short circuit current output in an Ussing epithelial voltage clamp apparatus (i.e., a Ussing chamber). Specifically, using an established Ussing Chamber procedure (Charles River Laboratories), hCFTR mRNA encapsulated in a liposome comprising ICE, DOPE, and a PEG-modified lipid was incubated for 2 or 4 hours on the apical (mucosal) or basolateral (serosal) sides, or both sides, of human bronchial epithelial cells. A buffer blank also was included as a control, for example, to assess chloride transport by endogenous CFTR in the cells. Next, Forskolin-induced chloride channel activity was measured using the Ussing chamber assay. Following the measure of the current change as indicative of chloride transport across the bronchial epithelial cell membrane, a CFTR inhibitor was added to the samples to show that current change was due to CFTR activity.

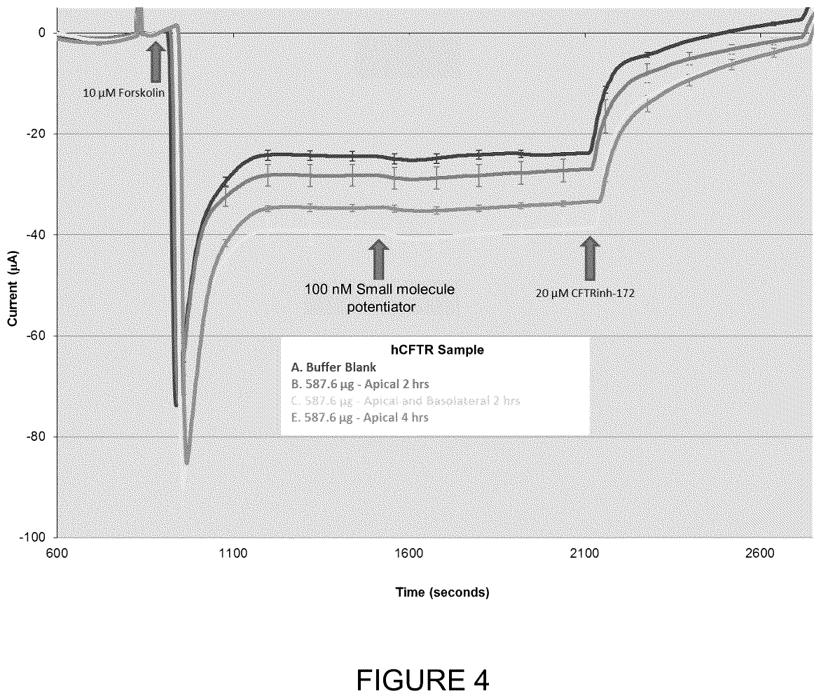

As shown in FIG. 4, compared to the control group (A. Buffer Blank), treatment of the apical (mucosal) epithelial surface for 2 or 4 hours (Samples B and E, respectively) and the apical and basolateral (serosal) epithelial surfaces for 2 hours (Sample C) with hCFTR mRNA provided a significant increase in chloride channel activity. Additionally, the chloride activity in all groups was inhibited by the CFTR inhibitor-172. The results of this study show that the hCFTR mRNA delivered in a liposome to human bronchial epithelial cells produced active CFTR protein in those cells. It also shows that the active CFTR protein produced from the hCFTR mRNA provided significantly increased chloride transport, compared to endogenous CFTR protein, across the cell membranes of the transfected human bronchial epithelial cells.

### Example 5

### Distribution of hCFTR after Single Dose of hCFTR mRNA/Liposome Composition in Primates

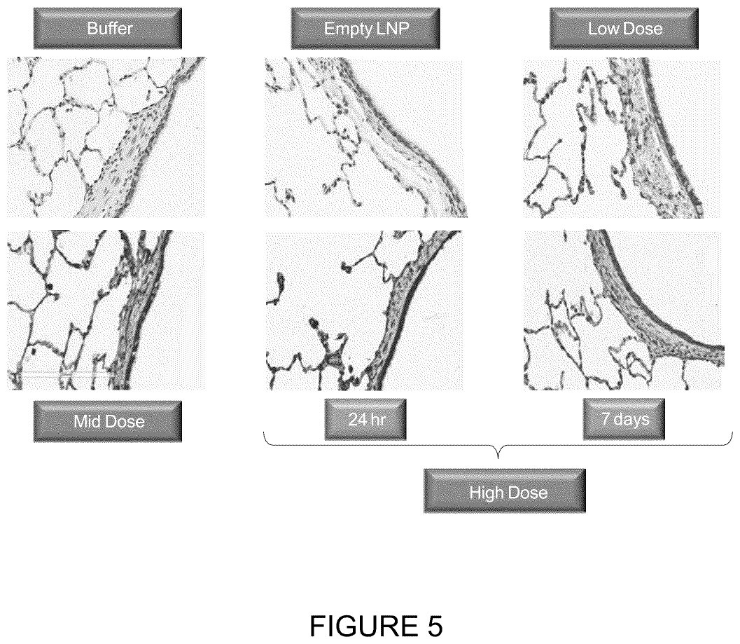

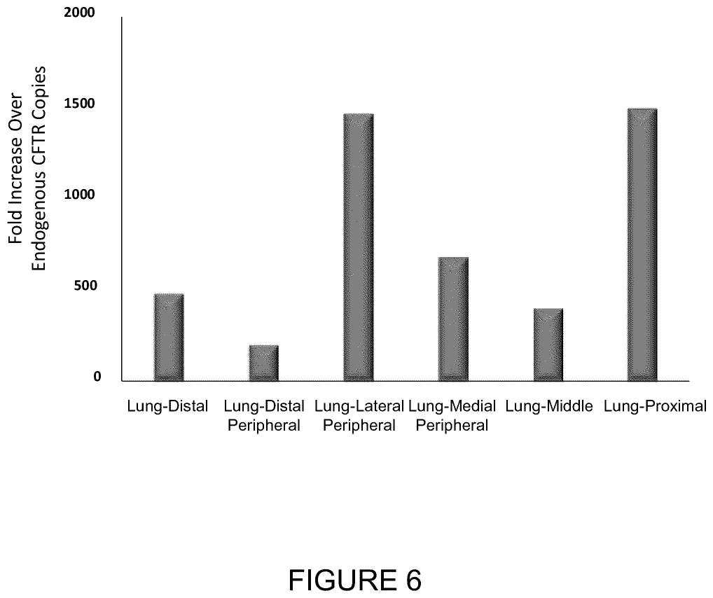

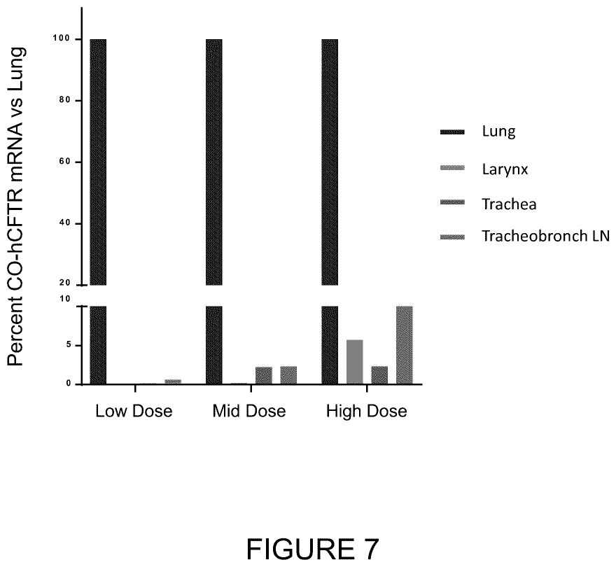

In the study in this Example, non-human primates (NHPs) were treated with a single aerosol exposure of a CO-hCFTR mRNA/liposome composition. As shown in FIG. 5, immunohistochemistry (IHC) staining of lung cross-sections demonstrate a dose-dependent increase in intensity for positive hCFTR protein detection. Additionally, when hCFTR mRNA was detected and quantified in tissues from the NHPs, widespread distribution was found throughout the lungs of the monkeys, with CO-hCFTR levels orders of magnitude (>200-fold) over endogenous CFTR (FIG. 6). Additionally, the lungs of the monkeys, which were the target organs, received >90% of the CO-CFTR dose that entered the airway, when compared to other respiratory tract tissues that were tested (FIG. 7).

$$Example 6. Treatment of Cystic Fibrosis Subjects with CO \text{-} hCFTR mRNA \text{/} Liposome Composition$$

The study in this example is designed to evaluate a CO-CFTR mRNA liposome composition in patients with cystic fibrosis.

A CO-hCFTR mRNA liposome composition (CO-hCFTR composition) is administered by nebulization to subjects with cystic fibrosis. The CO-hCFTR composition is dosed based on its content of CO-hCFTR mRNA. A nebulizer will be used to administer the CO-hCFTR composition by nebulization at a flow rate of approximately 0.3 mL/minute. The CO-hCFTR composition will be administered to subjects at the following 3 dose levels: 8.0, 16.0, or 24.0 mg of CO-hCFTR mRNA (nominal dose levels) either once or once per week for five weeks. Other subjects will be dosed with placebo control.

In order to receive treatment with administration of the CO-hCFTR composition, patients will have a confirmed diagnosis of CF as defined by all of the following: a sweat chloride value of >60 mmol/L by quantitative pilocarpine iontophoresis (documented in the subject's medical record), a confirmed disease-causing CFTR mutation (genotype confirmed at the screening visit), and chronic sinopulmonary disease and/or gastrointestinal/nutritional abnormalities consistent with CF disease; clinically stable CF disease, e.g., FEV₁≥50% and ≤90% of the predicted normal for age, gender, and height at screening, resting oxygen saturation ≥92% on room air (pulse oximetry), and body mass index≥17.5 kg/m² and weight≥40 kg. Subjects who are receiving lumacaftor/ivacaftor combination drug (ORKAMBI) will remain on it for the duration of the study preferably at a stable dose.

Procedures and tests that will be conducted both for screening subjects and during the study to evaluate the biological activity of the CO-CFTR mRNA liposome composition include: vital signs, pulse oximetry, physical examination, spirometry, clinical laboratory tests (serum chemistry, hematology, coagulation, urinalysis, CRP), ECG, chest x-ray, Cystic Fibrosis Questionnaire-Revised (CFQ-R), serum pregnancy test, AE and concomitant medication reporting, weight measurement, blood sampling for CO-hCFTR mRNA and ICE assays and blood sampling for immune response assays. Some subjects will also undergo bronchoscopy.

Bronchial epithelial cells obtained during bronchoscopies will be prepared for quantification of exogenous CO-hCFTR mRNA and endogenous CFTR mRNA by qPCR, and for a qualitative assessment of CFTR protein by Western blot analysis.

Additionally, during bronchoscopy, lower airway potential difference measurements will be performed to assess CFTR chloride channel activity in the bronchial epithelium. Potential difference measurements will be made at the lingula outlet of the left lung, as described by Dransfield et al. (Dransfield et al., Chest. (2013) 144:498-506). A stable potential with the mean value of a 10 second scoring interval after perfusion of each solution will recorded. CFTR activity will be estimated by the change in potential difference following perfusion with chloride-free isoproterenol.

The Cystic Fibrosis Questionnaire-Revised (CFQ-R; version for adolescents and adults [patients 14 years old and older]) will be completed subjects at in order to achieve both a baseline score and scores during the study for comparison. The results of the respiratory domain of the CFQ-R will be of primary interest; the minimal change from baseline representing a clinically important improvement in the respiratory domain was determined to be ≥4 (Quittner et al., Chest. (2009) 135:1610-8).

### Example 7

### CFTR Colocalization with Membrane Protein in Upper Airway Bronchial Cells

This example demonstrates CFTR protein expression to various lung tissues, including upper bronchial epithelium, lower bronchial epithelium, and alveolar tissue after inhalation administration of CFTR mRNA formulated in a lipid nanoparticle. This example further demonstrates through colocalization with the endogenous membrane tight junction protein, ZO-1, which is found in the cell membrane, that the CFTR protein expressed from the administered CFTR mRNA is localized in the cell membrane of lung tissue, including lung epithelial cells, such as upper airway bronchial epithelial cells and lower airway bronchial epithelial cells, as well in the cell membranes of alveolar cells.

Colocalization study protocol: The immunohistochemistry and colocalization study method described in this paragraph is common for Examples 7, 8 and 9. Lung delivery of the CFTR mRNA in primates was followed by immunohistochemistry to detect the protein expression and membrane colocalization in the upper airway bronchial cells and lower airway epithelial cells and deep alveolar lung. The primates in this study were grouped into five categories and were administered the following: (1) Control, Trehalose 10%; (2) Control, LNP vehicle; (3) CO-hCFTR low dose, 500 μg/kg, (4) CO-hCFTR medium dose, 750 μg/kg, (5) CO-hCFTR high dose, 1000 μg/kg. The mRNA-LNP formulation or controls (without mRNA) were administered daily. Accordingly, the animals were exposed for 60 minutes to the aerosol composition (Group 3, low dose, 500 μg/kg), 90 minutes of aerosol (Group 4, medium dose, 750 μg/kg), or 120 minutes of aerosol (Group 5, high dose) as described in Table 4. At the end of the study the animals were sacrificed and tissues collected for immunohistochemistry to detect the expression and localization of human CFTR protein in the lungs. For immunohistochemical detection of codon-optimized human CFTR protein, lung sections were incubated overnight at 4° C. with a 1:250 dilution of each of mouse monoclonal anti-CFTR antibody (MAB 25031, R & D Systems), and rabbit anti-ZO1 antibody (Ab214228, Abcam); or with anti-CFTR alone; and anti-ZO1 alone. Following overnight incubation, the sections were blocked with first blocking agent containing hydrogen peroxide, incubated with the secondary antibody (anti-mouse DyLight 594 for CFTR antibody, and anti-rabbit DyLight 488 antibody) and followed by second blocking in a solution containing 3% horse serum and 3% BSA and subjected to confocal microscopy for colocalization. DyLight 594 emits red signal, and DyLight 488 emits green, and upon colocalization of the two signals (and therefore the proteins each bind to), the optical merge yields yellow signal. (Color is not shown in the Figure).

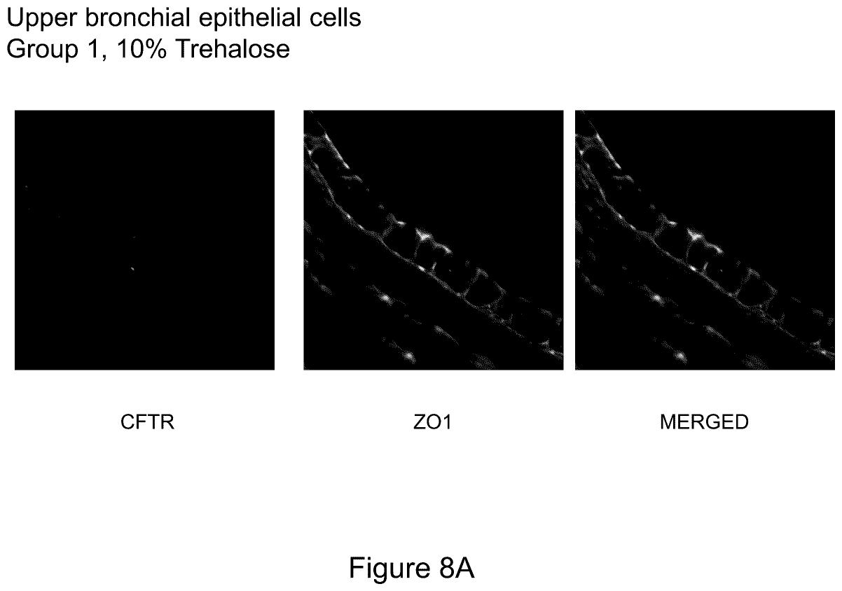

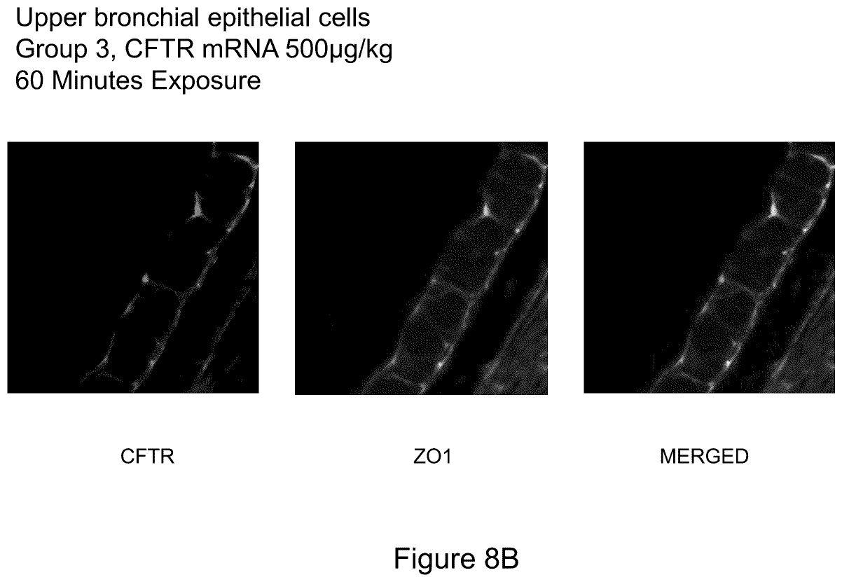

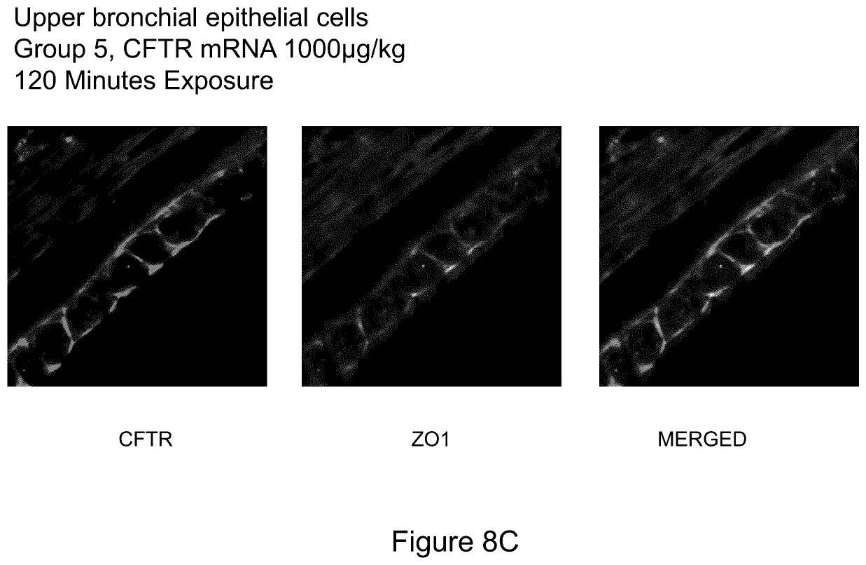

As shown in FIGS. 8A, 8B and 8C, CFTR protein translated from administered CFTR mRNA is expressed in the upper respiratory tract in bronchial epithelial cells. In the exemplary study, CFTR mRNA-derived protein exhibited a dose-dependent expression and colocalized with endogenous membrane tight junction protein ZO1 in upper bronchial epithelial cell membrane. FIG. 8A depicts representative microscopic images of bronchial epithelial cells from a control animal that received 10% trehalose alone, visualized for CFTR staining (left), ZO1 staining (middle) and optical merge of CFTR and ZO1 staining (right). FIG. 8B depicts representative microscopic images of bronchial epithelial cells of an animal that received low dose CFTR mRNA (500 μg/kg), visualized for CFTR staining (left), ZOlstaining (middle) and optical merge of CFTR and ZO1 staining (right). FIG. 8C depicts representative microscopic images of bronchial epithelial cells of an animal that received high dose CFTR mRNA (1000 μg/kg), visualized for CFTR staining (left), ZO1 staining (middle) and optical merge of CFTR and ZO1 staining (right). As indicated in the figure, the expression of CFTR was dose-dependent, that is, the immunostaining signal was stronger at the higher dose (FIG. 8C left image) than that of the low dose (FIG. 8B, left image). Also, as evident from the positioning of the CFTR and ZO1 signals, CFTR and ZO1 are expressed in the cell membrane of the epithelial cells. The two proteins colocalized yielding an intensified signal, which was yellow upon visual observation (FIGS. 8B and 8C right image). No red signal corresponding to CFTR was observed in FIG. 8A indicating the specificity of the staining. Also the merged optical signal was not intensified and monochromatic for ZO-1 signal alone. (Color is not shown in the Figure).

### Example 8

### CFTR Colocalization with Membrane Protein in Lower Airway Epithelial Cells

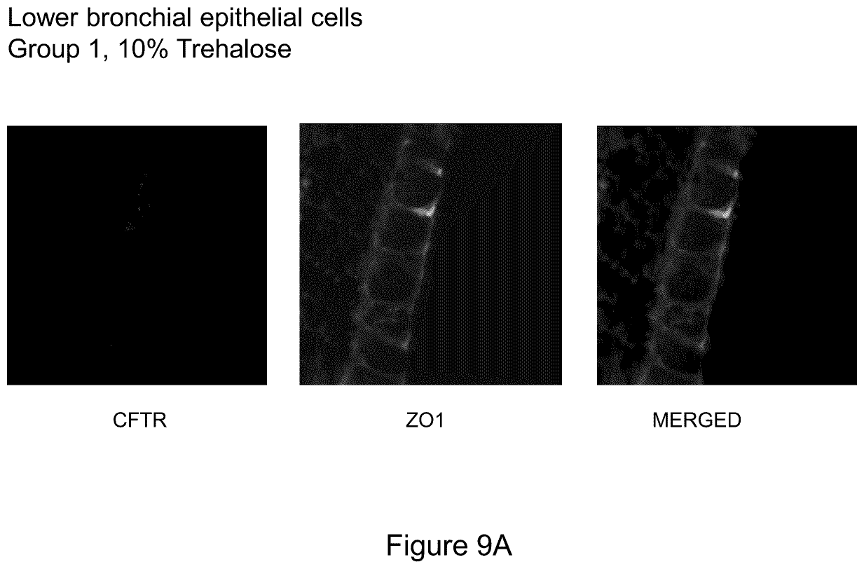

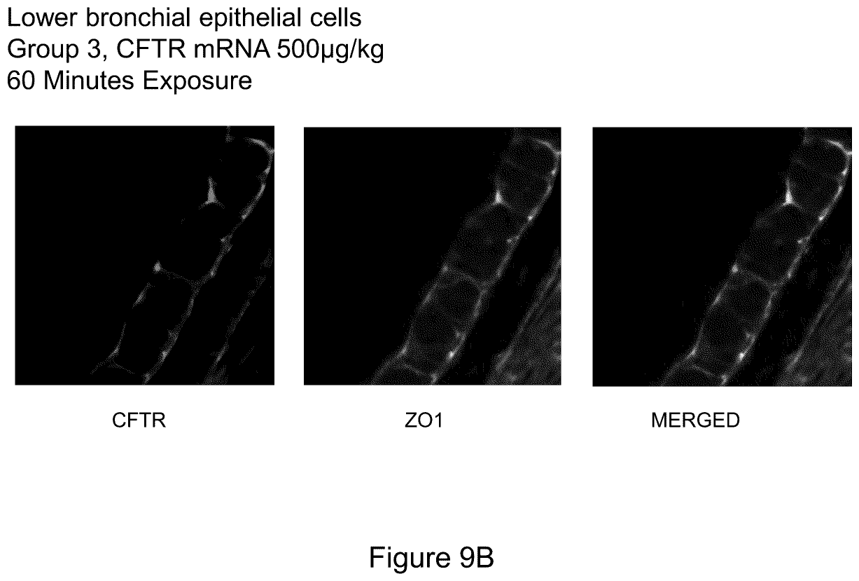

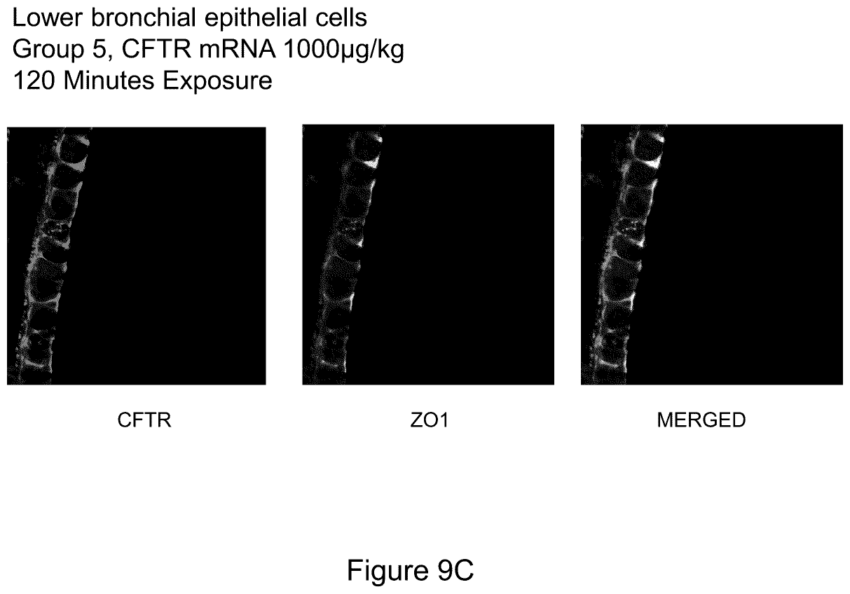

For successful therapeutic effect, the administered CFTR mRNA has to be delivered to the lower lung. As shown in FIGS. 9A, 9B and 9C, CFTR protein translated from administered CFTR mRNA was expressed in lower respiratory tract epithelial cells. CFTR mRNA-derived protein exhibited a dose-dependent expression and colocalization with endogenous membrane tight junction protein ZO1 protein in lower airway epithelial cell membrane. FIG. 9A shows representative microscopic images of a tissue section from the lower airway epithelial cell membrane from a control animal that received 10% trehalose alone, visualized for CFTR staining (left), ZO1 staining (middle) and optical merge of CFTR and ZO1 staining (right). FIG. 9B shows representative microscopic images of a tissue section from the lower airway epithelial cell membrane of an animal that received low dose CFTR mRNA (500 μg/kg), visualized for CFTR staining (left), ZOlstaining (middle) and optical merge of CFTR and ZO1 staining (right). FIG. 9C shows representative microscopic images of a tissue section from the lower airway of an animal that received high dose CFTR mRNA (1000 μg/kg), visualized for CFTR staining (left), ZO1 staining (middle) and optical merge of CFTR and ZO1 staining (right). The intensity of the CFTR expression was higher in the high dose (FIG. 9C, left image) compared to low dose (FIG. 9B, left image). The merged image in FIG. 9C was yellow indicating that the proteins colocalized, whereas FIG. 9A had no signal for CFTR (left) as was expected of the control. (Color is not shown in the Figure).

### Example 9

### CFTR mRNA Derived Protein Expression in Alveolar Lung

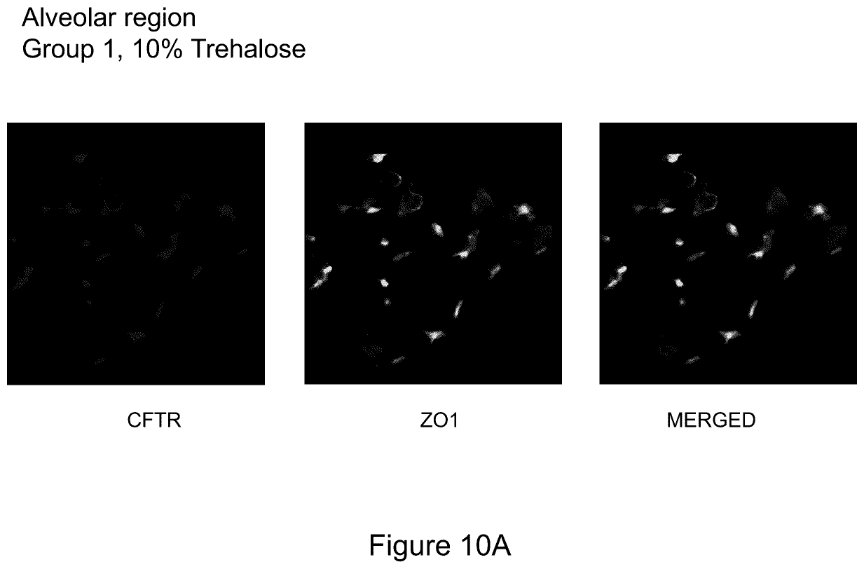

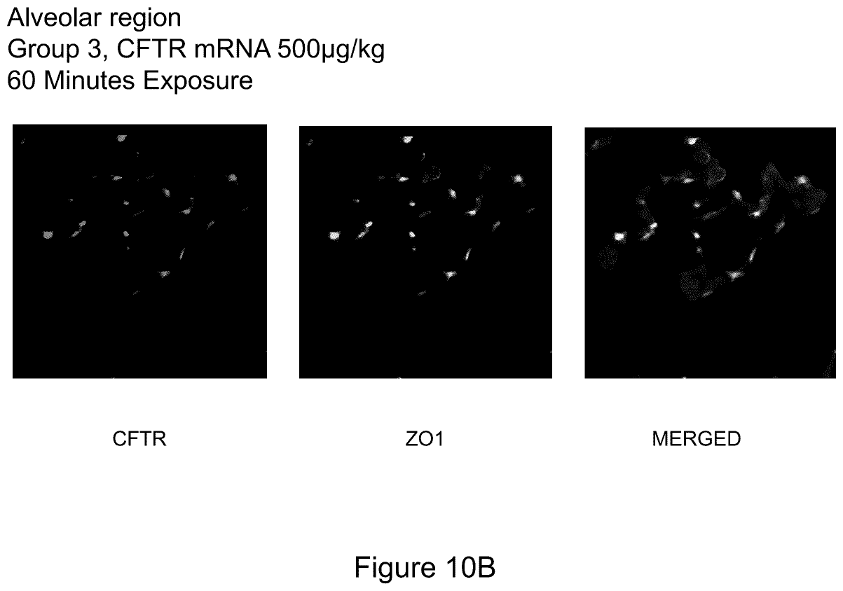

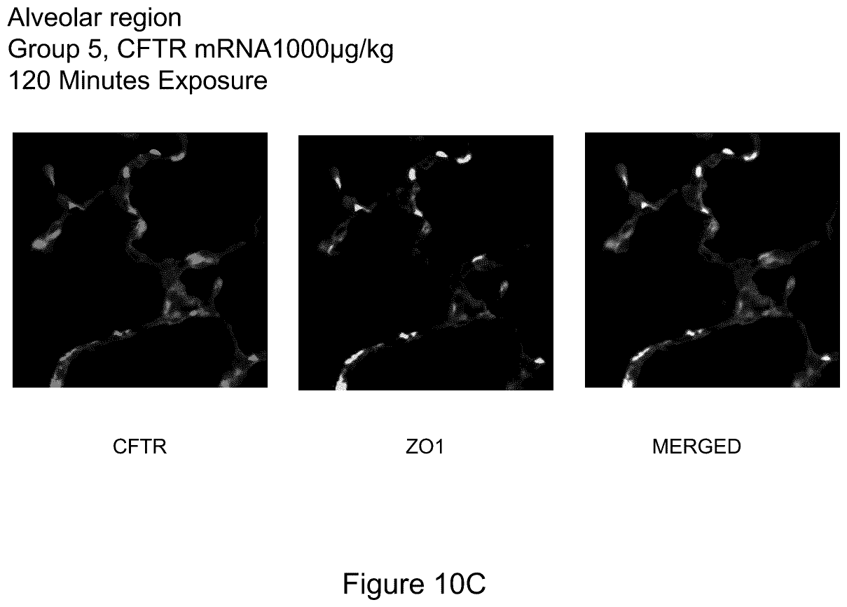

CFTR mRNA was successfully taken up by alveolar cells in the deep lung. As shown in FIGS. 10A, 10B and 10C, CFTR protein translated from administered CFTR mRNA was expressed in alveolar cell membranes and colocalized with endogenous membrane tight junction protein ZO1 protein in the alveolar cell membrane. FIG. 10A shows representative microscopic images of a tissue section from the lower airway epithelial cell membrane from a control animal that received 10% trehalose alone, visualized for CFTR staining (left), ZO1 staining (middle) and optical merge of CFTR and ZO1 staining (right). FIG. 10B shows representative microscopic images of a tissue section from the lower airway epithelial cell membrane of an animal that received low dose CFTR mRNA (500 μg/kg), visualized for CFTR staining (left), ZO1 staining (middle) and optical merge of CFTR and ZO1 staining (right). FIG. 10C shows representative microscopic images of a tissue section from the lower airway of an animal that received high dose CFTR mRNA (1000 μg/kg), visualized for CFTR staining (left), ZO1 staining (middle) and optical merge of CFTR and ZO1 staining (right). The intensity of the CFTR expression was higher in the high dose (FIG. 10C, left image) compared to low dose (FIG. 10B, left image). The merged image in FIGS. 10C was yellow indicating that the proteins colocalized, whereas FIG. 10A had no signal for CFTR (left) as was expected for the control. (Color is not shown in the Figure).

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating cystic fibrosis (CF), comprising a step of:
administering to a subject in need of treatment a composition comprising an mRNA encoding a Cystic Fibrosis Transmembrane Conductance Regulator (CFTR) protein,
wherein the mRNA encoding the CFTR protein comprises a polynucleotide sequence at least 80% identical to SEQ ID NO: 1,
wherein the mRNA is at a concentration of at least 0.4 mg/mL, and wherein the step of administering comprises inhalation.

2.-3. (canceled)

4. The method of claim 1, wherein the mRNA is at a concentration ranging from 0.4 mg/mL to 0.8 mg/mL.

5.-9. (canceled)

10. The method of claim 1, wherein the composition is nebulized prior to inhalation.

11. The method of claim 1, wherein the composition is stored as a frozen, sterile suspension prior to administering.

12. The method of claim 1, wherein the composition is stored in a single-use vial prior to administering.

13. The method of claim 12, wherein the single-use vial comprises less than 5.0 mL of the composition.

14. The method of claim 1, wherein the mRNA encoding the CFIR protein is at a dosage ranging from 8 mg to 24 mg.

15. The method of claim 1, wherein the mRNA encoding the CFTR protein further comprises a 5′ untranslated region (UTR) sequence of SEQ ID NO: 4.

16. The method of claim 1, wherein the mRNA encoding the CFTR protein further comprises a 3′ untranslated region (UTR) sequence of SEQ ID NO: 5 or SEQ ID NO: 6.

17. The method of claim 1, wherein the mRNA encoding the CFTR protein is encapsulated within a nanoparticle.

18. The method of claim 17, wherein the nanoparticle is a liposome.

19. The method of claim 18, wherein the liposome comprises one or more cationic lipids, one or more non-cationic lipids, and one or more PEG-modified lipids.

20.-21. (canceled)

22. The method of claim 18, wherein the liposome has a size less than about 100 nm.

23.-24. (canceled)

25. The method of claim 19, wherein the liposome comprises imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K).

26.-30. (canceled)

31. The method of claim 1, wherein the mRNA encoding the CFTR protein comprises a polynucleotide sequence at least 85%, at least 90% or at least 91% or at least 92% or at least 93% or at least 94% or at least 95% or at least 96% or at least 97% or at least 98% or at least 99% or 100% identical to any one of SEQ ID NO: 6 to SEQ ID NO: 27.

32. The method of claim 1, where the pharmaceutical composition is stored in lyophilized dry powder form.

33. The method of claim 1, where the pharmaceutical composition in lyophilized form is stable in frozen condition for 1, 2, 3, 4, 5 or 10 years without loss of pharmacological or biological activity of the mRNA encoding CFTR protein.

34.-35. (canceled)

36. A pharmaceutical composition comprising a lipid nanoparticle, wherein the lipid nanoparticle comprises mRNA encoding a Cystic Fibrosis Transmembrane Conductance Regulator (CFTR) protein, and one or more lyoprotectants selected from the group consisting of sucrose, trehalose, dextran and inulin.

37. The pharmaceutical composition of claim 36, wherein, the lipid nanoparticle has a size less than about 100 nm.

38. (canceled)

39. The pharmaceutical composition of claim 36, wherein, the lipid nanoparticle comprises C12-200, DOTAP (1,2-diolelyl-3-trimethlammonium propane), DODAP (1,2-diolelyl-3-dimethylammonium propane), DOTMA (1,2-di-O-octadecenyl-3-trimethylammonium propane), DlinDMA, DLin-KC2-DMA, HGT4003, or ICE ((3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-1H-cyclopenta[a]phenanthren-3-yl 3-(1H-imidazol-4-yl)propanoate).

40.-41. (canceled)

42. The pharmaceutical composition of claim 36, wherein, the lipid nanoparticle comprises imidazole cholesterol ester (ICE), 1,2-dioleoyl-sn-glycero-3-phosphoethanolamine (DOPE), and 1,2-dimyristoyl-sn-glycerol, methoxypolyethylene glycol (DMG-PEG-2K).

43.-45. (canceled)

46. A lyophilized formulation comprising the composition of claim 36.

47. A method of treating upper airway bronchial epithelial cells of a primate, comprising a step of administering by inhalation to the primate a nebulized pharmaceutical composition comprising an mRNA encoding a Cystic Fibrosis Transmembrane Conductance Regulator (CFTR) protein encapsulated in a lipid nanoparticle

48. A method of treating lower airway bronchial epithelial cells of a primate, comprising a step of administering by inhalation to the primate a nebulized pharmaceutical composition comprising an mRNA encoding a Cystic Fibrosis Transmembrane Conductance Regulator (CFTR) protein encapsulated in a lipid nanoparticle.

49. A method of treating alveolar cells of a primate, comprising a step of administering by inhalation to the primate a nebulized pharmaceutical composition comprising an mRNA encoding a Cystic Fibrosis Transmembrane Conductance Regulator (CFTR) protein encapsulated in a lipid nanoparticle.
